(19) United States
(12) Patent Application Publication
(10) Pub. No.: US 2024/0417755 A1
(43) Pub. Date: Dec. 19, 2024

(54) FUSION POLYPEPTIDES FOR GENETIC EDITING AND METHODS OF USE THEREOF

(71) Applicant: VOR BIOPHARMA INC., Cambridg, MA (US)

(72) Inventors: Elizabeth Paik, Cambridge, MA (US); Dane Hazelbaker, Cambridge, MA (US); Tirtha Chakraborty, Cambridge, MA (US)

(73) Assignee: VOR BIOPHARMA INC., Cambridge, MA (US)

(21) Appl. No.: 18/695,616

(22) PCT Filed: Sep. 27, 2022
(86) PCT No.: PCT/US2022/077080

§ 371 Date: Mar. 26, 2024

Related U.S. Application Data
(60) Provisional application No. 63/248,968, filed on Sep. 27, 2021.

(51) Int. Cl. C12N 15/90; C12N 9/22; C12N 9/78; C12N 15/11

(52) U.S. Cl. CPC C12N 15/907; C12N 9/22; C12N 9/78; C12N 15/11; C12Y 305/04002; C12Y 305/04005; C12N 2310/20

(57) ABSTRACT

Provided herein are fusion polypeptides comprising a Cpf1 domain lacking nuclease activity and an endonuclease domain. Also provided herein are fusion polypeptides further comprising a genomic modification domain, which in some embodiments is a base editor, such as a deaminase. Also provided herein are methods involving contacting the fusion polypeptides with a gRNA to form a genetic editing system directed to a target site sequence in the genome of a cell.

### RELATED APPLICATIONS

This application claims the benefit under 35 U.S.C. 119(e) of U.S. provisional application No. 63/248,968 filed on Sep. 27, 2021 which is incorporated by reference herein in its entirety.

### REFERENCE TO AN ELECTRONIC SEQUENCE LISTING

The contents of the electronic sequence listing (V029170023WO00-SEQ-CEW; Size: 225,278 bytes; and Date of Creation: Sep. 26, 2022) is herein incorporated by reference in its entirety.

### BACKGROUND

Clustered regulatory Interspaced Short Palindromic Repeats (CRISPR)/Cas systems a provide a platform for targeted gene editing in cells. Despite the versatility of the systems and associated tools for use, there are a number of limitations in these tools for the specific introduction of targeted modifications into the cell genome, for example, for modifying the coding sequence of a gene associated with a disease or disorder.

### SUMMARY

The disclosure is directed, in part, to fusion polypeptides comprising a Cpf1 domain that is catalytically inactive (lacks nuclease activity) and an endonuclease domain (e.g., from a restriction endonuclease, such as FokI) that function in directing single stranded DNA cleavage (i.e., nickase activity) to a target site in the genome of a cell.

Accordingly, in one aspect, the disclosure is directed to a fusion polypeptide comprising a Cpf1 domain that lacks nuclease activity, and an endonuclease domain.

In some embodiments, the endonuclease domain comprises a first DNA-cleavage domain of a restriction endonuclease, wherein the first DNA-cleavage domain is capable of forming a dimer with a second DNA-cleavage domain of a restriction endonuclease. In some embodiments, the endonuclease domain comprises a first DNA-cleavage domain of a restriction endonuclease and a second DNA-cleavage domain of a restriction endonuclease, wherein the first DNA-cleavage domain and second DNA-cleavage domain are capable of forming a dimer with one another. In some embodiments, the dimer of the first and second DNA-cleavage domain is capable of producing a single strand break in DNA.

In some embodiments, the restriction endonuclease is a type IIS restriction endonuclease or portion thereof. In some embodiments, the endonuclease domain comprises FokI or a portion thereof. In some embodiments, the first and/or second DNA-cleavage domain is a DNA cleavage domain of FokI or derived therefrom. In some embodiments, the endonuclease domain does not comprise the DNA binding domain of FokI and/or is not capable of forming and/or maintaining a complex with DNA in the absence of an accompanying Cpf1 domain. In some embodiments, the first DNA-cleavage domain or the second DNA-cleavage domain comprises one or more modifications relative to a corresponding wildtype sequence. In some embodiments, the one or more modifications alter activity of the endonuclease domain such that the endonuclease domain does not produce double strand breaks in DNA. In some embodiments, the one or more modifications decrease or eliminate endonuclease activity of the endonuclease domain. In some embodiments, the endonuclease domain comprises an amino acid sequence of any of SEQ ID NOs: 13 or 14, or a sequence with at least 80, 85, 90, 95, or 99% identity to any thereof.

In some embodiments, the Cpf1 domain comprises an amino acid sequence of a Cpf1 protein from Prevotella spp., Francisella spp., Acidaminococcus sp. (AsCpf1), Lachnospiraceae bacterium (LpCpf1), Eubacterium rectale, or an engineered Cpf1. In some embodiments, the Cpf1 domain comprises one or more amino acid modifications relative to a corresponding wildtype Cpf1 amino acid sequence. In some embodiments, the one or more modifications comprise one or more amino acid substitutions in the Cpf1 protein relative to the wildtype sequence. In some embodiments, the Cpf1 domain comprises a substitution at: one, two, three, or each of amino acids corresponding to positions 174, 542, 548, or 552 of the Acidaminococcus sp. Cpf1 amino acid sequence. In some embodiments, the Cpf1 domain comprises a substitution at: one, two, three, or each of amino acids corresponding to positions 169, 529, 535, or 538 of the MAD7™ Cpf1 amino acid sequence provided by SEQ ID NO: 1. In some embodiments, the one or more substitutions comprise an arginine at the position corresponding to position 174, an arginine at the position corresponding to position 542, a valine at the position corresponding to position 548, and/or an arginine at the position corresponding to position 552 of the Acidaminococcus sp. Cpf1 amino acid sequence provided by SEQ ID NO: 4.

In some embodiments, the one or more substitutions comprise an arginine at the position corresponding to position 169, an arginine at the position corresponding to position 529, a valine at the position corresponding to position 535, and/or an arginine at the position corresponding to position 538 of the MAD7™ Cpf1 amino acid sequence provided by SEQ ID NO: 1.

In some embodiments, the fusion polypeptide further comprises c) a genomic modification domain. In some embodiments, the genomic modification domain comprises a base editor. In some embodiments, the base editor is a cytosine base editor (CBE) or an adenine base editor (ABE). In some embodiments, the base editor comprises a cytidine deaminase or an adenine deaminase. In some embodiments, the base editor comprises both a cytidine deaminase and an adenine deaminase. In some embodiments, the genomic modification domain comprises an epigenetic modifier. In some embodiments, the epigenetic modifier comprises a DNA methyltransferase, a DNA methylase, a histone acetyltransferase, a histone deacetylase, a histone methyltransferase, a histone methylase, or a functional portion or combination of any thereof. In some embodiments, the genomic modification domain comprises an amino acid sequence of SEQ ID NO: 15, or a sequence with at least 80, 85, 90, 95, or 99% identity to any thereof.

In some embodiments, the Cpf1 domain is N-terminal of the endonuclease domain. In some embodiments, the endonuclease domain is N-terminal of the Cpf1 domain. In some embodiments, the genomic modification domain is N-terminal of the Cpf1 domain. In some embodiments, the genomic modification domain is N-terminal of the endonuclease domain. In some embodiments, the fusion comprises from N-terminus to C-terminus: the Cpf1 domain, the endonuclease domain, and the genomic modification domain. In some embodiments, the fusion comprises from N-terminus to C-terminus: the Cpf1 domain, the genomic modification domain, and the endonuclease domain. In some embodiments, the fusion comprises from N-terminus to C-terminus: the endonuclease domain, the Cpf1 domain, and the genomic modification domain. In some embodiments, the fusion comprises from N-terminus to C-terminus: the endonuclease domain, the genomic modification domain, and the Cpf1 domain. In some embodiments, the fusion comprises from N-terminus to C-terminus: the genomic modification domain, the Cpf1 domain, and the endonuclease domain. In some embodiments, the fusion comprises from N-terminus to C-terminus: the genomic modification domain, the endonuclease domain, and the Cpf1 domain.

In some embodiments, the fusion polypeptide further comprises one or more linker domains. In some embodiments, the linker is an XTEN linker.

In another aspect, the disclosure is directed to a nucleic acid comprising a nucleotide sequence encoding a fusion polypeptide described herein.

In another aspect, the disclosure is directed to a vector comprising a nucleic acid described herein.

In another aspect, the disclosure is directed to a cell comprising a fusion polypeptide, the nucleic acid, or vector described herein.

In another aspect, the disclosure is directed to a system comprising: a fusion polypeptide described herein; and a first gRNA comprising a targeting domain complementary to a first target sequence in the genome of a cell, wherein the fusion polypeptide is capable of forming and/or maintaining a ribonucleoprotein (RNP) complex with the first gRNA and the RNP complex is capable of binding the target sequence in the genome of a cell. In some embodiments, the system further comprises a second gRNA comprising a targeting domain complementary to a second target sequence in the genome of the cell, wherein the first and second target sequences are not the same. In some embodiments, the system further comprises a second fusion polypeptide comprising a) a Cpf1 domain that lacks nuclease activity, and b) a second endonuclease domain capable of forming a dimer with the first endonuclease domain.

In another aspect, the disclosure is directed to a ribonucleoprotein (RNP) complex comprising: a fusion polypeptide described herein; and a gRNA comprising a targeting domain complementary to a target sequence in the genome of a cell, wherein RNP complex is capable of binding the target sequence in the genome of a cell.

In another aspect, the disclosure is directed to a method comprising: i) contacting a cell with a fusion polypeptide or nucleic acid described herein; and ii) contacting the cell with a first gRNA comprising a targeting domain complementary to a first target sequence in the genome of a cell. In some embodiments, i) and ii) occur simultaneously or in close temporal proximity. In some embodiments, the method further comprises: iii) contacting the cell with a second gRNA (or nucleic acid encoding the same) comprising a targeting domain complementary to a second target sequence in the genome of a cell. In some embodiments, the method further comprises contacting the cell with a second fusion protein or nucleic acid described herein.

In another aspect, the disclosure is directed to a method, comprising: i) contacting a cell with a first fusion polypeptide described herein and a first gRNA comprising a targeting domain complementary to a first target sequence in the genome of a cell; and ii) contacting the cell with a second fusion polypeptide described herein and a second gRNA comprising a targeting domain complementary to a second target sequence in the genome of a cell, wherein the first target sequence and the second target sequence are not the same and the first fusion polypeptide and second fusion polypeptide are not the same.

In some embodiments, the first target sequence and the second target sequence are on different chromosomes of the genome of the cell. In some embodiments, the first target sequence and the second target sequence are on the same chromosome in the genome of the cell. In some embodiments, the first target sequence and the second target sequence are on the same DNA strand of the chromosome. In some embodiments, the first target sequence and the second target sequence are on different DNA strands of the chromosome. In some embodiments, the first target sequence and the second target sequence are separated by 10-10,000 nucleotides.

In some embodiments, the cell is a hematopoietic cell. In some embodiments, the cell is a hematopoietic stem cell. In some embodiments, the cell is a hematopoietic progenitor cell. In some embodiments, the cell is an immune effector cell. In some embodiments, the cell is a lymphocyte. In some embodiments, the cell is a T-lymphocyte.

In another aspect, the disclosure is directed to an engineered cell, or descendant thereof, produced by a method described herein.

In another aspect, the disclosure is directed to a cell population, comprising an engineered cell described herein.

In another aspect, the disclosure is directed to a chimeric polypeptide that lacks nuclease activity, comprising: a first portion comprising an amino acid sequence of a first Cpf1 protein, and a second portion comprising an amino acid sequence of a second Cpf1 protein, wherein the first Cpf1 protein and second Cpf1 protein are not the same. In some embodiments, the first Cpf1 protein is derived from a Cpf1 from Prevotella spp. or Francisella spp., Acidaminococcus sp. (AsCpf1), Lachnospiraceae bacterium (LpCpf1), or Eubacterium rectale, or MAD7™ as provided by Inscripta. In some embodiments, the second Cpf1 protein is derived from a Cpf1 from Prevotella spp. or Francisella spp., Acidaminococcus sp. (AsCpf1), Lachnospiraceae bacterium (LpCpf1), or Eubacterium rectale, or MAD7™ as provided by Inscripta. In some embodiments, the first Cpf1 protein comprises an Acidaminococcus sp. Cpf1 (AsCpf1) or portion thereof. In some embodiments, the second Cpf1 protein comprises MAD7™ or a portion thereof.

In some embodiments, the first Cpf1 protein and/or second Cpf1 protein comprise one or more modifications relative to the wildtype sequence of the first Cpf1 protein and/or second Cpf1 protein. In some embodiments, the one or more modifications comprise one or more amino acid substitutions in the first Cpf1 protein relative to the wildtype sequence of the first Cpf1 protein.

In some embodiments, the amino acid sequence comprising the first Cpf1 protein is at least 100 amino acids in length, or 100-1300 amino acids in length. In some embodiments, the amino acid sequence comprising the second Cpf1 protein is at least 100 amino acids in length, or 100-1300 amino acids in length. In some embodiments, the chimeric polypeptide further comprises a linker between the first portion and second portion. In some embodiments, the chimeric polypeptide is at least 800 amino acids in length, or 800-1500 amino acids in length.

In some embodiments, the amino acid sequence of the first Cpf1 protein comprises any of SEQ ID NOs: 1-9 or a sequence with at least 80, 85, 90, 95, or 99% identity to any thereof. In some embodiments, the amino acid sequence of the second Cpf1 protein comprises any of SEQ ID NOs: 1-9 or a sequence with at least 80, 85, 90, 95, or 99% identity to any thereof. In some embodiments, the chimeric polypeptide comprises an amino acid sequence of any of SEQ ID NOs: 24-31 or a sequence with at least 80, 85, 90, 95, or 99% identity to any thereof.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF THE DRAWINGS

FIG. 1 shows a schematic of an exemplary plasmid vector encoding an enhanced Cpf1 nuclease (enCpf1, enAsCpf1-(RVR)). The vector encodes a gRNA scaffold under the control of the U6 promoter. The enhanced Cpf1 is a Cpf1 nuclease from Acidaminococcus sp. BV3L6 containing the E174R/S542R/K548V/N552R mutations (referred to as “enAsCpf1-(RVR)”) under the control of the chicken beta-actin promoter and cytomegalovirus (CMV) enhancer sequence.

FIG. 2 shows a schematic of an exemplary plasmid vector encoding a fusion of a base editor and an enhanced Cpf1 nuclease. The vector encodes a gRNA scaffold under the control of the U6 promoter. The base editor-Cpf1 fusion is a fusion of the exemplary base editor APOBEC-1 fused to the N-terminus of the enhanced Cpf1 nuclease from Acidaminococcus sp. BV3L6 containing the E174R/S542R/K548V/N552R mutations under the control of the chicken beta-actin promoter and CMV enhancer sequence.

FIG. 3 shows a schematic of an exemplary plasmid vector encoding the MAD7^{T}M nuclease. The vector encodes a gRNA scaffold under the control of the U6 promoter. The gene encoding MAD7™ (Inscripta) is under the control of the chicken beta-actin promoter and CMV enhancer sequence.

FIG. 4 shows a schematic of an exemplary plasmid vector encoding an enhanced nuclease based on the MAD7™ nuclease. The vector encodes a gRNA scaffold under the control of the U6 promoter. The enhanced nuclease based on the MAD7™ nuclease contains the mutations K169R, D529F, K535V, and N538R under the control of the chicken beta-actin promoter and CMV enhancer sequence.

FIG. 5 shows a schematic of an exemplary plasmid vector encoding a fusion polypeptide comprising a Cpf1 domain lacking nuclease activity (dCpf1) and two FokI nuclease domains (Fok1 domain I and Fok1 domain II The vector encodes a gRNA scaffold under the control of the U6 promoter. The fusion polypeptide is a fusion of the two Fok1 nuclease domains to the C-terminus of a nuclease-dead Cpf1 enzyme from Acidaminococcus sp. BV3L6 containing a D908A mutation under control of the chicken beta actin promoter and CMV enhancer sequence. The FokI domains are separated from each other with a polypeptide linker and from dCpf1 with an XTEN linker.

FIG. 6 shows a schematic of an exemplary plasmid vector encoding a fusion polypeptide comprising a Cpf1 domain lacking nuclease activity (dCpf1) and two FokI nuclease domains (Fok1 domain I and Fok1 domain II). The vector encodes a gRNA scaffold under the control of the U6 promoter. The fusion polypeptide is a fusion of the two FokI nuclease domains to the N-terminus of a nuclease-dead Cpf1 enzyme from Acidaminococcus sp. BV3L6 containing a D908A mutation under control of the chicken beta-actin promoter and CMV enhancer sequence. The Fok1 domains are separated from each other with a polypeptide linker and from the dCpf1 with an XTEN linker.

FIG. 7 shows a schematic of an exemplary plasmid vector encoding a fusion polypeptide comprising a Cpf1 domain lacking nuclease activity (dCpf1) and two FokI nuclease domains (Fok1 domain 1 and Fok1 domain), wherein the Fok1 domain 1 contains a D450A mutation abrogating its nuclease activity. The vector encodes a gRNA scaffold under the control of the U6 promoter. The fusion polypeptide is a fusion of the two FokI nuclease domains to the C-terminus a nuclease-dead Cpf1 enzyme from Acidaminococcus sp. BV3L6 containing a D908A mutation under control of the chicken beta-actin promoter and CMV enhancer sequence. The Fok1 domains are separated from each other with a polypeptide linker and from the dCpf1 with an XTEN linker.

FIG. 8 shows a schematic of the exemplary plasmid vector encoding a fusion polypeptide comprising a Cpf1 domain lacking nuclease activity (dCpf1) and two FokI nuclease domains (Fok1 domain I and FokI domain II), wherein the FokI domain II contains a D450A mutation abrogating its nuclease activity. Fok1 domain I contains the wildtype D450 residue and has functional nuclease activity. The vector encodes a gRNA scaffold under the control of the U6 promoter. The fusion polypeptide is a fusion of the two Fok1 nuclease domains to the C-terminus of a nuclease-dead Cpf1 enzyme from Acidaminococcus sp. BV3L6 containing a D908A mutation under control of the chicken beta-actin promoter and CMV enhancer sequence. The Fok1 domains are separated from each other with a polypeptide linker and from the dCpf1 with an XTEN linker.

FIG. 9 shows a schematic of an exemplary plasmid vector encoding a fusion polypeptide comprising a Cpf1 domain lacking nuclease activity (dCpf1) and two FokI nuclease domains (Fok1 domain I and Fok1 domain II), wherein FokI domain I contains a D450A mutation abrogating its nuclease activity. Fok1 domain II contains the wildtype D450 residue and has functional nuclease activity. The vector encodes a gRNA scaffold under the control of the U6 promoter. The fusion polypeptide is a fusion of the two FokI nuclease domains to the N-terminus of a nuclease dead dCpf1 enzyme from Acidaminococcus sp. BV3L6 (AsCpf1) containing a D908A mutation under control of the chicken beta-actin promoter and a CMV enhancer sequence. The Fok1 domains are separated from each other with a polypeptide linker and from the dCpf1 with an XTEN linker.

FIG. 10 shows a schematic of an exemplary plasmid vector encoding a fusion polypeptide comprising Cpf1 domain lacking nuclease activity (dCpf1) and two FokI nuclease domains (FokI domain I and FokI domain II), wherein the FokI domain II has a D450A mutation abrogating its nuclease activity. The FokI domain I contains the wildtype D450 residue and has functional nuclease activity. The vector encodes a gRNA scaffold under the control of the U6 promoter. The fusion polypeptide is a fusion of the two FokI nuclease domains to the N-terminus of a nuclease dead (dCpf1) enzyme from Acidaminococcus sp. BV3L6 (AsCpf1) containing a D908A mutation under control of the chicken beta-actin promoter and a CMV enhancer sequence. The Fok1 domains are separated from each other with a polypeptide linker and from the dCpf1 with an XTEN linker.

FIG. 11 shows a schematic of an exemplary plasmid vector encoding a fusion polypeptide comprising base editing domain, a Cpf1 domain lacking nuclease activity (dCpf1) and two FokI nuclease domains (FokI domain I and Fok1 domain II), wherein FokI domain I has a D450A mutation abrogating its nuclease activity. The FokI domain II contains the wildtype D450 residue and has functional nuclease activity. The vector encodes a gRNA scaffold under the control of the U6 promoter. The fusion polypeptide is a fusion of the exemplary base editor APOBEC-1 to the N-terminus of the nuclease dead (dCpf1) enzyme from Acidaminococcus sp. BV3L6 (AsCpf1) containing a D908A mutation with the C-terminus of the dCpf1 fused to the two FokI nuclease domains under the control of the chicken beta-actin promoter and a CMV enhancer sequence. The Fok1 domains are separated from each other with a polypeptide linker and from the dCpf1 with an XTEN linker.

FIG. 12 shows a schematic of the exemplary plasmid vector encoding a base editing domain, an enhanced nuclease based on MAD7™ nuclease, and two FokI nuclease domains (FokI domain I and FokI domain II), wherein the FokI domain I has a D450A mutation abrogating its nuclease activity. The FokI domain II contains the wildtype D450 residue and has functional nuclease activity. The vector encodes a gRNA scaffold under the control of the U6 promoter. The fusion polypeptide is a fusion of the exemplary base editor APOBEC-1 to the N-terminus of an enhanced catalytically dead nuclease based on MAD7™ containing mutations K169R, D529F, K535V, N538R, and D877A fused to the two FokI nuclease domains under the control of the chicken beta-actin promoter and a CMV enhancer sequence. The Fok1 domains are separated from each other with a polypeptide linker and from the dCpf1 with an XTEN linker.

FIG. 13 shows a schematic of an exemplary fusion polypeptide described herein, wherein from N-terminus to C-terminus, the polypeptide comprises a base editor domain (e.g., TadA2.1), an endonuclease domain (e.g., FokI nuclease domain), and a Cas domain lacking nuclease activity (dCas, e.g., Casϕ/Cas12j).

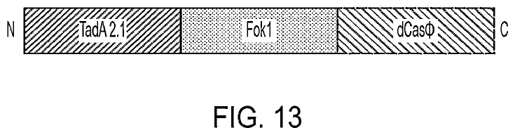

### DETAILED DESCRIPTION

Aspects of the present disclosure provide fusion polypeptides comprising a Cpf1 domain that is catalytically inactive (lacks nuclease activity) and an endonuclease domain (e.g., from a restriction endonuclease, such as FokI) that function in directing single stranded DNA cleavage (i.e., nickase activity) to a target site in the genome of a cell. In some embodiments, the fusion polypeptides further comprise a genomic modification domain, such as a base editor domain (e.g., a deaminase activity) that targets and deaminates a nucleobase, e.g., a cytosine or adenosine nucleobase of a C or A nucleotide, at the target site, which via cellular mismatch repair mechanisms, results in a modification, such as a change in the nucleobase from a C to a T nucleotide, or a change from an A to a G nucleotide.

Targeting of endonucleases to desired genomic target sites using transcription activator-like effector nucleases (TALENs) or zinc finger domains has been performed, and in the case of zinc finger nucleases (ZFNs), has been utilized to carry out genetic mutations (Ramirez, et al, Nucleic Acids Research (2012) 40 (12): 5560-68; Sun et al., Mol. BioSyst. (2014) 10: 446). However, generation of such constructs is laborious, may be cumbersome due to their large size (in the case of TALENS) and less efficient than genetic editing using CRISPR/Cas systems.

Precise genetic editing has been achieved, for example using base editors based primarily on a catalytically impaired Cas9 nuclease in which one of the nuclease domains of Cas9 is mutated such that the nuclease generates a single-strand DNA break. However, use of non-Cas9 nucleases, such as Cas12a/Cpf1 nucleases for such genomic targeting has been much more limited. Without wishing to be bound by any particular theory, it is thought that in contrast to the two separate nuclease domains of Cas9, Cas12a/Cpf1 does not have separate active sites for cleaving each DNA strand, making nickase variants of Cas12a/Cpf1 more challenging. See, e.g., Richter et al. Nat. Biotechnol. (2020) 38(7): 883-891.

### Fusion Polypeptides

Aspects of the present invention provide fusion polypeptides comprising a Cpf1/Cas12a domain without nuclease activity and an endonuclease domain, including systems and methods for using such fusion polypeptides for introducing targeted mutations into the genome of a target cell. The term “mutation,” as used herein, refers to a change (e.g., an insertion, deletion, inversion, or substitution) in a nucleic acid sequence as compared to a reference sequence, e.g., the corresponding sequence of a cell not having such a mutation, or the corresponding wild-type nucleic acid sequence.

In some embodiments, the cells produced using the fusion polypeptides described herein comprise more than one mutation (e.g., 2, 3, 4, 5, or more) mutations compared to a reference sequence, e.g., the corresponding sequence of a cell not having such a mutation, or the corresponding wild-type nucleic acid sequence. In some embodiments, a mutation to a gene (e.g., a target gene) results in a loss of expression of a protein encoded by the target gene in a cell harboring the mutation. In some embodiments, a mutation in a gene (e.g., a target gene) results in the expression of a variant form of a protein that is encoded by the target gene.

In some embodiments provided herein, the fusion polypeptides effect a mutation in a gene (e.g., a target gene) that results in a loss of expression of a protein encoded by the target gene in a cell harboring the mutation. In some embodiments, the fusion polypeptides effect a mutation in a gene (e.g., a target gene) results in the expression of a variant form of a protein that is encoded by the target gene. In some embodiments, a genetically engineered cell described herein is generated by using any of the fusion polypeptides described herein, for example under conditions suitable for the fusion polypeptide to be directed to target site in the genome of a cell (e.g., by a guide RNA (gRNA) described elsewhere herein) and for the endonuclease domain to cleave a phosphodiester bond in the DNA of the cell.

In some embodiments, the fusion polypeptides described herein generate genetically engineered cells via genome editing technology capable of introducing targeted changes, also referred to as “edits,” into the genome of a cell. In some embodiments, the genetically engineered cells comprise a plurality of edits in the genome of the cells.

The fusion polypeptides described herein comprise a Cpf1/Cas12a domain without nuclease activity and an endonuclease domain, and in some embodiments, may further comprises a genomic modification domain. In some embodiments, the fusion polypeptides comprise one or more linker domains, for example to join any of the domains of the polypeptide.

### Cpf1 Domain

In some aspects, the present disclosure provides a CRISPR-Cas-based system for targeting a fusion polypeptide comprising a Cpf1 domain lacking nuclease activity and an endonuclease domain to a genomic locus in a cell. As used herein, a “Cpf1 domain” refers to Cpf1 nuclease (also referred to as a Cas12 nuclease or Cas12a nuclease) or portion thereof or variant thereof. Cpf1 is considered to belong to the class 2 type V-A Cas nucleases. See, e.g., Strohkendl et al. Mol. Cell (2018) 71: 1-9. The Cas12/Cpf1 nucleases for use in the fusion polypeptides described herein refer to a polypeptide i) derived from a type II class 2 CRISPR/Cas nuclease that cleaves distal to a PAM site, and ii) capable of, in combination with a suitable gRNA, binding a target nucleic acid sequence (a target sequence).

In contrast to Cas9 nucleases, Cpf1 nucleases are directed to a target site requiring one gRNA molecule, a the CRISPR RNA (crRNA), rather than both a crRNA and tracrRNA sequence, and functions using a dual RuvC-Nuc domain (RuvC endonuclease and Nuc nuclease domain), whereas Cas9 has two nuclease domains (RuvC-Nuc and HNH). See, e.g., Gao et al. Cell Res. (2016) 26(8): 901-913.

In some embodiments, the Cpf1 domain is a portion of a Cpf1 enzyme comprising at least 10%, 20%, 30%, 40%, 50%, 60%, 70%, 80%, 90%, or more of the Cpf1 enzyme. In some embodiments, the Cpf1 domain is one or more domains of a Cpf1 enzyme.

Exemplary suitable Cpf1 nucleases include, without limitation, AsCas12a, FnCas12a, LbCas12a, PaCas12a, other Cpf1 orthologs, and Cas12a derivatives, such as the MAD7 system (MAD7™, Inscripta, Inc.), or the Alt-R Cas12a (Cpf1) Ultra nuclease (Alt-R® Cas12a Ultra; Integrated DNA Technologies, Inc.). See, e.g., Gill et al. LIPSCOMB 2017. In United States: Inscripta Inc.; Price et al. Biotechnol. Bioeng. (2020) 117(60): 1805-1816; PCT Publication Nos. WO 2016/166340; WO 2017/155407; WO 2018/083128; WO 2016/205711; WO 2017/035388; WO 2017/184768; WO2019/118516; WO2017/184768; WO 2018/098383; WO 2020/146297; and WO 2020/172502. In some embodiments, the Cpf1 domain is from Cas12a/Cpf1 obtained from Acidaminococcus sp. (referred to as “AsCas12a” or “AsCpf1”), such as Acidaminococcus sp. strain BV3L6.

Additional examples of Cas12 nucleases for use in the fusion polypeptides described herein include, without limitation, Cas12g, Cas12c, Cas12d, Cas12e, Cas12i, Cas12h, Casϕ/Cas12j and Cas12b.

Various Cas12/Cpf1 nucleases are known in the art and may be obtained from various sources and/or engineered/modified to modulate one or more activities or specificities of the enzymes. For example, the PAM sequence preferences and specificities of a Cas12/Cpf1 nucleases may be modified. In some embodiments, the Cas12/Cpf1 nuclease has been engineered/modified to recognize one or more PAM sequence. In some embodiments, the Cas12/Cpf1 nuclease has been engineered/modified to recognize one or more PAM sequence that is different than the PAM sequence the Cas12/Cpf1 nuclease recognizes without engineering/modification. In some embodiments, the Cas12/Cpf1 nuclease has been engineered/modified to reduce off-target activity of the enzyme.

In some embodiments, the Cpf1 domain comprises an amino acid sequence of, or is derived from, a Cpf1 protein from Prevotella spp., Francisella spp., Acidaminococcus sp. (AsCpf1), Lachnospiraceae bacterium (LpCpf1), Eubacterium rectale, or an engineered Cpf1. In some embodiments, the engineered Cpf1 is the MAD7 system (MAD7™, Inscripta, Inc.). Amino acid sequences of exemplary Cas12/Cpf1 nucleases are provided below.

Amino acid sequence of MAD7™ (Inscripta)
(SEQ ID NO: 1)
MNNGTNNFQNFIGISSLQKTLRNALIPTETTQQFIVKNGIIKEDELRGEN
RQILKDIMDDYYRGFISETLSSIDDIDWTSLFEKMEIQLKNGDNKDTLIK
EQTEYRKAIHKKFANDDRFKNMFSAKLISDILPEFVIHNNNYSASEKEEK
TQVIKLESRFATSFKDYFKNRANCESADDISSSSCHRIVNDNAEIFFSNA
LVYRRIVKSLSNDDINKISGDMKDSLKEMSLEEIYSYEKYGEFITQEGIS
FYNDICGKVNSFMNLYCQKNKENKNLYKLOKLHKQILCIADTSYEVPYKF
ESDEEVYQSVNGELDNISSKHIVERLRKIGDNYNGYNLDKIYIVSKFYES
VSQKTYRDWETINTALEIHYNNILPGNGKSKADKVKKAVKNDLOKSITEI
NELVSNYKLCSDDNIKAETYIHEISHILNNFEAQELKYNPEIHLVESELK
ASELKNVLDVIMNAFHWCSVFEMTEELVDKDNNFYAELEEIYDEIYPVIS
LYNLVRNYVTQKPYSTKKIKLNFGIPTLADGWSKSKEYSNNAIILMRDNL
YYLGIFNAKNKPDKKIIEGNTSENKGDYKKMIYNLLPGPNKMIPKVFLSS
KTGVETYKPSAYILEGYKONKHIKSSKDFDITFCHDLIDYFKNCIAIHPE
WKNFGFDFSDTSTYEDISGFYREVELQGYKIDWTYISEKDIDLLQEKGQL
YLFQIYNKDFSKKSTGNDNLHTMYLKNLFSEENLKDIVLKLNGEAEIFFR
KSSIKNPIIHKKGSILVNRTYEAEEKDQFGNIQIVRKNIPENIYQELYKY
FNDKSDKELSDEAAKLKNVVGHHEAATNIVKDYRYTYDKYFLHMPITINF
KANKTGFINDRILQYIAKEKDLHVIGIDRGERNLIYVSVIDTCGNIVEQK
SFNIVNGYDYQIKLKQQEGARQIARKEWKEIGKIKEIKEGYLSLVIHEIS
KMVIKYNAIIAMEDLSYGFKKGRFKVERQVYQKFETMLINKLNYLVFKDI
SITENGGLLKGYQLTYIPDKLKNVGHQCGCIFYVPAAYTSKIDPTTGFVN
IFKFKDLTVDAKREFIKKFDSIRYDSEKNLFCFTFDYNNFITQNTVMSKS
SWSVYTYGVRIKRRFVNGRFSNESDTIDITKDMEKTLEMTDINWRDGHDL
RQDIIDYEIVQHIFEIFRLTVQMRNSLSELEDRDYDRLISPVLNENNIFY
DSAKAGDALPKDADANGAYCIALKGLYEIKQITENWKEDGKFSRDKLKIS
NKDWFDFIQNKRYL

Residues K169, D529, K535, N538, and D877 are indicated in boldface and underlined. Variants of the MAD7™ sequence as provided above, or any suitable sequence of MAD™ known in the art (e.g., the sequence above without the N-terminal methionine, e.g., in the context of a fusion protein), are also embraced by the present disclosure. Such sequences include, for example, an MAD7™ sequence comprising an amino acid substitution at residue K169, D529, K535, N538, or D877, or two or more substitutions at any combination of these residues. In some embodiments, the MAD7™ sequence comprises an amino substitution at residue K169. In some embodiments, the amino acid substitution at residue K169 is a K169R substitution. In some embodiments, the MAD7™ sequence comprises an amino substitution at residue D529. In some embodiments, the amino acid substitution at residue D529 is a D529R substitution. In some embodiments, the MAD7™ sequence comprises an amino substitution at residue K535. In some embodiments, the amino acid substitution at residue K535 is a K535V substitution. In some embodiments, the MAD7™ sequence comprises an amino substitution at residue N538. In some embodiments, the amino acid substitution at residue N538 is a N538R substitution. In some embodiments, the MAD7™ sequence comprises an amino substitution at residue D877. In some embodiments, the amino acid substitution at residue D877 is a D877A substitution.

In some embodiments, the Cpf1 domain comprises an amino acid sequence of SEQ ID NO: 1 that is lacking the N-terminal methionine, e.g., in the context of a fusion protein), are also embraced by the present disclosure. In some embodiments, the Cpf1 domain comprises an amino acid sequence of SEQ ID NO: 1 comprising an amino acid substitution at residue K169, D529, K535, N538, or D877, or two or more substitutions at any combination of these residues. In some embodiments, the Cpf1 domain comprises an amino acid sequence of SEQ ID NO: 1 and comprises an amino substitution at residue K169. In some embodiments, the amino acid substitution at residue K169 is a K169R substitution. In some embodiments, the Cpf1 domain comprises an amino acid sequence of SEQ ID NO: 1 and comprises an amino substitution at residue D529. In some embodiments, the amino acid substitution at residue D529 is a D529R substitution. In some embodiments, the Cpf1 domain comprises an amino acid sequence of SEQ ID NO: 1 and comprises an amino substitution at residue K535. In some embodiments, the amino acid substitution at residue K535 is a K535V substitution. the Cpf1 domain comprises an amino acid sequence of SEQ ID NO: 1 and comprises an amino substitution at residue N538. In some embodiments, the amino acid substitution at residue N538 is a N538R substitution. In some embodiments, the Cpf1 domain comprises an amino acid sequence of SEQ ID NO: 1 and comprises an amino substitution at residue D877. In some embodiments, the amino acid substitution at residue D877 is a D877A substitution.

Amino acid sequence of MAD7™ (Inscripta) contain-
ing K169R, D529R, K535V, and N538R mutations
(SEQ ID NO: 2)
MNNGTNNFQNFIGISSLQKTLRNALIPTETTQQFIVKNGIIKEDELRGEN
RQILKDIMDDYYRGFISETLSSIDDIDWTSLFEKMEIQLKNGDNKDTLIK
EQTEYRKAIHKKFANDDRFKNMFSAKLISDILPEFVIHNNNYSASEKEEK
TQVIKLFSRFATSFKDYFRNRANCESADDISSSSCHRIVNDNAEIFFSNA
LVYRRIVKSLSNDDINKISGDMKDSLKEMSLEEIYSYEKYGEFITQEGIS
FYNDICGKVNSFMNLYCQKNKENKNLYKLQKLHKQILCIADTSYEVPYKF
ESDEEVYQSVNGELDNISSKHIVERLRKIGDNYNGYNLDKIYIVSKFYES
VSQKTYRDWETINTALEIHYNNILPGNGKSKADKVKKAVKNDLQKSITEI
NELVSNYKLCSDDNIKAETYIHEISHILNNFEAQELKYNPEIHLVESELK
ASELKNVLDVIMNAFHWCSVEMTEELVDKDNNFYAELEEIYDEIYPVISL
YNLVRNYVTQKPYSTKKIKLNFGIPTLARGWSKSVEYSRNAIILMRDNLY
YLGIFNAKNKPDKKIIEGNTSENKGDYKKMIYNLLPGPNKMIPKVFLSSK
TGVETYKPSAYILEGYKQNKHIKSSKDEDITFCHDLIDYFKNCIAIHPEW
KNFGFDFSDTSTYEDISGFYREVELQGYKIDWTYISEKDIDLLQEKGQLY
LFQIYNKDFSKKSTGNDNLHTMYLKNLFSEENLKDIVLKLNGEAEIFFRK
SSIKNPIIHKKGSILVNRTYEAEEKDQFGNIQIVRKNIPENIYQELYKYF
NDKSDKELSDEAAKLKNVVGHHEAATNIVKDYRYTYDKYFLHMPITINFK
ANKTGFINDRILQYIAKEKDLHVIGIDRGERNLIYVSVIDTCGNIVEQKS
FNIVNGYDYQIKLKQQEGARQIARKEWKEIGKIKEIKEGYLSLVIHEISK
MVIKYNAIIAMEDLSYGFKKGRFKVERQVYQKFETMLINKLNYLVFKDIS
ITENGGLLKGYQLTYIPDKLKNVGHQCGCIFYVPAAYTSKIDPTTGFVNI
FKFKDLTVDAKREFIKKFDSIRYDSEKNLFCFTFDYNNFITQNTVMSKSS
WSVYTYGVRIKRRFVNGRFSNESDTIDITKDMEKTLEMTDINWRDGHDLR
QDIIDYEIVQHIFEIFRLTVQMRNSLSELEDRDYDRLISPVLNENNIFYD
SAKAGDALPKDADANGAYCIALKGLYEIKQITENWKEDGKFSRDKLKISN
KDWFDFIQNKRYL
Amino acid sequence of MAD7™ (Inscripta) contain-
ing K169R, D529R, K535V, N538R, and D877A mutations
(SEQ ID NO: 3)
MNNGTNNFQNFIGISSLQKTLRNALIPTETTQQFIVKNGIIKEDELRGEN
RQILKDIMDDYYRGFISETLSSIDDIDWTSLFEKMEIQLKNGDNKDTLIK
EQTEYRKAIHKKFANDDRFKNMFSAKLISDILPEFVIHNNNYSASEKEEK
TQVIKLESRFATSFKDYFRNRANCESADDISSSSCHRIVNDNAEIFFSNA
LVYRRIVKSLSNDDINKISGDMKDSLKEMSLEEIYSYEKYGEFITQEGIS
FYNDICGKVNSFMNLYCQKNKENKNLYKLQKLHKQILCIADTSYEVPYKF
ESDEEVYQSVNGELDNISSKHIVERLRKIGDNYNGYNLDKIYIVSKFYES
VSQKTYRDWETINTALEIHYNNILPGNGKSKADKVKKAVKNDLQKSITEI
NELVSNYKLCSDDNIKAETYIHEISHILNNFEAQELKYNPEIHLVESELK
ASELKNVLDVIMNAFHWCSVFMTEELVDKDNNFYAELEEIYDEIYPVISL
YNLVRNYVTQKPYSTKKIKLNFGIPTLARGWSKSVEYSRNAIILMRDNLY
YLGIFNAKNKPDKKIIEGNTSENKGDYKKMIYNLLPGPNKMIPKVFLSSK
TGVETYKPSAYILEGYKQNKHIKSSKDFDITFCHDLIDYFKNCIAIHPEW
KNFGFDFSDTSTYEDISGFYREVELQGYKIDWTYISEKDIDLLQEKGQLY
LFQIYNKDFSKKSTGNDNLHTMYLKNLFSEENLKDIVLKLNGEAEIFFRK
SSIKNPIIHKKGSILVNRTYEAEEKDQFGNIQIVRKNIPENIYQELYKYF
NDKSDKELSDEAAKLKNVVGHHEAATNIVKDYRYTYDKYFLHMPITINFK
ANKTGFINDRILQYIAKEKDLHVIGIARGERNLIYVSVIDTCGNIVEQKS
FNIVNGYDYQIKLKQQEGARQIARKEWKEIGKIKEIKEGYLSLVIHEISK
MVIKYNAIIAMEDLSYGFKKGRFKVERQVYQKFETMLINKLNYLVFKDIS
ITENGGLLKGYQLTYIPDKLKNVGHQCGCIFYVPAAYTSKIDPTTGFVNI
FKFKDLTVDAKREFIKKFDSIRYDSEKNLFCFTEDYNNFITQNTVMSKSS
WSVYTYGVRIKRRFVNGRFSNESDTIDITKDMEKTLEMTDINWRDGHDLR
QDIIDYEIVQHIFEIFRLTVQMRNSLSELEDRDYDRLISPVLNENNIFYD
SAKAGDALPKDADANGAYCIALKGLYEIKQITENWKEDGKFSRDKLKISN
KDWFDFIQNKRYL
Exemplary amino acid sequence of Cpf1 from Acid-
aminococcus sp. corresponding to Uniprot Accession No. U2UMQ6.
(SEQ ID NO: 4)
MTQFEGFTNLYQVSKTLRFELIPQGKTLKHIQEQGFIEEDKARNDHYKEL
KPIIDRIYKTYADQCLQLVQLDWENLSAAIDSYRKEKTEETRNALIEEQA
TYRNAIHDYFIGRTDNLTDAINKRHAEIYKGLFKAELFNGKVLKQLGTVT
TTEHENALLRSFDKFTTYFSGFYENRKNVESAEDISTAIPHRIVQDNFPK
FKENCHIFTRLITAVPSLREHFENVKKAIGIFVSTSIEEVFSFPFYNQLL
TQTQIDLYNQLLGGISREAGTEKIKGLNEVLNLAIQKNDETAHIIASLPH
RFIPLFKQILSDRNTLSFILEEFKSDEEVIQSFCKYKTLLRNENVLETAE
ALFNELNSIDLTHIFISHKKLETISSALCDHWDTLRNALYERRISELTGK
ITKSAKEKVQRSLKHEDINLQEIISAAGKELSEAFKQKTSEILSHAHAAL
DQPLPTTLKKQEEKEILKSQLDSLLGLYHLLDWFAVDESNEVDPEFSARL
TGIKLEMEPSLSFYNKARNYATKKPYSVEKFKLNFQMPTLASGWDVNKEK
NNGAILFVKNGLYYLGIMPKQKGRYKALSFEPTEKTSEGFDKMYYDYFPD
AAKMIPKCSTQLKAVTAHFQTHTTPILLSNNFIEPLEITKEIYDLNNPEK
EPKKFQTAYAKKTGDQKGYREALCKWIDFTRDFLSKYTKTTSIDLSSLRP
SSQYKDLGEYYAELNPLLYHISFQRIAEKEIMDAVETGKLYLFQIYNKDF
AKGHHGKPNLHTLYWTGLFSPENLAKTSIKLNGQAELFYRPKSRMKRMAH
RLGEKMLNKKLKDQKTPIPDTLYQELYDYVNHRLSHDLSDEARALLPNVI
TKEVSHEIIKDRRFTSDKFFFHVPITLNYQAANSPSKFNQRVNAYLKEHP
ETPIIGIDRGERNLIYITVIDSTGKILEQRSLNTIQQFDYQKKLDNREKE
RVAARQAWSVVGTIKDLKQGYLSQVIHEIVDLMIHYQAVVVLENLNFGFK
SKRTGIAEKAVYQQFEKMLIDKLNCLVLKDYPAEKVGGVLNPYQLTDQFT
SFAKMGTQSGFLFYVPAPYTSKIDPLTGFVDPFVWKTIKNHESRKHFLEG
FDFLHYDVKTGDFILHFKMNRNLSFQRGLPGFMPAWDIVFEKNETQFDAK
GTPFIAGKRIVPVIENHRFTGRYRDLYPANELIALLEEKGIVERDGSNIL
PKLLENDDSHAIDTMVALIRSVLQMRNSNAATGEDYINSPVRDLNGVCFD
SRFQNPEWPMDADANGAYHIALKGQLLLNHLKESKDLKLQNGISNQDWLA
YIQELRN

Residues E174, S542, K548, N552, and D908 are indicated in boldface and underlined. Variants of the Cpf1 sequence as provided above, or any suitable sequence of Cpf1 known in the art (e.g., the sequence above without the N-terminal methionine, e.g., in the context of a fusion protein), are also embraced by the present disclosure. Such sequences include, for example, a Cpf1 sequence comprising an amino acid substitution at residue E174, S542, 40 K548, N552, and D908, or two or more substitutions at any combination of these residues. In some embodiments, the Cpf1 sequence comprises an amino substitution at residue E174. In some embodiments, the amino acid substitution at residue E174 is a E174R substitution. In some embodiments, the Cpf1 sequence comprises an amino substitution at residue S542. In some embodiments, the amino acid substitution at residue S542 is a S542R substitution. In some embodiments, the Cpf1 sequence comprises an amino substitution at residue K548. In some embodiments, the amino acid substitution at residue K548 is a K548V substitution. In some embodiments, the Cpf1 sequence comprises an amino substitution at residue N552. In some embodiments, the amino acid substitution at residue N552 is a N552R substitution. In some embodiments, the Cpf1 sequence comprises an amino substitution at residue D908. In some embodiments, the amino acid substitution at residue D908 is a D908A substitution.

In some embodiments, the Cpf1 domain comprises an amino acid sequence of SEQ ID NO: 4 that is lacking the N-terminal methionine, e.g., in the context of a fusion protein), are also embraced by the present disclosure. In some embodiments, the Cpf1 domain comprises an amino acid sequence of SEQ ID NO: 4 comprising an amino acid substitution at residue E174, S542, K548, N552, and D908, or two or more substitutions at any combination of these residues. In some embodiments, the Cpf1 domain comprises an amino acid sequence of SEQ ID NO: 4 and comprises an amino substitution at residue E174. In some embodiments, the amino acid substitution at residue E174 is a E174R substitution. In some embodiments, the Cpf1 domain comprises an amino acid sequence of SEQ ID NO: 4 and comprises an amino substitution at residue S542. In some embodiments, the amino acid substitution at residue S542 is a S542R substitution. In some embodiments, the Cpf1 domain comprises an amino acid sequence of SEQ ID NO: 4 and comprises a substitution at residue K548. In some embodiments, the amino acid substitution at residue K548 is a K548V substitution. In some embodiments, the Cpf1 domain comprises an amino acid sequence of SEQ ID NO: 4 and comprises an amino substitution at residue N552. In some embodiments, the amino acid substitution at residue N552 is a N552R substitution. In some embodiments, the Cpf1 domain comprises an amino acid sequence of SEQ ID NO: 4 and comprises an amino substitution at residue D908. In some embodiments, the amino acid substitution at residue D908 is a D908A substitution.

Exemplary amino acid sequence of Cpf1 from Acidaminococcus sp. containing
E174R, S542R, K548V, N552R, and D908A mutations
(SEQ ID NO: 5)
TQFEGFTNLYQVSKTLRFELIPQGKTLKHIQEQGFIEEDKARNDHYKELKPIIDRIYKTYADQCLQLVQLDWENL
SAAIDSYRKEKTEETRNALIEEQATYRNAIHDYFIGRTDNLTDAINKRHAEIYKGLFKAELFNGKVLKQLGTVTT
TEHENALLRSFDKFTTYFSGFYRNRKNVFSAEDISTAIPHRIVQDNFPKFKENCHIFTRLITAVPSLREHFENVK
KAIGIFVSTSIEEVFSFPFYNQLLTQTQIDLYNQLLGGISREAGTEKIKGLNEVLNLAIQKNDETAHIIASLPHR
FIPLFKQILSDRNILSFILEEFKSDEEVIQSFCKYKILLRNENVLETAEALFNELNSIDLTHIFISHKKLETISS
ALCDHWDTLRNALYERRISELTGKITKSAKEKVQRSLKHEDINLQEIISAAGKELSEAFKQKTSEILSHAHAALD
QPLPTTLKKQEEKEILKSQLDSLLGLYHLLDWFAVDESNEVDPEFSARLTGIKLEMEPSLSFYNKARNYATKKPY
SVEKFKLNFQMPTLARGWDVNVEKNRGAILFVKNGLYYLGIMPKQKGRYKALSFEPTEKTSEGEDKMYYDYFPDA
AKMIPKCSTQLKAVTAHFQTHTTPILLSNNFIEPLEITKEIYDLNNPEKEPKKFQTAYAKKTGDQKGYREALCKW
IDFTRDFLSKYTKITSIDLSSLRPSSQYKDLGEYYAELNPLLYHISFQRIAEKEIMDAVETGKLYLFQIYNKDFA
KGHHGKPNLHTLYWTGLFSPENLAKTSIKLNGQAELFYRPKSRMKRMAHRLGEKMLNKKLKDQKTPIPDTLYQEL
YDYVNHRLSHDLSDEARALLPNVITKEVSHEIIKDRRFTSDKFFFHVPITLNYQAANSPSKFNQRVNAYLKEHPE
TPIIGIARGERNLIYITVIDSTGKILEQRSLNTIQQFDYQKKLDNREKERVAARQAWSVVGTIKDLKQGYLSQVI
HEIVDLMIHYQAVVVLENLNFGFKSKRIGIAEKAVYQQFEKMLIDKLNCLVLKDYPAEKVGGVLNPYQLTDQFTS
FAKMGTQSGFLFYVPAPYTSKIDPLIGFVDPFVWKTIKNHESRKHFLEGFDFLHYDVKTGDFILHFKMNRNLSFQ
RGLPGFMPAWDIVFEKNETQFDAKGTPFIAGKRIVPVIENHRFTGRYRDLYPANELIALLEEKGIVERDGSNILP
KLLENDDSHAIDTMVALIRSVLQMRNSNAATGEDYINSPVRDLNGVCFDSRFQNPEWPMDADANGAYHIALKGQL
LLNHLKESKDLKLQNGISNQDWLAYIQELRN
Exemplary amino acid sequence of Cpf1 from Prevotella spp. corresponding
to Uniprot Accession No. A0A350PSL0.
(SEQ ID NO: 6)
MAKNFEDFKRLYPLSKTLRFEAKPIGATLDNIVKSGLLEEDEHRAASYVKVKKLIDEYHKVFIDRVLDNGCLPLD
DKGDNNSLAEYYESYVSKAQDEDAIKKFKEIQQNLLSIIAKKLTDDKAYANLFGNKLIESYKDKADKTKLIDSDL
IQFINTAESTQLVSMSQDEAKELVKEFWGFTTYFEGFFKNRKNMYTPEEKSTGIAYRLINENLPKFIDNMEAFKK
AIARPEIQANMEELYSNFSEYLNVESIQEMFLLDYYNMLLTQKQIDVYNAIIGGKTDDEHDVKIKGINEYINLYN
QQHKDDKLPKLKALFKQILSDRNAISWLPEEFNSDQEVLNAIKDCYERLAENVLGDKVLKSLLGSLADYSLDGIF
IRNDLQLTDISQKMFGNWGVIQNAIMQNIKHVAPARKHKESEEDYEKRIAGIFKKADSFSISYINDCLNEADPNN
AYFVENYFATFGAVNTPTMQRENLFALVQNAYTEVAALLHSDYPTVKHLAQDKANVSKIKALLDAIKSLQHFVKP
LLGKGDESDKDERFYGELASLWAELDTVTPLYNMIRNYMTRKPYSQKKIKLNFENPQLLGGWDANKEKDYATIIL
RRNGLYYLAIMDKDSRKLLGKAMPSDGECYEKMVYKFFKDVTTMIPKCSTQLKDVQAYFKVNTDDYVLNSKAFNR
PLTITKEVFDLNNVLYGKYKKFQKGYLTATGDNVGYTHAVNVWIKFCMDFLDSYDSTCIYDFSSLKPESYLSLDS
FYQDVNLLLYKLSFTDVSASFIDQLVEEGKMYLFQIYNKDFSEYSKGTPNMHTLYWKALFDERNLADVVYKLNGQ
AEMFYRKKSIENTHPTHPANHPILNKNKDNKKKESLFEYDLIKDRRYTVDKFMFHVPITMNFKSSGSENINQDVK
AYLRHADDMHIIGIDRGERHLLYLVVIDLQGNIKEQFSLNEIVNDYNGNTYHTNYHDLLDVREDERLKARQSWQT
IENIKELKEGYLSQVIHKITQLMVRYHAIVVLEDLSKGFMRSRQKVEKQVYQKFEKMLIDKLNYLVDKKTDVSTP
GGLLNAYQLTCKSDSSQKLGKQSGFLFYIPAWNTSKIDPVTGFVNLLDTHSLNSKEKIKAFFSKFDAIRYNKDKK
WFEFNLDYDKFGKKAEDTRTKWTLCTRGMRIDTFRNKEKNSQWDNQEVDLTTEMKSLLEHYYIDIHGNLKDAIST
QTDKAFFTGLLHILKLTLQMRNSITGTETDYLVSPVADENGIFYDSRSCGDQLPENADANGAYNIARKGLMLVEQ
IKDAEDLDNVKFDISNKAWLNFAQQKPYKNG
Exemplary amino acid sequence of Cpf1 from Francisella spp.
corresponding to Uniprot Accession No. A0Q7Q2.
(SEQ ID NO: 7)
MSIYQEFVNKYSLSKTLRFELIPQGKTLENIKARGLILDDEKRAKDYKKAKQIIDKYHQFFIEEILSSVCISEDL
LQNYSDVYFKLKKSDDDNLQKDFKSAKDTIKKQISEYIKDSEKFKNLFNQNLIDAKKGQESDLILWLKQSKDNGI
ELFKANSDITDIDEALEIIKSFKGWTTYFKGFHENRKNVYSSNDIPTSIIYRIVDDNLPKFLENKAKYESLKDKA
PEAINYEQIKKDLAEELTFDIDYKTSEVNQRVFSLDEVFEIANFNNYLNQSGITKENTIIGGKFVNGENTKRKGI
NEYINLYSQQINDKTLKKYKMSVLFKQILSDTESKSFVIDKLEDDSDVVTTMQSFYEQIAAFKTVEEKSIKETLS
LLFDDLKAQKLDLSKIYFKNDKSLTDLSQQVFDDYSVIGTAVLEYITQQIAPKNLDNPSKKEQELIAKKTEKAKY
LSLETIKLALEEFNKHRDIDKQCRFEEILANFAAIPMIFDEIAQNKDNLAQISIKYQNQGKKDLLQASAEDDVKA
IKDLLDQTNNLLHKLKIFHISQSEDKANILDKDEHFYLVFEECYFELANIVPLYNKIRNYITQKPYSDEKFKLNF
ENSTLANGWDKNKEPDNTAILFIKDDKYYLGVMNKKNNKIFDDKAIKENKGEGYKKIVYKLLPGANKMLPKVFFS
AKSIKFYNPSEDILRIRNHSTHTKNGSPQKGYEKFEFNIEDCRKFIDFYKQSISKHPEWKDFGFRESDTQRYNSI
DEFYREVENQGYKLTFENISESYIDSVVNQGKLYLFQIYNKDFSAYSKGRPNLHTLYWKALFDERNLQDVVYKLN
GEAELFYRKQSIPKKITHPAKEAIANKNKDNPKKESVFEYDLIKDKRFTEDKFFFHCPITINFKSSGANKENDEI
NLLLKEKANDVHILSIDRGERHLAYYTLVDGKGNIIKQDTFNIIGNDRMKTNYHDKLAAIEKDRDSARKDWKKIN
NIKEMKEGYLSQVVHEIAKLVIEYNAIVVFEDLNFGFKRGRFKVEKQVYQKLEKMLIEKLNYLVFKDNEFDKTGG
VLRAYQLTAPFETFKKMGKQTGIIYYVPAGFTSKICPVTGFVNQLYPKYESVSKSQEFFSKEDKICYNLDKGYFE
FSFDYKNFGDKAAKGKWTIASFGSRLINFRNSDKNHNWDTREVYPTKELEKLLKDYSIEYGHGECIKAAICGESD
KKFFAKLTSVLNTILQMRNSKTGTELDYLISPVADVNGNFFDSRQAPKNMPQDADANGAYHIGLKGLMLLGRIKN
NQEGKKLNLVIKNEEYFEFVQNRNN
Exemplary amino acid sequence of Cpf1 from Lachnospiraceae spp.
corresponding to Uniprot Accession No. A0A7C9H0Z9.
(SEQ ID NO: 8)
MNGNRIIVYREFVGVTPVAKTLRNELRPIGHTQEHIIHNGLIQEDELRQEKSTELKNIMDDYYREYIDKSLSGVT
DLDFTLLFELMNLVQSSPSKDNKKALEKEQSKMREQICTHMQSDSNYKNIFNAKFLKEILPDFIKNYNQYDAKDK
AGKLETLALFNGFSTYFTDFFEKRKNVFTKEAVSTSIAYRIVHENSLTFLANMTSYKKISEKALDEIEVIEKNNQ
DKMGDWELNQIFNPDFYNMVLIQSGIDFYNEICGVVNAHMNLYCQQTKNNYNLFKMRKLHKQILAYTSTSFEVPK
MFEDDMSVYNAVNAFIDETEKGNIIGKLKDIVNKYDELDEKRIYISKDFYETLSCFMSGNWNLITGCVENFYDEN
IHAKGKSKEEKVKKAVKEDKYKSINDVNDLVEKYIDEKERNEFKNSNAKQYIREISNIITDTETAHLEYDEHISL
IESEEKADEMKKRLDMYMNMYHWAKAFIVDEVLDRDEMFYSDIDDIYNILENIVPLYNRVRNYVTQKPYNSKKIK
LNFQSPTLANGWSQSKEFDNNAIILIRDNKYYLAIFNAKNKPDKKIIQGNSDKKNDNDYKKMVYNLLPGANKMLP
KVFLSKKGIETFKPSDYIISGYNAHKHIKTSENFDISFCRDLIDYFKNSIEKHAEWRKYEFKESATDSYNDISEF
YREVEMQGYRIDWTYISEADINKLDEEGKIYLFQIYNKDFAENSTGKENLHTMYFKNIFSEENLKDIIIKLNGQA
ELFYRRASVKNPVKHKKDSVLVNKTYKNQLDNGDVVRIPIPDDIYNEIYKMYNGYIKESDLSGAAKEYLDKVEVR
TAQKEIVKDYRYTVDKYFIHTPITINYKVAARNNVNDMAVKYIAQNDDIHVIGIDRGERNLIYISVIDSHGNIVK
QKSYNILNNYDYKKKLVEKEKTREYARKNWKSIGNIKELKEGYISGVVHEIAMLMVEYNAIIAMEDLNYGEKRGR
FKVERQVYQKFESMLINKLNYFASKGKSVDEPGGLLKGYQLTYVPDNIKNLGKQCGVIFYVPAAFTSKIDPSTGF
ISAFNFKSISTNDSRKQFFMQFDEIRYCAEKDMFSFGFDYNNEDTYNITMGKTQWTVYTNGERLQSEFNNARRTG
KTKSINLTETIKLLLEDNEINYADGHDVRIDMEKMDEDKNSEFFAQLLSLYKLTVQMRNSYTEAEEQEKGISYDK
IISPVINDEGEFFDSDNYKESDDKECKMPKDADANGAYCIALKGLYEVLKIKSEWTEDGFDRNCLKLPHAEWLDF
IQNKRYE
Exemplary amino acid sequence of Cpf1 from Eubacterium rectale
corresponding to Uniprot Accession No. A0A6L5T656.
(SEQ ID NO: 9)
MNNGTNNFQNFIGISSLQKTLRNALIPTETTQQFIVKNGIIKEDELRGENRQILKDIMDDYYRGFISETLSSIDD
IDWTSLFEKMEIQLKNGDNKDTLIKEQAEKRKAIYKKFADDDRFKNMFSAKLISDILEFVIHNNNYSASEKEEKT
QVIKLESRFATSFKDYFKNRANCESADDISSSSCHRIVNDNAEIFFSNALVYRRIVKNLSNDDINKISGDMKDSL
KEMSLDEIYSYEKYGEFITQEGISFYNDICGKVNSFMNLYCQKNKENKNLYKLRKLHKQILCIADTSYEVPYKFE
SDEEVYQSVNGELDNISSKHIVERLRKIGDNYNGYNLDKIYIVSRFYESVSQKTYRDWETINTALEIHYNNILPG
NGKSKADKVKKAVKNDLQKSITEINELVSNYKLCPDDNIKAETYIHEISHILNNFEAQELKYNPEIHLVESELKA
SELKNVLDVIMNAFHWCSVFMTEELVDKDNNFYAELEEIYDEIYPVISLYNLVRNYVTQKPYSTKKIKLNFGIPT
LADGWSKSKEYSNNAIILMRDNLYYLGIFNAKNKPDKKIIEGNTSENKGDYKKMIYNLLPGPNKMIPKVELSSKT
GVETYKPSAYILEGYKQNKHLKSSKDFDITFCRDLIDYFKNCIAIHPEWKNFGFDFSDTSTYEDISGFYREVELQ
GYKIDWTYISEKDIDLLQEKGQLYLFQIYNKDFSKKSIGNDNLHTMYLKNLFSEENLKDIVLKLNGEAEIFFRKS
SIKNPIIHKKGSILVNRTYEAEEKDQFGNIQIVRKTIPENIYQELYKYFNDKSDKELSDEAAKLKNVVGHHEAAT
NIVKDYRYTYDKYFLHMPITINFKANKISFINDRILQYIAKEKDLHVIGIDRGERNLIYVSVIDICGNIVEQKSF
NIVNGYDYQIKLKQQEGARQIARKEWKEIGKIKEIKEGYLSLVIHEISKMVIKYNAIIAMEDLSYGFKKGREKVE
RQVYQKFETMLINKLNYLVFKDISITENGGLLKGYQLTYIPDKLKNVGHQCGCIFYVPAAYTSKIDPTTGFVNIF
KFKDLTVDAKREFIKKFDSIRYDSDKNLFCFTFDYNNFITQNTVMSKSSWSVYTYGVRIKRRFVNGRESNESDTI
DITKDMEKTLEMTDINWRDGHDLRQDIIDYEIVQHIFEIFKLTVQMRNSLSELEDRDYDRLISPVLNENNIFYDS
AKAGDALPKDADANGAYCIALKGLYEIKQITENWKEDGKFSRDKLKISNKDWFDFIQNKRYL

Both naturally occurring and modified variants of Cpf1 enzymes are suitable for use according to aspects of this disclosure. For example, in some embodiments, a Cpf1 domain is modified to reduce or eliminate nuclease activity of the domain. A catalytically inactive Cas nuclease may be referred to as “dead Cas12” “dCas12,” “dead Cpf1,” or “dCpf1.” In some embodiments, the inactive Cas nuclease is “dead Casϕ” or “dCasϕ.” To generate a Cpf1 domain lacking nuclease activity, any mutation (e.g., an insertion, deletion, inversion, or substitution) of one or more amino acids of the Cpf1 may be made such that the nuclease activity is reduced as compared to a Cpf1 domain that does contain the mutation (e.g., a wild-type Cpf1 domain). In some embodiments, the Cpf1 domain does not have detectable nuclease activity. Exemplary mutations that reduce or eliminate nuclease activity of the Cpf1 enzyme are known in the art. See, e.g., Liu et al. Nature Communications (2017) 8: 2095. In some embodiments, the Cpf1 domain comprises a mutation of an amino acid residue corresponding to the aspartic acid residue at position 908 (referred to as “D908”) of Cpf1 from Acidaminococcus sp. (AsCpf1). In some embodiments, the Cpf1 domain comprises a mutation of an amino acid residue corresponding to the aspartic acid residue at position 908 (referred to as “D908”) of Cpf1 from Acidaminococcus sp. (AsCpf1) provided by SEQ ID NO: 4. In some embodiments, the Cpf1 domain comprises a substitution of an amino acid residue corresponding to the aspartic acid residue at position 908 of Cpf1 from Acidaminococcus sp. (AsCpf1) provided by SEQ ID NO: 4, to any other amino acid residue other than aspartic acid. In some embodiments, the Cpf1 domain comprises a substitution of an amino acid residue corresponding to the aspartic acid residue at position 908 of Cpf1 from Acidaminococcus sp. (AsCpf1) provided by SEQ ID NO: 4, to an alanine residue (referred to as “D908A”). In some embodiments, to generate a dead Casϕ lacking nuclease activity, the Casϕ protein is engineered to comprise D371A and D394A in the RuvC domain (see, e.g., Pausch et al. Science. (2020) 369: 333-337, incorporated by reference in its entirety).

In some embodiments, the Cpf1 domain is based on the MAD7™ enzyme (Inscripta). In such embodiments, an exemplary mutation that results in reduction or elimination of nuclease activity of the enzyme comprises a substitution of the aspartic acid residue at position 877 of MAD7™ provided by SEQ ID NO: 1, to any other amino acid residue other than aspartic acid. In some embodiments, the Cpf1 domain is based on the MAD7™ enzyme (Inscripta) and comprises a substitution of the aspartic acid residue at position 877 of MAD7™ provided by SEQ ID NO: 1, to an alanine residue (referred to as “D877A”).

In some embodiments, the Cpf1 domain comprises one or more mutations, for example to modulate genome editing activity, modulate editing efficiency, and/or reduce off target effects. See, e.g., Kleinstiver et al. Nature Biotech. (2019) 37: 276-282, incorporated by reference in its entirety. In some embodiments, the Cpf1 domain comprises one or more mutations relative to a corresponding wildtype Cpf1 nuclease. In some embodiments, the Cpf1 domain comprises one or more substitutions in the Cpf1 domain relative to a corresponding wildtype Cpf1 domain.

In some embodiments, the Cpf1 domain comprises a substitution of at least one (e.g., 1, 2, 3, 4, 5, 6, 7, 8, 9, 10 or more) amino acids of the Cpf1 domain relative to a corresponding wildtype Cpf1 domain. In some embodiments, the Cpf1 domain comprises a substitution of an amino acid at: one, two, three, or each of amino acids corresponding to positions 174, 542, 548, or 552 of the Acidaminococcus sp. Cpf1 amino acid sequence (referred to as E174, S542, K548, and N552). In some embodiments, the Cpf1 domain comprises a substitution of an amino acid residue corresponding to the glutamic acid at position 174 of Cpf1 from Acidaminococcus sp, to any other amino acid residue other than glutamic acid. In some embodiments, the Cpf1 domain comprises a substitution of an amino acid residue corresponding to the glutamic acid at position 174 of Cpf1 from Acidaminococcus sp, to an arginine residue (E174R). In some embodiments, the Cpf1 domain comprises a substitution of an amino acid residue corresponding to the serine at position 542 of Cpf1 from Acidaminococcus sp, to any other amino acid residue other than serine. In some embodiments, the Cpf1 domain comprises a substitution of an amino acid residue corresponding to the serine at position 542 of Cpf1 from Acidaminococcus sp, to an arginine residue (S542R). In some embodiments, the Cpf1 domain comprises a substitution of an amino acid residue corresponding to the lysine at position 548 of Cpf1 from Acidaminococcus sp, to any other amino acid residue other than lysine. In some embodiments, the Cpf1 domain comprises a substitution of an amino acid residue corresponding to the lysine at position 548 of Cpf1 from Acidaminococcus sp, to a valine residue (K548V). In some embodiments, the Cpf1 domain comprises a substitution of an amino acid residue corresponding to the asparagine at position 552 of Cpf1 from Acidaminococcus sp, to any other amino acid residue other than asparagine. In some embodiments, the comprises a substitution of an amino acid residue corresponding to the asparagine at position 552 of Cpf1 from Acidaminococcus sp, to a arginine residue (N552R).

In some embodiments, the Cpf1 domain comprises a substitution of an amino acid residue corresponding to each of positions 174, 542, 548, and 552 of Cpf1 from Acidaminococcus sp, to any other amino acid residue. In some embodiments, the Cpf1 domain comprises a substitution mutation corresponding to each of E174R, S542R, K548V, and N552R.

In some embodiments, the Cpf1 domain comprises a substitution of at least one (e.g., 1, 2, 3, 4, 5, 6, 7, 8, 9, 10 or more) amino acids of the Cpf1 domain relative to a corresponding wildtype MAD7™ Cpf1 amino acid sequence. In some embodiments, the Cpf1 domain comprises a substitution of an amino acid at: one, two, three, or each of amino acids corresponding to positions 169, 529, 535, or 538 of the MAD7™ Cpf1 amino acid sequence (referred to as E169, D529, K535, and N538).

In some embodiments, the Cpf1 domain comprises a substitution of an amino acid residue corresponding to the glutamic acid at position 169 of the MAD7™ Cpf1 amino acid sequence, to any other amino acid residue other than glutamic acid. In some embodiments, the Cpf1 domain comprises a substitution of an amino acid residue corresponding to the glutamic acid at position 169 of MAD7™ Cpf1 amino acid sequence to an arginine residue (E169R). In some embodiments, the Cpf1 domain comprises a substitution of an amino acid residue corresponding to the aspartic acid at position 529 of the MAD7™ Cpf1 amino acid sequence, to any other amino acid residue other than aspartic acid. In some embodiments, the Cpf1 domain comprises a substitution of an amino acid residue corresponding to the aspartic acid at position 529 of MAD7™ Cpf1 amino acid sequence to an arginine residue (D529R). In some embodiments, the Cpf1 domain comprises a substitution of an amino acid residue corresponding to the lysine at position 535 of the MAD7™ Cpf1 amino acid sequence, to any other amino acid residue other than lysine. In some embodiments, the Cpf1 domain comprises a substitution of an amino acid residue corresponding to the lysine at position 535 of MAD7™ Cpf1 amino acid sequence to a valine residue (K535V). In some embodiments, the Cpf1 domain comprises a substitution of an amino acid residue corresponding to the asparagine at position 538 of the MAD7™ Cpf1 amino acid sequence, to any other amino acid residue other than asparagine. In some embodiments, the Cpf1 domain comprises a substitution of an amino acid residue corresponding to the asparagine at position 538 of MAD7™ Cpf1 amino acid sequence to an arginine residue (N538R).

In some embodiments, the Cpf1 domain comprises a substitution of an amino acid residue corresponding to each of positions 169, 529, 535, or 538 of MAD7™ Cpf1 amino acid sequence to any other amino acid residue. In some embodiments, the Cpf1 domain comprises a substitution mutation corresponding to each of K169, D529R, K535V, and N538R.

In some embodiments, the amino acid sequence of the first Cpf1 protein comprises any of SEQ ID NOs: 1-9 or a sequence with at least 80%, 85%, 90%, 95%, 96%, 97%, 98%, 99% identity or higher to any thereof. In some embodiments, the amino acid sequence of the second Cpf1 protein comprises any of SEQ ID NOs: 1-9 or a sequence with at least 80%, 85%, 90%, 95%, 96%, 97%, 98%, 99% identity or higher to any thereof. In some embodiments, the chimeric polypeptide comprises an amino acid sequence of any of SEQ ID NOs: 1-9 or a sequence with at least 80%, 85%, 90%, 95%, 96%, 97%, 98%, 99% identity or higher to any thereof.

### Endonuclease Domain

The fusion polypeptides described herein comprise a Cpf1 domain that lacks nuclease activity and an endonuclease domain. In some embodiments, the fusion polypeptides comprise a Cpf1 domain that lacks nuclease activity, an endonuclease domain, and a genomic modification domain. As used herein, an “endonuclease domain” refers to an enzyme, or portion thereof, that is capable of cleaving a phosphodiester bond between two nucleotides, resulting in a single or double stranded break in the polynucleotide. In general, endonucleases may cleave between two nucleotides in a sequence-specific or a sequence-independent manner. In some embodiments, the endonuclease cleaves a phosphodiester bond between two nucleotides following recognition of a particular nucleotide sequence (i.e., a recognition site). In some embodiments, endonucleases that cleave between two nucleotides in a sequence-specific manner may be referred to as restriction enzymes or restriction endonucleases.

Endonucleases are typically categorized based on factors, such as the structure of the recognition site, position of cleavage relative to the recognition site, and whether endonuclease activity requires the presence of any enzyme cofactors. Examples of types of endonucleases include, Type 1 endonucleases, Type II endonucleases, Type III endonucleases, Type IV endonucleases, and Type V endonucleases.

In some embodiments, fusion polypeptides described herein comprise a Type II endonuclease or a domain thereof. Type II endonucleases form a homodimer and recognize and cleave nucleic acid at a position near (e.g., within 1, 2, 3, 4, or 5 nucleotides of the recognition site) or within the recognition site, resulting in a double stranded break of the polynucleotide. Subtypes of Type II endonucleases include, Type IIA, Type IIB, Type IIC, Type IIE, Type IIF, Type IIG, Type IIH, Type IIM, Type IIP, Type IIS, and Type IIT. See, e.g., Pingoud et al. Nucleic Acids Research (2014) 42(12): 7489-7527.

In some embodiments, the fusion polypeptides described herein comprise an endonuclease domain of a restriction endonuclease, such as a Type II endonuclease.

In some embodiments, fusion polypeptides described herein comprise a Type IIS endonuclease or a portion thereof. Type IIS restriction enzymes are characterized as being comprised of more than one subunit: a subunit comprising a DNA-binding domain and a subunit comprising a DNA-cleavage domain. Without wishing to be bound by any particular theory, it is generally thought that Type IIS endonucleases interact with a particular recognition site through the DNA-binding domain, form homodimers, and cleave the phosphodiester bond between two nucleotides near the recognition site. Non-limiting examples of Type IIS restriction enzymes include FokI, AcuI, AlwI, BaeI, BbsI, BbsI-HF, BbvI, BccI, BceAI, BcgI, BclVI, BcoDI, Bfil, BfuAI, BmrI, BpmI, BpuEI, BsaI-HFv2, BsaXI, BseRI, BsgI, BsmAI, BsmBI-v2, BsmFI, BsmI, BspCNI, BspMI, BspQI, BsrDI, BsrI, BtgZI, BtsCI, BtsI-v2, BtsIMutI, CspCI, Earl, EciI, Esp3I, FauI, HgaI, HphI, HpyAV, MboII, MlyI, MmeI, MnlI, NmeAIII, PaqCI, PleI, SapI, and SfaNI. In some embodiments, the fusion polypeptides described herein comprise a Fok1 endonuclease or a portion thereof.

In some embodiments, the endonuclease domain is a portion of a restriction endonuclease comprising at least 10%, 20%, 30%, 40%, 50%, 60%, 70%, 80%, 90%, or more of the restriction endonuclease enzyme. In some embodiments, the endonuclease domain is one or more domains of a restriction endonuclease, such as a DNA-cleavage domain, a dimerization domain, and a catalytic site.

In some embodiments, the endonuclease domain comprises a first DNA-cleavage domain that is capable of forming a dimer with a second DNA-cleavage domain, which may have the same amino acid sequence as the first DNA-cleavage domain, or a different amino acid sequence as compared to the first DNA-cleavage domain. In some embodiments, the endonuclease domain of the fusion polypeptide comprises a first DNA-cleavage domain is capable of forming a dimer with a second DNA-cleavage domain. In some embodiments, the endonuclease domain of the fusion polypeptide comprises a first DNA-cleavage domain is capable of forming a dimer with a second DNA-cleavage domain that is present in a separate polypeptide. In some embodiments, the endonuclease domain of the fusion polypeptide comprises a first DNA-cleavage domain and a second DNA-cleavage domain, wherein the first DNA-cleavage domain and second DNA-cleavage domain are capable of forming a dimer with one another (e.g., within the same fusion polypeptide). In some embodiments, the endonuclease domain of the fusion polypeptide does not include a DNA-binding domain of a restriction endonuclease.

In some embodiments, a dimer of the first DNA-cleavage domain and second DNA-cleavage domain generates a double-stranded break in a targeted polynucleotide. In some embodiments, a dimer of the first DNA-cleavage domain and second DNA-cleavage domain generates a double-stranded break in a targeted polynucleotide. Such single-stranded break activity may be referred to as a “nickase.” In some embodiments, a dimer of the first DNA-cleavage domain and second DNA-cleavage domain generates a double-stranded break in a targeted DNA.

In some embodiments, the endonuclease domain comprises FokI or a portion thereof. In some embodiments, the endonuclease domain comprises a DNA-cleavage domain of FokI. In some embodiments, the endonuclease domain does not include a DNA-binding domain of FokI. FokI is a Type IIS restriction enzyme isolated from Flavobacterium okeanokoites. Each monomer of wildtype FokI has a DNA-binding domain and a DNA-cleavage domain. Wild-type FokI forms a dimer in which each monomer cleaves a single strand of DNA, leading to a double stranded break in the targeted DNA. See, e.g., Wah et al. PNAS (1998) 95(18): 10564-10569. In some embodiments, the first DNA-cleavage domain and/or the second DNA-cleavage domain of the endonuclease domain is a DNA-cleavage domain of FokI or is derived from a DNA-cleavage domain of FokI. In some embodiments, the endonuclease domain does not comprise the DNA binding domain of FokI. In some embodiments, the endonuclease domain is not capable of forming and/or maintaining a complex with DNA in the absence of an accompanying Cpf1 domain.

In some embodiments, the endonuclease domain is genetically modified relative to a naturally occurring or wildtype endonuclease domain sequence. In some embodiments, the first DNA-cleavage domain and/or the second DNA-cleavage domain comprise one or more modifications (e.g., mutations, substitutions, deletions, insertions) relative to a corresponding wildtype DNA-cleavage domain sequence. In some embodiments, the first DNA-cleavage domain and/or the second DNA-cleavage domain comprise one or more modifications to modulate activity of the endonuclease domain (or DNA-cleavage domain) such that at least one of the first DNA-cleavage domain or the second DNA-cleavage domain has reduced or eliminated endonuclease activity (e.g., does not cleave a phosphodiester bond). In some embodiments, the first DNA-cleavage domain comprises one or more modifications such that the first DNA-cleavage domain has reduced or eliminated endonuclease activity (e.g., does not cleave a phosphodiester bond). In some embodiments, the second DNA-cleavage domain comprises one or more modifications such that the second DNA-cleavage domain has reduced or eliminated endonuclease activity (e.g., does not cleave a phosphodiester bond).

In some embodiments, the first DNA-cleavage domain comprises one or more modifications such that the first DNA-cleavage domain has reduced or eliminated endonuclease activity (e.g., does not cleave a phosphodiester bond) and the second DNA-cleavage domain comprises wildtype or substantially wildtype endonuclease activity (e.g., functional endonuclease activity, capable of cleaving a phosphodiesterase bond), such that a dimer of the first DNA-cleavage domain and second DNA-cleavage domain does not produce double stranded breaks in a targeted DNA. In some embodiments, the first DNA-cleavage domain comprises one or more modifications such that the first DNA-cleavage domain has reduced or eliminated endonuclease activity (e.g., does not cleave a phosphodiester bond) and the second DNA-cleavage domain comprises wildtype or substantially wildtype endonuclease activity (e.g., functional endonuclease activity, capable of cleaving a phosphodiesterase bond), such that a dimer of the first DNA-cleavage domain and second DNA-cleavage domain is capable of generating a single-stranded break in a targeted DNA (e.g., is a nickase). In some embodiments, the second DNA-cleavage domain comprises one or more modifications such that the second DNA-cleavage domain has reduced or eliminated endonuclease activity (e.g., does not cleave a phosphodiester bond) and the first DNA-cleavage domain comprises wildtype or substantially wildtype endonuclease activity (e.g., functional endonuclease activity, capable of cleaving a phosphodiesterase bond), such that a dimer of the first DNA-cleavage domain and second DNA-cleavage domain does not produce double stranded breaks in a targeted DNA. In some embodiments, the second DNA-cleavage domain comprises one or more modifications such that the second DNA-cleavage domain has reduced or eliminated endonuclease activity (e.g., does not cleave a phosphodiester bond) and the first DNA-cleavage domain comprises wildtype or substantially wildtype endonuclease activity (e.g., functional endonuclease activity, capable of cleaving a phosphodiesterase bond), such that a dimer of the first DNA-cleavage domain and second DNA-cleavage domain is capable of generating a single-stranded break in a targeted DNA (e.g., is a nickase).

In some embodiments, the first DNA-cleavage domain comprises one or more modifications that reduce or eliminate endonuclease activity of the first DNA-cleavage domain (e.g., does not cleave a phosphodiester bond). In some embodiments, the first DNA-cleavage domain comprises one or more mutations (e.g., 1, 2, 3, 4, 5 or more) that result in a DNA-cleavage domain having reduced or eliminated endonuclease activity. In some embodiments, the first DNA-cleavage domain comprises a mutation of one or more amino acids (e.g., 1, 2, 3, 4, 5 or more) that result in a DNA-cleavage domain having reduced or eliminated endonuclease activity. In some embodiments, the first DNA-cleavage domain comprises a mutation of one or more amino acids (e.g., 1, 2, 3, 4, 5 or more) in the catalytic site (active site) of the DNA-cleavage domain that result in a DNA-cleavage domain having reduced or eliminated endonuclease activity.

In some embodiments, the endonuclease domain comprises FokI or a portion thereof. In some embodiments, the endonuclease domain comprises a DNA-cleavage domain of FokI. In some embodiments, the endonuclease domain does not include a DNA-binding domain of FokI. In some embodiments, the endonuclease domain comprises a first DNA-cleavage domain from FokI. In some embodiments, the endonuclease domain comprises a second DNA-cleavage domain from FokI. In some embodiments, the first DNA-cleavage domain and/or the second DNA-cleavage domain from FokI comprises a mutation of one or more amino acids (e.g., 1, 2, 3, 4, 5 or more) that results in the DNA-cleavage domain having reduced or eliminated endonuclease activity, for example as compared to the wildtype DNA-cleavage domain from FokI (not comprising the mutation). Mutations in the DNA-cleavage domain to impair endonuclease activity of a monomer of a FokI dimer may direct DNA cleavage (nicking) to a particular DNA strand. See, e.g., Sanders et al. Nucleic Acids Res. (2009) 37(7): 2105-2115, incorporated by reference in its entirety.

In some embodiments, the endonuclease domain comprises an amino acid sequence of SEQ ID NOs: 10-14, or a sequence with at least 80%, 85%, 90%, 95%, 96%, 97%, 98%, 99% or higher identity to SEQ ID NOs: 10-14. In some embodiments, the first DNA-cleavage domain and/or the second DNA-cleavage domain comprises an amino acid sequence of SEQ ID NOs: 10-14, or a sequence with at least 80%, 85%, 90%, 95%, 96%, 97%, 98%, 99% or higher identity to SEQ ID NOs: 10-14.

In some embodiments, the first DNA-cleavage domain and/or the second DNA-cleavage domain comprises a DNA-cleavage domain from FokI. In some embodiments, the first DNA-cleavage domain and/or the second DNA-cleavage domain comprises an amino acid sequence of SEQ ID NOs: 10-14 and comprises a substitution mutation of one or more amino acids (e.g., 1, 2, 3, 4, 5 or more), for example in the catalytic site (active site) of the DNA-cleavage domain, as compared to SEQ ID NO: 10 or 11, respectively, that results in a DNA-cleavage domain having reduced or eliminated endonuclease activity. In some embodiments, the first DNA-cleavage domain and/or the second DNA-cleavage domain comprises an amino acid sequence of SEQ ID NOs: 10 or 11 and comprises a substitution of an aspartic acid residue at amino acid position number 450 (which may also be referred to as D450) of SEQ ID NO: 10. In some embodiments, the first DNA-cleavage domain and/or the second DNA-cleavage domain comprises an amino acid sequence of SEQ ID NOs: 10 or 11 and comprises a substitution of an aspartic acid residue at amino acid position number 450 to an alanine (which may be referred to as D450A). In some embodiments, the first DNA-cleavage domain and/or the second DNA-cleavage domain comprises a substitution of an amino acid residue corresponding to the aspartic acid residue at amino acid position number 450 (which may be referred to as D450) of SEQ ID NO: 10. Exemplary FokI and FokI cleavage domain sequences are provided with the aspartic acid residue at position 450 is indicated in boldface with underline, in SEQ ID NO: 10 and 11 below.

Exemplary amino acid sequence of FokI
(SEQ ID NO: 10)
MVSKIRTFGWVQNPGKFENLKRVVQVFDRNSKVHNEVKNIKIPTLVKESK
IQKELVAIMNQHDLIYTYKELVGTGTSIRSEAPCDAIIQATIADQGNKKG
YIDNWSSDGFLRWAHALGFIEYINKSDSFVITDVGLAYSKSADGSAIEKE
ILIEAISSYPPAIRILTLLEDGQHLTKFDLGKNLGFSGESGFTSLPEGIL
LDTLANAMPKDKGEIRNNWEGSSDKYARMIGGWLDKLGLVKQGKKEFIIP
TLGKPDNKEFISHAFKITGEGLKVLRRAKGSTKFTRVPKRVYWEMLATNL
TDKEYVRTRRALILEILIKAGSLKIEQIQDNLKKLGEDEVIETIENDIKG
LINTGIFIEIKGRFYQLKDHILQFVIPNRGVTKQLVKSELEEKKSELRHK
LKYVPHEYIELIEIARNSTQDRILEMKVMEFFMKVYGYRGKHLGGSRKPD
GAIYTVGSPIDYGVIVDTKAYSGGYNLPIGQADEMQRYVEENQTRNKHIN
PNEWWKVYPSSVTEFKFLFVSGHFKGNYKAQLTRLNHITNCNGAVLSVEE
LLIGGEMIKAGTLTLEEVRRKENNGEINF
Exemplary amino acid sequence of FokI DNA cleavage
domain
(SEQ ID NO: 11):
QLVKSELEEKKSELRHKLKYVPHEYIELIEIARNSTQDRILEMKVMEFFM
KVYGYRGKHLGGSRKPDGAIYTVGSPIDYGVIVDTKAYSGGYNLPIGQAD
EMQRYVEENQTRNKHINPNEWWKVYPSSVTEFKFLFVSGHFKGNYKAQLT
RLNHITNCNGAVLSVEELLIGGEMIKAGILTLEEVRRKENNGEINF
Exemplary amino acid sequence of FokI DNA cleavage
domain mutant (D450A)
(SEQ ID NO: 12)
QLVKSELEEKKSELRHKLKYVPHEYIELIEIARNSTQDRILEMKVMEFFM
KVYGYRGKHLGGSRKPAGAIYTVGSPIDYGVIVDTKAYSGGYNLPIGQAD
EMQRYVEENQTRNKHINPNEWWKVYPSSVTEFKFLFVSGHFKGNYKAQLT
RLNHITNCNGAVLSVEELLIGGEMIKAGTLTLEEVRRKENNGEINF

Exemplary amino acid sequence of an endonuclease domain comprising a FokI nickase (FokI DNA cleavage domain mutant (D450A) and FokI DNA cleavage domain separated by linker) (SEQ ID NO: 13). The first FokI DNA cleavage domain is shown in underline, a polypeptide linker is shown in italics, and a second FokI DNA cleavage domain is shown in boldface. The D450A mutation is shown in the first FokI DNA cleavage domain in boldface with double underline.

(SEQ ID NO: 13)
QLVKSELEEKKSELRHKLKYVPHEYIELIEIARNSTQDRILEMKVMEFFM
KVYGYRGKHLGGSRKPAGAIYTVGSPIDYGVIVDTKAYSGGYNLPIGQAD
EMQRYVEENQTRNKHINPNEWWKVYPSSVTEFKFLFVSGHFKGNYKAQLT
RLNHITNCNGAVLSVEELLIGGEMIKAGILTLEEVRRKENNGEINFGSGS
GSGSITRTTNPRNVVPKIYMSAGSIPLTTHITNSIQPTLWTIGSINGVAP
LAKSIKLGIPVTGSAYTDQTTAMVRKKVSVFMGSGSGSGSSQLVKSELEE
KKSELRHKLKYVPHEYIELIEIARNSTQDRILEMKVMEFFMKVYGYRGKH
LGGSRKPDGAIYTVGSPIDYGVIVDTKAYSGGYNLPIGQADEMQRYVEEN
QTRNKHINPNEWWKVYPSSVTEFKFLFVSGHFKGNYKAQLTRLNHITNCN
GAVLSVEELLIGGEMIKAGTLTLEEVRRKENNGEINF

Exemplary amino acid sequence of an endonuclease domain comprising a FokI nickase (FokI DNA cleavage domain and FokI DNA cleavage domain mutant (D450A) separated by a linker) (SEQ ID NO: 14). The first FokI DNA cleavage domain is shown in underline, a polypeptide linker is shown in italics, and a second FokI DNA cleavage domain is shown in boldface. The D450A mutation is shown in the second FokI DNA cleavage domain in boldface with underline.

(SEQ ID NO: 14)
QLVKSELEEKKSELRHKLKYVPHEYIELIEIARNSTQDRILEMKVMEFFM
KVYGYRGKHLGGSRKPDGAIYTVGSPIDYGVIVDTKAYSGGYNLPIGQAD
EMQRYVEENQTRNKHINPNEWWKVYPSSVTEFKFLFVSGHFKGNYKAQLT
RLNHITNCNGAVLSVEELLIGGEMIKAGILTLEEVRRKENNGEINFGSGS
GSGSITRTTNPRNVVPKIYMSAGSIPLTTHITNSIQPTLWTIGSINGVAP
LAKSIKLGIPVTGSAYTDQTTAMVRKKVSVFMGSGSGSGSSQLVKSELEE
KKSELRHKLKYVPHEYIELIEIARNSTQDRILEMKVMEFFMKVYGYRGKH
LGGSRKPAGAIYTVGSPIDYGVIVDTKAYSGGYNLPIGQADEMQRYVEEN
QTRNKHINPNEWWKVYPSSVTEFKFLFVSGHFKGNYKAQLTRLNHITNCN
GAVLSVEELLIGGEMIKAGTLTLEEVRRKENNGEINF

### Genomic Modification Domain

In some embodiments, the fusion polypeptides described herein comprise a Cpf1 domain that lacks nuclease activity, an endonuclease domain, and a genomic modification domain. As used herein, a “genomic modification domain” refers to an enzyme, or portion thereof, that is capable of effecting a modification on the genome of a host cell. Examples of genomic modification domains, including epigenetic modifiers (e.g. a DNA methyltransferase, a DNA methylase, a histone acetyltransferase, a histone deacetylase, a histone methyltransferase, a histone methylase, or a functional portion or combination of any thereof) and enzyme that modify nucleic acids or polynucleotides, and/or act on nucleic acids or polynucleotides, such as helicases, polymerases, nucleases, ligases, transcription factors.

In some embodiments, the genomic modification domain comprises a base editor, which may refer to an enzyme or portion thereof that modifies a nucleobase of a polynucleotide. In some embodiments, the genomic modification domain comprises more than one base editor, or base editing domain. In some embodiments, the genomic modification domain comprises a deaminase enzyme, or portion thereof, which is capable of catalyzing a deamination reaction. In general, a deaminase, such as a cytosine or adenosine deaminase, target and deaminates a specific nucleobase, e.g., a cytosine or adenosine nucleobase of a C or A nucleotide. In methods of “base editing” deamination of a specific nucleobase, via cellular mismatch repair mechanisms, results in a change from a C to a T nucleotide, or a change from an A to a G nucleotide. See, e.g., Komor et al. Nature (2016) 533: 420-424; Rees et al. Nat. Rev. Genet. (2018) 19(12): 770-788; Anzalone et al. Nat. Biotechnol. (2020) 38: 824-844.

Base editors typically comprise a catalytically inactive Cas nuclease fused to a functional domain, e.g., a deaminase domain. See, e.g., Eid et al. Biochem. J. (2018) 475(11): 1955-1964; Rees et al. Nature Reviews Genetics (2018) 19:770-788. The fusion polypeptides described herein comprise Cpf1 domain lacking nuclease activity, an endonuclease domain, and a genomic modification domain, which may be a base editing domain (e.g., a deaminase). In some embodiments, the fusion polypeptide comprises a cytidine deaminase, or portion thereof. Such fusion polypeptides may be referred to as cytosine base editors (CBE). In general, a cytidine deaminase catalyzes the hydrolysis of cytidine or deoxycytidine to uridine or deoxyuridine. In some embodiments, the cytidine deaminase catalyzes the hydrolysis of cytosine to uracil.

In some embodiments, the fusion polypeptide comprises an adenine deaminase, or portion thereof. Such fusion polypeptides may be referred to as adenine base editors (ABE). In general, an adenosine deaminase catalyzes the deamination of adenine in a deoxyadenosine residue. In some embodiments, the adenine deaminase catalyzes conversion of adenosine to inosine. In some embodiments, the adenine deaminase is a tRNA adenosine deaminase (TadA) or a variant thereof (e.g., an evolved variant such as TadA2.1).

In some embodiments, the fusion polypeptide comprises an adenine deaminase and a cytidine deaminase, or portions thereof. Such fusion polypeptides may be referred to as adenine and cytosine base editors.

In some embodiments, the fusion polypeptide comprises, from N-terminus to C-terminus, the Cas nuclease, the endonuclease domain, the adenine deaminase, and the cytidine deaminase. In some embodiments, the fusion polypeptide comprises, from N-terminus to C-terminus, the Cas nuclease, the endonuclease domain, the cytidine deaminase, and the adenine deaminase. In some embodiments, the fusion polypeptide comprises, from N-terminus to C-terminus, the Cas nuclease, the adenine deaminase, the endonuclease domain, and the cytidine deaminase. In some embodiments, the fusion polypeptide comprises, from N-terminus to C-terminus, the Cas nuclease, the adenine deaminase, the cytidine deaminase, and the endonuclease domain. In some embodiments, the fusion polypeptide comprises, from N-terminus to C-terminus, the Cas nuclease, the cytidine deaminase, the endonuclease domain, and the adenine deaminase. In some embodiments, the fusion polypeptide comprises, from N-terminus to C-terminus, the Cas nuclease, the cytidine deaminase, the adenine deaminase, and the endonuclease domain. In some embodiments, the fusion polypeptide comprises, from N-terminus to C-terminus, the endonuclease domain, the Cas nuclease, the adenine deaminase, and the cytidine deaminase. In some embodiments, the fusion polypeptide comprises, from N-terminus to C-terminus, the endonuclease domain, the Cas nuclease, the cytidine deaminase, and the adenine deaminase. In some embodiments, the fusion polypeptide comprises, from N-terminus to C-terminus, the endonuclease domain, the adenine deaminase, the Cas nuclease, and the cytidine deaminase. In some embodiments, the fusion polypeptide comprises, from N-terminus to C-terminus, the endonuclease domain, the adenine deaminase, the cytidine deaminase, and the Cas nuclease. In some embodiments, the fusion polypeptide comprises, from N-terminus to C-terminus, the endonuclease domain, the cytidine deaminase, the Cas nuclease, and the adenine deaminase. In some embodiments, the fusion polypeptide comprises, from N-terminus to C-terminus, the endonuclease domain, the cytidine deaminase, the adenine deaminase, and the Cas nuclease. In some embodiments, the fusion polypeptide comprises, from N-terminus to C-terminus, the adenine deaminase, the Cas nuclease, the endonuclease domain, and the cytidine deaminase. In some embodiments, the fusion polypeptide comprises, from N-terminus to C-terminus, the adenine deaminase, the Cas nuclease, the cytidine deaminase, and the endonuclease domain. In some embodiments, the fusion polypeptide comprises, from N-terminus to C-terminus, the adenine deaminase, the endonuclease domain, the Cas nuclease, and the cytidine deaminase. In some embodiments, the fusion polypeptide comprises, from N-terminus to C-terminus, the adenine deaminase, the endonuclease domain, the cytidine deaminase, and the Cas nuclease. In some embodiments, the fusion polypeptide comprises, from N-terminus to C-terminus, the adenine deaminase, the cytidine deaminase, the Cas nuclease, and the endonuclease domain. In some embodiments, the fusion polypeptide comprises, from N-terminus to C-terminus, the adenine deaminase, the cytidine deaminase, the endonuclease domain, and the Cas nuclease. In some embodiments, the fusion polypeptide comprises, from N-terminus to C-terminus, the cytidine deaminase, the Cas nuclease, the endonuclease domain, and the adenine deaminase. In some embodiments, the fusion polypeptide comprises, from N-terminus to C-terminus, the cytidine deaminase, the Cas nuclease, the adenine deaminase, and the endonuclease domain. In some embodiments, the fusion polypeptide comprises, from N-terminus to C-terminus, the cytidine deaminase, the endonuclease domain, the Cas nuclease, and the adenine deaminase. In some embodiments, the fusion polypeptide comprises, from N-terminus to C-terminus, the cytidine deaminase, the endonuclease domain, the adenine deaminase, and the Cas nuclease. In some embodiments, the fusion polypeptide comprises, from N-terminus to C-terminus, the cytidine deaminase, the adenine deaminase, the endonuclease domain, and the Cas nuclease. In some embodiments, the fusion polypeptide comprises, from N-terminus to C-terminus, the cytidine deaminase, the adenine deaminase, the Cas nuclease, and the endonuclease domain.

Cytidine deaminases and/or adenosine deaminases for use in the fusion polypeptides described herein may be obtained from any source known in the art. For example, in some embodiments, the cytidine deaminase and/or adenosine deaminase, or portion thereof, is from a naturally occurring deaminase or is a variant of a naturally occurring deaminase. In some embodiments, the cytidine deaminase and/or adenosine deaminase, or portion thereof, is an engineered or synthetic deaminase that is not naturally occurring.

Additional examples of suitable genomic modification domains for use in the fusion polypeptides described herein may be found, without limitation, in the exemplary base editors: BE1, BE2, BE3, HF-BE3, BE4, BE4max, AncBE4max, BE4-Gam, YE1-BE3, EE-BE3, YE2-BE3, YEE-CE3, VQR-BE3, VRER-BE3, SaBE3, SaBE4, SaBE4-Gam, Sa(KKH)-BE3, Target-AID, Target-AID-NG, AID, CDA1, APOBEC-1, APOBEC3G, xBE3, eA3A-BE3, BE-PLUS, TAM, CRISPR-X, ABE7.9, ABE7.10, ABE7.10*, xABE, ABESa, VQR-ABE, VRER-ABE, Sa(KKH)-ABE, and CRISPR-SKIP. Additional examples of base editors can be found, for example, in U.S. Publication No. 2018/0312825A1, U.S. Publication No. 2018/0312828A1, and PCT Publication No. WO 2018/165629A1, which are incorporated by reference herein in their entireties. In some embodiments, the genomic modification is a cytosine deaminase, such as APOBEC (also referred to as “apolipoprotein B editing complex catalytic subunit 1,” APOBEC-1), pmCDA1, or activation-induced cytidine deaminase (AID). In some embodiments, the genomic modification is an adenine deaminase, such as TadA. In some embodiments, the endonuclease comprises an uracil glycosylase inhibitor (UGI). In some embodiments, the endonuclease comprises an adenine base editor (ABE), for example an ABE evolved from the RNA adenine deaminase TadA.

In some embodiments, the genomic modification domain comprises an amino acid sequence of SEQ ID NOs: 15, or a sequence with at least 80, 85, 90, 95, or 99% identity to any thereof.

Exemplary amino acid sequence of APOBEC-1
(SEQ ID NO: 15)
GSSETGPVAVDPTLRRRIEPHEFEVFFDPRELRKETCLLYEINWGGRHSI
WRHTSQNTNKHVEVNFIEKFTTERYFCPNTRCSITWFLSWSPCGECSRAI
TEFLSRYPHVTLFIYIARLYHHADPRNRQGLRDLISSGVTIQIMTEQESG
YCWRNFVNYSPSNEAHWPRYPHLWVRLYVLELYCIILGLPPCLNILRRKQ
PQLTFFTIALQSCHYQRLPPHILWATGLK

### Linker Domains

Any of the fusion polypeptides described herein may further comprises one or more linker domains. A linker domain is an amino acid sequence by which two polypeptide domains may be joined. In general, a linker domain may be used, for example, to join adjacent domains or functional regions of a polypeptide and may allow a level of flexibility (or rigidity) such that the joined domains or regions are independently functional.

Exemplary linker domains are recited, for example, in Chen, et al, Adv Drug Deliv Rev (2013) Oct. 15 65(10): 1357-1369, however, one of skill in the art would not be limited by this disclosure. The linker may comprise any suitable amino acid sequence. In some embodiments, the linker domain is a flexible linker. Flexible linkers typically largely comprise small and/or polar amino acids, such as glycine (Gly) and serine (Ser) or threonine (Thr), respectively. This promotes flexibility and solubility in the resultant fusion polypeptide. Example flexible linker domains include, but are not limited to, glycine linkers (e.g., (Gly)s linkers) (SEQ ID NO: 54), serine linkers, glycine-serine linkers (e.g., (Gly-Gly-Gly-Ser). (SEQ ID NO: 55) and (Gly-Gly-Gly-Gly-Ser)₄ (SEQ ID NO: 56) linkers), and glycine-serine rich linkers (e.g., KESGSVSSEQLAQFRSLD (SEQ ID NO: 16), EGKSSGSGSESKST (SEQ ID NO: 17), and GSAGSAAGSGEF(SEQ ID NO: 18)).

In some embodiments, the linker domain is a Gly/Ser linker from about 1 to about 100, from about 3 to about 20, from about 5 to about 30, from about 5 to about 18, or from about 3 to about 8 amino acids in length and consists of glycine and/or serine residues in sequence. Accordingly, the Gly/Ser linker may consist of glycine and/or serine residues. Preferably, the Gly/Ser linker comprises the amino acid sequence of GGGGS (SEQ ID NO: 19), and multiple SEQ ID NO: 19 may be present within the linker. Any linker sequence may be used as a spacer between any two domains or functional regions of any of the fusion polypeptides described herein, such as between the Cpf1 domain and the endonuclease domain, and/or between a first DNA-cleavage domain and a second DNA-cleavage domain. In some, embodiments, the region linker is ([G]x[S]y)z(SEQ ID NO: 57), for example wherein x can be 1-10, 7 can be 1-3, and z can be 1-5. In some embodiments, the linker region comprises the amino acid sequence GGGGSGGGGS (SEQ ID NO: 20). In some embodiments, the linker region comprises the amino acid sequence GGGGSGGGGSGGGGS (SEQ ID NO: 21).

In some embodiments, the linker is an XTEN linker, which is an unstructured polypeptide consisting of hydrophilic residues of varying lengths. Amino acid sequences of XTEN peptides will be evident to one of skill in the art and can be found, for example, in U.S. Pat. No. 8,673,860, which is herein incorporated by reference. In some embodiments, the XTEN linker is provided by SEQ ID NO: 22.

Amino acid sequence of XTEN linker
(SEQ ID NO: 22)
SGSETPGTSESATPES
Amino acid sequence of exemplary linker domain
(SEQ ID NO: 23)
GSGSGSGSITRTTNPRNVVPKIYMSAGSIPLTTHITNSIQPTLWTIGSIN
GVAPLAKSIKLGIPVTGSAYTDQTTAMVRKKVSVFMGSGSGSGSS

In some embodiments, the linker domain is a rigid linker. Non-limiting examples of rigid linkers are known in the art and can be found, for example, in Tan, et al. Nat. Commun. (2019) 10: 439. Rigid linkers often include proline (Pro) residues, which contribute to rigidity of a protein sequence because the contain a secondary amine.

### Fusion Polypeptides

The domains described herein may be arranged in any order (from N-terminus to C-terminus) in a fusion polypeptides described herein, such that each of the domains is capable of performing its respective function.

In some embodiments, a fusion polypeptide described herein may comprise a Cpf1 domain that is located at the N-terminus of the endonuclease domain. In some embodiments, the endonuclease domain comprises a DNA-cleavage domain and the Cpf1 domain that is located N-terminal of the DNA-cleavage domain. In some embodiments, the endonuclease domain comprises a first DNA-cleavage domain and a second DNA-cleavage domain, and the Cpf1 domain that is located N-terminal of both the first and second DNA-cleavage domains.

In some embodiments, a fusion polypeptide described herein may comprise an endonuclease domain that is located at the N-terminus of the Cpf1 domain. In some embodiments, the endonuclease domain comprises a DNA-cleavage domain, and the DNA-cleavage domain that is located N-terminal of the Cpf1 domain. In some embodiments, the endonuclease domain comprises a first DNA-cleavage domain and a second DNA-cleavage domain, and both the first and second DNA-cleavage domain are located N-terminal of the Cpf1 domain.

Any of the fusion polypeptides described herein may further comprise a genomic modification domain. In some embodiments, a fusion polypeptide described herein may comprise a genomic modification domain that is located N-terminal of the Cpf1 domain. In some embodiments, a fusion polypeptide described herein may comprise a genomic modification domain that is located N-terminal of the endonuclease domain. In some embodiments, a fusion polypeptide described herein may comprise a genomic modification that is located between the Cpf1 domain and the endonuclease domain.

In some embodiments, the fusion polypeptide comprises, from N-terminus to C-terminus, the Cpf1 domain, the endonuclease domain, and the genomic modification domain. In some embodiments, the fusion polypeptide comprises, from N-terminus to C-terminus, the Cpf1 domain, the genomic modification domain, and the endonuclease domain. In some embodiments, the fusion polypeptide comprises from, N-terminus to C-terminus, the endonuclease domain, the Cpf1 domain, and the genomic modification domain. In some embodiments, the fusion polypeptide comprises, from N-terminus to C-terminus, the endonuclease domain, the genomic modification domain, and the Cpf1 domain. In some embodiments, the fusion polypeptide comprises, from N-terminus to C-terminus, the genomic modification domain, the Cpf1 domain, and the endonuclease domain. In some embodiments, the fusion polypeptide comprises, from N-terminus to C-terminus, the genomic modification domain, the endonuclease domain, and the Cpf1 domain.

In some embodiments, the fusion polypeptide comprises, from N-terminus to C-terminus, the Cpf1 domain comprising any of SEQ ID NOs: 3 or 5, the endonuclease domain, and the genomic modification domain. In some embodiments, the fusion polypeptide comprises, from N-terminus to C-terminus, the Cpf1 domain comprising any of SEQ ID NOs: 3 or 5, the genomic modification domain, and the endonuclease domain. In some embodiments, the fusion polypeptide comprises from, N-terminus to C-terminus, the endonuclease domain, the Cpf1 domain comprising any of SEQ ID NOs: 3 or 5, and the genomic modification domain. In some embodiments, the fusion polypeptide comprises, from N-terminus to C-terminus, the endonuclease domain, the genomic modification domain, and the Cpf1 domain comprising any of SEQ ID NOs: 3 or 5. In some embodiments, the fusion polypeptide comprises, from N-terminus to C-terminus, the genomic modification domain, the Cpf1 domain comprising any of SEQ ID NOs: 3 or 5, and the endonuclease domain. In some embodiments, the fusion polypeptide comprises, from N-terminus to C-terminus, the genomic modification domain, the endonuclease domain, and the Cpf1 domain comprising any of SEQ ID NOs: 3 or 5.

In some embodiments, the fusion polypeptide comprises, from N-terminus to C-terminus, the Cpf1 domain, the endonuclease domain, and the deamination domain. In some embodiments, the fusion polypeptide comprises, from N-terminus to C-terminus, the Cpf1 domain, a deamination domain, and the endonuclease domain. In some embodiments, the fusion polypeptide comprises from, N-terminus to C-terminus, the endonuclease domain, the Cpf1 domain, and the deamination domain. In some embodiments, the fusion polypeptide comprises, from N-terminus to C-terminus, the endonuclease domain, the deamination domain, and the Cpf1 domain. In some embodiments, the fusion polypeptide comprises, from N-terminus to C-terminus, a deamination domain, the Cpf1 domain, and the endonuclease domain. In some embodiments, the fusion polypeptide comprises, from N-terminus to C-terminus, the deamination domain, the endonuclease domain, and the Cpf1 domain.

In some embodiments, the fusion polypeptide comprises, from N-terminus to C-terminus, the Cpf1 domain comprising any of SEQ ID NOs: 3 or 5, the endonuclease domain, and the deamination domain. In some embodiments, the fusion polypeptide comprises, from N-terminus to C-terminus, the Cpf1 domain comprising any of SEQ ID NOs: 3 or 5, the deamination domain, and the endonuclease domain. In some embodiments, the fusion polypeptide comprises from, N-terminus to C-terminus, the endonuclease domain, the Cpf1 domain comprising any of SEQ ID NOs: 3 or 5, and the deamination domain. In some embodiments, the fusion polypeptide comprises, from N-terminus to C-terminus, the endonuclease domain, the deamination domain, and the Cpf1 domain comprising any of SEQ ID NOs: 3 or 5. In some embodiments, the fusion polypeptide comprises, from N-terminus to C-terminus, the deamination domain, the Cpf1 domain comprising any of SEQ ID NOs: 3 or 5, and the endonuclease domain. In some embodiments, the fusion polypeptide comprises, from N-terminus to C-terminus, the deamination domain, the endonuclease domain, and the Cpf1 domain comprising any of SEQ ID NOs: 3 or 5.

Any of the fusion polypeptides described herein may further comprise one or more linker domains. In some embodiments, the fusion polypeptide comprises a linker domain between the Cpf1 domain and the endonuclease domain. In some embodiments, the fusion polypeptide comprises a linker domain between the Cpf1 domain and the genomic modification domain. In some embodiments, the fusion polypeptide comprises a linker domain between the endonuclease domain and the genomic modification domain.

In some embodiments, the endonuclease domain comprises a linker domain between a first DNA-cleavage domain and a second DNA-cleavage domain.

Amino acid sequences of exemplary fusion polypeptides of the present disclosure are provided below.

### 1. Construct A

An exemplary fusion polypeptide, as described herein, comprises a first FokI DNA cleavage domain, a polypeptide linker, a second FokI DNA cleavage domain comprising a D450A mutation, an XTEN linker, and a Cpf1 domain that lacks nuclease activity.

In some embodiments, the fusion polypeptide comprises an amino acid sequence shown in SEQ ID NO: 24, or an amino acid sequence that is at least 70%, at least 75%, at least 80%, at least 85%, at least 90%, at least 95%, or at least 99% identical to the amino acid sequence shown in SEQ ID NO: 24. In SEQ ID NO: 24 below, the first FokI DNA cleavage domain is shown in underline, the polypeptide linker is shown in italics, the second FokI DNA cleavage domain containing an D450A mutation is shown in underline (with mutation indicated in boldface), the XTEN linker shown in italics, and the AsCpf1 lacking nuclease activity is shown in boldface. ‘FokII’, as used in sequence descriptions herein, refers to the second FokI DNA cleavage domain in an exemplary construct (from N to C), regardless of the presence or absence of a mutation in the first or second FokI DNA cleavage domains.

Amino acid sequence of Construct A: FokI (D450)-polypeptide linker-FokII (D450A)-
XTEN linker-AsCpf1 (A)
(SEQ ID NO: 24)
MQLVKSELEEKKSELRHKLKYVPHEYIELIEIARNSTQDRILEMKVMEFFMKVYGYRGKHLGGSRKPDGAIYTVG
SPIDYGVIVDTKAYSGGYNLPIGQADEMQRYVEENQTRNKHINPNEWWKVYPSSVTEFKFLFVSGHFKGNYKAQL
TRLNHITNCNGAVLSVEELLIGGEMIKAGTLTLEEVRRKFNNGEINFGSGSGSGSITRTINPRNVVPKIYMSAGS
IPLTTHITNSIQPTLWTIGSINGVAPLAKSIKLGIPVTGSAYTDQTTAMVRKKVSVFMGSGSGSGSSQLVKSELE
EKKSELRHKLKYVPHEYIELIEIARNSTQDRILEMKVMEFFMKVYGYRGKHLGGSRKPAGAIYTVGSPIDYGVIV
DTKAYSGGYNLPIGQADEMQRYVEENQTRNKHINPNEWWKVYPSSVTEFKFLFVSGHFKGNYKAQLTRINHITNC
NGAVLSVEELLIGGEMIKAGTLTLEEVRRKFNNGEINFSGSETPGTSESATPESTQFEGFTNLYQVSKTLRFELI
PQGKTLKHIQEQGFIEEDKARNDHYKELKPIIDRIYKTYADQCLQLVQLDWENLSAAIDSYRKEKTEETRNALIE
EQATYRNAIHDYFIGRTDNLTDAINKRHAEIYKGLFKAELFNGKVLKQLGTVTTTEHENALLRSFDKFTTYFSGF
YRNRKNVFSAEDISTAIPHRIVQDNFPKFKENCHIFTRLITAVPSLREHFENVKKAIGIFVSTSIEEVFSFPFYN
QLLTQTQIDLYNQLLGGISREAGTEKIKGLNEVLNLAIQKNDETAHIIASLPHRFIPLFKQILSDRNTLSFILEE
FKSDEEVIQSFCKYKTLLRNENVLETAEALFNELNSIDLTHIFISHKKLETISSALCDHWDTLRNALYERRISEL
TGKITKSAKEKVQRSLKHEDINLQEIISAAGKELSEAFKQKTSEILSHAHAALDQPLPTTLKKQEEKEILKSQLD
SLLGLYHLLDWFAVDESNEVDPEFSARLTGIKLEMEPSLSFYNKARNYATKKPYSVEKFKLNFQMPTLARGWDVN
VEKNRGAILFVKNGLYYLGIMPKQKGRYKALSFEPTEKTSEGFDKMYYDYFPDAAKMIPKCSTQLKAVTAHFQTH
TTPILLSNNFIEPLEITKEIYDLNNPEKEPKKFQTAYAKKTGDQKGYREALCKWIDFTRDFLSKYTKTTSIDLSS
LRPSSQYKDLGEYYAELNPLLYHISFQRIAEKEIMDAVETGKLYLFQIYNKDFAKGHHGKPNLHTLYWTGLFSPE
NLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKFFFHVPITLNYQAANSPSKENQRVNAYLKEHPETPIIGIARGERNLIYITVIDS
TGKILEQRSLNTIQQFDYQKKLDNREKERVAARQAWSVVGTIKDLKQGYLSQVIHEIVDLMIHYQAVVVLENLNF
GFKSKRTGIAEKAVYQQFEKMLIDKLNCLVLKDYPAEKVGGVLNPYQLTDQFTSFAKMGTQSGFLFYVPAPYTSK
IDPLTGFVDPFVWKTIKNHESRKHFLEGFDFLHYDVKTGDFILHFKMNRNLSFQRGLPGFMPAWDIVFEKNETQF
DAKGTPFIAGKRIVPVIENHRFTGRYRDLYPANELIALLEEKGIVERDGSNILPKLLENDDSHAIDTMVALIRSV
LQMRNSNAATGEDYINSPVRDLNGVCFDSRFQNPEWPMDADANGAYHIALKGQLLLNHLKESKDLKLQNGISNQD
WLAYIQELRN

### 2. Construct B

An exemplary fusion polypeptide, as described herein, comprises an APOBEC-1 base editor, a Cpf1 domain that lacks nuclease activity, an XTEN linker, a first FokI DNA cleavage domain comprising a D450A mutation, a polypeptide linker, and a second FokI DNA cleavage domain.

In some embodiments, the fusion polypeptide comprises an amino acid sequence shown in SEQ ID NO: 25, or an amino acid sequence that is at least 70%, at least 75%, at least 80%, at least 85%, at least 90%, at least 95%, or at least 99% identical to the amino acid sequence shown in SEQ ID NO: 25. In SEQ ID NO: 25 below, the APOBEC-1 base editor is shown in underline, a linker sequence is shown in italics, the AsCpf1 lacking nuclease activity is shown in boldface, the XTEN linker shown in italics, first FokI DNA cleavage domain containing an D450A mutation is shown in underline (with mutation indicated in boldface), the polypeptide linker is shown in italics, the second FokI DNA cleavage domain is shown in underline.

Amino acid sequence of Construct B: APOBEC Base Editor-AsCpf1 (D908A)-XTEN-
FokI (D450A)-polypeptide linker-FokII (D450)
(SEQ ID NO: 25)
GSSETGPVAVDPTLRRRIEPHEFEVFFDPRELRKETCLLYEINWGGRHSIWRHTSQNTNKHVEVNFIEKFTTERY
FCPNTRCSITWFLSWSPCGECSRAITEFLSRYPHVTLFIYIARLYHHADPRNRQGLRDLISSGVTIQIMTEQESG
YCWRNFVNYSPSNEAHWPRYPHLWVRLYVLELYCIILGLPPCLNILRRKQPQLTFFTIALQSCHYQRLPPHILWA
TGLKSGGSSGGSSGSETPGTSESATPESSGGSSGGSTQFEGFTNLYQVSKTLRFELIPQGKTLKHIQEQGFIEED
KARNDHYKELKPIIDRIYKTYADQCLQLVQLDWENLSAAIDSYRKEKTEETRNALIEEQATYRNAIHDYFIGRTD
NLTDAINKRHAEIYKGLFKAELFNGKVLKQLGTVTTTEHENALLRSFDKFTTYFSGFYRNRKNVFSAEDISTAIP
HRIVQDNFPKFKENCHIFTRLITAVPSLREHFENVKKAIGIFVSTSIEEVFSFPFYNQLLTQTQIDLYNQLLGGI
SREAGTEKIKGLNEVLNLAIQKNDETAHIIASLPHRFIPLFKQILSDRNTLSFILEEFKSDEEVIQSFCKYKTLL
RNENVLETAEALFNELNSIDLTHIFISHKKLETISSALCDHWDTLRNALYERRISELTGKITKSAKEKVQRSLKH
EDINLQEIISAAGKELSEAFKQKTSEILSHAHAALDQPLPTTLKKQEEKEILKSQLDSLLGLYHLLDWFAVDESN
EVDPEFSARLTGIKLEMEPSLSFYNKARNYATKKPYSVEKFKLNFQMPTLARGWDVNVEKNRGAILFVKNGLYYL
GIMPKQKGRYKALSFEPTEKTSEGFDKMYYDYFPDAAKMIPKCSTQLKAVTAHFQTHTTPILLSNNFIEPLEITK
EIYDLNNPEKEPKKFQTAYAKKTGDQKGYREALCKWIDFTRDFLSKYTKTTSIDLSSLRPSSQYKDLGEYYAELN
PLLYHISFQRIAEKEIMDAVETGKLYLFQIYNKDFAKGHHGKPNLHTLYWTGLFSPENLAKTSIKLNGQAELFYR
PKSRMKRMAHRLGEKMLNKKLKDQKTPIPDTLYQELYDYVNHRLSHDLSDEARALLPNVITKEVSHEIIKDRRFT
SDKFFFHVPITLNYQAANSPSKENQRVNAYLKEHPETPIIGIARGERNLIYITVIDSTGKILEQRSLNTIQQFDY
QKKLDNREKERVAARQAWSVVGTIKDLKQGYLSQVIHEIVDLMIHYQAVVVLENLNFGFKSKRTGIAEKAVYQQF
EKMLIDKLNCLVLKDYPAEKVGGVLNPYQLTDQFTSFAKMGTQSGFLFYVPAPYTSKIDPLTGFVDPFVWKTIKN
HESRKHFLEGFDFLHYDVKTGDFILHFKMNRNLSFQRGLPGFMPAWDIVFEKNETQFDAKGTPFIAGKRIVPVIE
NHRFTGRYRDLYPANELIALLEEKGIVERDGSNILPKLLENDDSHAIDTMVALIRSVLQMRNSNAATGEDYINSP
VRDLNGVCFDSRFQNPEWPMDADANGAYHIALKGQLLLNHLKESKDLKLQNGISNQDWLAYIQELRNSGSETPGT
SESATPESQLVKSELEEKKSELRHKLKYVPHEYIELIEIARNSTQDRILEMKVMEFFMKVYGYRGKHLGGSRKPA
GAIYTVGSPIDYGVIVDTKAYSGGYNLPIGQADEMQRYVEENQTRNKHINPNEWWKVYPSSVTEFKFLFVSGHFK
GNYKAQLTRLNHITNCNGAVLSVEELLIGGEMIKAGTLTLEEVRRKFNNGEINFGSGSGSGSITRTTNPRNVVPK
IYMSAGSIPLTTHITNSIQPTLWTIGSINGVAPLAKSIKLGIPVTGSAYTDQTTAMVRKKVSVFMGSGSGSGSSQ
LVKSELEEKKSELRHKLKYVPHEYIELIEIARNSTQDRILEMKVMEFFMKVYGYRGKHLGGSRKPDGAIYTVGSP
IDYGVIVDTKAYSGGYNLPIGQADEMQRYVEENQTRNKHINPNEWWKVYPSSVTEFKFLFVSGHFKGNYKAQLTR
LNHITNCNGAVLSVEELLIGGEMIKAGTLTLEEVRRKFNNGEINF

### 3. Construct C

An exemplary fusion polypeptide, as described herein, comprises an APOBEC-1 base editor, a MAD7™-based domain that lacks nuclease activity, an XTEN linker, a first FokI DNA cleavage domain comprising a D450A mutation, a polypeptide linker, and a second FokI DNA cleavage domain.

In some embodiments, the fusion polypeptide comprises an amino acid sequence shown in SEQ ID NO: 26, or an amino acid sequence that is at least 70%, at least 75%, at least 80%, at least 85%, at least 90%, at least 95%, or at least 99% identical to the amino acid sequence shown in SEQ ID NO: 26. In SEQ ID NO: 26 below, the APOBEC-1 base editor is shown in underline, a linker sequence is shown in italics, the Mad7™-based domain lacking nuclease activity is shown in boldface, the XTEN linker shown in italics, first FokI DNA cleavage domain containing an D450A mutation is shown in underline (with mutation indicated in boldface), the polypeptide linker is shown in italics, the second FokI DNA cleavage domain is shown in underline.

Amino acid sequence of Construct C: APOBEC Base Editor-MAD7TM(D908A)-XTEN-
FokI (D450A)-polypeptide linker-FokII (D450)
(SEQ ID NO: 26)
GSSETGPVAVDPTLRRRIEPHEFEVFFDPRELRKETCLLYEINWGGRHSIWRHTSQNTNKHVEVNFIEKFTTERY
FCPNTRCSITWFLSWSPCGECSRAITEFLSRYPHVTLFIYIARLYHHADPRNRQGLRDLISSGVTIQIMTEQESG
YCWRNFVNYSPSNEAHWPRYPHLWVRLYVLELYCIILGLPPCLNILRRKQPQLTFFTIALQSCHYQRLPPHILWA
TGLKSGGSSGGSSGSETPGTSESATPESSGGSSGGSNNGTNNFQNFIGISSLQKTLRNALIPTETTQQFIVKNGI
IKEDELRGENRQILKDIMDDYYRGFISETLSSIDDIDWTSLFEKMEIQLKNGDNKDTLIKEQTEYRKAIHKKFAN
DDRFKNMESAKLISDILPEFVIHNNNYSASEKEEKTQVIKLFSRFATSFKDYFRNRANCESADDISSSSCHRIVN
DNAEIFFSNALVYRRIVKSLSNDDINKISGDMKDSLKEMSLEEIYSYEKYGEFITQEGISFYNDICGKVNSFMNL
YCQKNKENKNLYKLQKLHKQILCIADTSYEVPYKFESDEEVYQSVNGELDNISSKHIVERLRKIGDNYNGYNLDK
IYIVSKFYESVSQKTYRDWETINTALEIHYNNILPGNGKSKADKVKKAVKNDLQKSITEINELVSNYKLCSDDNI
KAETYIHEISHILNNFEAQELKYNPEIHLVESELKASELKNVLDVIMNAFHWCSVFMTEELVDKDNNFYAELEEI
YDEIYPVISLYNLVRNYVTQKPYSTKKIKLNFGIPTLARGWSKSVEYSRNAIILMRDNLYYLGIFNAKNKPDKKI
IEGNTSENKGDYKKMIYNLLPGPNKMIPKVFLSSKTGVETYKPSAYILEGYKQNKHIKSSKDFDITFCHDLIDYF
KNCIAIHPEWKNFGFDFSDTSTYEDISGFYREVELQGYKIDWTYISEKDIDLLQEKGQLYLFQIYNKDESKKSTG
NDNLHTMYLKNLFSEENLKDIVLKLNGEAEIFFRKSSIKNPIIHKKGSILVNRTYEAEEKDQFGNIQIVRKNIPE
NIYQELYKYFNDKSDKELSDEAAKLKNVVGHHEAATNIVKDYRYTYDKYFLHMPITINFKANKTGFINDRILQYI
AKEKDLHVIGIARGERNLIYVSVIDTCGNIVEQKSFNIVNGYDYQIKLKQQEGARQIARKEWKEIGKIKEIKEGY
LSLVIHEISKMVIKYNAIIAMEDLSYGFKKGRFKVERQVYQKFETMLINKLNYLVFKDISITENGGLLKGYQLTY
IPDKLKNVGHQCGCIFYVPAAYTSKIDPTTGFVNIFKFKDLTVDAKREFIKKFDSIRYDSEKNLFCFTFDYNNFI
TQNTVMSKSSWSVYTYGVRIKRRFVNGRESNESDTIDITKDMEKTLEMTDINWRDGHDLRQDIIDYEIVQHIFEI
FRLTVQMRNSLSELEDRDYDRLISPVLNENNIFYDSAKAGDALPKDADANGAYCIALKGLYEIKQITENWKEDGK
FSRDKLKISNKDWEDFIQNKRYLSGSETPGTSESATPESQLVKSELEEKKSELRHKLKYVPHEYIELIEIARNST
QDRILEMKVMEFFMKVYGYRGKHLGGSRKPAGAIYTVGSPIDYGVIVDTKAYSGGYNLPIGQADEMQRYVEENQT
RNKHINPNEWWKVYPSSVTEFKFLFVSGHFKGNYKAQLTRLNHITNCNGAVLSVEELLIGGEMIKAGTLTLEEVR
RKFNNGEINFGSGSGSGSITRTTNPRNVVPKIYMSAGSIPLTTHITNSIQPTLWTIGSINGVAPLAKSIKLGIPV
TGSAYTDQTTAMVRKKVSVFMGSGSGSGSSQLVKSELEEKKSELRHKLKYVPHEYIELIEIARNSTQDRILEMKV
MEFFMKVYGYRGKHLGGSRKPDGAIYTVGSPIDYGVIVDTKAYSGGYNLPIGQADEMQRYVEENQTRNKHINPNE
WWKVYPSSVTEFKFLFVSGHFKGNYKAQLTRLNHITNCNGAVLSVEELLIGGEMIKAGTLTLEEVRRKFNNGEIN
F

### 4. Construct E

An exemplary fusion polypeptide, as described herein, comprises a Cpf1 domain that lacks nuclease activity, an XTEN linker, a first FokI DNA cleavage domain, a polypeptide linker, and a second FokI DNA cleavage domain.

In some embodiments, the fusion polypeptide comprises an amino acid sequence shown in SEQ ID NO: 27, or an amino acid sequence that is at least 70%, at least 75%, at least 80%, at least 85%, at least 90%, at least 95%, or at least 99% identical to the amino acid sequence shown in SEQ ID NO: 27. In SEQ ID NO: 27 below, the Cpf1 domain lacking nuclease activity is shown in boldface, the XTEN linker shown in italics, first FokI DNA cleavage domain is shown in underline, the polypeptide linker is shown in italics, the second FokI DNA cleavage domain is shown in underline.

Amino acid sequence of Construct E: Cpf1 (D908A)-XTEN-FokI- polypeptide linker-
FokII
(SEQ ID NO: 27)
TQFEGFTNLYQVSKTLRFELIPQGKTLKHIQEQGFIEEDKARNDHYKELKPIIDRIYKTYADQCLQLVQLDWENL
SAAIDSYRKEKTEETRNALIEEQATYRNAIHDYFIGRTDNLTDAINKRHAEIYKGLFKAELFNGKVLKQLGTVTT
TEHENALLRSFDKFTTYFSGFYRNRKNVFSAEDISTAIPHRIVQDNFPKFKENCHIFTRLITAVPSLREHFENVK
KAIGIFVSTSIEEVFSFPFYNQLLTQTQIDLYNQLLGGISREAGTEKIKGLNEVLNLAIQKNDETAHIIASLPHR
FIPLFKQILSDRNTLSFILEEFKSDEEVIQSFCKYKTLLRNENVLETAEALFNELNSIDLTHIFISHKKLETISS
ALCDHWDTLRNALYERRISELTGKITKSAKEKVQRSLKHEDINLQEIISAAGKELSEAFKQKTSEILSHAHAALD
QPLPTTLKKQEEKEILKSQLDSLLGLYHLLDWFAVDESNEVDPEFSARLTGIKLEMEPSLSFYNKARNYATKKPY
SVEKFKLNFQMPTLARGWDVNVEKNRGAILFVKNGLYYLGIMPKQKGRYKALSFEPTEKTSEGEDKMYYDYFPDA
AKMIPKCSTQLKAVTAHFQTHTTPILLSNNFIEPLEITKEIYDLNNPEKEPKKFQTAYAKKTGDQKGYREALCKW
IDFTRDFLSKYTKTTSIDLSSLRPSSQYKDLGEYYAELNPLLYHISFQRIAEKEIMDAVETGKLYLFQIYNKDFA
KGHHGKPNLHTLYWTGLFSPENLAKTSIKLNGQAELFYRPKSRMKRMAHRLGEKMLNKKLKDQKTPIPDTLYQEL
YDYVNHRLSHDLSDEARALLPNVITKEVSHEIIKDRRFTSDKFFFHVPITLNYQAANSPSKENQRVNAYLKEHPE
TPIIGIARGERNLIYITVIDSTGKILEQRSLNTIQQFDYQKKLDNREKERVAARQAWSVVGTIKDLKQGYLSQVI
HEIVDLMIHYQAVVVLENLNFGFKSKRTGIAEKAVYQQFEKMLIDKLNCLVLKDYPAEKVGGVLNPYQLTDQFTS
FAKMGTQSGFLFYVPAPYTSKIDPLTGFVDPFVWKTIKNHESRKHFLEGEDFLHYDVKTGDFILHFKMNRNLSFQ
RGLPGFMPAWDIVFEKNETQFDAKGTPFIAGKRIVPVIENHRFTGRYRDLYPANELIALLEEKGIVERDGSNILP
KLLENDDSHAIDTMVALIRSVLQMRNSNAATGEDYINSPVRDLNGVCFDSRFQNPEWPMDADANGAYHIALKGQL
LLNHLKESKDLKLQNGISNQDWLAYIQELRNSGSETPGTSESATPESQLVKSELEEKKSELRHKLKYVPHEYIEL
IEIARNSTQDRILEMKVMEFFMKVYGYRGKHLGGSRKPDGAIYTVGSPIDYGVIVDTKAYSGGYNLPIGQADEMQ
RYVEENQTRNKHINPNEWWKVYPSSVTEFKFLFVSGHFKGNYKAQLTRLNHITNCNGAVLSVEELLIGGEMIKAG
TLTLEEVRRKFNNGEINFGSGSGSGSITRTTNPRNVVPKIYMSAGSIPLTTHITNSIQPTLWTIGSINGVAPLAK
SIKLGIPVTGSAYTDQTTAMVRKKVSVEMGSGSGSGSSQLVKSELEEKKSELRHKLKYVPHEYIELIEIARNSTQ
DRILEMKVMEFFMKVYGYRGKHLGGSRKPDGAIYTVGSPIDYGVIVDTKAYSGGYNLPIGQADEMQRYVEENQTR
NKHINPNEWWKVYPSSVTEFKFLFVSGHFKGNYKAQLTRLNHITNCNGAVLSVEELLIGGEMIKAGTLTLEEVRR
KFNNGEINF

### 5. Construct F

An exemplary fusion polypeptide, as described herein, comprises a first FokI DNA cleavage domain, a polypeptide linker, and a second FokI DNA cleavage domain, an XTEN linker, and a Cpf1 domain that lacks nuclease activity.

In some embodiments, the fusion polypeptide comprises an amino acid sequence shown in SEQ ID NO: 28, or an amino acid sequence that is at least 70%, at least 75%, at least 80%, at least 85%, at least 90%, at least 95%, or at least 99% identical to the amino acid sequence shown in SEQ ID NO: 28. In SEQ ID NO: 28 below, the first FokI DNA cleavage domain is shown in underline, the polypeptide linker is shown in italics, the second FokI DNA cleavage domain is shown in underline, the XTEN linker shown in italics, and the Cpf1 domain lacking nuclease activity is shown in boldface.

Amino acid sequence of Construct F: FokI-polypeptide linker-FokII-XTEN-Cpf1
(D908A)
(SEQ ID NO: 28)
QLVKSELEEKKSELRHKLKYVPHEYIELIEIARNSTQDRILEMKVMEFFMKVYGYRGKHLGGSRKPDGAIYTVGS
PIDYGVIVDTKAYSGGYNLPIGQADEMQRYVEENQTRNKHINPNEWWKVYPSSVTEFKFLFVSGHFKGNYKAQLT
RLNHITNCNGAVLSVEELLIGGEMIKAGTLTLEEVRRKFNNGEINFGSGSGSGSITRTTNPRNVVPKIYMSAGSI
PLTTHITNSIQPTLWTIGSINGVAPLAKSIKLGIPVTGSAYTDQTTAMVRKKVSVFMGSGSGSGSSQLVKSELEE
KKSELRHKLKYVPHEYIELIEIARNSTQDRILEMKVMEFFMKVYGYRGKHLGGSRKPDGAIYTVGSPIDYGVIVD
TKAYSGGYNLPIGQADEMQRYVEENQTRNKHINPNEWWKVYPSSVTEFKFLFVSGHFKGNYKAQLTRLNHITNCN
GAVLSVEELLIGGEMIKAGTLTLEEVRRKFNNGEINFSGSETPGTSESATPESTQFEGFTNLYQVSKTLRFELIP
QGKTLKHIQEQGFIEEDKARNDHYKELKPIIDRIYKTYADQCLQLVQLDWENLSAAIDSYRKEKTEETRNALIEE
QATYRNAIHDYFIGRTDNLTDAINKRHAEIYKGLFKAELFNGKVLKQLGTVTTTEHENALLRSFDKFTTYFSGFY
RNRKNVFSAEDISTAIPHRIVQDNFPKFKENCHIFTRLITAVPSLREHFENVKKAIGIFVSTSIEEVFSFPFYNQ
LLTQTQIDLYNQLLGGISREAGTEKIKGLNEVLNLAIQKNDETAHIIASLPHRFIPLFKQILSDRNTLSFILEEF
KSDEEVIQSFCKYKTLLRNENVLETAEALFNELNSIDLTHIFISHKKLETISSALCDHWDTLRNALYERRISELT
GKITKSAKEKVQRSLKHEDINLQEIISAAGKELSEAFKQKTSEILSHAHAALDQPLPTTLKKQEEKEILKSQLDS
LLGLYHLLDWFAVDESNEVDPEFSARLTGIKLEMEPSLSFYNKARNYATKKPYSVEKFKLNFQMPTLARGWDVNV
EKNRGAILFVKNGLYYLGIMPKQKGRYKALSFEPTEKTSEGFDKMYYDYFPDAAKMIPKCSTQLKAVTAHFQTHT
TPILLSNNFIEPLEITKEIYDLNNPEKEPKKFQTAYAKKTGDQKGYREALCKWIDFTRDFLSKYTKTTSIDLSSL
RPSSQYKDLGEYYAELNPLLYHISFQRIAEKEIMDAVETGKLYLFQIYNKDFAKGHHGKPNLHTLYWTGLESPEN
LAKTSIKLNGQAELFYRPKSRMKRMAHRLGEKMLNKKLKDQKTPIPDTLYQELYDYVNHRLSHDLSDEARALLPN
VITKEVSHEIIKDRRFTSDKFFFHVPITLNYQAANSPSKENQRVNAYLKEHPETPIIGIARGERNLIYITVIDST
GKILEQRSLNTIQQFDYQKKLDNREKERVAARQAWSVVGTIKDLKQGYLSQVIHEIVDLMIHYQAVVVLENLNFG
FKSKRTGIAEKAVYQQFEKMLIDKLNCLVLKDYPAEKVGGVLNPYQLTDQFTSFAKMGTQSGFLFYVPAPYTSKI
DPLTGFVDPFVWKTIKNHESRKHFLEGFDFLHYDVKTGDFILHFKMNRNLSFQRGLPGEMPAWDIVFEKNETQFD
AKGTPFIAGKRIVPVIENHRFTGRYRDLYPANELIALLEEKGIVERDGSNILPKLLENDDSHAIDTMVALIRSVL
QMRNSNAATGEDYINSPVRDLNGVCFDSRFQNPEWPMDADANGAYHIALKGQLLLNHLKESKDLKLQNGISNQDW
LAYIQELRN

### 6. Construct G

An exemplary fusion polypeptide, as described herein, comprises a Cpf1 domain that lacks nuclease activity, an XTEN linker, a first FokI DNA cleavage domain (D450A), a polypeptide linker, and a second FokI DNA cleavage domain.

In some embodiments, the fusion polypeptide comprises an amino acid sequence shown in SEQ ID NO: 29, or an amino acid sequence that is at least 70%, at least 75%, at least 80%, at least 85%, at least 90%, at least 95%, or at least 99% identical to the amino acid sequence shown in SEQ ID NO: 29. In SEQ ID NO: 29 below, the Cpf1 domain lacking nuclease activity is shown in boldface, the XTEN linker shown in italics, first FokI DNA cleavage domain containing an D450A mutation is shown in underline (with mutation indicated in boldface), the polypeptide linker is shown in italics, and the second FokI DNA cleavage domain is shown in underline.

Amino acid sequence of Construct G: Cpf1 (D908A)-XTEN-FokI (D450A)-polypeptide
linker-FokII
(SEQ ID NO: 29)
TQFEGFTNLYQVSKTLRFELIPQGKTLKHIQEQGFIEEDKARNDHYKELKPIIDRIYKTYADQCLQLVQLDWENL
SAAIDSYRKEKTEETRNALIEEQATYRNAIHDYFIGRTDNLTDAINKRHAEIYKGLFKAELFNGKVLKQLGTVTT
TEHENALLRSFDKFTTYFSGFYRNRKNVFSAEDISTAIPHRIVQDNFPKFKENCHIFTRLITAVPSLREHFENVK
KAIGIFVSTSIEEVFSFPFYNQLLTQTQIDLYNQLLGGISREAGTEKIKGLNEVLNLAIQKNDETAHIIASLPHR
FIPLFKQILSDRNTLSFILEEFKSDEEVIQSFCKYKTLLRNENVLETAEALENELNSIDLTHIFISHKKLETISS
ALCDHWDTLRNALYERRISELTGKITKSAKEKVQRSLKHEDINLQEIISAAGKELSEAFKQKTSEILSHAHAALD
QPLPTTLKKQEEKEILKSQLDSLLGLYHLLDWFAVDESNEVDPEFSARLTGIKLEMEPSLSFYNKARNYATKKPY
SVEKFKLNFQMPTLARGWDVNVEKNRGAILFVKNGLYYLGIMPKQKGRYKALSFEPTEKTSEGEDKMYYDYFPDA
AKMIPKCSTQLKAVTAHFQTHTTPILLSNNFIEPLEITKEIYDLNNPEKEPKKFQTAYAKKTGDQKGYREALCKW
IDFTRDFLSKYTKTTSIDLSSLRPSSQYKDLGEYYAELNPLLYHISFQRIAEKEIMDAVETGKLYLFQIYNKDFA
KGHHGKPNLHTLYWTGLFSPENLAKTSIKLNGQAELFYRPKSRMKRMAHRLGEKMLNKKLKDQKTPIPDTLYQEL
YDYVNHRLSHDLSDEARALLPNVITKEVSHEIIKDRRFTSDKFFFHVPITLNYQAANSPSKENQRVNAYLKEHPE
TPIIGIARGERNLIYITVIDSTGKILEQRSLNTIQQFDYQKKLDNREKERVAARQAWSVVGTIKDLKQGYLSQVI
HEIVDLMIHYQAVVVLENLNFGFKSKRTGIAEKAVYQQFEKMLIDKLNCLVLKDYPAEKVGGVLNPYQLTDQFTS
FAKMGTQSGFLFYVPAPYTSKIDPLTGFVDPFVWKTIKNHESRKHFLEGFDFLHYDVKTGDFILHFKMNRNLSFQ
RGLPGFMPAWDIVFEKNETQFDAKGTPFIAGKRIVPVIENHRFTGRYRDLYPANELIALLEEKGIVERDGSNILP
KLLENDDSHAIDTMVALIRSVLQMRNSNAATGEDYINSPVRDLNGVCFDSRFQNPEWPMDADANGAYHIALKGQL
LLNHLKESKDLKLQNGISNQDWLAYIQELRNSGSETPGTSESATPESQLVKSELEEKKSELRHKLKYVPHEYIEL
IEIARNSTQDRILEMKVMEFFMKVYGYRGKHLGGSRKPAGAIYTVGSPIDYGVIVDTKAYSGGYNLPIGQADEMQ
RYVEENQTRNKHINPNEWWKVYPSSVTEFKFLFVSGHFKGNYKAQLTRLNHITNCNGAVLSVEELLIGGEMIKAG
TLTLEEVRRKFNNGEINFGSGSGSGSITRTTNPRNVVPKIYMSAGSIPLTTHITNSIQPTLWTIGSINGVAPLAK
SIKLGIPVTGSAYTDQTTAMVRKKVSVFMGSGSGSGSSQLVKSELEEKKSELRHKLKYVPHEYIELIEIARNSTQ
DRILEMKVMEFFMKVYGYRGKHLGGSRKPDGAIYTVGSPIDYGVIVDTKAYSGGYNLPIGQADEMQRYVEENQTR
NKHINPNEWWKVYPSSVTEFKFLFVSGHFKGNYKAQLTRLNHITNCNGAVLSVEELLIGGEMIKAGTLTLEEVRR
KFNNGEINF

### 7. Construct H

An exemplary fusion polypeptide, as described herein, comprises a Cpf1 domain that lacks nuclease activity, an XTEN linker, a first FokI DNA cleavage domain, a polypeptide linker, and a second FokI DNA cleavage domain (D450A).

In some embodiments, the fusion polypeptide comprises an amino acid sequence shown in SEQ ID NO: 30, or an amino acid sequence that is at least 70%, at least 75%, at least 80%, at least 85%, at least 90%, at least 95%, or at least 99% identical to the amino acid sequence shown in SEQ ID NO: 30. In SEQ ID NO: 30 below, the Cpf1 domain lacking nuclease activity is shown in boldface, the XTEN linker shown in italics, first FokI DNA cleavage domain is shown in underline, the polypeptide linker is shown in italics, and the second FokI DNA cleavage domain containing an D450A mutation is shown in underline (with mutation indicated in boldface).

Amino acid sequence of Construct H: Cpf1 (D908A)-XTEN-FokI-polypeptide linker-
FokII (D450A)
(SEQ ID NO: 30)
TQFEGFTNLYQVSKTLRFELIPQGKTLKHIQEQGFIEEDKARNDHYKELKPIIDRIYKTYADQCLQLVQLDWENL
SAAIDSYRKEKTEETRNALIEEQATYRNAIHDYFIGRTDNLTDAINKRHAEIYKGLFKAELFNGKVLKQLGTVTT
TEHENALLRSEDKFTTYFSGFYRNRKNVFSAEDISTAIPHRIVQDNFPKFKENCHIFTRLITAVPSLREHFENVK
KAIGIFVSTSIEEVFSFPFYNQLLTQTQIDLYNQLLGGISREAGTEKIKGLNEVLNLAIQKNDETAHIIASLPHR
FIPLFKQILSDRNTLSFILEEFKSDEEVIQSFCKYKTLLRNENVLETAEALFNELNSIDLTHIFISHKKLETISS
ALCDHWDTLRNALYERRISELTGKITKSAKEKVQRSLKHEDINLQEIISAAGKELSEAFKQKTSEILSHAHAALD
QPLPTTLKKQEEKEILKSQLDSLLGLYHLLDWFAVDESNEVDPEFSARLTGIKLEMEPSLSFYNKARNYATKKPY
SVEKFKLNFQMPTLARGWDVNVEKNRGAILFVKNGLYYLGIMPKQKGRYKALSFEPTEKTSEGFDKMYYDYFPDA
AKMIPKCSTQLKAVTAHFQTHTTPILLSNNFIEPLEITKEIYDLNNPEKEPKKFQTAYAKKTGDQKGYREALCKW
IDFTRDFLSKYTKTTSIDLSSLRPSSQYKDLGEYYAELNPLLYHISFQRIAEKEIMDAVETGKLYLFQIYNKDFA
KGHHGKPNLHTLYWTGLFSPENLAKTSIKLNGQAELFYRPKSRMKRMAHRLGEKMLNKKLKDQKTPIPDTLYQEL
YDYVNHRLSHDLSDEARALLPNVITKEVSHEIIKDRRFTSDKFFFHVPITLNYQAANSPSKENQRVNAYLKEHPE
TPIIGIARGERNLIYITVIDSTGKILEQRSLNTIQQFDYQKKLDNREKERVAARQAWSVVGTIKDLKQGYLSQVI
HEIVDLMIHYQAVVVLENLNFGFKSKRTGIAEKAVYQQFEKMLIDKLNCLVLKDYPAEKVGGVLNPYQLTDQFTS
FAKMGTQSGFLFYVPAPYTSKIDPLTGFVDPFVWKTIKNHESRKHFLEGFDFLHYDVKTGDFILHFKMNRNLSFQ
RGLPGFMPAWDIVFEKNETQFDAKGTPFIAGKRIVPVIENHRFTGRYRDLYPANELIALLEEKGIVERDGSNILP
KLLENDDSHAIDTMVALIRSVLQMRNSNAATGEDYINSPVRDLNGVCFDSRFQNPEWPMDADANGAYHIALKGQL
LLNHLKESKDLKLQNGISNQDWLAYIQELRNSGSETPGTSESATPESQLVKSELEEKKSELRHKLKYVPHEYIEL
IEIARNSTQDRILEMKVMEFFMKVYGYRGKHLGGSRKPDGAIYTVGSPIDYGVIVDTKAYSGGYNLPIGQADEMQ
RYVEENQTRNKHINPNEWWKVYPSSVTEFKFLFVSGHFKGNYKAQLTRLNHITNCNGAVLSVEELLIGGEMIKAG
TLTLEEVRRKFNNGEINFGSGSGSGSITRTTNPRNVVPKIYMSAGSIPLTTHITNSIQPTLWTIGSINGVAPLAK
SIKLGIPVTGSAYTDQTTAMVRKKVSVFMGSGSGSGSSQLVKSELEEKKSELRHKLKYVPHEYIELIEIARNSTQ
DRILEMKVMEFFMKVYGYRGKHLGGSRKPAGAIYTVGSPIDYGVIVDTKAYSGGYNLPIGQADEMQRYVEENQTR
NKHINPNEWWKVYPSSVTEFKFLFVSGHFKGNYKAQLTRLNHITNCNGAVLSVEELLIGGEMIKAGTLTLEEVRR
KFNNGEINE

### 8. Construct I

An exemplary fusion polypeptide, as described herein, comprises a first FokI DNA cleavage domain (D450A), a polypeptide linker, a second FokI DNA cleavage domain, an XTEN linker, and a Cpf1 domain that lacks nuclease activity.

In some embodiments, the fusion polypeptide comprises an amino acid sequence shown in SEQ ID NO: 31, or an amino acid sequence that is at least 70%, at least 75%, at least 80%, at least 85%, at least 90%, at least 95%, or at least 99% identical to the amino acid sequence shown in SEQ ID NO: 31. In SEQ ID NO: 31 below, the first FokI DNA cleavage domain containing an D450A mutation is shown in underline (with mutation indicated in boldface), the polypeptide linker is shown in italics, the second FokI DNA cleavage domain, the XTEN linker shown in italics, and the Cpf1 domain lacking nuclease activity is shown in boldface.

Amino acid sequence of Construct I: FokI (D450A)-polypeptide linker-FokII-XTEN-
Cpf1 (D908A)
(SEQ ID NO: 31)
MQLVKSELEEKKSELRHKLKYVPHEYIELIEIARNSTQDRILEMKVMEFFMKVYGYRGKHLGGSRKPAGAIYTVG
SPIDYGVIVDTKAYSGGYNLPIGQADEMQRYVEENQTRNKHINPNEWWKVYPSSVTEFKFLFVSGHFKGNYKAQL
TRLNHITNCNGAVLSVEELLIGGEMIKAGTLTLEEVRRKFNNGEINFGSGSGSGSITRTTNPRNVVPKIYMSAGS
IPLTTHITNSIQPTLWTIGSINGVAPLAKSIKLGIPVTGSAYTDQTTAMVRKKVSVFMGSGSGSGSSQLVKSELE
EKKSELRHKLKYVPHEYIELIEIARNSTQDRILEMKVMEFFMKVYGYRGKHLGGSRKPDGAIYTVGSPIDYGVIV
DTKAYSGGYNLPIGQADEMQRYVEENQTRNKHINPNEWWKVYPSSVTEFKFLFVSGHFKGNYKAQLTRINHITNC
NGAVLSVEELLIGGEMIKAGTLTLEEVRRKFNNGEINFSGSETPGTSESATPESTQFEGFTNLYQVSKTLRFELI
PQGKTLKHIQEQGFIEEDKARNDHYKELKPIIDRIYKTYADQCLQLVQLDWENLSAAIDSYRKEKTEETRNALIE
EQATYRNAIHDYFIGRTDNLTDAINKRHAEIYKGLFKAELFNGKVLKQLGTVTTTEHENALLRSEDKFTTYFSGE
YRNRKNVFSAEDISTAIPHRIVQDNFPKFKENCHIFTRLITAVPSLREHFENVKKAIGIFVSTSIEEVFSFPFYN
QLLTQTQIDLYNQLLGGISREAGTEKIKGLNEVLNLAIQKNDETAHIIASLPHRFIPLFKQILSDRNTLSFILEE
FKSDEEVIQSFCKYKTLLRNENVLETAEALFNELNSIDLTHIFISHKKLETISSALCDHWDTLRNALYERRISEL
TGKITKSAKEKVQRSLKHEDINLQEIISAAGKELSEAFKQKTSEILSHAHAALDQPLPTTLKKQEEKEILKSQLD
SLLGLYHLLDWFAVDESNEVDPEFSARLTGIKLEMEPSLSFYNKARNYATKKPYSVEKFKLNFQMPTLARGWDVN
VEKNRGAILFVKNGLYYLGIMPKQKGRYKALSFEPTEKTSEGFDKMYYDYFPDAAKMIPKCSTQLKAVTAHFQTH
TTPILLSNNFIEPLEITKEIYDLNNPEKEPKKFQTAYAKKTGDQKGYREALCKWIDFTRDELSKYTKTTSIDLSS
LRPSSQYKDLGEYYAELNPLLYHISFQRIAEKEIMDAVETGKLYLFQIYNKDFAKGHHGKPNLHTLYWTGLESPE
NLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKFFFHVPITLNYQAANSPSKENQRVNAYLKEHPETPIIGIARGERNLIYITVIDS
TGKILEQRSLNTIQQFDYQKKLDNREKERVAARQAWSVVGTIKDLKQGYLSQVIHEIVDLMIHYQAVVVLENLNE
GFKSKRTGIAEKAVYQQFEKMLIDKLNCLVLKDYPAEKVGGVLNPYQLTDQFTSFAKMGTQSGELFYVPAPYTSK
IDPLTGFVDPFVWKTIKNHESRKHFLEGFDFLHYDVKTGDFILHFKMNRNLSFQRGLPGEMPAWDIVFEKNETQF
DAKGTPFIAGKRIVPVIENHRFTGRYRDLYPANELIALLEEKGIVERDGSNILPKLLENDDSHAIDTMVALIRSV
LQMRNSNAATGEDYINSPVRDLNGVCFDSRFQNPEWPMDADANGAYHIALKGQLLLNHLKESKDLKLQNGISNQD
WLAYIQELRN

### Nucleic Acids and Vectors

Also provided herein are nucleic acids comprising a nucleotide sequence encoding any of the fusion polypeptides described herein. In some embodiments, any nucleotide sequences herein may be codon-optimized. Without being bound to a particular theory or mechanism, it is believed that codon optimization of the nucleotide sequence increases the translation efficiency of the mRNA transcripts. Codon optimization of the nucleotide sequence may involve substituting a native codon for another codon that encodes the same amino acid, but can be translated by tRNA that is more readily available within a cell, thus increasing translation efficiency. Optimization of the nucleotide sequence may also reduce secondary mRNA structures that would interfere with translation, thus increasing translation efficiency. In an embodiment of the invention, the codon-optimized nucleotide sequence may comprise, consist, or consist essentially of any one of the nucleic acid sequences described herein.

Any of the nucleic acids of described herein may be recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

A recombinant nucleic acid may be one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques, such as those described in Green et al., supra. The nucleic acids can be constructed based on chemical synthesis and/or enzymatic ligation reactions using procedures known in the art. See, for example, Green et al., supra. For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methyl guanine, 1-methylinosine, 2,2-dimethylguanine, 2-methyladenine, 2-methylguanine, 3-methylcytosine, 5-methylcytosine, N6-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the invention can be purchased from companies, such as Macromolecular Resources (Fort Collins, CO) and Synthegen (Houston, TX).

Also provided herein are isolated or purified nucleic acids comprising a nucleotide sequence which is complementary to the nucleotide sequence of any of the nucleic acids described herein or a nucleotide sequence which hybridizes under stringent conditions to the nucleotide sequence of any of the nucleic acids described herein.

The nucleotide sequence which hybridizes under stringent conditions may hybridize under high stringency conditions. The term “high stringency conditions” refers to a nucleotide sequence that specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-10 bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C. Such high stringency conditions tolerate little, if any, mismatch between the nucleotide sequence and the template or target strand, and are particularly suitable for detecting expression of any of the CARs described herein. It is generally appreciated that conditions can be rendered more stringent by the addition of increasing amounts of formamide.

The present disclosure also provides nucleic acids comprising a nucleotide sequence that is at least about 70% or more, e.g., about 80%, about 90%, about 91%, about 92%, about 93%, about 94%, about 95%, about 96%, about 97%, about 98%, or about 99% identical to any of the nucleic acids described herein, such as any one of SEQ ID NOs: 32-39.

Nucleic acid sequences of exemplary fusion polypeptides of the present disclosure are provided below.

Exemplary nucleic acid sequence of Construct A: FokI (D450)-polypeptide linker-FokII
(D450A)-XTEN linker-AsCpf1 (A)
(SEQ ID NO: 32)
atgcaactggtgaagagcgagctggaagagaagaaaagcgagctcagacataagctgaagtacgttccccacgaa
tacattgaactgatagaaatcgctagaaacagtacgcaagacagaatactggaaatgaaggtgatggagttcttc
atgaaggtttacggctatcgtggcaaacacctcgggggctcccggaagcccgacggggctatctacaccgtgggc
agtcccatcgactatggcgtgatcgtggacaccaaagcttatagcggcggatataatctccccatcggccaagcc
gatgagatgcagaggtatgtggaggagaaccaaacaagaaacaagcatatcaaccccaacgagtggtggaaggtt
tatcctagctcggtgaccgagtttaagttcctattcgtgtctggccacttcaagggcaactataaggcacagctc
actagactgaatcatatcacgaattgcaacggcgccgtgttatccgtggaggagctactgatcggcggagagatg
atcaaagccggcaccctgaccctggaagaggtgagaagaaagtttaacaatggcgaaataaatttcggcagcgga
agtggaagcggctccatcactagaaccaccaaccctagaaacgtggtgcccaagatctacatgagcgccggcagc
atccccctgaccacccacatcaccaactcaattcagcccaccctgtggaccatcggcagcatcaacggcgtggcc
cccctggccaagagcatcaagctgggcatccccgtgaccggcagcgcctacaccgatcagaccaccgccatggtg
agaaagaaggtgagcgtgttcatgggcagcggcagcgggagcggctcatcgcagctggttaagagcgagttagaa
gaaaaaaagagcgaactgcggcataaactgaagtatgtcccacacgagtacatcgaactgatcgagatcgcgaga
aactctacccaagacagaattctggagatgaaagtaatggaatttttcatgaaggtgtatggatatagagggaag
cacctgggtggcagcagaaaacccgccggcgccatctacactgtggggagccccatagactatggtgtgatcgtg
gataccaaggcgtatagcggcggttacaatctgcccattgggcaagcggacgagatgcaaagatatgtggaagag
aatcagacgaggaacaagcacattaaccctaatgagtggtggaaggtctaccctagctccgttaccgagttcaag
ttcctgtttgtgagcgggcattttaagggcaactacaaggcacagctgacccgcctgaaccacataacaaactgc
aacggtgccgtgctgagcgtagaagagttgctaatcggcggcgagatgatcaaggccggcacgctaaccctcgaa
gaggtgcgcagaaagttcaataacggcgaaatcaatttcagcggcagcgagactcccgggacctcagagtccgcc
acacccgaaagtacacagttcgagggctttaccaacctgtatcaggtgagcaagacactgcggtttgagctgatc
ccacagggcaagaccctgaagcacatccaggagcagggcttcatcgaggaggacaaggcccgcaatgatcactac
aaggagctgaagcccatcatcgatcggatctacaagacctatgccgaccagtgcctgcagctggtgcagctggat
tgggagaacctgagcgccgccatcgactcctatagaaaggagaaaaccgaggagacaaggaacgccctgatcgag
gagcaggccacatatcgcaatgccatccacgactacttcatcggccggacagacaacctgaccgatgccatcaat
aagagacacgccgagatctacaagggcctgttcaaggccgagctgtttaatggcaaggtgctgaagcagctgggc
accgtgaccacaaccgagcacgagaacgccctgctgcggagcttcgacaagtttacaacctacttctccggcttt
tatagaaacaggaagaacgtgttcagcgccgaggatatcagcacagccatcccacaccgcatcgtgcaggacaac
ttccccaagtttaaggagaattgtcacatcttcacacgcctgatcaccgccgtgcccagcctgcgggagcacttt
gagaacgtgaagaaggccatcggcatcttcgtgagcacctccatcgaggaggtgttttccttccctttttataac
cagctgctgacacagacccagatcgacctgtataaccagctgctgggaggaatctctcgggaggcaggcaccgag
aagatcaagggcctgaacgaggtgctgaatctggccatccagaagaatgatgagacagcccacatcatcgcctcc
ctgccacacagattcatccccctgtttaagcagatcctgtccgataggaacaccctgtctttcatcctggaggag
tttaagagcgacgaggaagtgatccagtccttctgcaagtacaagacactgctgagaaacgagaacgtgctggag
acagccgaggccctgtttaacgagctgaacagcatcgacctgacacacatcttcatcagccacaagaagctggag
acaatcagcagcgccctgtgcgaccactgggatacactgaggaatgccctgtatgagcggagaatctccgagctg
acaggcaagatcaccaagtctgccaaggagaaggtgcagcgcagcctgaagcacgaggatatcaacctgcaggag
atcatctctgccgcaggcaaggagctgagcgaggccttcaagcagaaaaccagcgagatcctgtcccacgcacac
gccgccctggatcagccactgcctacaaccctgaagaagcaggaggagaaggagatcctgaagtctcagctggac
agcctgctgggcctgtaccacctgctggactggtttgccgtggatgagtccaacgaggtggaccccgagttctct
gcccggctgaccggcatcaagctggagatggagccttctctgagcttctacaacaaggccagaaattatgccacc
aagaagccctactccgtggagaagttcaagctgaactttcagatgcctacactggccagaggctgggacgtgaat
gtggagaagaacagaggcgccatcctgtttgtgaagaacggcctgtactatctgggcatcatgccaaagcagaag
ggcaggtataaggccctgagcttcgagcccacagagaaaaccagcgagggctttgataagatgtactatgactac
ttccctgatgccgccaagatgatcccaaagtgcagcacccagctgaaggccgtgacagcccactttcagacccac
acaacccccatcctgctgtccaacaatttcatcgagcctctggagatcacaaaggagatctacgacctgaacaat
cctgagaaggagccaaagaagtttcagacagcctacgccaagaaaaccggcgaccagaagggctacagagaggcc
ctgtgcaagtggatcgacttcacaagggattttctgtccaagtataccaagacaacctctatcgatctgtctagc
ctgcggccatcctctcagtataaggacctgggcgagtactatgccgagctgaatcccctgctgtaccacatcagc
ttccagagaatcgccgagaaggagatcatggatgccgtggagacaggcaagctgtacctgttccagatctataac
aaggactttgccaagggccaccacggcaagcctaatctgcacacactgtattggaccggcctgttttctccagag
aacctggccaagacaagcatcaagctgaatggccaggccgagctgttctaccgccctaagtccaggatgaagagg
atggcacaccggctgggagagaagatgctgaacaagaagctgaaggatcagaaaaccccaatccccgacaccctg
taccaggagctgtacgactatgtgaatcacagactgtcccacgacctgtctgatgaggccagggccctgctgccc
aacgtgatcaccaaggaggtgtctcacgagatcatcaaggataggcgctttaccagcgacaagttctttttccac
gtgcctatcacactgaactatcaggccgccaattccccatctaagttcaaccagagggtgaatgcctacctgaag
gagcaccccgagacacctatcatcggcatcgcccggggcgagagaaacctgatctatatcacagtgatcgactcc
accggcaagatcctggagcagcggagcctgaacaccatccagcagtttgattaccagaagaagctggacaacagg
gagaaggagagggtggcagcaaggcaggcctggtctgtggtgggcacaatcaaggatctgaagcagggctatctg
agccaggtcatccacgagatcgtggacctgatgatccactaccaggccgtggtggtgctggagaacctgaatttc
ggctttaagagcaagaggaccggcatcgccgagaaggccgtgtaccagcagttcgagaagatgctgatcgataag
ctgaattgcctggtgctgaaggactatccagcagagaaagtgggaggcgtgctgaacccataccagctgacagac
cagttcacctcctttgccaagatgggcacccagtctggcttcctgttttacgtgcctgccccatatacatctaag
atcgatcccctgaccggcttcgtggaccccttcgtgtggaaaaccatcaagaatcacgagagccgcaagcacttc
ctggagggcttcgactttctgcactacgacgtgaaaaccggcgacttcatcctgcactttaagatgaacagaaat
ctgtccttccagaggggcctgcccggctttatgcctgcatgggatatcgtgttcgagaagaacgagacacagttt
gacgccaagggcacccctttcatcgccggcaagagaatcgtgccagtgatcgagaatcacagattcaccggcaga
taccgggacctgtatcctgccaacgagctgatcgccctgctggaggagaagggcatcgtgttcagggatggctcc
aacatcctgccaaagctgctggagaatgacgattctcacgccatcgacaccatggtggccctgatccgcagcgtg
ctgcagatgcggaactccaatgccgccacaggcgaggactatatcaacagccccgtgcgcgatctgaatggcgtg
tgcttcgactcccggtttcagaacccagagtggcccatggacgccgatgccaatggcgcctaccacatcgccctg
aagggccagctgctgctgaatcacctgaaggagagcaaggatctgaagctgcagaacggcatctccaatcaggac
tggctggcctacatccaggagctgcgcaac
Exemplary nucleic acid sequence of Construct B: APOBEC Base Editor-AsCpf1 (D908A)-
XTEN-FokI (D450A)-polypeptide linker-FokII (D450)
(SEQ ID NO: 33)
atgggcagctcagagactggcccagtggctgtggaccccacattgagacggcggatcgagccccatgagtttgag
gtattcttcgatccgagagagctccgcaaggagacctgcctgctttacgaaattaattgggggggccggcactcc
atttggcgacatacatcacagaacactaacaagcacgtcgaagtcaacttcatcgagaagttcacgacagaaaga
tatttctgtccgaacacaaggtgcagcattacctggtttctcagctggagcccatgcggcgaatgtagtagggcc
atcactgaattcctgtcaaggtatccccacgtcactctgtttatttacatcgcaaggctgtaccaccacgctgac
ccccgcaatcgacaaggcctgcgggatttgatctcttcaggtgtgactatccaaattatgactgagcaggagtca
ggatactgctggagaaactttgtgaattatagcccgagtaatgaagcccactggcctaggtatccccatctgtgg
gtacgactgtacgttcttgaactgtactgcatcatactgggcctgcctccttgtctcaacattctgagaaggaag
cagccacagctgacattctttaccatcgctcttcagtcttgtcattaccagcgactgcccccacacattctctgg
gccaccgggttgaaatctggtggttcttctggtggttctagcggcagcgagactcccgggacctcagagtccgcc
acacccgaaagttccggagggagtagcggcgggtctacacagttcgagggctttaccaacctgtatcaggtgagc
aagacactgcggtttgagctgatcccacagggcaagaccctgaagcacatccaggagcagggcttcatcgaggag
gacaaggcccgcaatgatcactacaaggagctgaagcccatcatcgatcggatctacaagacctatgccgaccag
tgcctgcagctggtgcagctggattgggagaacctgagcgccgccatcgactcctatagaaaggagaaaaccgag
gagacaaggaacgccctgatcgaggagcaggccacatatcgcaatgccatccacgactacttcatcggccggaca
gacaacctgaccgatgccatcaataagagacacgccgagatctacaagggcctgttcaaggccgagctgtttaat
ggcaaggtgctgaagcagctgggcaccgtgaccacaaccgagcacgagaacgccctgctgcggagcttcgacaag
tttacaacctacttctccggcttttatagaaacaggaagaacgtgttcagcgccgaggatatcagcacagccatc
ccacaccgcatcgtgcaggacaacttccccaagtttaaggagaattgtcacatcttcacacgcctgatcaccgcc
gtgcccagcctgcgggagcactttgagaacgtgaagaaggccatcggcatcttcgtgagcacctccatcgaggag
gtgttttccttccctttttataaccagctgctgacacagacccagatcgacctgtataaccagctgctgggagga
atctctcgggaggcaggcaccgagaagatcaagggcctgaacgaggtgctgaatctggccatccagaagaatgat
gagacagcccacatcatcgcctccctgccacacagattcatccccctgtttaagcagatcctgtccgataggaac
accctgtctttcatcctggaggagtttaagagcgacgaggaagtgatccagtccttctgcaagtacaagacactg
ctgagaaacgagaacgtgctggagacagccgaggccctgtttaacgagctgaacagcatcgacctgacacacatc
ttcatcagccacaagaagctggagacaatcagcagcgccctgtgcgaccactgggatacactgaggaatgccctg
tatgagcggagaatctccgagctgacaggcaagatcaccaagtctgccaaggagaaggtgcagcgcagcctgaag
cacgaggatatcaacctgcaggagatcatctctgccgcaggcaaggagctgagcgaggccttcaagcagaaaacc
agcgagatcctgtcccacgcacacgccgccctggatcagccactgcctacaaccctgaagaagcaggaggagaag
gagatcctgaagtctcagctggacagcctgctgggcctgtaccacctgctggactggtttgccgtggatgagtcc
aacgaggtggaccccgagttctctgcccggctgaccggcatcaagctggagatggagccttctctgagcttctac
aacaaggccagaaattatgccaccaagaagccctactccgtggagaagttcaagctgaactttcagatgcctaca
ctggccagaggctgggacgtgaatgtggagaagaacagaggcgccatcctgtttgtgaagaacggcctgtactat
ctgggcatcatgccaaagcagaagggcaggtataaggccctgagcttcgagcccacagagaaaaccagcgagggc
tttgataagatgtactatgactacttccctgatgccgccaagatgatcccaaagtgcagcacccagctgaaggcc
gtgacagcccactttcagacccacacaacccccatcctgctgtccaacaatttcatcgagcctctggagatcaca
aaggagatctacgacctgaacaatcctgagaaggagccaaagaagtttcagacagcctacgccaagaaaaccggc
gaccagaagggctacagagaggccctgtgcaagtggatcgacttcacaagggattttctgtccaagtataccaag
acaacctctatcgatctgtctagcctgcggccatcctctcagtataaggacctgggcgagtactatgccgagctg
aatcccctgctgtaccacatcagcttccagagaatcgccgagaaggagatcatggatgccgtggagacaggcaag
ctgtacctgttccagatctataacaaggactttgccaagggccaccacggcaagcctaatctgcacacactgtat
tggaccggcctgttttctccagagaacctggccaagacaagcatcaagctgaatggccaggccgagctgttctac
cgccctaagtccaggatgaagaggatggcacaccggctgggagagaagatgctgaacaagaagctgaaggatcag
aaaaccccaatccccgacaccctgtaccaggagctgtacgactatgtgaatcacagactgtcccacgacctgtct
gatgaggccagggccctgctgcccaacgtgatcaccaaggaggtgtctcacgagatcatcaaggataggcgcttt
accagcgacaagttctttttccacgtgcctatcacactgaactatcaggccgccaattccccatctaagttcaac
cagagggtgaatgcctacctgaaggagcaccccgagacacctatcatcggcatcgcccggggcgagagaaacctg
atctatatcacagtgatcgactccaccggcaagatcctggagcagcggagcctgaacaccatccagcagtttgat
taccagaagaagctggacaacagggagaaggagagggtggcagcaaggcaggcctggtctgtggtgggcacaatc
aaggatctgaagcagggctatctgagccaggtcatccacgagatcgtggacctgatgatccactaccaggccgtg
gtggtgctggagaacctgaatttcggctttaagagcaagaggaccggcatcgccgagaaggccgtgtaccagcag
ttcgagaagatgctgatcgataagctgaattgcctggtgctgaaggactatccagcagagaaagtgggaggcgtg
ctgaacccataccagctgacagaccagttcacctcctttgccaagatgggcacccagtctggcttcctgttttac
gtgcctgccccatatacatctaagatcgatcccctgaccggcttcgtggaccccttcgtgtggaaaaccatcaag
aatcacgagagccgcaagcacttcctggagggcttcgactttctgcactacgacgtgaaaaccggcgacttcatc
ctgcactttaagatgaacagaaatctgtccttccagaggggcctgcccggctttatgcctgcatgggatatcgtg
ttcgagaagaacgagacacagtttgacgccaagggcacccctttcatcgccggcaagagaatcgtgccagtgatc
gagaatcacagattcaccggcagataccgggacctgtatcctgccaacgagctgatcgccctgctggaggagaag
ggcatcgtgttcagggatggctccaacatcctgccaaagctgctggagaatgacgattctcacgccatcgacacc
atggtggccctgatccgcagcgtgctgcagatgcggaactccaatgccgccacaggcgaggactatatcaacagc
cccgtgcgcgatctgaatggcgtgtgcttcgactcccggtttcagaacccagagtggcccatggacgccgatgcc
aatggcgcctaccacatcgccctgaagggccagctgctgctgaatcacctgaaggagagcaaggatctgaagctg
cagaacggcatctccaatcaggactggctggcctacatccaggagctgcgcaacagcggcagcgagactcccggg
acctcagagtccgccacacccgaaagtcaactggtgaagagcgagctggaagagaagaaaagcgagctcagacat
aagctgaagtacgttccccacgaatacattgaactgatagaaatcgctagaaacagtacgcaagacagaatactg
gaaatgaaggtgatggagttcttcatgaaggtttacggctatcgtggcaaacacctcgggggctcccggaagccc
gccggggctatctacaccgtgggcagtcccatcgactatggcgtgatcgtggacaccaaagcttatagcggcgga
tataatctccccatcggccaagccgatgagatgcagaggtatgtggaggagaaccaaacaagaaacaagcatatc
aaccccaacgagtggtggaaggtttatcctagctcggtgaccgagtttaagttcctattcgtgtctggccacttc
aagggcaactataaggcacagctcactagactgaatcatatcacgaattgcaacggcgccgtgttatccgtggag
gagctactgatcggcggagagatgatcaaagccggcaccctgaccctggaagaggtgagaagaaagtttaacaat
ggcgaaataaatttcggcagcggaagtggaagcggctccatcactagaaccaccaaccctagaaacgtggtgccc
aagatctacatgagcgccggcagcatccccctgaccacccacatcaccaactcaattcagcccaccctgtggacc
atcggcagcatcaacggcgtggcccccctggccaagagcatcaagctgggcatccccgtgaccggcagcgcctac
accgatcagaccaccgccatggtgagaaagaaggtgagcgtgttcatgggcagcggcagcgggagcggctcatcg
cagctggttaagagcgagttagaagaaaaaaagagcgaactgcggcataaactgaagtatgtcccacacgagtac
atcgaactgatcgagatcgcgagaaactctacccaagacagaattctggagatgaaagtaatggaatttttcatg
aaggtgtatggatatagagggaagcacctgggtggcagcagaaaacccgacggcgccatctacactgtggggagc
cccatagactatggtgtgatcgtggataccaaggcgtatagcggcggttacaatctgcccattgggcaagcggac
gagatgcaaagatatgtggaagagaatcagacgaggaacaagcacattaaccctaatgagtggtggaaggtctac
cctagctccgttaccgagttcaagttcctgtttgtgagcgggcattttaagggcaactacaaggcacagctgacc
cgcctgaaccacataacaaactgcaacggtgccgtgctgagcgtagaagagttgctaatcggcggcgagatgatc
aaggccggcacgctaaccctcgaagaggtgcgcagaaagttcaataacggcgaaatcaatttc
Exemplary nucleic acid sequence of Construct C: APOBEC Base Editor-MAD7 ™ (D908A)-
XTEN-FokI (D450A)-polypeptide linker-FokII (D450)
(SEQ ID NO: 34)
atgggcagctcagagactggcccagtggctgtggaccccacattgagacggcggatcgagccccatgagtttgag
gtattcttcgatccgagagagctccgcaaggagacctgcctgctttacgaaattaattgggggggccggcactcc
atttggcgacatacatcacagaacactaacaagcacgtcgaagtcaacttcatcgagaagttcacgacagaaaga
tatttctgtccgaacacaaggtgcagcattacctggtttctcagctggagcccatgcggcgaatgtagtagggcc
atcactgaattcctgtcaaggtatccccacgtcactctgtttatttacatcgcaaggctgtaccaccacgctgac
ccccgcaatcgacaaggcctgcgggatttgatctcttcaggtgtgactatccaaattatgactgagcaggagtca
ggatactgctggagaaactttgtgaattatagcccgagtaatgaagcccactggcctaggtatccccatctgtgg
gtacgactgtacgttcttgaactgtactgcatcatactgggcctgcctccttgtctcaacattctgagaaggaag
cagccacagctgacattctttaccatcgctcttcagtcttgtcattaccagcgactgcccccacacattctctgg
gccaccgggttgaaatctggtggttcttctggtggttctagcggcagcgagactcccgggacctcagagtccgcc
acacccgaaagttccggagggagtagcggcgggtctaataacggaactaataacttccaaaacttcatcgggatc
agttccttgcagaaaactctccggaatgctctcatcccaactgagactactcagcagttcattgttaagaatgga
atcataaaagaggacgagcttaggggggaaaataggcaaatcctcaaggatatcatggatgactattataggggc
tttatatccgagacactgagcagcattgatgatatagactggacctctcttttcgaaaagatggaaatacaactt
aaaaatggagataacaaggacaccctgataaaggaacagaccgaatataggaaggcaattcataaaaagtttgct
aacgatgataggtttaaaaacatgttctcagcaaaactcatttcagatatactgcccgaattcgttatccacaac
aacaactactccgctagcgaaaaagaggaaaagacccaagtcataaagctgttctctcgattcgcgacgagtttt
aaagattatttccgaaatcgcgcaaactgtttctcagctgatgatatcagcagctcatcctgtcatcggatcgtt
aacgataatgctgaaatcttcttctccaatgcacttgtttataggcgcattgttaaatctctctcaaacgatgat
atcaataagatttccggcgatatgaaggacagtcttaaggagatgagcctcgaagagatatactcatacgagaaa
tatggcgaatttatcacccaggaagggatttccttctataatgacatttgcggcaaagtcaattccttcatgaac
ctgtattgccaaaaaaataaagaaaacaagaacctctataagctgcaaaagttgcataagcaaatactttgtatc
gcggatacaagctatgaagttccctacaagttcgagagtgatgaggaggtgtatcaatctgtcaatggtttcctt
gataatatttcttctaagcatattgttgaacgactccgaaagataggagacaactataatggatacaatttggat
aaaatctacatcgtgtctaaattttacgagagtgtgtcacaaaaaacatatagagactgggagacaattaatacc
gccctggagatacattacaacaatatacttcccgggaacgggaagtctaaggcagacaaggtgaagaaagccgtg
aagaacgacttgcaaaagtcaattaccgaaatcaatgagcttgtttcaaactataaactttgttcagatgacaat
attaaagccgaaacctatattcatgaaatctctcatattctgaataactttgaggcgcaagaactgaaatataac
ccagaaatacacctcgttgagtccgaactgaaagcaagcgaactgaaaaatgttttggacgtgataatgaacgct
tttcattggtgctcagtctttatgacagaggagcttgttgacaaggataacaatttctatgcggaactggaagag
atttacgacgaaatctatccggtcatatccctgtataacctggttcgcaactatgtcacgcaaaaaccatacagc
acgaagaagattaaactgaactttggtattccgacgctggcccgcggatggtcaaaatctgttgaatactcacga
aatgccataatcctgatgcgagataacctctactaccttggaatctttaatgctaaaaataaacccgataaaaaa
attatcgaagggaacacgagtgaaaacaaaggtgattataaaaaaatgatatataatctgcttccaggaccaaat
aagatgatacccaaagttttcctttcttcaaagaccggcgtcgagacatataaaccatccgcgtacatacttgaa
ggctacaaacaaaataaacatatcaaatcatctaaggattttgacattacgttctgtcatgatttgattgactat
ttcaaaaattgcatagccattcatccagagtggaaaaactttgggtttgacttctctgataccagtacatatgaa
gacataagtggattttaccgagaagtagagctccaaggttataaaatagactggacctatatatctgaaaaggat
atagaccttttgcaagagaagggacagctttatcttttccaaatctacaacaaagacttcagtaagaaaagtacc
gggaatgacaatcttcataccatgtatctgaagaacctgttctccgaagaaaatctgaaggacatagtcctgaag
cttaatggcgaagcggaaatttttttccgaaagagctctattaagaaccccataatacataagaagggaagcatt
ctcgttaatcgaacgtatgaggccgaagagaaagatcaatttgggaatatccaaatcgttcgaaagaacatacca
gaaaatatttaccaagaattgtacaaatattttaacgataaaagcgacaaagaactgtctgatgaagctgctaag
ctgaaaaacgtcgtcggccatcatgaggccgcgacgaatatagtcaaggattaccgatatacatacgataagtat
ttcctgcatatgcccatcactatcaactttaaggcaaataagactggattcattaatgacagaatactgcaatac
atagctaaagaaaaagatttgcatgttattggcattgccaggggtgagcgcaatcttatctatgtaagcgtcatt
gatacttgcgggaatatcgtagagcagaagtcatttaatattgtaaatgggtacgattaccaaatcaagttgaag
cagcaagagggagcacgacagattgcccgcaaggagtggaaagagatcggaaagataaaggagatcaaggagggg
tatttgtcccttgttatacacgaaatttccaagatggtaatcaagtacaacgctataattgctatggaggatctc
tcctatggatttaaaaagggaagatttaaagtcgagcggcaggtatatcagaaatttgaaacaatgcttattaat
aaacttaattatctcgttttcaaagacattagtatcaccgaaaacggtgggctgttgaagggctatcaacttacg
tacataccagataagcttaagaatgtgggtcaccaatgcggatgcatattctacgtgcccgcagcttatacaagc
aaaatcgacccaacaacgggtttcgtaaacatatttaagttcaaggatctcaccgtggatgccaagcgagagttc
ataaaaaaatttgactcaatcagatatgactcagaaaagaatcttttttgttttaccttcgactacaataatttc
attacacaaaatacggttatgagcaagtcatcctggtccgtatatacgtatggagtgcgcataaagcggagattc
gttaacgggcgattttctaatgagtccgatacaatcgatataacaaaggatatggaaaaaactctggaaatgact
gatataaattggagggacggtcatgacctcaggcaagacattatcgattatgagatcgtgcaacatatttttgag
atctttcggttgactgtccaaatgaggaactctctgtctgaattggaagatagggactacgatcgcctgataagc
cccgtgttgaacgagaataacatattctacgattccgcgaaagccggggatgcgctccctaaggacgccgatgca
aatggggcctattgtattgctttgaaagggctgtacgaaatcaaacagatcaccgaaaactggaaagaagacggg
aagtttagtcgggataaactgaagatatccaacaaggactggtttgactttatccaaaataagcgatatttgagc
ggcagcgagactcccgggacctcagagtccgccacacccgaaagtcaactggtgaagagcgagctggaagagaag
aaaagcgagctcagacataagctgaagtacgttccccacgaatacattgaactgatagaaatcgctagaaacagt
acgcaagacagaatactggaaatgaaggtgatggagttcttcatgaaggtttacggctatcgtggcaaacacctc
gggggctcccggaagcccgccggggctatctacaccgtgggcagtcccatcgactatggcgtgatcgtggacacc
aaagcttatagcggcggatataatctccccatcggccaagccgatgagatgcagaggtatgtggaggagaaccaa
acaagaaacaagcatatcaaccccaacgagtggtggaaggtttatcctagctcggtgaccgagtttaagttccta
ttcgtgtctggccacttcaagggcaactataaggcacagctcactagactgaatcatatcacgaattgcaacggc
gccgtgttatccgtggaggagctactgatcggcggagagatgatcaaagccggcaccctgaccctggaagaggtg
agaagaaagtttaacaatggcgaaataaatttcggcagcggaagtggaagcggctccatcactagaaccaccaac
cctagaaacgtggtgcccaagatctacatgagcgccggcagcatccccctgaccacccacatcaccaactcaatt
cagcccaccctgtggaccatcggcagcatcaacggcgtggcccccctggccaagagcatcaagctgggcatcccc
gtgaccggcagcgcctacaccgatcagaccaccgccatggtgagaaagaaggtgagcgtgttcatgggcagcggc
agcgggagcggctcatcgcagctggttaagagcgagttagaagaaaaaaagagcgaactgcggcataaactgaag
tatgtcccacacgagtacatcgaactgatcgagatcgcgagaaactctacccaagacagaattctggagatgaaa
gtaatggaatttttcatgaaggtgtatggatatagagggaagcacctgggtggcagcagaaaacccgacggcgcc
atctacactgtggggagccccatagactatggtgtgatcgtggataccaaggcgtatagcggcggttacaatctg
cccattgggcaagcggacgagatgcaaagatatgtggaagagaatcagacgaggaacaagcacattaaccctaat
gagtggtggaaggtctaccctagctccgttaccgagttcaagttcctgtttgtgagcgggcattttaagggcaac
tacaaggcacagctgacccgcctgaaccacataacaaactgcaacggtgccgtgctgagcgtagaagagttgcta
atcggcggcgagatgatcaaggccggcacgctaaccctcgaagaggtgcgcagaaagttcaataacggcgaaatc
aatttc
Exemplary nucleic acid sequence of Construct E: Cpf1 (D908A)-XTEN-FokI-polypeptide
linker-FokII
(SEQ ID NO: 35)
atgacacagttcgagggctttaccaacctgtatcaggtgagcaagacactgcggtttgagctgatcccacagggc
aagaccctgaagcacatccaggagcagggcttcatcgaggaggacaaggcccgcaatgatcactacaaggagctg
aagcccatcatcgatcggatctacaagacctatgccgaccagtgcctgcagctggtgcagctggattgggagaac
ctgagcgccgccatcgactcctatagaaaggagaaaaccgaggagacaaggaacgccctgatcgaggagcaggcc
acatatcgcaatgccatccacgactacttcatcggccggacagacaacctgaccgatgccatcaataagagacac
gccgagatctacaagggcctgttcaaggccgagctgtttaatggcaaggtgctgaagcagctgggcaccgtgacc
acaaccgagcacgagaacgccctgctgcggagcttcgacaagtttacaacctacttctccggcttttatagaaac
aggaagaacgtgttcagcgccgaggatatcagcacagccatcccacaccgcatcgtgcaggacaacttccccaag
tttaaggagaattgtcacatcttcacacgcctgatcaccgccgtgcccagcctgcgggagcactttgagaacgtg
aagaaggccatcggcatcttcgtgagcacctccatcgaggaggtgttttccttccctttttataaccagctgctg
acacagacccagatcgacctgtataaccagctgctgggaggaatctctcgggaggcaggcaccgagaagatcaag
ggcctgaacgaggtgctgaatctggccatccagaagaatgatgagacagcccacatcatcgcctccctgccacac
agattcatccccctgtttaagcagatcctgtccgataggaacaccctgtctttcatcctggaggagtttaagagc
gacgaggaagtgatccagtccttctgcaagtacaagacactgctgagaaacgagaacgtgctggagacagccgag
gccctgtttaacgagctgaacagcatcgacctgacacacatcttcatcagccacaagaagctggagacaatcagc
agcgccctgtgcgaccactgggatacactgaggaatgccctgtatgagcggagaatctccgagctgacaggcaag
atcaccaagtctgccaaggagaaggtgcagcgcagcctgaagcacgaggatatcaacctgcaggagatcatctct
gccgcaggcaaggagctgagcgaggccttcaagcagaaaaccagcgagatcctgtcccacgcacacgccgccctg
gatcagccactgcctacaaccctgaagaagcaggaggagaaggagatcctgaagtctcagctggacagcctgctg
ggcctgtaccacctgctggactggtttgccgtggatgagtccaacgaggtggaccccgagttctctgcccggctg
accggcatcaagctggagatggagccttctctgagcttctacaacaaggccagaaattatgccaccaagaagccc
tactccgtggagaagttcaagctgaactttcagatgcctacactggccagaggctgggacgtgaatgtggagaag
aacagaggcgccatcctgtttgtgaagaacggcctgtactatctgggcatcatgccaaagcagaagggcaggtat
aaggccctgagcttcgagcccacagagaaaaccagcgagggctttgataagatgtactatgactacttccctgat
gccgccaagatgatcccaaagtgcagcacccagctgaaggccgtgacagcccactttcagacccacacaaccccc
atcctgctgtccaacaatttcatcgagcctctggagatcacaaaggagatctacgacctgaacaatcctgagaag
gagccaaagaagtttcagacagcctacgccaagaaaaccggcgaccagaagggctacagagaggccctgtgcaag
tggatcgacttcacaagggattttctgtccaagtataccaagacaacctctatcgatctgtctagcctgcggcca
tcctctcagtataaggacctgggcgagtactatgccgagctgaatcccctgctgtaccacatcagcttccagaga
atcgccgagaaggagatcatggatgccgtggagacaggcaagctgtacctgttccagatctataacaaggacttt
gccaagggccaccacggcaagcctaatctgcacacactgtattggaccggcctgttttctccagagaacctggcc
aagacaagcatcaagctgaatggccaggccgagctgttctaccgccctaagtccaggatgaagaggatggcacac
cggctgggagagaagatgctgaacaagaagctgaaggatcagaaaaccccaatccccgacaccctgtaccaggag
ctgtacgactatgtgaatcacagactgtcccacgacctgtctgatgaggccagggccctgctgcccaacgtgatc
accaaggaggtgtctcacgagatcatcaaggataggcgctttaccagcgacaagttctttttccacgtgcctatc
acactgaactatcaggccgccaattccccatctaagttcaaccagagggtgaatgcctacctgaaggagcacccc
gagacacctatcatcggcatcgcccggggcgagagaaacctgatctatatcacagtgatcgactccaccggcaag
atcctggagcagcggagcctgaacaccatccagcagtttgattaccagaagaagctggacaacagggagaaggag
agggtggcagcaaggcaggcctggtctgtggtgggcacaatcaaggatctgaagcagggctatctgagccaggtc
atccacgagatcgtggacctgatgatccactaccaggccgtggtggtgctggagaacctgaatttcggctttaag
agcaagaggaccggcatcgccgagaaggccgtgtaccagcagttcgagaagatgctgatcgataagctgaattgc
ctggtgctgaaggactatccagcagagaaagtgggaggcgtgctgaacccataccagctgacagaccagttcacc
tcctttgccaagatgggcacccagtctggcttcctgttttacgtgcctgccccatatacatctaagatcgatccc
ctgaccggcttcgtggaccccttcgtgtggaaaaccatcaagaatcacgagagccgcaagcacttcctggagggc
ttcgactttctgcactacgacgtgaaaaccggcgacttcatcctgcactttaagatgaacagaaatctgtccttc
cagaggggcctgcccggctttatgcctgcatgggatatcgtgttcgagaagaacgagacacagtttgacgccaag
ggcacccctttcatcgccggcaagagaatcgtgccagtgatcgagaatcacagattcaccggcagataccgggac
ctgtatcctgccaacgagctgatcgccctgctggaggagaagggcatcgtgttcagggatggctccaacatcctg
ccaaagctgctggagaatgacgattctcacgccatcgacaccatggtggccctgatccgcagcgtgctgcagatg
cggaactccaatgccgccacaggcgaggactatatcaacagccccgtgcgcgatctgaatggcgtgtgcttcgac
tcccggtttcagaacccagagtggcccatggacgccgatgccaatggcgcctaccacatcgccctgaagggccag
ctgctgctgaatcacctgaaggagagcaaggatctgaagctgcagaacggcatctccaatcaggactggctggcc
tacatccaggagctgcgcaacagcggcagcgagactcccgggacctcagagtccgccacacccgaaagtcaactg
gtgaagagcgagctggaagagaagaaaagcgagctcagacataagctgaagtacgttccccacgaatacattgaa
ctgatagaaatcgctagaaacagtacgcaagacagaatactggaaatgaaggtgatggagttcttcatgaaggtt
tacggctatcgtggcaaacacctcgggggctcccggaagcccgacggggctatctacaccgtgggcagtcccatc
gactatggcgtgatcgtggacaccaaagcttatagcggcggatataatctccccatcggccaagccgatgagatg
cagaggtatgtggaggagaaccaaacaagaaacaagcatatcaaccccaacgagtggtggaaggtttatcctagc
tcggtgaccgagtttaagttcctattcgtgtctggccacttcaagggcaactataaggcacagctcactagactg
aatcatatcacgaattgcaacggcgccgtgttatccgtggaggagctactgatcggcggagagatgatcaaagcc
ggcaccctgaccctggaagaggtgagaagaaagtttaacaatggcgaaataaatttcggcagcggaagtggaagc
ggctccatcactagaaccaccaaccctagaaacgtggtgcccaagatctacatgagcgccggcagcatccccctg
accacccacatcaccaactcaattcagcccaccctgtggaccatcggcagcatcaacggcgtggcccccctggcc
aagagcatcaagctgggcatccccgtgaccggcagcgcctacaccgatcagaccaccgccatggtgagaaagaag
gtgagcgtgttcatgggcagcggcagcgggagcggctcatcgcagctggttaagagcgagttagaagaaaaaaag
agcgaactgcggcataaactgaagtatgtcccacacgagtacatcgaactgatcgagatcgcgagaaactctacc
caagacagaattctggagatgaaagtaatggaatttttcatgaaggtgtatggatatagagggaagcacctgggt
ggcagcagaaaacccgacggcgccatctacactgtggggagccccatagactatggtgtgatcgtggataccaag
gcgtatagcggcggttacaatctgcccattgggcaagcggacgagatgcaaagatatgtggaagagaatcagacg
aggaacaagcacattaaccctaatgagtggtggaaggtctaccctagctccgttaccgagttcaagttcctgttt
gtgagcgggcattttaagggcaactacaaggcacagctgacccgcctgaaccacataacaaactgcaacggtgcc
gtgctgagcgtagaagagttgctaatcggcggcgagatgatcaaggccggcacgctaaccctcgaagaggtgcgc
agaaagttcaataacggcgaaatcaatttc
Exemplary nucleic acid sequence of Construct F: FokI-polypeptide linker-FokII-XTEN-
Cpf1 (D908A)
(SEQ ID NO: 36)
atgcaactggtgaagagcgagctggaagagaagaaaagcgagctcagacataagctgaagtacgttccccacgaa
tacattgaactgatagaaatcgctagaaacagtacgcaagacagaatactggaaatgaaggtgatggagttcttc
atgaaggtttacggctatcgtggcaaacacctcgggggctcccggaagcccgacggggctatctacaccgtgggc
agtcccatcgactatggcgtgatcgtggacaccaaagcttatagcggcggatataatctccccatcggccaagcc
gatgagatgcagaggtatgtggaggagaaccaaacaagaaacaagcatatcaaccccaacgagtggtggaaggtt
tatcctagctcggtgaccgagtttaagttcctattcgtgtctggccacttcaagggcaactataaggcacagctc
actagactgaatcatatcacgaattgcaacggcgccgtgttatccgtggaggagctactgatcggcggagagatg
atcaaagccggcaccctgaccctggaagaggtgagaagaaagtttaacaatggcgaaataaatttcggcagcgga
agtggaagcggctccatcactagaaccaccaaccctagaaacgtggtgcccaagatctacatgagcgccggcagc
atccccctgaccacccacatcaccaactcaattcagcccaccctgtggaccatcggcagcatcaacggcgtggcc
cccctggccaagagcatcaagctgggcatccccgtgaccggcagcgcctacaccgatcagaccaccgccatggtg
agaaagaaggtgagcgtgttcatgggcagcggcagcgggagcggctcatcgcagctggttaagagcgagttagaa
gaaaaaaagagcgaactgcggcataaactgaagtatgtcccacacgagtacatcgaactgatcgagatcgcgaga
aactctacccaagacagaattctggagatgaaagtaatggaatttttcatgaaggtgtatggatatagagggaag
cacctgggtggcagcagaaaacccgacggcgccatctacactgtggggagccccatagactatggtgtgatcgtg
gataccaaggcgtatagcggcggttacaatctgcccattgggcaagcggacgagatgcaaagatatgtggaagag
aatcagacgaggaacaagcacattaaccctaatgagtggtggaaggtctaccctagctccgttaccgagttcaag
ttcctgtttgtgagcgggcattttaagggcaactacaaggcacagctgacccgcctgaaccacataacaaactgc
aacggtgccgtgctgagcgtagaagagttgctaatcggcggcgagatgatcaaggccggcacgctaaccctcgaa
gaggtgcgcagaaagttcaataacggcgaaatcaatttcagcggcagcgagactcccgggacctcagagtccgcc
acacccgaaagtacacagttcgagggctttaccaacctgtatcaggtgagcaagacactgcggtttgagctgatc
ccacagggcaagaccctgaagcacatccaggagcagggcttcatcgaggaggacaaggcccgcaatgatcactac
aaggagctgaagcccatcatcgatcggatctacaagacctatgccgaccagtgcctgcagctggtgcagctggat
tgggagaacctgagcgccgccatcgactcctatagaaaggagaaaaccgaggagacaaggaacgccctgatcgag
gagcaggccacatatcgcaatgccatccacgactacttcatcggccggacagacaacctgaccgatgccatcaat
aagagacacgccgagatctacaagggcctgttcaaggccgagctgtttaatggcaaggtgctgaagcagctgggc
accgtgaccacaaccgagcacgagaacgccctgctgcggagcttcgacaagtttacaacctacttctccggcttt
tatagaaacaggaagaacgtgttcagcgccgaggatatcagcacagccatcccacaccgcatcgtgcaggacaac
ttccccaagtttaaggagaattgtcacatcttcacacgcctgatcaccgccgtgcccagcctgcgggagcacttt
gagaacgtgaagaaggccatcggcatcttcgtgagcacctccatcgaggaggtgttttccttccctttttataac
cagctgctgacacagacccagatcgacctgtataaccagctgctgggaggaatctctcgggaggcaggcaccgag
aagatcaagggcctgaacgaggtgctgaatctggccatccagaagaatgatgagacagcccacatcatcgcctcc
ctgccacacagattcatccccctgtttaagcagatcctgtccgataggaacaccctgtctttcatcctggaggag
tttaagagcgacgaggaagtgatccagtccttctgcaagtacaagacactgctgagaaacgagaacgtgctggag
acagccgaggccctgtttaacgagctgaacagcatcgacctgacacacatcttcatcagccacaagaagctggag
acaatcagcagcgccctgtgcgaccactgggatacactgaggaatgccctgtatgagcggagaatctccgagctg
acaggcaagatcaccaagtctgccaaggagaaggtgcagcgcagcctgaagcacgaggatatcaacctgcaggag
atcatctctgccgcaggcaaggagctgagcgaggccttcaagcagaaaaccagcgagatcctgtcccacgcacac
gccgccctggatcagccactgcctacaaccctgaagaagcaggaggagaaggagatcctgaagtctcagctggac
agcctgctgggcctgtaccacctgctggactggtttgccgtggatgagtccaacgaggtggaccccgagttctct
gcccggctgaccggcatcaagctggagatggagccttctctgagcttctacaacaaggccagaaattatgccacc
aagaagccctactccgtggagaagttcaagctgaactttcagatgcctacactggccagaggctgggacgtgaat
gtggagaagaacagaggcgccatcctgtttgtgaagaacggcctgtactatctgggcatcatgccaaagcagaag
ggcaggtataaggccctgagcttcgagcccacagagaaaaccagcgagggctttgataagatgtactatgactac
ttccctgatgccgccaagatgatcccaaagtgcagcacccagctgaaggccgtgacagcccactttcagacccac
acaacccccatcctgctgtccaacaatttcatcgagcctctggagatcacaaaggagatctacgacctgaacaat
cctgagaaggagccaaagaagtttcagacagcctacgccaagaaaaccggcgaccagaagggctacagagaggcc
ctgtgcaagtggatcgacttcacaagggattttctgtccaagtataccaagacaacctctatcgatctgtctagc
ctgcggccatcctctcagtataaggacctgggcgagtactatgccgagctgaatcccctgctgtaccacatcagc
ttccagagaatcgccgagaaggagatcatggatgccgtggagacaggcaagctgtacctgttccagatctataac
aaggactttgccaagggccaccacggcaagcctaatctgcacacactgtattggaccggcctgttttctccagag
aacctggccaagacaagcatcaagctgaatggccaggccgagctgttctaccgccctaagtccaggatgaagagg
atggcacaccggctgggagagaagatgctgaacaagaagctgaaggatcagaaaaccccaatccccgacaccctg
taccaggagctgtacgactatgtgaatcacagactgtcccacgacctgtctgatgaggccagggccctgctgccc
aacgtgatcaccaaggaggtgtctcacgagatcatcaaggataggcgctttaccagcgacaagttctttttccac
gtgcctatcacactgaactatcaggccgccaattccccatctaagttcaaccagagggtgaatgcctacctgaag
gagcaccccgagacacctatcatcggcatcgcccggggcgagagaaacctgatctatatcacagtgatcgactcc
accggcaagatcctggagcagcggagcctgaacaccatccagcagtttgattaccagaagaagctggacaacagg
gagaaggagagggtggcagcaaggcaggcctggtctgtggtgggcacaatcaaggatctgaagcagggctatctg
agccaggtcatccacgagatcgtggacctgatgatccactaccaggccgtggtggtgctggagaacctgaatttc
ggctttaagagcaagaggaccggcatcgccgagaaggccgtgtaccagcagttcgagaagatgctgatcgataag
ctgaattgcctggtgctgaaggactatccagcagagaaagtgggaggcgtgctgaacccataccagctgacagac
cagttcacctcctttgccaagatgggcacccagtctggcttcctgttttacgtgcctgccccatatacatctaag
atcgatcccctgaccggcttcgtggaccccttcgtgtggaaaaccatcaagaatcacgagagccgcaagcacttc
ctggagggcttcgactttctgcactacgacgtgaaaaccggcgacttcatcctgcactttaagatgaacagaaat
ctgtccttccagaggggcctgcccggctttatgcctgcatgggatatcgtgttcgagaagaacgagacacagttt
gacgccaagggcacccctttcatcgccggcaagagaatcgtgccagtgatcgagaatcacagattcaccggcaga
taccgggacctgtatcctgccaacgagctgatcgccctgctggaggagaagggcatcgtgttcagggatggctcc
aacatcctgccaaagctgctggagaatgacgattctcacgccatcgacaccatggtggccctgatccgcagcgtg
ctgcagatgcggaactccaatgccgccacaggcgaggactatatcaacagccccgtgcgcgatctgaatggcgtg
tgcttcgactcccggtttcagaacccagagtggcccatggacgccgatgccaatggcgcctaccacatcgccctg
aagggccagctgctgctgaatcacctgaaggagagcaaggatctgaagctgcagaacggcatctccaatcaggac
tggctggcctacatccaggagctgcgcaac
Exemplary nucleic acid sequence of Construct G: Cpf1 (D908A)-XTEN-FokI (D450A)-
polypeptide linker-FokII
(SEQ ID NO: 37)
atgacacagttcgagggctttaccaacctgtatcaggtgagcaagacactgcggtttgagctgatcccacagggc
aagaccctgaagcacatccaggagcagggcttcatcgaggaggacaaggcccgcaatgatcactacaaggagctg
aagcccatcatcgatcggatctacaagacctatgccgaccagtgcctgcagctggtgcagctggattgggagaac
ctgagcgccgccatcgactcctatagaaaggagaaaaccgaggagacaaggaacgccctgatcgaggagcaggcc
acatatcgcaatgccatccacgactacttcatcggccggacagacaacctgaccgatgccatcaataagagacac
gccgagatctacaagggcctgttcaaggccgagctgtttaatggcaaggtgctgaagcagctgggcaccgtgacc
acaaccgagcacgagaacgccctgctgcggagcttcgacaagtttacaacctacttctccggcttttatagaaac
aggaagaacgtgttcagcgccgaggatatcagcacagccatcccacaccgcatcgtgcaggacaacttccccaag
tttaaggagaattgtcacatcttcacacgcctgatcaccgccgtgcccagcctgcgggagcactttgagaacgtg
aagaaggccatcggcatcttcgtgagcacctccatcgaggaggtgttttccttccctttttataaccagctgctg
acacagacccagatcgacctgtataaccagctgctgggaggaatctctcgggaggcaggcaccgagaagatcaag
ggcctgaacgaggtgctgaatctggccatccagaagaatgatgagacagcccacatcatcgcctccctgccacac
agattcatccccctgtttaagcagatcctgtccgataggaacaccctgtctttcatcctggaggagtttaagagc
gacgaggaagtgatccagtccttctgcaagtacaagacactgctgagaaacgagaacgtgctggagacagccgag
gccctgtttaacgagctgaacagcatcgacctgacacacatcttcatcagccacaagaagctggagacaatcagc
agcgccctgtgcgaccactgggatacactgaggaatgccctgtatgagcggagaatctccgagctgacaggcaag
atcaccaagtctgccaaggagaaggtgcagcgcagcctgaagcacgaggatatcaacctgcaggagatcatctct
gccgcaggcaaggagctgagcgaggccttcaagcagaaaaccagcgagatcctgtcccacgcacacgccgccctg
gatcagccactgcctacaaccctgaagaagcaggaggagaaggagatcctgaagtctcagctggacagcctgctg
ggcctgtaccacctgctggactggtttgccgtggatgagtccaacgaggtggaccccgagttctctgcccggctg
accggcatcaagctggagatggagccttctctgagcttctacaacaaggccagaaattatgccaccaagaagccc
tactccgtggagaagttcaagctgaactttcagatgcctacactggccagaggctgggacgtgaatgtggagaag
aacagaggcgccatcctgtttgtgaagaacggcctgtactatctgggcatcatgccaaagcagaagggcaggtat
aaggccctgagcttcgagcccacagagaaaaccagcgagggctttgataagatgtactatgactacttccctgat
gccgccaagatgatcccaaagtgcagcacccagctgaaggccgtgacagcccactttcagacccacacaaccccc
atcctgctgtccaacaatttcatcgagcctctggagatcacaaaggagatctacgacctgaacaatcctgagaag
gagccaaagaagtttcagacagcctacgccaagaaaaccggcgaccagaagggctacagagaggccctgtgcaag
tggatcgacttcacaagggattttctgtccaagtataccaagacaacctctatcgatctgtctagcctgcggcca
tcctctcagtataaggacctgggcgagtactatgccgagctgaatcccctgctgtaccacatcagcttccagaga
atcgccgagaaggagatcatggatgccgtggagacaggcaagctgtacctgttccagatctataacaaggacttt
gccaagggccaccacggcaagcctaatctgcacacactgtattggaccggcctgttttctccagagaacctggcc
aagacaagcatcaagctgaatggccaggccgagctgttctaccgccctaagtccaggatgaagaggatggcacac
cggctgggagagaagatgctgaacaagaagctgaaggatcagaaaaccccaatccccgacaccctgtaccaggag
ctgtacgactatgtgaatcacagactgtcccacgacctgtctgatgaggccagggccctgctgcccaacgtgatc
accaaggaggtgtctcacgagatcatcaaggataggcgctttaccagcgacaagttctttttccacgtgcctatc
acactgaactatcaggccgccaattccccatctaagttcaaccagagggtgaatgcctacctgaaggagcacccc
gagacacctatcatcggcatcgcccggggcgagagaaacctgatctatatcacagtgatcgactccaccggcaag
atcctggagcagcggagcctgaacaccatccagcagtttgattaccagaagaagctggacaacagggagaaggag
agggtggcagcaaggcaggcctggtctgtggtgggcacaatcaaggatctgaagcagggctatctgagccaggtc
atccacgagatcgtggacctgatgatccactaccaggccgtggtggtgctggagaacctgaatttcggctttaag
agcaagaggaccggcatcgccgagaaggccgtgtaccagcagttcgagaagatgctgatcgataagctgaattgc
ctggtgctgaaggactatccagcagagaaagtgggaggcgtgctgaacccataccagctgacagaccagttcacc
tcctttgccaagatgggcacccagtctggcttcctgttttacgtgcctgccccatatacatctaagatcgatccc
ctgaccggcttcgtggaccccttcgtgtggaaaaccatcaagaatcacgagagccgcaagcacttcctggagggc
ttcgactttctgcactacgacgtgaaaaccggcgacttcatcctgcactttaagatgaacagaaatctgtccttc
cagaggggcctgcccggctttatgcctgcatgggatatcgtgttcgagaagaacgagacacagtttgacgccaag
ggcacccctttcatcgccggcaagagaatcgtgccagtgatcgagaatcacagattcaccggcagataccgggac
ctgtatcctgccaacgagctgatcgccctgctggaggagaagggcatcgtgttcagggatggctccaacatcctg
ccaaagctgctggagaatgacgattctcacgccatcgacaccatggtggccctgatccgcagcgtgctgcagatg
cggaactccaatgccgccacaggcgaggactatatcaacagccccgtgcgcgatctgaatggcgtgtgcttcgac
tcccggtttcagaacccagagtggcccatggacgccgatgccaatggcgcctaccacatcgccctgaagggccag
ctgctgctgaatcacctgaaggagagcaaggatctgaagctgcagaacggcatctccaatcaggactggctggcc
tacatccaggagctgcgcaacagcggcagcgagactcccgggacctcagagtccgccacacccgaaagtcaactg
gtgaagagcgagctggaagagaagaaaagcgagctcagacataagctgaagtacgttccccacgaatacattgaa
ctgatagaaatcgctagaaacagtacgcaagacagaatactggaaatgaaggtgatggagttcttcatgaaggtt
tacggctatcgtggcaaacacctcgggggctcccggaagcccgccggggctatctacaccgtgggcagtcccatc
gactatggcgtgatcgtggacaccaaagcttatagcggcggatataatctccccatcggccaagccgatgagatg
cagaggtatgtggaggagaaccaaacaagaaacaagcatatcaaccccaacgagtggtggaaggtttatcctagc
tcggtgaccgagtttaagttcctattcgtgtctggccacttcaagggcaactataaggcacagctcactagactg
aatcatatcacgaattgcaacggcgccgtgttatccgtggaggagctactgatcggcggagagatgatcaaagcc
ggcaccctgaccctggaagaggtgagaagaaagtttaacaatggcgaaataaatttcggcagcggaagtggaagc
ggctccatcactagaaccaccaaccctagaaacgtggtgcccaagatctacatgagcgccggcagcatccccctg
accacccacatcaccaactcaattcagcccaccctgtggaccatcggcagcatcaacggcgtggcccccctggcc
aagagcatcaagctgggcatccccgtgaccggcagcgcctacaccgatcagaccaccgccatggtgagaaagaag
gtgagcgtgttcatgggcagcggcagcgggagcggctcatcgcagctggttaagagcgagttagaagaaaaaaag
agcgaactgcggcataaactgaagtatgtcccacacgagtacatcgaactgatcgagatcgcgagaaactctacc
caagacagaattctggagatgaaagtaatggaatttttcatgaaggtgtatggatatagagggaagcacctgggt
ggcagcagaaaacccgacggcgccatctacactgtggggagccccatagactatggtgtgatcgtggataccaag
gcgtatagcggcggttacaatctgcccattgggcaagcggacgagatgcaaagatatgtggaagagaatcagacg
aggaacaagcacattaaccctaatgagtggtggaaggtctaccctagctccgttaccgagttcaagttcctgttt
gtgagcgggcattttaagggcaactacaaggcacagctgacccgcctgaaccacataacaaactgcaacggtgcc
gtgctgagcgtagaagagttgctaatcggcggcgagatgatcaaggccggcacgctaaccctcgaagaggtgcgc
agaaagttcaataacggcgaaatcaatttc
Exemplary nucleic acid sequence of Construct H: Cpf1 (D908A)-XTEN-FokI-
polypeptide linker-FokII (D450A)
(SEQ ID NO: 38)
atgacacagttcgagggctttaccaacctgtatcaggtgagcaagacactgcggtttgagctgatcccacagggc
aagaccctgaagcacatccaggagcagggcttcatcgaggaggacaaggcccgcaatgatcactacaaggagctg
aagcccatcatcgatcggatctacaagacctatgccgaccagtgcctgcagctggtgcagctggattgggagaac
ctgagcgccgccatcgactcctatagaaaggagaaaaccgaggagacaaggaacgccctgatcgaggagcaggcc
acatatcgcaatgccatccacgactacttcatcggccggacagacaacctgaccgatgccatcaataagagacac
gccgagatctacaagggcctgttcaaggccgagctgtttaatggcaaggtgctgaagcagctgggcaccgtgacc
acaaccgagcacgagaacgccctgctgcggagcttcgacaagtttacaacctacttctccggcttttatagaaac
aggaagaacgtgttcagcgccgaggatatcagcacagccatcccacaccgcatcgtgcaggacaacttccccaag
tttaaggagaattgtcacatcttcacacgcctgatcaccgccgtgcccagcctgcgggagcactttgagaacgtg
aagaaggccatcggcatcttcgtgagcacctccatcgaggaggtgttttccttccctttttataaccagctgctg
acacagacccagatcgacctgtataaccagctgctgggaggaatctctcgggaggcaggcaccgagaagatcaag
ggcctgaacgaggtgctgaatctggccatccagaagaatgatgagacagcccacatcatcgcctccctgccacac
agattcatccccctgtttaagcagatcctgtccgataggaacaccctgtctttcatcctggaggagtttaagagc
gacgaggaagtgatccagtccttctgcaagtacaagacactgctgagaaacgagaacgtgctggagacagccgag
gccctgtttaacgagctgaacagcatcgacctgacacacatcttcatcagccacaagaagctggagacaatcagc
agcgccctgtgcgaccactgggatacactgaggaatgccctgtatgagcggagaatctccgagctgacaggcaag
atcaccaagtctgccaaggagaaggtgcagcgcagcctgaagcacgaggatatcaacctgcaggagatcatctct
gccgcaggcaaggagctgagcgaggccttcaagcagaaaaccagcgagatcctgtcccacgcacacgccgccctg
gatcagccactgcctacaaccctgaagaagcaggaggagaaggagatcctgaagtctcagctggacagcctgctg
ggcctgtaccacctgctggactggtttgccgtggatgagtccaacgaggtggaccccgagttctctgcccggctg
accggcatcaagctggagatggagccttctctgagcttctacaacaaggccagaaattatgccaccaagaagccc
tactccgtggagaagttcaagctgaactttcagatgcctacactggccagaggctgggacgtgaatgtggagaag
aacagaggcgccatcctgtttgtgaagaacggcctgtactatctgggcatcatgccaaagcagaagggcaggtat
aaggccctgagcttcgagcccacagagaaaaccagcgagggctttgataagatgtactatgactacttccctgat
gccgccaagatgatcccaaagtgcagcacccagctgaaggccgtgacagcccactttcagacccacacaaccccc
atcctgctgtccaacaatttcatcgagcctctggagatcacaaaggagatctacgacctgaacaatcctgagaag
gagccaaagaagtttcagacagcctacgccaagaaaaccggcgaccagaagggctacagagaggccctgtgcaag
tggatcgacttcacaagggattttctgtccaagtataccaagacaacctctatcgatctgtctagcctgcggcca
tcctctcagtataaggacctgggcgagtactatgccgagctgaatcccctgctgtaccacatcagcttccagaga
atcgccgagaaggagatcatggatgccgtggagacaggcaagctgtacctgttccagatctataacaaggacttt
gccaagggccaccacggcaagcctaatctgcacacactgtattggaccggcctgttttctccagagaacctggcc
aagacaagcatcaagctgaatggccaggccgagctgttctaccgccctaagtccaggatgaagaggatggcacac
cggctgggagagaagatgctgaacaagaagctgaaggatcagaaaaccccaatccccgacaccctgtaccaggag
ctgtacgactatgtgaatcacagactgtcccacgacctgtctgatgaggccagggccctgctgcccaacgtgatc
accaaggaggtgtctcacgagatcatcaaggataggcgctttaccagcgacaagttctttttccacgtgcctatc
acactgaactatcaggccgccaattccccatctaagttcaaccagagggtgaatgcctacctgaaggagcacccc
gagacacctatcatcggcatcgcccggggcgagagaaacctgatctatatcacagtgatcgactccaccggcaag
atcctggagcagcggagcctgaacaccatccagcagtttgattaccagaagaagctggacaacagggagaaggag
agggtggcagcaaggcaggcctggtctgtggtgggcacaatcaaggatctgaagcagggctatctgagccaggtc
atccacgagatcgtggacctgatgatccactaccaggccgtggtggtgctggagaacctgaatttcggctttaag
agcaagaggaccggcatcgccgagaaggccgtgtaccagcagttcgagaagatgctgatcgataagctgaattgc
ctggtgctgaaggactatccagcagagaaagtgggaggcgtgctgaacccataccagctgacagaccagttcacc
tcctttgccaagatgggcacccagtctggcttcctgttttacgtgcctgccccatatacatctaagatcgatccc
ctgaccggcttcgtggaccccttcgtgtggaaaaccatcaagaatcacgagagccgcaagcacttcctggagggc
ttcgactttctgcactacgacgtgaaaaccggcgacttcatcctgcactttaagatgaacagaaatctgtccttc
cagaggggcctgcccggctttatgcctgcatgggatatcgtgttcgagaagaacgagacacagtttgacgccaag
ggcacccctttcatcgccggcaagagaatcgtgccagtgatcgagaatcacagattcaccggcagataccgggac
ctgtatcctgccaacgagctgatcgccctgctggaggagaagggcatcgtgttcagggatggctccaacatcctg
ccaaagctgctggagaatgacgattctcacgccatcgacaccatggtggccctgatccgcagcgtgctgcagatg
cggaactccaatgccgccacaggcgaggactatatcaacagccccgtgcgcgatctgaatggcgtgtgcttcgac
tcccggtttcagaacccagagtggcccatggacgccgatgccaatggcgcctaccacatcgccctgaagggccag
ctgctgctgaatcacctgaaggagagcaaggatctgaagctgcagaacggcatctccaatcaggactggctggcc
tacatccaggagctgcgcaacagcggcagcgagactcccgggacctcagagtccgccacacccgaaagtcaactg
gtgaagagcgagctggaagagaagaaaagcgagctcagacataagctgaagtacgttccccacgaatacattgaa
ctgatagaaatcgctagaaacagtacgcaagacagaatactggaaatgaaggtgatggagttcttcatgaaggtt
tacggctatcgtggcaaacacctcgggggctcccggaagcccgacggggctatctacaccgtgggcagtcccatc
gactatggcgtgatcgtggacaccaaagcttatagcggcggatataatctccccatcggccaagccgatgagatg
cagaggtatgtggaggagaaccaaacaagaaacaagcatatcaaccccaacgagtggtggaaggtttatcctagc
tcggtgaccgagtttaagttcctattcgtgtctggccacttcaagggcaactataaggcacagctcactagactg
aatcatatcacgaattgcaacggcgccgtgttatccgtggaggagctactgatcggcggagagatgatcaaagcc
ggcaccctgaccctggaagaggtgagaagaaagtttaacaatggcgaaataaatttcggcagcggaagtggaagc
ggctccatcactagaaccaccaaccctagaaacgtggtgcccaagatctacatgagcgccggcagcatccccctg
accacccacatcaccaactcaattcagcccaccctgtggaccatcggcagcatcaacggcgtggcccccctggcc
aagagcatcaagctgggcatccccgtgaccggcagcgcctacaccgatcagaccaccgccatggtgagaaagaag
gtgagcgtgttcatgggcagcggcagcgggagcggctcatcgcagctggttaagagcgagttagaagaaaaaaag
agcgaactgcggcataaactgaagtatgtcccacacgagtacatcgaactgatcgagatcgcgagaaactctacc
caagacagaattctggagatgaaagtaatggaatttttcatgaaggtgtatggatatagagggaagcacctgggt
ggcagcagaaaacccgccggcgccatctacactgtggggagccccatagactatggtgtgatcgtggataccaag
gcgtatagcggcggttacaatctgcccattgggcaagcggacgagatgcaaagatatgtggaagagaatcagacg
aggaacaagcacattaaccctaatgagtggtggaaggtctaccctagctccgttaccgagttcaagttcctgttt
gtgagcgggcattttaagggcaactacaaggcacagctgacccgcctgaaccacataacaaactgcaacggtgcc
gtgctgagcgtagaagagttgctaatcggcggcgagatgatcaaggccggcacgctaaccctcgaagaggtgcgc
agaaagttcaataacggcgaaatcaatttc
Exemplary nucleic acid sequence of Construct I: FokI (D450A)-polypeptide linker-
FokII-XTEN-Cpf1 (D908A)
(SEQ ID NO: 39)
atgcaactggtgaagagcgagctggaagagaagaaaagcgagctcagacataagctgaagtacgttccccacgaa
tacattgaactgatagaaatcgctagaaacagtacgcaagacagaatactggaaatgaaggtgatggagttcttc
atgaaggtttacggctatcgtggcaaacacctcgggggctcccggaagcccgccggggctatctacaccgtgggc
agtcccatcgactatggcgtgatcgtggacaccaaagcttatagcggcggatataatctccccatcggccaagcc
gatgagatgcagaggtatgtggaggagaaccaaacaagaaacaagcatatcaaccccaacgagtggtggaaggtt
tatcctagctcggtgaccgagtttaagttcctattcgtgtctggccacttcaagggcaactataaggcacagctc
actagactgaatcatatcacgaattgcaacggcgccgtgttatccgtggaggagctactgatcggcggagagatg
atcaaagccggcaccctgaccctggaagaggtgagaagaaagtttaacaatggcgaaataaatttcggcagcgga
agtggaagcggctccatcactagaaccaccaaccctagaaacgtggtgcccaagatctacatgagcgccggcagc
atccccctgaccacccacatcaccaactcaattcagcccaccctgtggaccatcggcagcatcaacggcgtggcc
cccctggccaagagcatcaagctgggcatccccgtgaccggcagcgcctacaccgatcagaccaccgccatggtg
agaaagaaggtgagcgtgttcatgggcagcggcagcgggagcggctcatcgcagctggttaagagcgagttagaa
gaaaaaaagagcgaactgcggcataaactgaagtatgtcccacacgagtacatcgaactgatcgagatcgcgaga
aactctacccaagacagaattctggagatgaaagtaatggaatttttcatgaaggtgtatggatatagagggaag
cacctgggtggcagcagaaaacccgacggcgccatctacactgtggggagccccatagactatggtgtgatcgtg
gataccaaggcgtatagcggcggttacaatctgcccattgggcaagcggacgagatgcaaagatatgtggaagag
aatcagacgaggaacaagcacattaaccctaatgagtggtggaaggtctaccctagctccgttaccgagttcaag
ttcctgtttgtgagcgggcattttaagggcaactacaaggcacagctgacccgcctgaaccacataacaaactgc
aacggtgccgtgctgagcgtagaagagttgctaatcggcggcgagatgatcaaggccggcacgctaaccctcgaa
gaggtgcgcagaaagttcaataacggcgaaatcaatttcagcggcagcgagactcccgggacctcagagtccgcc
acacccgaaagtacacagttcgagggctttaccaacctgtatcaggtgagcaagacactgcggtttgagctgatc
ccacagggcaagaccctgaagcacatccaggagcagggcttcatcgaggaggacaaggcccgcaatgatcactac
aaggagctgaagcccatcatcgatcggatctacaagacctatgccgaccagtgcctgcagctggtgcagctggat
tgggagaacctgagcgccgccatcgactcctatagaaaggagaaaaccgaggagacaaggaacgccctgatcgag
gagcaggccacatatcgcaatgccatccacgactacttcatcggccggacagacaacctgaccgatgccatcaat
aagagacacgccgagatctacaagggcctgttcaaggccgagctgtttaatggcaaggtgctgaagcagctgggc
accgtgaccacaaccgagcacgagaacgccctgctgcggagcttcgacaagtttacaacctacttctccggcttt
tatagaaacaggaagaacgtgttcagcgccgaggatatcagcacagccatcccacaccgcatcgtgcaggacaac
ttccccaagtttaaggagaattgtcacatcttcacacgcctgatcaccgccgtgcccagcctgcgggagcacttt
gagaacgtgaagaaggccatcggcatcttcgtgagcacctccatcgaggaggtgttttccttccctttttataac
cagctgctgacacagacccagatcgacctgtataaccagctgctgggaggaatctctcgggaggcaggcaccgag
aagatcaagggcctgaacgaggtgctgaatctggccatccagaagaatgatgagacagcccacatcatcgcctcc
ctgccacacagattcatccccctgtttaagcagatcctgtccgataggaacaccctgtctttcatcctggaggag
tttaagagcgacgaggaagtgatccagtccttctgcaagtacaagacactgctgagaaacgagaacgtgctggag
acagccgaggccctgtttaacgagctgaacagcatcgacctgacacacatcttcatcagccacaagaagctggag
acaatcagcagcgccctgtgcgaccactgggatacactgaggaatgccctgtatgagcggagaatctccgagctg
acaggcaagatcaccaagtctgccaaggagaaggtgcagcgcagcctgaagcacgaggatatcaacctgcaggag
atcatctctgccgcaggcaaggagctgagcgaggccttcaagcagaaaaccagcgagatcctgtcccacgcacac
gccgccctggatcagccactgcctacaaccctgaagaagcaggaggagaaggagatcctgaagtctcagctggac
agcctgctgggcctgtaccacctgctggactggtttgccgtggatgagtccaacgaggtggaccccgagttctct
gcccggctgaccggcatcaagctggagatggagccttctctgagcttctacaacaaggccagaaattatgccacc
aagaagccctactccgtggagaagttcaagctgaactttcagatgcctacactggccagaggctgggacgtgaat
gtggagaagaacagaggcgccatcctgtttgtgaagaacggcctgtactatctgggcatcatgccaaagcagaag
ggcaggtataaggccctgagcttcgagcccacagagaaaaccagcgagggctttgataagatgtactatgactac
ttccctgatgccgccaagatgatcccaaagtgcagcacccagctgaaggccgtgacagcccactttcagacccac
acaacccccatcctgctgtccaacaatttcatcgagcctctggagatcacaaaggagatctacgacctgaacaat
cctgagaaggagccaaagaagtttcagacagcctacgccaagaaaaccggcgaccagaagggctacagagaggcc
ctgtgcaagtggatcgacttcacaagggattttctgtccaagtataccaagacaacctctatcgatctgtctagc
ctgcggccatcctctcagtataaggacctgggcgagtactatgccgagctgaatcccctgctgtaccacatcagc
ttccagagaatcgccgagaaggagatcatggatgccgtggagacaggcaagctgtacctgttccagatctataac
aaggactttgccaagggccaccacggcaagcctaatctgcacacactgtattggaccggcctgttttctccagag
aacctggccaagacaagcatcaagctgaatggccaggccgagctgttctaccgccctaagtccaggatgaagagg
atggcacaccggctgggagagaagatgctgaacaagaagctgaaggatcagaaaaccccaatccccgacaccctg
taccaggagctgtacgactatgtgaatcacagactgtcccacgacctgtctgatgaggccagggccctgctgccc
aacgtgatcaccaaggaggtgtctcacgagatcatcaaggataggcgctttaccagcgacaagttctttttccac
gtgcctatcacactgaactatcaggccgccaattccccatctaagttcaaccagagggtgaatgcctacctgaag
gagcaccccgagacacctatcatcggcatcgcccggggcgagagaaacctgatctatatcacagtgatcgactcc
accggcaagatcctggagcagcggagcctgaacaccatccagcagtttgattaccagaagaagctggacaacagg
gagaaggagagggtggcagcaaggcaggcctggtctgtggtgggcacaatcaaggatctgaagcagggctatctg
agccaggtcatccacgagatcgtggacctgatgatccactaccaggccgtggtggtgctggagaacctgaatttc
ggctttaagagcaagaggaccggcatcgccgagaaggccgtgtaccagcagttcgagaagatgctgatcgataag
ctgaattgcctggtgctgaaggactatccagcagagaaagtgggaggcgtgctgaacccataccagctgacagac
cagttcacctcctttgccaagatgggcacccagtctggcttcctgttttacgtgcctgccccatatacatctaag
atcgatcccctgaccggcttcgtggaccccttcgtgtggaaaaccatcaagaatcacgagagccgcaagcacttc
ctggagggcttcgactttctgcactacgacgtgaaaaccggcgacttcatcctgcactttaagatgaacagaaat
ctgtccttccagaggggcctgcccggctttatgcctgcatgggatatcgtgttcgagaagaacgagacacagttt
gacgccaagggcacccctttcatcgccggcaagagaatcgtgccagtgatcgagaatcacagattcaccggcaga
taccgggacctgtatcctgccaacgagctgatcgccctgctggaggagaagggcatcgtgttcagggatggctcc
aacatcctgccaaagctgctggagaatgacgattctcacgccatcgacaccatggtggccctgatccgcagcgtg
ctgcagatgcggaactccaatgccgccacaggcgaggactatatcaacagccccgtgcgcgatctgaatggcgtg
tgcttcgactcccggtttcagaacccagagtggcccatggacgccgatgccaatggcgcctaccacatcgccctg
aagggccagctgctgctgaatcacctgaaggagagcaaggatctgaagctgcagaacggcatctccaatcaggac
tggctggcctacatccaggagctgcgcaac

The nucleic acids can comprise any isolated or purified nucleotide sequence which encodes any of fusion polypeptides, portions, or functional variants thereof. Alternatively, 50 the nucleotide sequence can comprise a nucleotide sequence which is degenerate to any of the sequences or a combination of degenerate sequences.

Also provided are vectors comprising said nucleic acids. Nucleic acids provided in the present disclosure include nucleic acid sequences which encode proteins, guide RNAs (gRNAs), and selection cassettes (i.e. ampicillin resistance cassettes and puromycin resistance cassettes), as well as nucleic acid sequences which control the expression of the same, i.e. promoters, enhancers, polyA signals etc.

Nucleic acids provided in the present disclosure include features directed to promoting or controlling replication of said nucleic acids in systems for manufacturing said nucleic acids. In some embodiments, nucleic acids for modifying cells are produced in insect cells, yeast cells, or bacterial cells.

Nucleic acids encoding any of the fusion polyproteins described herein can be incorporated into a vector, such as a recombinant expression vector. As described herein, the terms “recombinant expression vector” and “vector” may be used interchangeably and refer to a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, lentiviral vectors, and the like.

In some embodiments, vectors are not naturally-occurring as a whole. However, parts of the vectors can be naturally-occurring. The inventive recombinant expression vectors can comprise any type of nucleotides, including, but not limited to DNA and RNA, which can be single-stranded or double-stranded, synthesized or obtained in part from natural sources, and which can contain natural, non-natural or altered nucleotides. In some embodiments, the vector is a DNA vector. In some embodiments, the vector is an RNA vector. The vectors can comprise naturally-occurring or non-naturally-occurring internucleotide linkages, or both types of linkages. In some embodiments, a non-naturally occurring or altered nucleotides or internucleotide linkages do not hinder the transcription or replication of the vector.

The vector may be any suitable recombinant expression vector, and can be used to transform or transfect any suitable host cell. Suitable vectors include those designed for propagation and expansion or for expression or both, such as plasmids and viruses. A vector can be selected from the group consisting of the pUC series (Fermentas Life Sciences, Glen Burnie, MD), the pBluescript series (Stratagene, LaJolla, CA), the pET series (Novagen, Madison, WI), the pGEX series (Pharmacia Biotech, Uppsala, Sweden), and the pEX series (Clontech, Palo Alto, CA). Bacteriophage vectors, such as LGT1O, λGT11, LZapII (Stratagene), XEMBT4, and λNMI149, also can be used. Examples of plant expression vectors include pBIO1, pBI101.2, pBI101.3, pBH21 and pBIN19 (Clontech). Examples of animal expression vectors include pEUK-CI, pMAM, and pMAMneo (Clontech). The recombinant expression vector may be a viral vector, e.g., an adenoviral vector, a retroviral vector, or a lentiviral vector.

In some embodiments, the vectors of the invention can be prepared using standard recombinant DNA techniques described in, for example, Green et al., supra. Constructs of expression vectors, which are circular or linear, can be prepared to contain a replication system functional in a prokaryotic or eukaryotic host cell. Replication systems can be derived, e.g., from ColEl, 2μ plasmid, λ, SV40, bovine papilloma virus, and the like.

A recombinant expression vector may comprise regulatory sequences, such as transcription and translation initiation and termination codons, which are specific to the type of host cell (e.g., bacterium, fungus, plant, or animal) into which the vector is to be introduced, as appropriate, and taking into consideration whether the vector is DNA- or RNA-based. A recombinant expression vector may also comprise restriction sites to facilitate cloning.

A vector can include one or more marker genes, which allow for selection of transformed or transfected host cells. Marker genes include biocide resistance, e.g., resistance to antibiotics, heavy metals, etc., complementation in an auxotrophic host to provide prototrophy, and the like. Suitable marker genes for the inventive expression vectors include, for instance, neomycin/G418 resistance genes, hygromycin resistance genes, histidinol resistance genes, tetracycline resistance genes, puromycin resistance genes, and ampicillin resistance genes.

In some embodiments, a recombinant expression vector can comprise a native or nonnative promoter operably linked to the nucleotide sequence encoding the fusion polypeptide or to the nucleotide sequence which is complementary to or which hybridizes to the nucleotide sequence encoding the fusion polypeptide. The selection of promoters, e.g., strong, weak, inducible, etc, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, a SFFV promoter, an EF1α promoter, an SV40 promoter, an RSV promoter, a U6 promoter, a beta actin promoter, or a promoter found in the long-terminal repeat of the murine stem cell virus.

Selection of a promoter for a particular type of polymerase may be desired. As will be understood by one of ordinary skill in the art, transcription in eukaryotic cells is typically performed by three types of RNA polymerases, RNA pol I, and RNA pol II, and RNA pol III. See, e.g., Butler et al. Genes & Dev. (2002) 16: 2583-2592. In some embodiments, the vector comprises an RNA pol I promoter. In some embodiments, the vector comprises an RNA pol II promoter. In some embodiments, the vector comprises an RNA pol III promoter.

Examples of RNA pol II promoters include, without limitation, CMV promoter, CAG promoter, CAGGS promoter, ubiquitin promoter, GAPDH promoter, RSV LTR promoter, EF1A promoter, PGK promoter, UbiC promoter, actin promoter, dihydrofolate promoter, B29 promoter, Desmin promoter, Endoglin promoter, FLT-1 promoter, GFPA promoter, and SYN1 promoter. In some embodiments, the vector comprises a CMV promoter.

Examples of RNA pol III promoters include, without limitation, H1 promoter, U6 promoter, 7SK promoter, 7SK1 promoter, 7SK2 promoter, 7SK3 promoter, and U3 promoter. In some embodiments, the vector comprises a U6 promoter.

Further, the vectors can be made to include a suicide gene. As used herein, the term “suicide gene” refers to a gene that causes the cell expressing the suicide gene to die. A suicide gene can be a gene that confers sensitivity to an agent, e.g., a drug, upon the cell in which the gene is expressed, and causes the cell to die when the cell is contacted with or exposed to the agent. Suicide genes are known in the art and include, for example, the Herpes Simplex Virus (HSV) thymidine kinase (TK) gene, cytosine deaminase, purine nucleoside phosphorylase, and nitroreductase.

In some embodiments, a nucleic acid encoding any of the fusion polypeptides described herein is operably linked to another nucleic acid sequence. As used herein, the term “operably linked” refers to a functional linkage between, for example, a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence (e.g., encoding any of the fusion polypeptides described herein). Generally, operably linked DNA sequences are contiguous and, where necessary to join two protein coding regions, in the same reading frame.

The vectors described herein can be designed for transient expression, stable expression, or for both. Alternatively or in addition, the recombinant expression vectors can be made for constitutive expression or for inducible expression.

Any of the vectors described herein may further comprise one or more additional regulatory elements to modulate expression level and/or stability of the fusion polypeptides expressed from said vectors. Examples of additional regulatory elements include, enhancer sequences, polyA termination sequences (e.g., from BGH, SV40), S/MAR elements, and other posttranscriptional and cis-regulatory elements.

In some embodiments, the vector comprises a cis-regulatory element, such as from hepatitis B virus (HPRE) or Woodchuck hepatitis virus, which are though to increase transgene expression by promoting mRNA exportation from the nucleus to the cytoplasm, enhancing 3′ end processing and stability. See, e.g., Sun et al. DNA Cell Biol. (2009) 28(5): 233-250. In some embodiments, the vector comprises a Woodchuck hepatitis posttranscriptional regulatory element (WPRE). In some embodiments, the vector comprises a hepatitis posttranscriptional regulatory element (HPRE).

Any of the vectors described herein may also comprise one or more guide RNAs (gRNAs), which may function, for example, to guide any of the fusion polypeptides described herein to a target sequence in the genome of a host cell.

In some examples, the vectors described herein comprise a promoter operably linked to a coding sequence of any of the fusion polypeptides described herein. In some examples, the vectors described herein comprise a promoter operably linked to a coding sequence of any of the fusion polypeptides described herein, linked to one or more additional regulatory elements. An example composition of a vector comprises an RNA pol II promoter operably linked to a coding sequence of any of the fusion polypeptides described herein, linked to one or more additional regulatory elements. In one example, the vector comprises an RNA pol II promoter operably linked to a coding sequence of any of the fusion polypeptides described herein, linked to one or more additional regulatory elements (e.g., HPRE or WPRE).

In some examples, the vectors described herein comprise a first promoter operably linked to a sequence encoding a gRNA, a second promoter operably linked to a coding sequence of any of the fusion polypeptides described herein, linked to one or more additional regulatory elements. An example composition of a vector comprises an RNA pol III promoter operably linked to a sequence encoding a gRNA, an RNA pol II promoter operably linked to a coding sequence of any of the fusion polypeptides described herein, linked to one or more additional regulatory elements. In one example, the vector comprises an RNA pol III promoter operably linked to a sequence encoding a gRNA, an RNA pol II promoter operably linked to a coding sequence of any of the fusion polypeptides described herein, linked to one or more additional regulatory elements (e.g., HPRE or WPRE).

In some examples, the vectors described herein comprise a first promoter operably linked to a coding sequence of any of the fusion polypeptides described herein, linked to one or more additional regulatory elements, and a second promoter operably linked to a sequence encoding a gRNA. An example composition of a vector comprises an RNA pol II promoter operably linked to a coding sequence of any of the fusion polypeptides described herein, linked to one or more additional regulatory elements, and an RNA pol III promoter operably linked to a sequence encoding a gRNA. In one example, the vector comprises an RNA pol II promoter operably linked to a coding sequence of any of the fusion polypeptides described herein, linked to one or more additional regulatory elements (e.g., HPRE or WPRE), and an RNA pol III promoter operably linked to a sequence encoding a gRNA.

In some embodiments, the vector is any of the exemplary vectors set forth by of any one of SEQ ID NOs: 45-53. The present disclosure also provides vectors comprising a nucleic acid sequence that is at least about 70% or more, e.g., about 80%, about 90%, about 91%, about 92%, about 93%, about 94%, about 95%, about 96%, about 97%, about 98%, or about 99% identical to any of the vectors described herein, such as any one of SEQ ID NOs: 45-53.

### Methods and Systems of Use of the Fusion Polypeptides

Some aspects of this disclosure provide fusion polypeptides, systems, ribonucleoprotein (RNP) complexes, and methods for generating the genetically engineered cells described herein, e.g., genetically engineered cells comprising a modification in their genome, such as a modification that results in a loss of expression or regulation of a protein, or expression of a variant form of a protein.

The present disclosure provides a system for introducing targeted genomic modifications into a cell of interest. In some embodiments, the system comprises any of the fusion polypeptides described herein and at least one guide RNA (gRNA) that directs or targets the fusion polypeptide to a target site (target sequence) in the genome of the cell. In some embodiments, any of the fusion polypeptides described herein are capable of forming and/or maintaining a ribonucleoprotein (RNP) complex with a gRNA and the RNP complex is capable of binding the target sequence in the genome of a cell. In some embodiments, the system further comprises one or more additional gRNAs that direct or target the fusion polypeptide to additional target site(s) (target sequence) in the genome of the cell.

In some embodiments, the system comprises a fusion polypeptide comprising a Cpf1 domain that lacks nuclease activity and an endonuclease domain that comprises a first DNA-cleavage domain that is capable of forming a dimer with a second DNA-cleavage domain that is present on a separate fusion polypeptide. In such embodiments, the system may further comprise a second fusion polypeptide comprising a Cpf1 domain that lacks nuclease activity and a second endonuclease domain comprising the second DNA-cleavage domain. In some embodiments, the method further comprises contacting the cell with a second fusion polypeptide, or nucleic acid encoding the same.

In some embodiments, the first and second steps detailed above occur simultaneously or in close temporal proximity. In some embodiments, all steps detailed above, if taken, occur simultaneously or in close temporal proximity.

In some aspects, the present disclosure provides methods involving contacting a cell with any of the fusion polypeptides described herein and contacting the cell with a gRNA comprising a targeting domain complementary to a first target sequence in the genome of a cell. In some embodiments, the method further comprises contacting the cell with a second comprising a targeting domain complementary to a second target sequence in the genome of a cell wherein the first target sequence and the second target sequence are not the same and the first fusion polypeptide and second fusion polypeptide are not the same.

In some embodiments, the first target sequence and the second target sequence are on different chromosomes of the genome of the cell. In some embodiments, the first target sequence and the second target sequence are on the same chromosome in the genome of the cell. In some embodiments, the first target sequence and the second target sequence are on the same DNA strand of the chromosome. In some embodiments, the first target sequence and the second target sequence are on different DNA strands of the chromosome. In some embodiments, the first target sequence and the second target sequence are separated by 10-10,000 nucleotides. In some embodiments, the first target sequence and the second target sequence are separated by 10, 20, 30, 40, 50, 60, 70, 80, 90, 100, 150, 200, 250, 300, 350, 400, 450, 500, 550, 600, 650, 700, 750, 800, 850, 900, 950, 1000, 1500, 2000, 2500, 3000, 3500, 4000, 4500, 5000, 5500, 6000, 6500, 7000, 7500, 8000, 8500, 9000, 9500, or 10,000 nucleotides.

The fusion polypeptides and/or gRNAs described herein can be delivered to a cell in any manner suitable. Various suitable methods for the delivery of a system, e.g., comprising an RNP including a fusion polypeptide and gRNA, may include any suitable method such as, electroporation of RNP into a cell, electroporation of mRNA encoding any of the fusion polypeptides and a gRNA into a cell, various protein or nucleic acid transfection methods, and delivery of encoding RNA or DNA via viral vectors, such as, for example, retroviral (e.g., lentiviral) vectors. Any suitable delivery method is embraced by this disclosure, and the disclosure is not limited in this respect.

In some embodiments, a fusion polypeptide/gRNA complex (RNP complex) is formed, e.g., in vitro, and the cell is contacted with the RNP complex, e.g., via electroporation of the RNP complex into the cell. In some embodiments, the cell is contacted with fusion polypeptide and gRNA separately, and the RNP complex is formed within the cell. In some embodiments, the cell is contacted with a nucleic acid, e.g., a DNA or RNA, encoding the fusion polypeptide, and/or with a nucleic acid encoding the gRNA, or both. In some embodiments, the nucleic acid encoding the fusion polypeptide and/or the nucleic acid encoding the gRNA is an mRNA or an mRNA analog.

In some aspects, the present disclosure provides guide RNAs (gRNAs) that are suitable to target any of the fusion polypeptides described herein to a suitable target site in the genome of a cell. The terms “guide RNA” and “gRNA” are used interchangeably herein and refer to a nucleic acid, typically an RNA, that is bound by an RNA-guided nuclease and promotes the specific targeting or homing of the RNA-guided nuclease to a target nucleic acid, e.g., a target site within the genome of a cell. A gRNA typically comprises at least two domains: a “binding domain,” also sometimes referred to as “gRNA scaffold” or “gRNA backbone” that mediates binding to an RNA-guided nuclease (also referred to as the “binding domain”), and a “targeting domain” that mediates the targeting of the gRNA-bound RNA-guided nuclease to a target site. Some gRNAs comprise additional domains, e.g., complementarity domains, or stem-loop domains. The structures and sequences of naturally occurring gRNA binding domains and engineered variants thereof are well known to those of skill in the art.

Suitable gRNAs for use with CRISPR/Cas nucleases, such as Cpf1 nucleases, typically comprise a single RNA molecule, as the naturally occurring Cpf1 guide RNA comprises a single RNA molecule. A suitable gRNA may thus be unimolecular (having a single RNA molecule), sometimes referred to herein as single guide RNAs (sgRNAs), or modular (comprising more than one, and typically two, separate RNA molecules). Some exemplary suitable Cpf1 gRNA scaffold sequences are provided herein, and additional suitable gRNA scaffold sequences will be apparent to the skilled artisan based on the present disclosure.

In some embodiments, e.g., in some embodiments where a Cpf1 nuclease is used, a gRNA, may comprise, from 5′ to 3′:
a CRISPR RNA (crRNA) sequence for a CRISPR/Cas nuclease, containing:
   a proximal domain;
   a first complementarity domain;
   a linking domain; and
   a second complementarity domain (which is complementary to the first complementarity domain); and
   a targeting domain corresponding to a target site sequence.

Some exemplary suitable Cpf1 gRNA scaffold sequences are provided herein, and additional suitable gRNA scaffold sequences will be apparent to the skilled artisan based on the present disclosure. Such additional suitable scaffold sequences include, without limitation, those recited in Jinek, et al. Science (2012) 337(6096):816-821, Ran, et al. Nature Protocols (2013) 8:2281-2308, PCT Publication No. WO 2014/093694, and PCT Publication No. WO 2013/176772, incorporate by reference in their entirety.

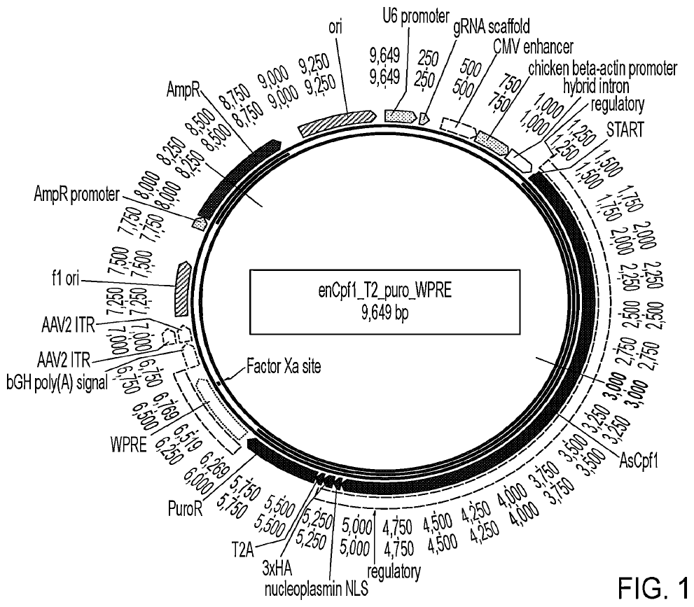

A gRNA as provided herein typically comprises a targeting domain that binds to a target site in the genome of a cell. The target site is typically a double-stranded DNA sequence comprising the PAM sequence and, on the same strand as, and directly adjacent to, the PAM sequence, the target domain. The targeting domain of the gRNA typically comprises an RNA sequence that corresponds to the target domain sequence in that it resembles the sequence of the target domain, sometimes with one or more mismatches, but typically comprises an RNA instead of a DNA sequence. The targeting domain of the gRNA thus base-pairs (in full or partial complementarity) with the sequence of the double-stranded target site that is complementary to the sequence of the target domain, and thus with the strand complementary to the strand that comprises the PAM sequence. It will be understood that the targeting domain of the gRNA typically does not include the PAM sequence. It will further be understood that the location of the PAM may be 5′ or 3′ of the target domain sequence, depending on the nuclease employed. For example, the PAM is typically 3′ of the target domain sequences for Cas9 nucleases, and 5′ of the target domain sequence for Cas12a nucleases. For an illustration of the location of the PAM and the mechanism of gRNA binding a target site, see, e.g., FIG. 1 of Vanegas et al., Fungal Biol Biotechnol. 2019; 6: 6, which is incorporated by reference herein. For additional illustration and description of the mechanism of gRNA targeting an RNA-guided nuclease to a target site, see Fu Y et al, Nat Biotechnol (2014) (doi: 10.1038/nbt.2808) and Sternberg S H et al., Nature (2014) (doi: 10.1038/naturel3011), both incorporated herein by reference.

The targeting domain may comprise a nucleotide sequence that corresponds to the sequence of the target domain, i.e., the DNA sequence directly adjacent to the PAM sequence (e.g., 5′ of the PAM sequence for Cas9 nucleases, or 3′ of the PAM sequence for Cas12a nucleases). The targeting domain sequence typically comprises between 17 and 30 nucleotides and corresponds fully with the target domain sequence (i.e., without any mismatch nucleotides), or may comprise one or more, but typically not more than 4, mismatches. As the targeting domain is part of an RNA molecule, the gRNA, it will typically comprise ribonucleotides, while the DNA targeting domain will comprise deoxyribonucleotides.

The structure of a typical Cas12a gRNA can be found, for example in FIG. 1 of Zetsche et al. Cell (2015) 163(3): 759-771, which is incorporated by reference herein in its entirety. An exemplary illustration of a Cas12a target site, comprising a 22 nucleotide target domain, and a TTN PAM sequence, as well as of a gRNA comprising a targeting domain that fully corresponds to the target domain (and thus base-pairs with full complementarity with the DNA strand complementary to the strand comprising the target domain and PAM) is provided below:

```
                        [ PAM ][ target domain (DNA) ]
                    5′-T-T-N-N-N-N-N-N-N-N-N-N-N-N-N-N-N-N-N-N-N-N-N-N-N-3′ (DNA)
                    3′-A-A-N-N-N-N-N-N-N-N-N-N-N-N-N-N-N-N-N-N-N-N-N-N-N-5′ (DNA)
                                      | | | | | | | | | | | | | | | | | | | | | |
5′-[gRNA scaffold]-N-N-N-N-N-N-N-N-N-N-N-N-N-N-N-N-N-N-N-N-N-N-3′ (RNA)
    [binding domain][ targeting domain (RNA) ]
```

In some embodiments, the Cas12a PAM sequence is 5′-T-T-T-V-3′. In some embodiments, the Cas12a PAM sequence is 5′-T-T-V-3′.

While not wishing to be bound by theory, at least in some embodiments, it is believed that the length and complementarity of the targeting domain with the target sequence contributes to specificity of the interaction of the gRNA/Cas molecule complex with a target nucleic acid. In some embodiments, the targeting domain of a gRNA provided herein is 5 to 50 nucleotides in length. In some embodiments, the targeting domain is 15 to 25 nucleotides in length. In some embodiments, the targeting domain is 18 to 22 nucleotides in length. In some embodiments, the targeting domain is 19-21 nucleotides in length. In some embodiments, the targeting domain is 15 nucleotides in length. In some embodiments, the targeting domain is 16 nucleotides in length. In some embodiments, the targeting domain is 17 nucleotides in length. In some embodiments, the targeting domain is 18 nucleotides in length. In some embodiments, the targeting domain is 19 nucleotides in length. In some embodiments, the targeting domain is 20 nucleotides in length. In some embodiments, the targeting domain is 21 nucleotides in length. In some embodiments, the targeting domain is 22 nucleotides in length. In some embodiments, the targeting domain is 23 nucleotides in length. In some embodiments, the targeting domain is 24 nucleotides in length. In some embodiments, the targeting domain is 25 nucleotides in length. In some embodiments, the targeting domain fully corresponds, without mismatch, to a target domain sequence provided herein, or a part thereof. In some embodiments, the targeting domain of a gRNA provided herein comprises 1 mismatch relative to a target domain sequence provided herein. In some embodiments, the targeting domain comprises 2 mismatches relative to the target domain sequence. In some embodiments, the target domain comprises 3 mismatches relative to the target domain sequence.

In some embodiments, a targeting domain comprises a core domain and a secondary targeting domain, e.g., as described in PCT Publication No. WO 2015/157070, which is incorporated by reference in its entirety. In some embodiments, the core domain comprises about 8 to about 13 nucleotides from the 3′ end of the targeting domain (e.g., the most 3′ 8 to 13 nucleotides of the targeting domain). In some embodiments, the secondary domain is positioned 5′ to the core domain. In some embodiments, the core domain corresponds fully with the target domain sequence, or a part thereof. In other embodiments, the core domain may comprise one or more nucleotides that are mismatched with the corresponding nucleotide of the target domain sequence.

The sequence and placement of the above-mentioned domains are described in more detail in PCT Publication No. WO 2015/157070, which is herein incorporated by reference in its entirety, including p. 88-112 therein.

A linking domain may serve to link the first complementarity domain with the second complementarity domain of a unimolecular gRNA. The linking domain can link the first and second complementarity domains covalently or non-covalently. In some embodiments, the linkage is covalent. In some embodiments, the linking domain is, or comprises, a covalent bond interposed between the first complementarity domain and the second complementarity domain. In some embodiments, the linking domain comprises one or more, e.g., 2, 3, 4, 5, 6, 7, 8, 9, or 10 nucleotides. In some embodiments, the linking domain comprises at least one non-nucleotide bond, e.g., as disclosed in PCT Publication No. WO 2018/126176, the entire contents of which are incorporated herein by reference.

In some embodiments, the second complementarity domain of the targeting domain is complementary, at least in part, with the first complementarity domain, and in an embodiment, has sufficient complementarity to the second complementarity domain to form a duplexed region under at least some physiological conditions. In some embodiments, the second complementarity domain can include a sequence that lacks complementarity with the first complementarity domain, e.g., a sequence that loops out from the duplexed region. In some embodiments, the second complementarity domain is 5 to 27 nucleotides in length. In some embodiments, the second complementarity domain is longer than the first complementarity region. In an embodiment, the complementary domain is 5, 6, 7, 8, 9, 10, 11, 12, 13, 14, 15, 16, 17, 18, 19, 20, 21, 22, 23, 24 or 25 nucleotides in length. In some embodiments, the second complementarity domain comprises 3 subdomains, which, in the 5′ to 3′ direction are: a 5′ subdomain, a central subdomain, and a 3′ subdomain. In some embodiments, the 5′ subdomain is 3 to 25, e.g., 4 to 22, 4 to 18, or 4 to 10, or 3, 4, 5, 6, 7, 8, 9, 10, 11, 12, 13, 14, 15, 16, 17, 18, 19, 20, 21, 22, 23, 24, or 25 nucleotides in length. In some embodiments, the central subdomain is 1, 2, 3, 4 or 5, e.g., 3, nucleotides in length. In some embodiments, the 3′ subdomain is 4 to 9, e.g., 4, 5, 6, 7, 8 or 9 nucleotides in length. In some embodiments, the 5′ subdomain and the 3′ subdomain of the first complementarity domain, are respectively, complementary, e.g., fully complementary, with the 3′ subdomain and the 5′ subdomain of the second complementarity domain.

In some embodiments, a gRNA may comprise one or more nucleotides that are chemically modified. Chemical modifications of gRNAs have previously been described, and suitable chemical modifications include any modifications that are beneficial for gRNA function and do not measurably increase any undesired characteristics, e.g., off-target effects, of a given gRNA. Suitable chemical modifications include, for example, those that make a gRNA less susceptible to endo- or exonuclease catalytic activity, and include, without limitation, phosphorothioate backbone modifications, 2′-O-Me-modifications (e.g., at one or both of the 3′ and 5′ termini), 2′F-modifications, replacement of the ribose sugar with the bicyclic nucleotide-cEt, 3′thioPACE (MSP) modifications, or any combination thereof. Additional suitable gRNA modifications will be apparent to the skilled artisan based on this disclosure, and such suitable gRNA modifications include, without limitation, those described, e.g., in Rahdar et al. PNAS (2015) 112 (51) E7110-E7117 and Hendel et al., Nat Biotechnol. (2015); 33(9): 985-989, each of which is incorporated herein by reference in its entirety.

For example, a gRNA provided herein may comprise one or more 2′-O modified nucleotide, e.g., a 2′-O-methyl nucleotide. In some embodiments, the gRNA comprises a 2′-O modified nucleotide, e.g., 2′-O-methyl nucleotide at the 5′ end of the gRNA. In some embodiments, the gRNA comprises a 2′-O modified nucleotide, e.g., 2′-O-methyl nucleotide at the 3′ end of the gRNA. In some embodiments, the gRNA comprises a 2′-O-modified nucleotide, e.g., a 2′-O-methyl nucleotide at both the 5′ and 3′ ends of the gRNA. In some embodiments, the gRNA is 2′-O-modified, e.g. 2′-O-methyl-modified at the nucleotide at the 5′ end of the gRNA, the second nucleotide from the 5′ end of the gRNA, and the third nucleotide from the 5′ end of the gRNA. In some embodiments, the gRNA is 2′-O-modified, e.g., 2′-O-methyl-modified at the nucleotide at the 3′ end of the gRNA, the second nucleotide from the 3′ end of the gRNA, and the third nucleotide from the 3′ end of the gRNA. In some embodiments, the gRNA is 2′-O-modified, e.g., 2′-O-methyl-modified at the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA. In some embodiments, the gRNA is 2′-O-modified, e.g., 2′-O-methyl-modified at the second nucleotide from the 3′ end of the gRNA, the third nucleotide from the 3′ end of the gRNA, and at the fourth nucleotide from the 3′ end of the gRNA. In some embodiments, the nucleotide at the 3′ end of the gRNA is not chemically modified. In some embodiments, the nucleotide at the 3′ end of the gRNA does not have a chemically modified sugar. In some embodiments, the gRNA is 2′-O-modified, e.g., 2′-O-methyl-modified, at the nucleotide at the 5′ end of the gRNA, the second nucleotide from the 5′ end of the gRNA, the third nucleotide from the 5′ end of the gRNA, the second nucleotide from the 3′ end of the gRNA, the third nucleotide from the 3′ end of the gRNA, and the fourth nucleotide from the 3′ end of the gRNA. In some embodiments, the 2′-O-methyl nucleotide comprises a phosphate linkage to an adjacent nucleotide. In some embodiments, the 2′-O-methyl nucleotide comprises a phosphorothioate linkage to an adjacent nucleotide. In some embodiments, the 2′-O-methyl nucleotide comprises a thioPACE linkage to an adjacent nucleotide.

In some embodiments, a gRNA provided herein may comprise one or more 2′-O-modified and 3′phosphorous-modified nucleotide, e.g., a 2′-O-methyl 3′phosphorothioate nucleotide. In some embodiments, the gRNA comprises a 2′-O-modified and 3′phosphorous-modified, e.g., 2′-O-methyl 3′phosphorothioate nucleotide at the 5′ end of the gRNA. In some embodiments, the gRNA comprises a 2′-O-modified and 3′phosphorous-modified, e.g., 2′-O-methyl 3′phosphorothioate nucleotide at the 3′ end of the gRNA. In some embodiments, the gRNA comprises a 2′-O-modified and 3′phosphorous-modified, e.g., 2′-O-methyl 3′phosphorothioate nucleotide at the 5′ and 3′ ends of the gRNA. In some embodiments, the gRNA comprises a backbone in which one or more non-bridging oxygen atoms has been replaced with a sulfur atom. In some embodiments, the gRNA is 2′-O-modified and 3′phosphorous-modified, e.g., 2′-O-methyl 3′phosphorothioate-modified at the nucleotide at the 5′ end of the gRNA, the second nucleotide from the 5′ end of the gRNA, and the third nucleotide from the 5′ end of the gRNA. In some embodiments, the gRNA is 2′-O-modified and 3′phosphorous-modified, e.g., 2′-O-methyl 3′phosphorothioate-modified at the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g., 2′-O-methyl 3′phosphorothioate-modified at the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g., 2′-O-methyl 3′phosphorothioate-modified at the second nucleotide from the 3′ end of the gRNA, the third nucleotide from the 3′ end of the gRNA, and the fourth nucleotide from the 3′ end of the gRNA. In some embodiments, the nucleotide at the 3′ end of the gRNA is not chemically modified. In some embodiments, the nucleotide at the 3′ end of the gRNA does not have a chemically modified sugar. In some embodiments, the gRNA is 2′-O-modified and 3′phosphorous-modified, e.g., 2′-O-methyl 3′phosphorothioate-modified at the nucleotide at the 5′ end of the gRNA, the second nucleotide from the 5′ end of the gRNA, the third nucleotide from the 5′ end of the gRNA, the second nucleotide from the 3′ end of the gRNA, the third nucleotide from the 3′ end of the gRNA, and the fourth nucleotide from the 3′ end of the gRNA.

In some embodiments, a gRNA provided herein may comprise one or more 2′-O-modified and 3′-phosphorous-modified, e.g., 2′-O-methyl 3′thioPACE nucleotide. In some embodiments, the gRNA comprises a 2′-O-modified and 3′phosphorous-modified, e.g., 2′-O-methyl 3′thioPACE nucleotide at the 5′ end of the gRNA. In some embodiments, the gRNA comprises a 2′-O-modified and 3′phosphorous-modified, e.g., 2′-O-methyl 3′thioPACE nucleotide at the 3′ end of the gRNA. In some embodiments, the gRNA comprises a 2′-O-modified and 3′phosphorous-modified, e.g., 2′-O-methyl 3′thioPACE nucleotide at the 5′ and 3′ ends of the gRNA. In some embodiments, the gRNA comprises a backbone in which one or more non-bridging oxygen atoms have been replaced with a sulfur atom and one or more non-bridging oxygen atoms have been replaced with an acetate group. In some embodiments, the gRNA is 2′-O-modified and 3′phosphorous-modified, e.g., 2′-O-methyl 3′ thioPACE-modified at the nucleotide at the 5′ end of the gRNA, the second nucleotide from the 5′ end of the gRNA, and the third nucleotide from the 5′ end of the gRNA. In some embodiments, the gRNA is 2′-O-modified and 3′phosphorous-modified, e.g., 2′-O-methyl 3′thioPACE-modified at the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g., 2′-O-methyl 3′thioPACE-modified at the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g., 2′-O-methyl 3′thioPACE-modified at the second nucleotide from the 3′ end of the gRNA, the third nucleotide from the 3′ end of the gRNA, and the fourth nucleotide from the 3′ end of the gRNA. In some embodiments, the nucleotide at the 3′ end of the gRNA is not chemically modified. In some embodiments, the nucleotide at the 3′ end of the gRNA does not have a chemically modified sugar. In some embodiments, the gRNA is 2′-O-modified and 3′phosphorous-modified, e.g. 2′-O-methyl 3′thioPACE-modified at the nucleotide at the 5′ end of the gRNA, the second nucleotide from the 5′ end of the gRNA, the third nucleotide from the 5′ end of the gRNA, the second nucleotide from the 3′ end of the gRNA, the third nucleotide from the 3′ end of the gRNA, and the fourth nucleotide from the 3′ end of the gRNA.

In some embodiments, a gRNA provided herein comprises a chemically modified backbone. In some embodiments, the gRNA comprises a phosphorothioate linkage. In some embodiments, one or more non-bridging oxygen atoms have been replaced with a sulfur atom. In some embodiments, the nucleotide at the 5′ end of the gRNA, the second nucleotide from the 5′ end of the gRNA, and the third nucleotide from the 5′ end of the gRNA each comprise a phosphorothioate linkage. In some embodiments, the nucleotide at the 3′ end of the gRNA, the second nucleotide from the 3′ end of the gRNA, and the third nucleotide from the 3′ end of the gRNA each comprise a phosphorothioate linkage. In some embodiments, the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA each comprise a phosphorothioate linkage. In some embodiments, the second nucleotide from the 3′ end of the gRNA, the third nucleotide from the 3′ end of the gRNA, and at the fourth nucleotide from the 3′ end of the gRNA each comprise a phosphorothioate linkage. In some embodiments, the nucleotide at the 5′ end of the gRNA, the second nucleotide from the 5′ end of the gRNA, the third nucleotide from the 5′ end, the second nucleotide from the 3′ end of the gRNA, the third nucleotide from the 3′ end of the gRNA, and the fourth nucleotide from the 3′ end of the gRNA each comprise a phosphorothioate linkage.

In some embodiments, a gRNA provided herein comprises a thioPACE linkage. In some embodiments, the gRNA comprises a backbone in which one or more non-bridging oxygen atoms have been replaced with a sulfur atom and one or more non-bridging oxygen atoms have been replaced with an acetate group. In some embodiments, the nucleotide at the 5′ end of the gRNA, the second nucleotide from the 5′ end of the gRNA, and the third nucleotide from the 5′ end of the gRNA each comprise a thioPACE linkage. In some embodiments, the nucleotide at the 3′ end of the gRNA, the second nucleotide from the 3′ end of the gRNA, and the third nucleotide from the 3′ end of the gRNA each comprise a thioPACE linkage. In some embodiments, the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA each comprise a thioPACE linkage. In some embodiments, the second nucleotide from the 3′ end of the gRNA, the third nucleotide from the 3′ end of the gRNA, and at the fourth nucleotide from the 3′ end of the gRNA each comprise a thioPACE linkage. In some embodiments, the nucleotide at the 5′ end of the gRNA, the second nucleotide from the 5′ end of the gRNA, the third nucleotide from the 5′ end, the second nucleotide from the 3′ end of the gRNA, the third nucleotide from the 3′ end of the gRNA, and the fourth nucleotide from the 3′ end of the gRNA each comprise a thioPACE linkage.

In some embodiments, a gRNA described herein comprises one or more 2′-O-methyl-3′-phosphorothioate nucleotides, e.g., at least 1, 2, 3, 4, 5, or 6 2′-O-methyl-3′-phosphorothioate nucleotides. In some embodiments, a gRNA described herein comprises modified nucleotides (e.g., 2′-O-methyl-3′-phosphorothioate nucleotides) at one or more of the three terminal positions and the 5′ end and/or at one or more of the three terminal positions and the 3′ end. In some embodiments, the nucleotide at the 5′ end of the gRNA, the second nucleotide from the 5′ end of the gRNA, the third nucleotide from the 5′ end, the second nucleotide from the 3′ end of the gRNA, the third nucleotide from the 3′ end of the gRNA, and the fourth nucleotide from the 3′ end of the gRNA each comprise a 2′-O-methyl-3′-phosphorothioate nucleotides. In some embodiments, the gRNA may comprise one or more modified nucleotides, e.g., as described in PCT Publication Nos. WO 2017/214460, WO 2016/089433, and WO 2016/164356, which are incorporated by reference their entirety.

The gRNAs provided herein can be delivered to a cell in any manner suitable. Various suitable methods for the delivery of CRISPR/Cas systems, e.g., comprising an RNP including a gRNA bound to any of the fusion polypeptides described herein, have been described, and exemplary suitable methods include, without limitation, electroporation of a RNP into a cell, electroporation of mRNA encoding any of the fusion polypeptides described herein and a gRNA into a cell, various protein or nucleic acid transfection methods, and delivery of encoding RNA or DNA via viral vectors, such as, for example, retroviral (e.g., lentiviral) vectors. Any suitable delivery method is embraced by this disclosure, and the disclosure is not limited in this respect.

### Cells

The fusion polypeptides, methods, and strategies provided herein may be applied to any cell or cell type capable of being genetically engineered using the fusion polypeptides and methods described herein. The skilled artisan will understand, however, that the provision of such examples is for the purpose of illustrating some specific embodiments, and additional suitable cells and cell types will be apparent to the skilled artisan based on the present disclosure, which is not limited in this respect.

In some embodiments, the cell is a eukaryotic cell. In some embodiments, the cell is a mammalian cell, yeast cell, fungal cell, or plant cell. In some embodiments, the cell is a mammalian cell, such as a non-human primate cell, a rodent (e.g., mouse or rat) cell, a bovine cell, a porcine cell, an equine cell, or a cell of a domestic animal. In some embodiments, the cell is a human cell or a mouse cell. In some embodiments, the cells may be obtained from a subject, such as a human subject (e.g., a healthy human subject or a human subject having a disease).

In some embodiments, the cells are hematopoietic cells, e.g., hematopoietic stem cells (HSC) or hematopoietic progenitor cells (HPC). In some embodiments, the cells provided herein are hematopoietic stem or progenitor cells. Hematopoietic stem cells (HSCs) are typically capable of giving rise to both myeloid and lymphoid progenitor cells that further give rise to myeloid cells (e.g., monocytes, macrophages, neutrophils, basophils, dendritic cells, erythrocytes, platelets, etc.) and lymphoid cells (e.g., T cells, B cells, NK cells), respectively. HSCs are characterized by the expression off one or more cell surface markers, such as CD34 (e.g., CD34+), which can be used for the identification and/or isolation of HSCs, and absence of cell surface markers associated with commitment to a cell lineage. In some embodiments, the HSCs are peripheral blood HSCs. Methods of obtaining cells, such as hematopoietic stem cells are described, e.g., in PCT Application No. PCT/US2016/057339, which is herein incorporated by reference in its entirety.

In some embodiments, the cells provided herein are immune effector cells. In some embodiments, the immune effector cell is a lymphocyte. In some embodiments, the immune effector cell is a T-lymphocyte. In some embodiments, the T-lymphocyte is an alpha/beta T-lymphocyte. In some embodiments, the T-lymphocyte is a gamma/delta T-lymphocyte. In some embodiments, the immune effector cell is a natural killer T (NKT cell). In some embodiments, the immune effector cell is a natural killer (NK) cell.

In some embodiments, the cell is a stem cell. In some embodiments, the stem cell is selected from the group consisting of an embryonic stem cell (ESC), an induced pluripotent stem cell (iPSC), a mesenchymal stem cell, or a tissue-specific stem cell.

In some embodiments, a genetically engineered cell provided herein comprises only one genomic modification, e.g., a genomic modification that results in a loss of expression of a protein, for example a protein encoded by or regulated by the target site sequence, or expression of a variant form of the protein. It will be understood that the gene editing methods provided herein may result in genomic modifications in one or both alleles of a target genetic loci. In some embodiments, genetically engineered cells comprising a genomic modification in both alleles of a given genetic locus are preferred.

In some embodiments, a genetically engineered cell provided herein comprises two or more genomic modifications. For example, a population of genetically engineered cells can comprise a plurality of different mutations.

As will be evident to one of ordinary skill in the art, the fusion polypeptides and methods described herein may be used to modify any genetic locus in a cell, including for example protein-coding, non-protein coding, chromosomal, and extra-chromosomal sequences. Accordingly, targeting domains of the gRNAs may be designed to target any genetic locus (i.e., a target site sequence), such as a target site sequence adjacent to a PAM sequence for a corresponding CRISPR/Cas nuclease.

In some embodiments, the targeting domain of a gRNA for use with the fusion polypeptides described herein targets a cell surface protein, such as a Type 0, Type 1, or Type 2 cell surface protein. In some embodiments, the targeting domain targets BCMA, CD19, CD20, CD30, ROR1, B7H6, B7H3, CD23, CD33, CD38, C-type lectin like molecule-1 (CLL-1), CS1, EMR2, IL-5, L1-CAM, PSCA, PSMA, CD138, CD133, CD70, CD5, CD6, CD7, CD13, NKG2D, NKG2D ligand, CLECi2A, CD11, CD117, CD123, CD56, CD34, CD14, CD66b, CD41, CD61, CD62, CD235a, CD146, CD326, LMP2, CD22, CD52, CD10, CD3/TCR, CD79/BCR, and/or CD26.

In some embodiments, the targeting domain of a gRNA for use with the fusion polypeptides described herein targets a cell surface protein associated with a neoplastic or malignant disease or disorder, e.g., with a specific type of cancer, such as, without limitation, CD20, CD22 (Non-Hodgkin's lymphoma, B-cell lymphoma, chronic lymphocytic leukemia (CLL)), CD52 (B-cell CLL), CD33 (Acute myelogenous leukemia (AML)), CD10 (gp100) (Common (pre-B) acute lymphocytic leukemia and malignant melanoma), CD3/T-cell receptor (TCR) (T-cell lymphoma and leukemia), CD79/B-cell receptor (BCR) (B-cell lymphoma and leukemia), CD26 (epithelial and lymphoid malignancies), human leukocyte antigen (HLA)-DR, HLA-DP, and HLA-DQ (lymphoid malignancies), RCAS1 (gynecological carcinomas, biliary adenocarcinomas and ductal adenocarcinomas of the pancreas) as well as prostate specific membrane antigen.

Additional non-limiting examples of cell surface proteins include CD1a, CD1b, CD1c, CD1d, CD1e, CD2, CD3, CD3d, CD3e, CD3g, CD4, CD5, CD6, CD7, CD8a, CD8b, CD9, CD10, CD11a, CD11b, CD11c, CD11d, CDw12, CD13, CD14, CD15, CD16, CD16b, CD17, CD18, CD19, CD20, CD21, CD22, CD23, CD24, CD25, CD26, CD27, CD28, CD29, CD30, CD31, CD32a, CD32b, CD32c, CD34, CD35, CD36, CD37, CD38, CD39, CD40, CD41, CD42a, CD42b, CD42c, CD42d, CD43, CD44, CD45, CD45RA, CD45RB, CD45RC, CD45RO, CD46, CD47, CD48, CD49a, CD49b, CD49c, CD49d, CD49e, CD49f, CD50, CD51, CD52, CD53, CD54, CD55, CD56, CD57, CD58, CD59, CD60a, CD61, CD62E, CD62L, CD62P, CD63, CD64a, CD65, CD65s, CD66a, CD66b, CD66c, CD66F, CD68, CD69, CD70, CD71, CD72, CD73, CD74, CD75, CD75S, CD77, CD79a, CD79b, CD80, CD81, CD82, CD83, CD84, CD85A, CD85C, CD85D, CD85E, CD85F, CD85G, CD85H, CD85I, CD85J, CD85K, CD86, CD87, CD88, CD89, CD90, CD91, CD92, CD93, CD94, CD95, CD96, CD97, CD98, CD99, CD99R, CD100, CD101, CD102, CD103, CD104, CD105, CD106, CD107a, CD107b, CD108, CD109, CD110, CD111, CD112, CD113, CD114, CD115, CD116, CD117, CD118, CD119, CD120a, CD120b, CD121a, CD121b, CD121a, CD121b, CD122, CD123, CD124, CD125, CD126, CD127, CD129, CD130, CD131, CD132, CD133, CD134, CD135, CD136, CD137, CD138, CD139, CD140a, CD140b, CD141, CD142, CD143, CD14, CDw145, CD146, CD147, CD148, CD150, CD152, CD152, CD153, CD154, CD155, CD156a, CD156b, CD156c, CD157, CD158b1, CD158b2, CD158d, CD158e1/e2, CD158f, CD158g, CD158h, CD158i, CD158j, CD158k, CD159a, CD159c, CD160, CD161, CD163, CD164, CD165, CD166, CD167a, CD168, CD169, CD170, CD171, CD172a, CD172b, CD172g, CD173, CD174, CD175, CD175s, CD176, CD177, CD178, CD179a, CD179b, CD180, CD181, CD182, CD183, CD184, CD185, CD186, CD191, CD192, CD193, CD194, CD195, CD196, CD197, CDw198, CDw199, CD200, CD201, CD202b, CD203c, CD204, CD205, CD206, CD207, CD208, CD209, CD210a, CDw210b, CD212, CD213a1, CD213a2, CD215, CD217, CD218a, CD218b, CD220, CD221, CD222, CD223, CD224, CD225, CD226, CD227, CD228, CD229, CD230, CD231, CD232, CD233, CD234, CD235a, CD235b, CD236, CD236R, CD238, CD239, CD240, CD241, CD242, CD243, CD244, CD245, CD246, CD247, CD248, CD249, CD252, CD253, CD254, CD256, CD257, CD258, CD261, CD262, CD263, CD264, CD265, CD266, CD267, CD268, CD269, CD270, CD272, CD272, CD273, CD274, CD275, CD276, CD277, CD278, CD279, CD280, CD281, CD282, CD283, CD284, CD286, CD288, CD289, CD290, CD292, CDw293, CD294, CD295, CD296, CD297, CD298, CD299, CD300a, CD300c, CD300e, CD301, CD302, CD303, CD304, CD305, CD306, CD307a, CD307b, CD307c, CD307d, CD307e, CD309, CD312, CD314, CD315, CD316, CD317, CD318, CD319, CD320, CD321, CD322, CD324, CD325, CD326, CD327, CD328, CD329, CD331, CD332, CD333, CD334, CD335, CD336, CD337, CD338, CD339, CD340, CD344, CD349, CD350, CD351, CD352, CD353, CD354, CD355, CD357, CD358, CD359, CD360, CD361, CD362, CD363, CD364, CD365, CD366, CD367, CD368, CD369, CD370, or CD371. See also examples of lineage-specific cell-surface antigens from BD Biosciences Human CD Marker Chart: bdbiosciences.com/content/dam/bdb/campaigns/reagent-education/BD_Reagents_CDMarkerHuman_Poster.pdf (incorporated by reference in its entirety).

### Methods of Administration to Subjects in Need Thereof

Some aspects of this disclosure provide methods comprising administering to a subject in need thereof a composition described herein, e.g., a cell genetically engineered using the fusion polypeptides and methods described herein, a population of cells or descendants thereof, or a pharmaceutical composition comprising the same. The cell, population of cells, or descendants thereof may comprise one or more modifications (e.g., genetic modifications) relative to a wildtype cell. In some embodiments, the cell, population of cells, or descendants thereof comprise a modification to a first gene relative to a wildtype cell of the same type. In some embodiments, the cell, population of cells, or descendants thereof comprise a modification to a second gene relative to a wildtype cell of the same type. In some embodiments, the cell, population of cells, or descendants thereof may comprise one or more modifications (e.g., genetic modifications) relative to a disease cell, such as a cell associated with a disease or disorder (e.g., cancer cell). Genes modified may correspond to any genetic locus targetable by the methods described herein, such as any of the exemplary genes or proteins described herein.

In some embodiments, the methods further involve administering to the subject a therapeutically effective amount of at least one agent that targets a product encoded by a wildtype copy of the modified gene. Without wishing to be bound by theory, by administering an agent that targets a product encoded by a wildtype copy of the modified gene in combination with a cell, population of cells, or descendants thereof comprising the modified gene, it is possible to target cells within a subject with the agent (e.g., disease cells, e.g., cancer cells) while not targeting or targeting to a lesser degree the cell, population of cells, or descendants thereof. For example, such a method may be used to selectively ablate or kill a target cell population in a subject while in combination replenishing the subject with new cells not vulnerable to the agent. As a further example, such a method may administer the agent as a part of the cell, population of cells, or descendants thereof (e.g., a CAR-T therapeutic), and would thus avoid or decrease cell fratricide. In some embodiments, administration of the at least one agent targeting the product encoded by the wildtype copy of the modified gene occurs simultaneously or in temporal proximity with administration of the cell, population or descendant thereof, or the pharmaceutical composition. In some embodiments, administration of the at least one agent targeting the product encoded by the wildtype copy of the modified gene occurs after administration of the cell, population or descendant thereof, or the pharmaceutical composition. In some embodiments, administration of the at least one agent targeting the product encoded by the wildtype copy of the modified gene occurs before administration of the cell, population or descendant thereof, or the pharmaceutical composition. In some embodiments, where the cell, population of cells, or descendants thereof comprises a modification to a first gene and a second gene relative to a wildtype cell of the same type, the method may comprise administering one or more (e.g., two agents) targeting the products of the first gene and the second gene (e.g., wildtype copies of the first gene and the second gene).

A subject in need thereof is, in some embodiments, a subject undergoing or about to undergo an immunotherapy targeting a product of the first gene and/or second gene. A subject in need thereof is, in some embodiments, a subject having or having been diagnosed with, a malignancy, such as caner (e.g., cancer associated with the presence of cancer stem cells, a hematopoietic malignancy, a cancer characterized by expression of a product of the first and/or second gene. In some embodiments, a subject having such a malignancy may be a candidate for administration of the agent, such as an immunotherapeutic, targeting a product of the first gene and/or second gene, but the risk of detrimental on-target, off-disease effects may outweigh the benefit, expected or observed, to the subject. In some such embodiments, administration of genetically engineered cells as described herein, results in an amelioration of the detrimental on-target, off-disease effects, as the genetically engineered cells provided herein are not targeted efficiently by the agent.

In some embodiments, the malignancy is a hematologic malignancy, or a cancer of the blood. In some embodiments, the malignancy is a lymphoid malignancy or a myeloid malignancy.

In some embodiments, the malignancy is an autoimmune disease or disorder. Examples of autoimmune disorders include, without limitation, rheumatoid arthritis, multiple sclerosis, leukemia, graft-versus host disease, lupus, and psoriasis.

In some embodiments, the malignancy is graft-versus host disease.

Also within the scope of the present disclosure are malignancies that are considered to be relapsed and/or refractory, such as relapsed or refractory hematological malignancies. A subject in need thereof is, in some embodiments, a subject undergoing or that will undergo an immune effector cell therapy targeting a product of the first gene and/or second gene, e.g., CAR-T cell therapy, wherein the immune effector cells express a CAR targeting the product, and wherein at least a subset of the immune effector cells also express the product on their cell surface. As used herein, the term “fratricide” refers to self-killing. For example, cells of a population of cells kill or induce killing of cells of the same population. In some embodiments, cells of the immune effector cell therapy kill or induce killing of other cells of the immune effector cell therapy.

In such embodiments, fratricide ablates a portion of or the entire population of immune effector cells before a desired clinical outcome, e.g., ablation of malignant cells expressing the product within the subject, can be achieved. In some such embodiments, using genetically engineered immune effector cells, as provided herein, e.g., immune effector cells that do not express the product or do not express a variant of the product recognized by the CAR, as the immune effector cells forming the basis of the immune effector cell therapy, will avoid such fratricide and the associated negative impact on therapy outcome. In such embodiments, genetically engineered immune effector cells, as provided herein, e.g., immune effector cells that do not express the product or do not express a variant of the product recognized by the CAR, may be further modified to also express the agent (e.g., a CAR targeting the product). In some embodiments, the immune effector cells may be lymphocytes, e.g., T-lymphocytes, such as, for example alpha/beta T lymphocytes, gamma/delta T-lymphocytes, or natural killer T cells. In some embodiments, the immune effect or cells may be natural killer (NK) cells.

In some embodiments, an effective number of genetically engineered cells as described herein, comprising modifications in their genome is administered to a subject in need thereof, e.g., a subject undergoing or that will undergo a therapy targeting a product of the first gene and/or second gene, wherein the therapy is associated or is at risk of being associated with a detrimental on-target, off-disease effect, e.g., in the form of cytotoxicity towards healthy cells in the subject that express the product. In some embodiments, an effective number of such genetically engineered cells may be administered to the subject in combination with the agent targeting a product encoded by a first gene or a second gene.

It is understood that when genetically modified cells and agents targeting a product encoded by a first gene or a second gene (e.g., an immunotherapeutic agent) are administered in combination, the cells and the agent may be administered at the same time or at different times, e.g., in temporal proximity.

For example, in some embodiments, administration in combination includes administration in the same course of treatment, e.g., in the course of treating a subject with an agent targeting a product (e.g., immunotherapy), the subject may be administered an effective number of genetically engineered cells, simultaneously, concurrently, or sequentially, e.g., before, during, or after the treatment with the agent, and/or in any order with respect to each other and the cells, population of cells, or descendants thereof. Furthermore, the cells and the agent may be admixed or in separate volumes or dosage forms.

In some embodiments, the agent that targets a product encoded by the first gene or a wildtype copy thereof is an immunotherapeutic agent. In some embodiments, the agent that targets a product encoded by the first gene or a wild-type copy thereof comprises an antigen binding fragment that binds the product encoded by the first gene or a wildtype copy thereof. In some embodiments, the agent that targets a product encoded by the first gene or a wild-type copy thereof comprises an antigen binding fragment that binds the product encoded by the second gene or a wildtype copy thereof.

In some embodiments, the agent is an immune cell that expresses a chimeric antigen receptor, which comprises an antigen-binding fragment (e.g., a single-chain antibody) capable of binding to a product produced by the first gene or a wild-type copy thereof. In some embodiments, the agent is an immune cell that expresses a chimeric antigen receptor, which comprises an antigen-binding fragment (e.g., a single-chain antibody) capable of binding to a product produced by the second gene or a wild-type copy thereof. The immune cell may be, e.g., a T cell (e.g., a CD4+ or CD8+ T cell) or an NK cell.

A Chimeric Antigen Receptor (CAR) can comprise a recombinant polypeptide comprising at least an extracellular antigen binding domain, a transmembrane domain, and a cytoplasmic signaling domain comprising a functional signaling domain, e.g., one derived from a stimulatory molecule. In one some embodiments, the cytoplasmic signaling domain further comprises one or more functional signaling domains derived from at least one costimulatory molecule, such as 4-1BB (i.e., CD137), CD27, and/or CD28, or fragments of those molecules. The extracellular antigen binding domain of the CAR may comprise an antibody fragment that binds a product encoded by the first gene or a wildtype copy thereof, a product encoded by the second gene or a wildtype copy thereof, or both. The antibody fragment can comprise one or more CDRs, the variable regions (or portions thereof), the constant regions (or portions thereof), or combinations of any of the foregoing.

A chimeric antigen receptor (CAR) typically comprises an antigen-binding domain, e.g., comprising an antibody fragment, fused to a CAR framework, which may comprise a hinge region (e.g., from CD8 or CD28), a transmembrane domain (e.g., from CD8 or CD28), one or more costimulatory domains (e.g., CD28 or 4-1BB), and a signaling domain (e.g., CD3zeta). Exemplary sequences of CAR domains and components are provided, for example in PCT Publication No. WO 2019/178382, and in Table 1 below.

TABLE 1
Exemplary components of a chimeric receptor

| Chimeric receptor component | Amino acid sequence |
|---|---|
| Antigen-binding fragment | Light chain-Linker-Heavy chain |
| CD28 costimulatory domain | IEVMYPPPYLDNEKSNGTIIHVKGKHLCP |
| | SPLFPGPSKPFWVLVVVGGVLACYSLLVTV |
| | AFIIFWVRSKRSRLLHSDYMNMTPRRPGPT |
| | RKHYQPYAPPRDFAAYRS (SEQ ID NO: 40) |
| CD8alpha transmembrane | IYIWAPLAGTCGVLLLSLVITLYC |
| domain | (SEQ ID NO: 41) |
| CD28 transmembrane domain | FWVLVVVGGVLACYSLLVTVAFII |
| | FWVRSKRSRLLHSDYMNMTPRR |
| | PGPTRKHYQPYAPPRDFAAYRS |
| | (SEQ ID NO: 42) |
| 4-1BB intracellular domain | KRGRKKLLYIFKQPFMRVQTTQEEDGCS |
| | CRFPEEEEGGCEL (SEQ ID NO: 43) |
| CD3 cytoplasmic signaling | RVKFSRSADAPAYQQGQNQLYNELNLG |
| domain | RREEYDVLDKRRGRDPEMGGKPQRRKNP |
| | QEGLYNELQKDKMAEAYSEIGMKGERRR |
| | GKGHDGLYQGLSTATKDTYDALHMQALPPR |
| | (SEQ ID NO: 44) |

In some embodiments, the number of genetically engineered cells provided herein, e.g., HSCs, HPCs, or immune effector cells (e.g., CAR-expressing cells) that are administered to a subject in need thereof, is within the range of 10⁶-10¹¹. However, amounts below or above this exemplary range are also within the scope of the present disclosure. For example, in some embodiments, the number of genetically engineered cells provided herein, e.g., HSCs, HPCs, or immune effector cells (e.g., CAR-expressing cells) that are administered to a subject in need thereof is about 10⁶, about 10⁷, about 10⁸, about 10⁹, about 10¹⁰, or about 10¹¹. In some embodiments, the number of genetically engineered cells provided herein, e.g., HSCs, HPCs, or immune effector cells (e.g., CAR-expressing cells) that are administered to a subject in need thereof, is within the range of 10⁶-10⁹, within the range of 10⁶-10⁸, within the range of 10⁷-10⁹, within the range of about 10⁷-10¹⁰, within the range of 10⁸-10¹⁰, or within the range of 10⁹-10¹¹.

In some embodiments, the agent that targets a product encoded by the first gene or a wildtype copy thereof is an antibody-drug conjugate (ADC). The ADC may be a molecule comprising an antibody or antigen-binding fragment thereof conjugated to a toxin or drug molecule. Binding of the antibody or fragment thereof to the corresponding antigen allows for delivery of the toxin or drug molecule to a cell that presents the antigen on the cell surface (e.g., target cell), thereby resulting in death of the target cell.

Toxins or drugs compatible for use in antibody-drug conjugates are known in the art and will be evident to one of ordinary skill in the art. See, e.g., Peters et al. Biosci. Rep.(2015) 35(4): e00225; Beck et al. Nature Reviews Drug Discovery (2017) 16:315-337; Marin-Acevedo et al. J. Hematol. Oncol. (2018)11: 8; Elgundi et al. Advanced Drug Delivery Reviews (2017) 122: 2-19.

In some embodiments, the antibody-drug conjugate may further comprise a linker (e.g., a peptide linker, such as a cleavable linker) attaching the antibody and drug molecule.

Examples of suitable toxins or drugs for antibody-drug conjugates include, without limitation, the toxins and drugs comprised in brentuximab vedotin, glembatumumab vedotin/CDX-011, depatuxizumab mafodotin/ABT-414, PSMA ADC, polatuzumab vedotin/RG7596/DCDS4501A, denintuzumab mafodotin/SGN-CD19A, AGS-16C3F, CDX-014, RG7841/DLYE5953A, RG7882/DMUC406A, RG7986/DCDS0780A, SGN-LIV1A, enfortumab vedotin/ASG-22ME, AG-15ME, AGS67E, telisotuzumab vedotin/ABBV-399, ABBV-221, ABBV-085, GSK-2857916, tisotumab vedotin/HuMax-TF-ADC, HuMax-Axl-ADC, pinatuzumab vedotin/RG7593/DCDT2980S, lifastuzumab vedotin/RG7599/DNIB0600A, indusatumab vedotin/MLN-0264/TAK-264, vandortuzumab vedotin/RG7450/DSTP3086S, sofituzumab vedotin/RG7458/DMUC5754A, RG7600/DMOT4039A, RG7336/DEDN6526A, ME1547, PF-06263507/ADC 5T4, trastuzumab emtansine/T-DM1, mirvetuximab soravtansine/IMGN853, coltuximab ravtansine/SAR3419, naratuximab emtansine/IMGN529, indatuximab ravtansine/BT-062, anetumab ravtansine/BAY 94-9343, SAR408701, SAR428926, AMG 224, PCA062, HKT288, LY3076226, SAR566658, lorvotuzumab mertansine/IMGN901, cantuzumab mertansine/SB-408075, cantuzumab ravtansine/IMGN242, laprituximab emtansine/IMGN289, IMGN388, bivatuzumab mertansine, AVE9633, BJIB015, MLN2704, AMG 172, AMG 595, LOP 628, vadastuximab talirine/SGN-CD33A, SGN-CD70A, SGN-CD19B, SGN-CD123A, SGN-CD352A, rovalpituzumab tesirine/SC16LD6.5, SC-002, SC-003, ADCT-301/HuMax-TAC-PBD, ADCT-402, MEDI3726/ADC-401, IMGN779, IMGN632, gemtuzumab ozogamicin, inotuzumab ozogamicin/CMC-544, PF-06647263, CMD-193, CMB-401, trastuzumab duocarmazine/SYD985, BMS-936561/MDX-1203, sacituzumab govitecan/IMMU-132, labetuzumab govitecan/IMMU-130, DS-8201a, U3-1402, milatuzumab doxorubicin/IMMU-110/hLL1-DOX, BMS-986148, RC48-ADC/hertuzumab-vc-MMAE, PF-06647020, PF-06650808, PF-06664178/RN927C, lupartumab amadotin/BAY1129980, aprutumab ixadotin/BAY1187982, ARX788, AGS62P1, XMT-1522, AbGn-10⁷, MEDI4276, DSTA4637S/RG7861. Anti-CD30 antibody drug conjugates are known in the art, for example, Bradley et al. Am. J. Health Syst. Pharm. (2013) 70(7): 589-97; Shen et al. mAbs (2019) 11(6): 1149-1161.

In some embodiments, binding of the antibody-drug conjugate to an epitope of the cell-surface protein (e.g., cell-surface lineage-specific cell-surface protein) induces internalization of the antibody-drug conjugate, and the drug (or toxin) may be released intracellularly. In some embodiments, binding of the antibody-drug conjugate to the epitope of a cell-surface lineage-specific protein induces internalization of the toxin or drug, which allows the toxin or drug to kill the cells expressing the lineage-specific protein (target cells). In some embodiments, binding of the antibody-drug conjugate to the epitope of a cell-surface lineage-specific protein induces internalization of the toxin or drug, which may regulate the activity of the cell expressing the lineage-specific protein (target cells). The type of toxin or drug used in the antibody-drug conjugates described herein is not limited to any specific type.

Aspects of the disclosure also provide kits, for example kits comprising reagents, e.g., for producing a genetically engineered cell. In some embodiments, the kit comprises any of the fusion polypeptides described herein and a gRNA comprising a targeting domain complementary to a target sequence in the genome of a cell. In some embodiments, the fusion polypeptide and the gRNA form a ribonucleoprotein (RNP) complex under conditions suitable to bind a target domain in the genome of a cell or plurality of cells. In some embodiments, the kit comprises any of the fusion polypeptides described herein and a second gRNA comprising a targeting domain complementary to a second target sequence in the genome of a cell. In some embodiments, the second gRNA and fusion polypeptide form a ribonucleoprotein (RNP) complex under conditions suitable to bind a second target domain in the genome of a cell or plurality of cells.

In some embodiments, the kit comprises instructions for a method of contacting a cell or plurality of cells with a gRNA and any of the fusion polypeptides described herein. In some embodiments, the instructions provide that the cell or plurality of cells is contacted with the fusion polypeptide prior to contacting the cell or plurality of cells with the gRNA. In some embodiments, the instructions provide that the cell or plurality of cells is contacted with the gRNA prior to contacting the cell or plurality of cells with the fusion polypeptide.

In some embodiments, the kit comprises a cell or plurality of cells. In some embodiments, the kit does not comprise a cell or plurality of cells (e.g., the cell or plurality of cells recited by the instructions is acquired by other means).

SEQUENCES

Nucleic acid sequences of exemplary vector sequences are provided below.
Construct A-(SEQ ID NO: 45)
gagggcctatttcccatgattccttcatatttgcatatacgatacaaggctgttagagagataattggaattaat
ttgactgtaaacacaaagatattagtacaaaatacgtgacgtagaaagtaataatttcttgggtagtttgcagtt
ttaaaattatgttttaaaatggactatcatatgcttaccgtaacttgaaagtatttcgatttcttggctttatat
atcttgtggaaaggacgaaacaccgggtcttcgagaagacctgttttagagctagaaatagcaagttaaaataag
gctagtccgttatcaacttgaaaaagtggcaccgagtcggtgcttttttgttttagagctagaaatagcaagtta
aaataaggctagtccgtttttagcgcgtgcgccaattctgcagacaaatggctctagaggtacccgttacataac
ttacggtaaatggcccgcctggctgaccgcccaacgacccccgcccattgacgtcaatagtaacgccaataggga
ctttccattgacgtcaatgggtggagtatttacggtaaactgcccacttggcagtacatcaagtgtatcatatgc
caagtacgccccctattgacgtcaatgacggtaaatggcccgcctggcattgtgcccagtacatgaccttatggg
actttcctacttggcagtacatctacgtattagtcatcgctattaccatggtcgaggtgagccccacgttctgct
tcactctccccatctcccccccctccccacccccaattttgtatttatttattttttaattattttgtgcagcga
tgggggcggggggggggggggggcgcgcgccaggcggggcggggcggggcgaggggcggggcggggcgaggcgga
gaggtgcggcggcagccaatcagagcggcgcgctccgaaagtttccttttatggcgaggcggcggcggcggcggc
cctataaaaagcgaagcgcgcggcgggcgggagtcgctgcgcgctgccttcgccccgtgccccgctccgccgccg
cctcgcgccgcccgccccggctctgactgaccgcgttactcccacaggtgagcgggcgggacggcccttctcctc
cgggctgtaattagctgagcaagaggtaagggtttaagggatggttggttggtggggtattaatgtttaattacc
tggagcacctgcctgaaatcactttttttcaggttggaccggtgccaccatgcaactggtgaagagcgagctgga
agagaagaaaagcgagctcagacataagctgaagtacgttccccacgaatacattgaactgatagaaatcgctag
aaacagtacgcaagacagaatactggaaatgaaggtgatggagttcttcatgaaggtttacggctatcgtggcaa
acacctcgggggctcccggaagcccgacggggctatctacaccgtgggcagtcccatcgactatggcgtgatcgt
ggacaccaaagcttatagcggcggatataatctccccatcggccaagccgatgagatgcagaggtatgtggagga
gaaccaaacaagaaacaagcatatcaaccccaacgagtggtggaaggtttatcctagctcggtgaccgagtttaa
gttcctattcgtgtctggccacttcaagggcaactataaggcacagctcactagactgaatcatatcacgaattg
caacggcgccgtgttatccgtggaggagctactgatcggcggagagatgatcaaagccggcaccctgaccctgga
agaggtgagaagaaagtttaacaatggcgaaataaatttcggcagcggaagtggaagcggctccatcactagaac
caccaaccctagaaacgtggtgcccaagatctacatgagcgccggcagcatccccctgaccacccacatcaccaa
ctcaattcagcccaccctgtggaccatcggcagcatcaacggcgtggcccccctggccaagagcatcaagctggg
catccccgtgaccggcagcgcctacaccgatcagaccaccgccatggtgagaaagaaggtgagcgtgttcatggg
cagcggcagcgggagcggctcatcgcagctggttaagagcgagttagaagaaaaaaagagcgaactgcggcataa
actgaagtatgtcccacacgagtacatcgaactgatcgagatcgcgagaaactctacccaagacagaattctgga
gatgaaagtaatggaatttttcatgaaggtgtatggatatagagggaagcacctgggtggcagcagaaaacccgc
cggcgccatctacactgtggggagccccatagactatggtgtgatcgtggataccaaggcgtatagcggcggtta
caatctgcccattgggcaagcggacgagatgcaaagatatgtggaagagaatcagacgaggaacaagcacattaa
ccctaatgagtggtggaaggtctaccctagctccgttaccgagttcaagttcctgtttgtgagcgggcattttaa
gggcaactacaaggcacagctgacccgcctgaaccacataacaaactgcaacggtgccgtgctgagcgtagaaga
gttgctaatcggcggcgagatgatcaaggccggcacgctaaccctcgaagaggtgcgcagaaagttcaataacgg
cgaaatcaatttcagcggcagcgagactcccgggacctcagagtccgccacacccgaaagtacacagttcgaggg
ctttaccaacctgtatcaggtgagcaagacactgcggtttgagctgatcccacagggcaagaccctgaagcacat
ccaggagcagggcttcatcgaggaggacaaggcccgcaatgatcactacaaggagctgaagcccatcatcgatcg
gatctacaagacctatgccgaccagtgcctgcagctggtgcagctggattgggagaacctgagcgccgccatcga
ctcctatagaaaggagaaaaccgaggagacaaggaacgccctgatcgaggagcaggccacatatcgcaatgccat
ccacgactacttcatcggccggacagacaacctgaccgatgccatcaataagagacacgccgagatctacaaggg
cctgttcaaggccgagctgtttaatggcaaggtgctgaagcagctgggcaccgtgaccacaaccgagcacgagaa
cgccctgctgcggagcttcgacaagtttacaacctacttctccggcttttatagaaacaggaagaacgtgttcag
cgccgaggatatcagcacagccatcccacaccgcatcgtgcaggacaacttccccaagtttaaggagaattgtca
catcttcacacgcctgatcaccgccgtgcccagcctgcgggagcactttgagaacgtgaagaaggccatcggcat
cttcgtgagcacctccatcgaggaggtgttttccttccctttttataaccagctgctgacacagacccagatcga
cctgtataaccagctgctgggaggaatctctcgggaggcaggcaccgagaagatcaagggcctgaacgaggtgct
gaatctggccatccagaagaatgatgagacagcccacatcatcgcctccctgccacacagattcatccccctgtt
taagcagatcctgtccgataggaacaccctgtctttcatcctggaggagtttaagagcgacgaggaagtgatcca
gtccttctgcaagtacaagacactgctgagaaacgagaacgtgctggagacagccgaggccctgtttaacgagct
gaacagcatcgacctgacacacatcttcatcagccacaagaagctggagacaatcagcagcgccctgtgcgacca
ctgggatacactgaggaatgccctgtatgagcggagaatctccgagctgacaggcaagatcaccaagtctgccaa
ggagaaggtgcagcgcagcctgaagcacgaggatatcaacctgcaggagatcatctctgccgcaggcaaggagct
gagcgaggccttcaagcagaaaaccagcgagatcctgtcccacgcacacgccgccctggatcagccactgcctac
aaccctgaagaagcaggaggagaaggagatcctgaagtctcagctggacagcctgctgggcctgtaccacctgct
ggactggtttgccgtggatgagtccaacgaggtggaccccgagttctctgcccggctgaccggcatcaagctgga
gatggagccttctctgagcttctacaacaaggccagaaattatgccaccaagaagccctactccgtggagaagtt
caagctgaactttcagatgcctacactggccagaggctgggacgtgaatgtggagaagaacagaggcgccatcct
gtttgtgaagaacggcctgtactatctgggcatcatgccaaagcagaagggcaggtataaggccctgagcttcga
gcccacagagaaaaccagcgagggctttgataagatgtactatgactacttccctgatgccgccaagatgatccc
aaagtgcagcacccagctgaaggccgtgacagcccactttcagacccacacaacccccatcctgctgtccaacaa
tttcatcgagcctctggagatcacaaaggagatctacgacctgaacaatcctgagaaggagccaaagaagtttca
gacagcctacgccaagaaaaccggcgaccagaagggctacagagaggccctgtgcaagtggatcgacttcacaag
ggattttctgtccaagtataccaagacaacctctatcgatctgtctagcctgcggccatcctctcagtataagga
cctgggcgagtactatgccgagctgaatcccctgctgtaccacatcagcttccagagaatcgccgagaaggagat
catggatgccgtggagacaggcaagctgtacctgttccagatctataacaaggactttgccaagggccaccacgg
caagcctaatctgcacacactgtattggaccggcctgttttctccagagaacctggccaagacaagcatcaagct
gaatggccaggccgagctgttctaccgccctaagtccaggatgaagaggatggcacaccggctgggagagaagat
gctgaacaagaagctgaaggatcagaaaaccccaatccccgacaccctgtaccaggagctgtacgactatgtgaa
tcacagactgtcccacgacctgtctgatgaggccagggccctgctgcccaacgtgatcaccaaggaggtgtctca
cgagatcatcaaggataggcgctttaccagcgacaagttctttttccacgtgcctatcacactgaactatcaggc
cgccaattccccatctaagttcaaccagagggtgaatgcctacctgaaggagcaccccgagacacctatcatcgg
catcgcccggggcgagagaaacctgatctatatcacagtgatcgactccaccggcaagatcctggagcagcggag
cctgaacaccatccagcagtttgattaccagaagaagctggacaacagggagaaggagagggtggcagcaaggca
ggcctggtctgtggtgggcacaatcaaggatctgaagcagggctatctgagccaggtcatccacgagatcgtgga
cctgatgatccactaccaggccgtggtggtgctggagaacctgaatttcggctttaagagcaagaggaccggcat
cgccgagaaggccgtgtaccagcagttcgagaagatgctgatcgataagctgaattgcctggtgctgaaggacta
tccagcagagaaagtgggaggcgtgctgaacccataccagctgacagaccagttcacctcctttgccaagatggg
cacccagtctggcttcctgttttacgtgcctgccccatatacatctaagatcgatcccctgaccggcttcgtgga
ccccttcgtgtggaaaaccatcaagaatcacgagagccgcaagcacttcctggagggcttcgactttctgcacta
cgacgtgaaaaccggcgacttcatcctgcactttaagatgaacagaaatctgtccttccagaggggcctgcccgg
ctttatgcctgcatgggatatcgtgttcgagaagaacgagacacagtttgacgccaagggcacccctttcatcgc
cggcaagagaatcgtgccagtgatcgagaatcacagattcaccggcagataccgggacctgtatcctgccaacga
gctgatcgccctgctggaggagaagggcatcgtgttcagggatggctccaacatcctgccaaagctgctggagaa
tgacgattctcacgccatcgacaccatggtggccctgatccgcagcgtgctgcagatgcggaactccaatgccgc
cacaggcgaggactatatcaacagccccgtgcgcgatctgaatggcgtgtgcttcgactcccggtttcagaaccc
agagtggcccatggacgccgatgccaatggcgcctaccacatcgccctgaagggccagctgctgctgaatcacct
gaaggagagcaaggatctgaagctgcagaacggcatctccaatcaggactggctggcctacatccaggagctgcg
caacaaaaggccggcggccacgaaaaaggccggccaggcaaaaaagaaaaagggatcctacccatacgatgttcc
agattacgcttatccctacgacgtgcctgattatgcatacccatatgatgtccccgactatgccgagggcagagg
aagtctgctaacatgcggtgacgtcgaggagaatcctggcccaatgaccgagtacaagcccacggtgcgcctcgc
cacccgcgacgacgtccccagggccgtacgcaccctcgccgccgcgttcgccgactaccccgccacgcgccacac
cgtcgatccggaccgccacatcgagcgggtcaccgagctgcaagaactcttcctcacgcgcgtcgggctcgacat
cggcaaggtgtgggtcgcggacgacggcgccgcggtggcggtctggaccacgccggagagcgtcgaagcgggggc
ggtgttcgccgagatcggcccgcgcatggccgagttgagcggttcccggctggccgcgcagcaacagatggaagg
cctcctggcgccgcaccggcccaaggagcccgcgtggttcctggccaccgtcggagtctcgcccgaccaccaggg
caagggtctgggcagcgccgtcgtgctccccggagtggaggcggccgagcgcgccggggtgcccgccttcctgga
gacctccgcgccccgcaacctccccttctacgagcggctcggcttcaccgtcaccgccgacgtcgaggtgcccga
aggaccgcgcacctggtgcatgacccgcaagcccggtgcctgaactagtcctgcaggcatgcaagcttgatatca
agcttatcgataatcaacctctggattacaaaatttgtgaaagattgactggtattcttaactatgttgctcctt
ttacgctatgtggatacgctgctttaatgcctttgtatcatgctattgcttcccgtatggctttcattttctcct
ccttgtataaatcctggttgctgtctctttatgaggagttgtggcccgttgtcaggcaacgtggcgtggtgtgca
ctgtgtttgctgacgcaacccccactggttggggcattgccaccacctgtcagctcctttccgggactttcgctt
tccccctccctattgccacggcggaactcatcgccgcctgccttgcccgctgctggacaggggctcggctgttgg
gcactgacaattccgtggtgttgtcggggaaatcatcgtcctttccttggctgctcgcctgtgttgccacctgga
ttctgcgcgggacgtccttctgctacgtcccttcggccctcaatccagcggaccttccttcccgcggcctgctgc
cggctctgcggcctcttccgcgtcttcgccttcgccctcagacgagtcggatctccctttgggccgcctccccgc
atcgataccgtcgacctcgagggaattaattcgagctcggtacctttaagaccgatgacttacaaggcagctgta
gatcttagccactttttaaaagaaattaactgtgccttctagttgccagccatctgttgtttgcccctcccccgt
gccttccttgaccctggaaggtgccactcccactgtcctttcctaataaaatgaggaaattgcatcgcattgtct
gagtaggtgtcattctattctggggggtggggtggggcaggacagcaagggggaggattgggaagagaatagcag
gcatgctggggagcggccgcaggaacccctagtgatggagttggccactccctctctgcgcgctcgctcgctcac
tgaggccgggcgaccaaaggtcgcccgacgcccgggctttgcccgggcggcctcagtgagcgagcgagcgcgcag
ctgcctgcaggggcgcctgatgcggtattttctccttacgcatctgtgcggtatttcacaccgcatacgtcaaag
caaccatagtacgcgccctgtagcggcgcattaagcgcggcgggtgtggtggttacgcgcagcgtgaccgctaca
cttgccagcgccttagcgcccgctcctttcgctttcttcccttcctttctcgccacgttcgccggctttccccgt
caagctctaaatcgggggctccctttagggttccgatttagtgctttacggcacctcgaccccaaaaaacttgat
ttgggtgatggttcacgtagtgggccatcgccctgatagacggtttttcgccctttgacgttggagtccacgttc
tttaatagtggactcttgttccaaactggaacaacactcaactctatctcgggctattcttttgatttataaggg
attttgccgatttcggtctattggttaaaaaatgagctgatttaacaaaaatttaacgcgaattttaacaaaata
ttaacgtttacaattttatggtgcactctcagtacaatctgctctgatgccgcatagttaagccagccccgacac
ccgccaacacccgctgacgcgccctgacgggcttgtctgctcccggcatccgcttacagacaagctgtgaccgtc
tccgggagctgcatgtgtcagaggttttcaccgtcatcaccgaaacgcgcgagacgaaagggcctcgtgatacgc
ctatttttataggttaatgtcatgataataatggtttcttagacgtcaggtggcacttttcggggaaatgtgcgc
ggaacccctatttgtttatttttctaaatacattcaaatatgtatccgctcatgagacaataaccctgataaatg
cttcaataatattgaaaaaggaagagtatgagtattcaacatttccgtgtcgcccttattcccttttttgcggca
ttttgccttcctgtttttgctcacccagaaacgctggtgaaagtaaaagatgctgaagatcagttgggtgcacga
gtgggttacatcgaactggatctcaacagcggtaagatccttgagagttttcgccccgaagaacgttttccaatg
atgagcacttttaaagttctgctatgtggcgcggtattatcccgtattgacgccgggcaagagcaactcggtcgc
cgcatacactattctcagaatgacttggttgagtactcaccagtcacagaaaagcatcttacggatggcatgaca
gtaagagaattatgcagtgctgccataaccatgagtgataacactgcggccaacttacttctgacaacgatcgga
ggaccgaaggagctaaccgcttttttgcacaacatgggggatcatgtaactcgccttgatcgttgggaaccggag
ctgaatgaagccataccaaacgacgagcgtgacaccacgatgcctgtagcaatggcaacaacgttgcgcaaacta
ttaactggcgaactacttactctagcttcccggcaacaattaatagactggatggaggcggataaagttgcagga
ccacttctgcgctcggcccttccggctggctggtttattgctgataaatctggagccggtgagcgtggaagccgc
ggtatcattgcagcactggggccagatggtaagccctcccgtatcgtagttatctacacgacggggagtcaggca
actatggatgaacgaaatagacagatcgctgagataggtgcctcactgattaagcattggtaactgtcagaccaa
gtttactcatatatactttagattgatttaaaacttcatttttaatttaaaaggatctaggtgaagatccttttt
gataatctcatgaccaaaatcccttaacgtgagttttcgttccactgagcgtcagaccccgtagaaaagatcaaa
ggatcttcttgagatcctttttttctgcgcgtaatctgctgcttgcaaacaaaaaaaccaccgctaccagcggtg
gtttgtttgccggatcaagagctaccaactctttttccgaaggtaactggcttcagcagagcgcagataccaaat
actgttcttctagtgtagccgtagttaggccaccacttcaagaactctgtagcaccgcctacatacctcgctctg
ctaatcctgttaccagtggctgctgccagtggcgataagtcgtgtcttaccgggttggactcaagacgatagtta
ccggataaggcgcagcggtcgggctgaacggggggttcgtgcacacagcccagcttggagcgaacgacctacacc
gaactgagatacctacagcgtgagctatgagaaagcgccacgcttcccgaagggagaaaggcggacaggtatccg
gtaagcggcagggtcggaacaggagagcgcacgagggagcttccagggggaaacgcctggtatctttatagtcct
gtcgggtttcgccacctctgacttgagcgtcgatttttgtgatgctcgtcaggggggcggagcctatggaaaaac
gccagcaacgcggcctttttacggttcctggccttttgctggccttttgctcacatgt
Construct B-(SEQ ID NO: 46)
gagggcctatttcccatgattccttcatatttgcatatacgatacaaggctgttagagagataattggaattaat
ttgactgtaaacacaaagatattagtacaaaatacgtgacgtagaaagtaataatttcttgggtagtttgcagtt
ttaaaattatgttttaaaatggactatcatatgcttaccgtaacttgaaagtatttcgatttcttggctttatat
atcttgtggaaaggacgaaacaccgggtcttcgagaagacctgttttagagctagaaatagcaagttaaaataag
gctagtccgttatcaacttgaaaaagtggcaccgagtcggtgcttttttgttttagagctagaaatagcaagtta
aaataaggctagtccgtttttagcgcgtgcgccaattctgcagacaaatggctctagaggtacccgttacataac
ttacggtaaatggcccgcctggctgaccgcccaacgacccccgcccattgacgtcaatagtaacgccaataggga
ctttccattgacgtcaatgggtggagtatttacggtaaactgcccacttggcagtacatcaagtgtatcatatgc
caagtacgccccctattgacgtcaatgacggtaaatggcccgcctggcattgtgcccagtacatgaccttatggg
actttcctacttggcagtacatctacgtattagtcatcgctattaccatggtcgaggtgagccccacgttctgct
tcactctccccatctcccccccctccccacccccaattttgtatttatttattttttaattattttgtgcagcga
tgggggcggggggggggggggggcgcgcgccaggcggggcggggcggggcgaggggcggggcggggcgaggcgga
gaggtgcggcggcagccaatcagagcggcgcgctccgaaagtttccttttatggcgaggcggcggcggcggcggc
cctataaaaagcgaagcgcgcggcgggcgggagtcgctgcgcgctgccttcgccccgtgccccgctccgccgccg
cctcgcgccgcccgccccggctctgactgaccgcgttactcccacaggtgagcgggcgggacggcccttctcctc
cgggctgtaattagctgagcaagaggtaagggtttaagggatggttggttggtggggtattaatgtttaattacc
tggagcacctgcctgaaatcactttttttcaggttggaccggtgccaccatgggcagctcagagactggcccagt
ggctgtggaccccacattgagacggcggatcgagccccatgagtttgaggtattcttcgatccgagagagctccg
caaggagacctgcctgctttacgaaattaattgggggggccggcactccatttggcgacatacatcacagaacac
taacaagcacgtcgaagtcaacttcatcgagaagttcacgacagaaagatatttctgtccgaacacaaggtgcag
cattacctggtttctcagctggagcccatgcggcgaatgtagtagggccatcactgaattcctgtcaaggtatcc
ccacgtcactctgtttatttacatcgcaaggctgtaccaccacgctgacccccgcaatcgacaaggcctgcggga
tttgatctcttcaggtgtgactatccaaattatgactgagcaggagtcaggatactgctggagaaactttgtgaa
ttatagcccgagtaatgaagcccactggcctaggtatccccatctgtgggtacgactgtacgttcttgaactgta
ctgcatcatactgggcctgcctccttgtctcaacattctgagaaggaagcagccacagctgacattctttaccat
cgctcttcagtcttgtcattaccagcgactgcccccacacattctctgggccaccgggttgaaatctggtggttc
ttctggtggttctagcggcagcgagactcccgggacctcagagtccgccacacccgaaagttccggagggagtag
cggcgggtctacacagttcgagggctttaccaacctgtatcaggtgagcaagacactgcggtttgagctgatccc
acagggcaagaccctgaagcacatccaggagcagggcttcatcgaggaggacaaggcccgcaatgatcactacaa
ggagctgaagcccatcatcgatcggatctacaagacctatgccgaccagtgcctgcagctggtgcagctggattg
ggagaacctgagcgccgccatcgactcctatagaaaggagaaaaccgaggagacaaggaacgccctgatcgagga
gcaggccacatatcgcaatgccatccacgactacttcatcggccggacagacaacctgaccgatgccatcaataa
gagacacgccgagatctacaagggcctgttcaaggccgagctgtttaatggcaaggtgctgaagcagctgggcac
cgtgaccacaaccgagcacgagaacgccctgctgcggagcttcgacaagtttacaacctacttctccggctttta
tagaaacaggaagaacgtgttcagcgccgaggatatcagcacagccatcccacaccgcatcgtgcaggacaactt
ccccaagtttaaggagaattgtcacatcttcacacgcctgatcaccgccgtgcccagcctgcgggagcactttga
gaacgtgaagaaggccatcggcatcttcgtgagcacctccatcgaggaggtgttttccttccctttttataacca
gctgctgacacagacccagatcgacctgtataaccagctgctgggaggaatctctcgggaggcaggcaccgagaa
gatcaagggcctgaacgaggtgctgaatctggccatccagaagaatgatgagacagcccacatcatcgcctccct
gccacacagattcatccccctgtttaagcagatcctgtccgataggaacaccctgtctttcatcctggaggagtt
taagagcgacgaggaagtgatccagtccttctgcaagtacaagacactgctgagaaacgagaacgtgctggagac
agccgaggccctgtttaacgagctgaacagcatcgacctgacacacatcttcatcagccacaagaagctggagac
aatcagcagcgccctgtgcgaccactgggatacactgaggaatgccctgtatgagcggagaatctccgagctgac
aggcaagatcaccaagtctgccaaggagaaggtgcagcgcagcctgaagcacgaggatatcaacctgcaggagat
catctctgccgcaggcaaggagctgagcgaggccttcaagcagaaaaccagcgagatcctgtcccacgcacacgc
cgccctggatcagccactgcctacaaccctgaagaagcaggaggagaaggagatcctgaagtctcagctggacag
cctgctgggcctgtaccacctgctggactggtttgccgtggatgagtccaacgaggtggaccccgagttctctgc
ccggctgaccggcatcaagctggagatggagccttctctgagcttctacaacaaggccagaaattatgccaccaa
gaagccctactccgtggagaagttcaagctgaactttcagatgcctacactggccagaggctgggacgtgaatgt
ggagaagaacagaggcgccatcctgtttgtgaagaacggcctgtactatctgggcatcatgccaaagcagaaggg
caggtataaggccctgagcttcgagcccacagagaaaaccagcgagggctttgataagatgtactatgactactt
ccctgatgccgccaagatgatcccaaagtgcagcacccagctgaaggccgtgacagcccactttcagacccacac
aacccccatcctgctgtccaacaatttcatcgagcctctggagatcacaaaggagatctacgacctgaacaatcc
tgagaaggagccaaagaagtttcagacagcctacgccaagaaaaccggcgaccagaagggctacagagaggccct
gtgcaagtggatcgacttcacaagggattttctgtccaagtataccaagacaacctctatcgatctgtctagcct
gcggccatcctctcagtataaggacctgggcgagtactatgccgagctgaatcccctgctgtaccacatcagctt
ccagagaatcgccgagaaggagatcatggatgccgtggagacaggcaagctgtacctgttccagatctataacaa
ggactttgccaagggccaccacggcaagcctaatctgcacacactgtattggaccggcctgttttctccagagaa
cctggccaagacaagcatcaagctgaatggccaggccgagctgttctaccgccctaagtccaggatgaagaggat
ggcacaccggctgggagagaagatgctgaacaagaagctgaaggatcagaaaaccccaatccccgacaccctgta
ccaggagctgtacgactatgtgaatcacagactgtcccacgacctgtctgatgaggccagggccctgctgcccaa
cgtgatcaccaaggaggtgtctcacgagatcatcaaggataggcgctttaccagcgacaagttctttttccacgt
gcctatcacactgaactatcaggccgccaattccccatctaagttcaaccagagggtgaatgcctacctgaagga
gcaccccgagacacctatcatcggcatcgcccggggcgagagaaacctgatctatatcacagtgatcgactccac
cggcaagatcctggagcagcggagcctgaacaccatccagcagtttgattaccagaagaagctggacaacaggga
gaaggagagggtggcagcaaggcaggcctggtctgtggtgggcacaatcaaggatctgaagcagggctatctgag
ccaggtcatccacgagatcgtggacctgatgatccactaccaggccgtggtggtgctggagaacctgaatttcgg
ctttaagagcaagaggaccggcatcgccgagaaggccgtgtaccagcagttcgagaagatgctgatcgataagct
gaattgcctggtgctgaaggactatccagcagagaaagtgggaggcgtgctgaacccataccagctgacagacca
gttcacctcctttgccaagatgggcacccagtctggcttcctgttttacgtgcctgccccatatacatctaagat
cgatcccctgaccggcttcgtggaccccttcgtgtggaaaaccatcaagaatcacgagagccgcaagcacttcct
ggagggcttcgactttctgcactacgacgtgaaaaccggcgacttcatcctgcactttaagatgaacagaaatct
gtccttccagaggggcctgcccggctttatgcctgcatgggatatcgtgttcgagaagaacgagacacagtttga
cgccaagggcacccctttcatcgccggcaagagaatcgtgccagtgatcgagaatcacagattcaccggcagata
ccgggacctgtatcctgccaacgagctgatcgccctgctggaggagaagggcatcgtgttcagggatggctccaa
catcctgccaaagctgctggagaatgacgattctcacgccatcgacaccatggtggccctgatccgcagcgtgct
gcagatgcggaactccaatgccgccacaggcgaggactatatcaacagccccgtgcgcgatctgaatggcgtgtg
cttcgactcccggtttcagaacccagagtggcccatggacgccgatgccaatggcgcctaccacatcgccctgaa
gggccagctgctgctgaatcacctgaaggagagcaaggatctgaagctgcagaacggcatctccaatcaggactg
gctggcctacatccaggagctgcgcaacagcggcagcgagactcccgggacctcagagtccgccacacccgaaag
tcaactggtgaagagcgagctggaagagaagaaaagcgagctcagacataagctgaagtacgttccccacgaata
cattgaactgatagaaatcgctagaaacagtacgcaagacagaatactggaaatgaaggtgatggagttcttcat
gaaggtttacggctatcgtggcaaacacctcgggggctcccggaagcccgccggggctatctacaccgtgggcag
tcccatcgactatggcgtgatcgtggacaccaaagcttatagcggcggatataatctccccatcggccaagccga
tgagatgcagaggtatgtggaggagaaccaaacaagaaacaagcatatcaaccccaacgagtggtggaaggttta
tcctagctcggtgaccgagtttaagttcctattcgtgtctggccacttcaagggcaactataaggcacagctcac
tagactgaatcatatcacgaattgcaacggcgccgtgttatccgtggaggagctactgatcggcggagagatgat
caaagccggcaccctgaccctggaagaggtgagaagaaagtttaacaatggcgaaataaatttcggcagcggaag
tggaagcggctccatcactagaaccaccaaccctagaaacgtggtgcccaagatctacatgagcgccggcagcat
ccccctgaccacccacatcaccaactcaattcagcccaccctgtggaccatcggcagcatcaacggcgtggcccc
cctggccaagagcatcaagctgggcatccccgtgaccggcagcgcctacaccgatcagaccaccgccatggtgag
aaagaaggtgagcgtgttcatgggcagcggcagcgggagcggctcatcgcagctggttaagagcgagttagaaga
aaaaaagagcgaactgcggcataaactgaagtatgtcccacacgagtacatcgaactgatcgagatcgcgagaaa
ctctacccaagacagaattctggagatgaaagtaatggaatttttcatgaaggtgtatggatatagagggaagca
cctgggtggcagcagaaaacccgacggcgccatctacactgtggggagccccatagactatggtgtgatcgtgga
taccaaggcgtatagcggcggttacaatctgcccattgggcaagcggacgagatgcaaagatatgtggaagagaa
tcagacgaggaacaagcacattaaccctaatgagtggtggaaggtctaccctagctccgttaccgagttcaagtt
cctgtttgtgagcgggcattttaagggcaactacaaggcacagctgacccgcctgaaccacataacaaactgcaa
cggtgccgtgctgagcgtagaagagttgctaatcggcggcgagatgatcaaggccggcacgctaaccctcgaaga
ggtgcgcagaaagttcaataacggcgaaatcaatttcaaaaggccggcggccacgaaaaaggccggccaggcaaa
aaagaaaaagggatcctacccatacgatgttccagattacgcttatccctacgacgtgcctgattatgcataccc
atatgatgtccccgactatgccgagggcagaggaagtctgctaacatgcggtgacgtcgaggagaatcctggccc
aatgaccgagtacaagcccacggtgcgcctcgccacccgcgacgacgtccccagggccgtacgcaccctcgccgc
cgcgttcgccgactaccccgccacgcgccacaccgtcgatccggaccgccacatcgagcgggtcaccgagctgca
agaactcttcctcacgcgcgtcgggctcgacatcggcaaggtgtgggtcgcggacgacggcgccgcggtggcggt
ctggaccacgccggagagcgtcgaagcgggggcggtgttcgccgagatcggcccgcgcatggccgagttgagcgg
ttcccggctggccgcgcagcaacagatggaaggcctcctggcgccgcaccggcccaaggagcccgcgtggttcct
ggccaccgtcggagtctcgcccgaccaccagggcaagggtctgggcagcgccgtcgtgctccccggagtggaggc
ggccgagcgcgccggggtgcccgccttcctggagacctccgcgccccgcaacctccccttctacgagcggctcgg
cttcaccgtcaccgccgacgtcgaggtgcccgaaggaccgcgcacctggtgcatgacccgcaagcccggtgcctg
aactagtcctgcaggcatgcaagcttgatatcaagcttatcgataatcaacctctggattacaaaatttgtgaaa
gattgactggtattcttaactatgttgctccttttacgctatgtggatacgctgctttaatgcctttgtatcatg
ctattgcttcccgtatggctttcattttctcctccttgtataaatcctggttgctgtctctttatgaggagttgt
ggcccgttgtcaggcaacgtggcgtggtgtgcactgtgtttgctgacgcaacccccactggttggggcattgcca
ccacctgtcagctcctttccgggactttcgctttccccctccctattgccacggcggaactcatcgccgcctgcc
ttgcccgctgctggacaggggctcggctgttgggcactgacaattccgtggtgttgtcggggaaatcatcgtcct
ttccttggctgctcgcctgtgttgccacctggattctgcgcgggacgtccttctgctacgtcccttcggccctca
atccagcggaccttccttcccgcggcctgctgccggctctgcggcctcttccgcgtcttcgccttcgccctcaga
cgagtcggatctccctttgggccgcctccccgcatcgataccgtcgacctcgagggaattaattcgagctcggta
cctttaagaccgatgacttacaaggcagctgtagatcttagccactttttaaaagaaattaactgtgccttctag
ttgccagccatctgttgtttgcccctcccccgtgccttccttgaccctggaaggtgccactcccactgtcctttc
ctaataaaatgaggaaattgcatcgcattgtctgagtaggtgtcattctattctggggggtggggtggggcagga
cagcaagggggaggattgggaagagaatagcaggcatgctggggagcggccgcaggaacccctagtgatggagtt
ggccactccctctctgcgcgctcgctcgctcactgaggccgggcgaccaaaggtcgcccgacgcccgggctttgc
ccgggcggcctcagtgagcgagcgagcgcgcagctgcctgcaggggcgcctgatgcggtattttctccttacgca
tctgtgcggtatttcacaccgcatacgtcaaagcaaccatagtacgcgccctgtagcggcgcattaagcgcggcg
ggtgtggtggttacgcgcagcgtgaccgctacacttgccagcgccttagcgcccgctcctttcgctttcttccct
tcctttctcgccacgttcgccggctttccccgtcaagctctaaatcgggggctccctttagggttccgatttagt
gctttacggcacctcgaccccaaaaaacttgatttgggtgatggttcacgtagtgggccatcgccctgatagacg
gtttttcgccctttgacgttggagtccacgttctttaatagtggactcttgttccaaactggaacaacactcaac
tctatctcgggctattcttttgatttataagggattttgccgatttcggtctattggttaaaaaatgagctgatt
taacaaaaatttaacgcgaattttaacaaaatattaacgtttacaattttatggtgcactctcagtacaatctgc
tctgatgccgcatagttaagccagccccgacacccgccaacacccgctgacgcgccctgacgggcttgtctgctc
ccggcatccgcttacagacaagctgtgaccgtctccgggagctgcatgtgtcagaggttttcaccgtcatcaccg
aaacgcgcgagacgaaagggcctcgtgatacgcctatttttataggttaatgtcatgataataatggtttcttag
acgtcaggtggcacttttcggggaaatgtgcgcggaacccctatttgtttatttttctaaatacattcaaatatg
tatccgctcatgagacaataaccctgataaatgcttcaataatattgaaaaaggaagagtatgagtattcaacat
ttccgtgtcgcccttattcccttttttgcggcattttgccttcctgtttttgctcacccagaaacgctggtgaaa
gtaaaagatgctgaagatcagttgggtgcacgagtgggttacatcgaactggatctcaacagcggtaagatcctt
gagagttttcgccccgaagaacgttttccaatgatgagcacttttaaagttctgctatgtggcgcggtattatcc
cgtattgacgccgggcaagagcaactcggtcgccgcatacactattctcagaatgacttggttgagtactcacca
gtcacagaaaagcatcttacggatggcatgacagtaagagaattatgcagtgctgccataaccatgagtgataac
actgcggccaacttacttctgacaacgatcggaggaccgaaggagctaaccgcttttttgcacaacatgggggat
catgtaactcgccttgatcgttgggaaccggagctgaatgaagccataccaaacgacgagcgtgacaccacgatg
cctgtagcaatggcaacaacgttgcgcaaactattaactggcgaactacttactctagcttcccggcaacaatta
atagactggatggaggcggataaagttgcaggaccacttctgcgctcggcccttccggctggctggtttattgct
gataaatctggagccggtgagcgtggaagccgcggtatcattgcagcactggggccagatggtaagccctcccgt
atcgtagttatctacacgacggggagtcaggcaactatggatgaacgaaatagacagatcgctgagataggtgcc
tcactgattaagcattggtaactgtcagaccaagtttactcatatatactttagattgatttaaaacttcatttt
taatttaaaaggatctaggtgaagatcctttttgataatctcatgaccaaaatcccttaacgtgagttttcgttc
cactgagcgtcagaccccgtagaaaagatcaaaggatcttcttgagatcctttttttctgcgcgtaatctgctgc
ttgcaaacaaaaaaaccaccgctaccagcggtggtttgtttgccggatcaagagctaccaactctttttccgaag
gtaactggcttcagcagagcgcagataccaaatactgttcttctagtgtagccgtagttaggccaccacttcaag
aactctgtagcaccgcctacatacctcgctctgctaatcctgttaccagtggctgctgccagtggcgataagtcg
tgtcttaccgggttggactcaagacgatagttaccggataaggcgcagcggtcgggctgaacggggggttcgtgc
acacagcccagcttggagcgaacgacctacaccgaactgagatacctacagcgtgagctatgagaaagcgccacg
cttcccgaagggagaaaggcggacaggtatccggtaagcggcagggtcggaacaggagagcgcacgagggagctt
ccagggggaaacgcctggtatctttatagtcctgtcgggtttcgccacctctgacttgagcgtcgatttttgtga
tgctcgtcaggggggcggagcctatggaaaaacgccagcaacgcggcctttttacggttcctggccttttgctgg
ccttttgctcacatgt
Construct C (SEQ ID NO: 47)
gagggcctatttcccatgattccttcatatttgcatatacgatacaaggctgttagagagataattggaattaat
ttgactgtaaacacaaagatattagtacaaaatacgtgacgtagaaagtaataatttcttgggtagtttgcagtt
ttaaaattatgttttaaaatggactatcatatgcttaccgtaacttgaaagtatttcgatttcttggctttatat
atcttgtggaaaggacgaaacaccgggtcttcgagaagacctgttttagagctagaaatagcaagttaaaataag
gctagtccgttatcaacttgaaaaagtggcaccgagtcggtgcttttttgttttagagctagaaatagcaagtta
aaataaggctagtccgtttttagcgcgtgcgccaattctgcagacaaatggctctagaggtacccgttacataac
ttacggtaaatggcccgcctggctgaccgcccaacgacccccgcccattgacgtcaatagtaacgccaataggga
ctttccattgacgtcaatgggtggagtatttacggtaaactgcccacttggcagtacatcaagtgtatcatatgc
caagtacgccccctattgacgtcaatgacggtaaatggcccgcctggcattgtgcccagtacatgaccttatggg
actttcctacttggcagtacatctacgtattagtcatcgctattaccatggtcgaggtgagccccacgttctgct
tcactctccccatctcccccccctccccacccccaattttgtatttatttattttttaattattttgtgcagcga
tgggggcggggggggggggggggcgcgcgccaggcggggcggggcggggcgaggggcggggcggggcgaggcgga
gaggtgcggcggcagccaatcagagcggcgcgctccgaaagtttccttttatggcgaggcggcggcggcggcggc
cctataaaaagcgaagcgcgcggcgggcgggagtcgctgcgcgctgccttcgccccgtgccccgctccgccgccg
cctcgcgccgcccgccccggctctgactgaccgcgttactcccacaggtgagcgggcgggacggcccttctcctc
cgggctgtaattagctgagcaagaggtaagggtttaagggatggttggttggtggggtattaatgtttaattacc
tggagcacctgcctgaaatcactttttttcaggttggaccggtgccaccatgggcagctcagagactggcccagt
ggctgtggaccccacattgagacggcggatcgagccccatgagtttgaggtattcttcgatccgagagagctccg
caaggagacctgcctgctttacgaaattaattgggggggccggcactccatttggcgacatacatcacagaacac
taacaagcacgtcgaagtcaacttcatcgagaagttcacgacagaaagatatttctgtccgaacacaaggtgcag
cattacctggtttctcagctggagcccatgcggcgaatgtagtagggccatcactgaattcctgtcaaggtatcc
ccacgtcactctgtttatttacatcgcaaggctgtaccaccacgctgacccccgcaatcgacaaggcctgcggga
tttgatctcttcaggtgtgactatccaaattatgactgagcaggagtcaggatactgctggagaaactttgtgaa
ttatagcccgagtaatgaagcccactggcctaggtatccccatctgtgggtacgactgtacgttcttgaactgta
ctgcatcatactgggcctgcctccttgtctcaacattctgagaaggaagcagccacagctgacattctttaccat
cgctcttcagtcttgtcattaccagcgactgcccccacacattctctgggccaccgggttgaaatctggtggttc
ttctggtggttctagcggcagcgagactcccgggacctcagagtccgccacacccgaaagttccggagggagtag
cggcgggtctaataacggaactaataacttccaaaacttcatcgggatcagttccttgcagaaaactctccggaa
tgctctcatcccaactgagactactcagcagttcattgttaagaatggaatcataaaagaggacgagcttagggg
ggaaaataggcaaatcctcaaggatatcatggatgactattataggggctttatatccgagacactgagcagcat
tgatgatatagactggacctctcttttcgaaaagatggaaatacaacttaaaaatggagataacaaggacaccct
gataaaggaacagaccgaatataggaaggcaattcataaaaagtttgctaacgatgataggtttaaaaacatgtt
ctcagcaaaactcatttcagatatactgcccgaattcgttatccacaacaacaactactccgctagcgaaaaaga
ggaaaagacccaagtcataaagctgttctctcgattcgcgacgagttttaaagattatttccgaaatcgcgcaaa
ctgtttctcagctgatgatatcagcagctcatcctgtcatcggatcgttaacgataatgctgaaatcttcttctc
caatgcacttgtttataggcgcattgttaaatctctctcaaacgatgatatcaataagatttccggcgatatgaa
ggacagtcttaaggagatgagcctcgaagagatatactcatacgagaaatatggcgaatttatcacccaggaagg
gatttccttctataatgacatttgcggcaaagtcaattccttcatgaacctgtattgccaaaaaaataaagaaaa
caagaacctctataagctgcaaaagttgcataagcaaatactttgtatcgcggatacaagctatgaagttcccta
caagttcgagagtgatgaggaggtgtatcaatctgtcaatggtttccttgataatatttcttctaagcatattgt
tgaacgactccgaaagataggagacaactataatggatacaatttggataaaatctacatcgtgtctaaatttta
cgagagtgtgtcacaaaaaacatatagagactgggagacaattaataccgccctggagatacattacaacaatat
acttcccgggaacgggaagtctaaggcagacaaggtgaagaaagccgtgaagaacgacttgcaaaagtcaattac
cgaaatcaatgagcttgtttcaaactataaactttgttcagatgacaatattaaagccgaaacctatattcatga
aatctctcatattctgaataactttgaggcgcaagaactgaaatataacccagaaatacacctcgttgagtccga
actgaaagcaagcgaactgaaaaatgttttggacgtgataatgaacgcttttcattggtgctcagtctttatgac
agaggagcttgttgacaaggataacaatttctatgcggaactggaagagatttacgacgaaatctatccggtcat
atccctgtataacctggttcgcaactatgtcacgcaaaaaccatacagcacgaagaagattaaactgaactttgg
tattccgacgctggcccgcggatggtcaaaatctgttgaatactcacgaaatgccataatcctgatgcgagataa
cctctactaccttggaatctttaatgctaaaaataaacccgataaaaaaattatcgaagggaacacgagtgaaaa
caaaggtgattataaaaaaatgatatataatctgcttccaggaccaaataagatgatacccaaagttttcctttc
ttcaaagaccggcgtcgagacatataaaccatccgcgtacatacttgaaggctacaaacaaaataaacatatcaa
atcatctaaggattttgacattacgttctgtcatgatttgattgactatttcaaaaattgcatagccattcatcc
agagtggaaaaactttgggtttgacttctctgataccagtacatatgaagacataagtggattttaccgagaagt
agagctccaaggttataaaatagactggacctatatatctgaaaaggatatagaccttttgcaagagaagggaca
gctttatcttttccaaatctacaacaaagacttcagtaagaaaagtaccgggaatgacaatcttcataccatgta
tctgaagaacctgttctccgaagaaaatctgaaggacatagtcctgaagcttaatggcgaagcggaaattttttt
ccgaaagagctctattaagaaccccataatacataagaagggaagcattctcgttaatcgaacgtatgaggccga
agagaaagatcaatttgggaatatccaaatcgttcgaaagaacataccagaaaatatttaccaagaattgtacaa
atattttaacgataaaagcgacaaagaactgtctgatgaagctgctaagctgaaaaacgtcgtcggccatcatga
ggccgcgacgaatatagtcaaggattaccgatatacatacgataagtatttcctgcatatgcccatcactatcaa
ctttaaggcaaataagactggattcattaatgacagaatactgcaatacatagctaaagaaaaagatttgcatgt
tattggcattgccaggggtgagcgcaatcttatctatgtaagcgtcattgatacttgcgggaatatcgtagagca
gaagtcatttaatattgtaaatgggtacgattaccaaatcaagttgaagcagcaagagggagcacgacagattgc
ccgcaaggagtggaaagagatcggaaagataaaggagatcaaggaggggtatttgtcccttgttatacacgaaat
ttccaagatggtaatcaagtacaacgctataattgctatggaggatctctcctatggatttaaaaagggaagatt
taaagtcgagcggcaggtatatcagaaatttgaaacaatgcttattaataaacttaattatctcgttttcaaaga
cattagtatcaccgaaaacggtgggctgttgaagggctatcaacttacgtacataccagataagcttaagaatgt
gggtcaccaatgcggatgcatattctacgtgcccgcagcttatacaagcaaaatcgacccaacaacgggtttcgt
aaacatatttaagttcaaggatctcaccgtggatgccaagcgagagttcataaaaaaatttgactcaatcagata
tgactcagaaaagaatcttttttgttttaccttcgactacaataatttcattacacaaaatacggttatgagcaa
gtcatcctggtccgtatatacgtatggagtgcgcataaagcggagattcgttaacgggcgattttctaatgagtc
cgatacaatcgatataacaaaggatatggaaaaaactctggaaatgactgatataaattggagggacggtcatga
cctcaggcaagacattatcgattatgagatcgtgcaacatatttttgagatctttcggttgactgtccaaatgag
gaactctctgtctgaattggaagatagggactacgatcgcctgataagccccgtgttgaacgagaataacatatt
ctacgattccgcgaaagccggggatgcgctccctaaggacgccgatgcaaatggggcctattgtattgctttgaa
agggctgtacgaaatcaaacagatcaccgaaaactggaaagaagacgggaagtttagtcgggataaactgaagat
atccaacaaggactggtttgactttatccaaaataagcgatatttgagcggcagcgagactcccgggacctcaga
gtccgccacacccgaaagtcaactggtgaagagcgagctggaagagaagaaaagcgagctcagacataagctgaa
gtacgttccccacgaatacattgaactgatagaaatcgctagaaacagtacgcaagacagaatactggaaatgaa
ggtgatggagttcttcatgaaggtttacggctatcgtggcaaacacctcgggggctcccggaagcccgccggggc
tatctacaccgtgggcagtcccatcgactatggcgtgatcgtggacaccaaagcttatagcggcggatataatct
ccccatcggccaagccgatgagatgcagaggtatgtggaggagaaccaaacaagaaacaagcatatcaaccccaa
cgagtggtggaaggtttatcctagctcggtgaccgagtttaagttcctattcgtgtctggccacttcaagggcaa
ctataaggcacagctcactagactgaatcatatcacgaattgcaacggcgccgtgttatccgtggaggagctact
gatcggcggagagatgatcaaagccggcaccctgaccctggaagaggtgagaagaaagtttaacaatggcgaaat
aaatttcggcagcggaagtggaagcggctccatcactagaaccaccaaccctagaaacgtggtgcccaagatcta
catgagcgccggcagcatccccctgaccacccacatcaccaactcaattcagcccaccctgtggaccatcggcag
catcaacggcgtggcccccctggccaagagcatcaagctgggcatccccgtgaccggcagcgcctacaccgatca
gaccaccgccatggtgagaaagaaggtgagcgtgttcatgggcagcggcagcgggagcggctcatcgcagctggt
taagagcgagttagaagaaaaaaagagcgaactgcggcataaactgaagtatgtcccacacgagtacatcgaact
gatcgagatcgcgagaaactctacccaagacagaattctggagatgaaagtaatggaatttttcatgaaggtgta
tggatatagagggaagcacctgggtggcagcagaaaacccgacggcgccatctacactgtggggagccccataga
ctatggtgtgatcgtggataccaaggcgtatagcggcggttacaatctgcccattgggcaagcggacgagatgca
aagatatgtggaagagaatcagacgaggaacaagcacattaaccctaatgagtggtggaaggtctaccctagctc
cgttaccgagttcaagttcctgtttgtgagcgggcattttaagggcaactacaaggcacagctgacccgcctgaa
ccacataacaaactgcaacggtgccgtgctgagcgtagaagagttgctaatcggcggcgagatgatcaaggccgg
cacgctaaccctcgaagaggtgcgcagaaagttcaataacggcgaaatcaatttcaaaaggccggcggccacgaa
aaaggccggccaggcaaaaaagaaaaagggatcctacccatacgatgttccagattacgcttatccctacgacgt
gcctgattatgcatacccatatgatgtccccgactatgccgagggcagaggaagtctgctaacatgcggtgacgt
cgaggagaatcctggcccaatgaccgagtacaagcccacggtgcgcctcgccacccgcgacgacgtccccagggc
cgtacgcaccctcgccgccgcgttcgccgactaccccgccacgcgccacaccgtcgatccggaccgccacatcga
gcgggtcaccgagctgcaagaactcttcctcacgcgcgtcgggctcgacatcggcaaggtgtgggtcgcggacga
cggcgccgcggtggcggtctggaccacgccggagagcgtcgaagcgggggcggtgttcgccgagatcggcccgcg
catggccgagttgagcggttcccggctggccgcgcagcaacagatggaaggcctcctggcgccgcaccggcccaa
ggagcccgcgtggttcctggccaccgtcggagtctcgcccgaccaccagggcaagggtctgggcagcgccgtcgt
gctccccggagtggaggcggccgagcgcgccggggtgcccgccttcctggagacctccgcgccccgcaacctccc
cttctacgagcggctcggcttcaccgtcaccgccgacgtcgaggtgcccgaaggaccgcgcacctggtgcatgac
ccgcaagcccggtgcctgaactagtcctgcaggcatgcaagcttgatatcaagcttatcgataatcaacctctgg
attacaaaatttgtgaaagattgactggtattcttaactatgttgctccttttacgctatgtggatacgctgctt
taatgcctttgtatcatgctattgcttcccgtatggctttcattttctcctccttgtataaatcctggttgctgt
ctctttatgaggagttgtggcccgttgtcaggcaacgtggcgtggtgtgcactgtgtttgctgacgcaaccccca
ctggttggggcattgccaccacctgtcagctcctttccgggactttcgctttccccctccctattgccacggcgg
aactcatcgccgcctgccttgcccgctgctggacaggggctcggctgttgggcactgacaattccgtggtgttgt
cggggaaatcatcgtcctttccttggctgctcgcctgtgttgccacctggattctgcgcgggacgtccttctgct
acgtcccttcggccctcaatccagcggaccttccttcccgcggcctgctgccggctctgcggcctcttccgcgtc
ttcgccttcgccctcagacgagtcggatctccctttgggccgcctccccgcatcgataccgtcgacctcgaggga
attaattcgagctcggtacctttaagaccgatgacttacaaggcagctgtagatcttagccactttttaaaagaa
attaactgtgccttctagttgccagccatctgttgtttgcccctcccccgtgccttccttgaccctggaaggtgc
cactcccactgtcctttcctaataaaatgaggaaattgcatcgcattgtctgagtaggtgtcattctattctggg
gggtggggtggggcaggacagcaagggggaggattgggaagagaatagcaggcatgctggggagcggccgcagga
acccctagtgatggagttggccactccctctctgcgcgctcgctcgctcactgaggccgggcgaccaaaggtcgc
ccgacgcccgggctttgcccgggcggcctcagtgagcgagcgagcgcgcagctgcctgcaggggcgcctgatgcg
gtattttctccttacgcatctgtgcggtatttcacaccgcatacgtcaaagcaaccatagtacgcgccctgtagc
ggcgcattaagcgcggcgggtgtggtggttacgcgcagcgtgaccgctacacttgccagcgccttagcgcccgct
cctttcgctttcttcccttcctttctcgccacgttcgccggctttccccgtcaagctctaaatcgggggctccct
ttagggttccgatttagtgctttacggcacctcgaccccaaaaaacttgatttgggtgatggttcacgtagtggg
ccatcgccctgatagacggtttttcgccctttgacgttggagtccacgttctttaatagtggactcttgttccaa
actggaacaacactcaactctatctcgggctattcttttgatttataagggattttgccgatttcggtctattgg
ttaaaaaatgagctgatttaacaaaaatttaacgcgaattttaacaaaatattaacgtttacaattttatggtgc
actctcagtacaatctgctctgatgccgcatagttaagccagccccgacacccgccaacacccgctgacgcgccc
tgacgggcttgtctgctcccggcatccgcttacagacaagctgtgaccgtctccgggagctgcatgtgtcagagg
ttttcaccgtcatcaccgaaacgcgcgagacgaaagggcctcgtgatacgcctatttttataggttaatgtcatg
ataataatggtttcttagacgtcaggtggcacttttcggggaaatgtgcgcggaacccctatttgtttatttttc
taaatacattcaaatatgtatccgctcatgagacaataaccctgataaatgcttcaataatattgaaaaaggaag
agtatgagtattcaacatttccgtgtcgcccttattcccttttttgcggcattttgccttcctgtttttgctcac
ccagaaacgctggtgaaagtaaaagatgctgaagatcagttgggtgcacgagtgggttacatcgaactggatctc
aacagcggtaagatccttgagagttttcgccccgaagaacgttttccaatgatgagcacttttaaagttctgcta
tgtggcgcggtattatcccgtattgacgccgggcaagagcaactcggtcgccgcatacactattctcagaatgac
ttggttgagtactcaccagtcacagaaaagcatcttacggatggcatgacagtaagagaattatgcagtgctgcc
ataaccatgagtgataacactgcggccaacttacttctgacaacgatcggaggaccgaaggagctaaccgctttt
ttgcacaacatgggggatcatgtaactcgccttgatcgttgggaaccggagctgaatgaagccataccaaacgac
gagcgtgacaccacgatgcctgtagcaatggcaacaacgttgcgcaaactattaactggcgaactacttactcta
gcttcccggcaacaattaatagactggatggaggcggataaagttgcaggaccacttctgcgctcggcccttccg
gctggctggtttattgctgataaatctggagccggtgagcgtggaagccgcggtatcattgcagcactggggcca
gatggtaagccctcccgtatcgtagttatctacacgacggggagtcaggcaactatggatgaacgaaatagacag
atcgctgagataggtgcctcactgattaagcattggtaactgtcagaccaagtttactcatatatactttagatt
gatttaaaacttcatttttaatttaaaaggatctaggtgaagatcctttttgataatctcatgaccaaaatccct
taacgtgagttttcgttccactgagcgtcagaccccgtagaaaagatcaaaggatcttcttgagatccttttttt
ctgcgcgtaatctgctgcttgcaaacaaaaaaaccaccgctaccagcggtggtttgtttgccggatcaagagcta
ccaactctttttccgaaggtaactggcttcagcagagcgcagataccaaatactgttcttctagtgtagccgtag
ttaggccaccacttcaagaactctgtagcaccgcctacatacctcgctctgctaatcctgttaccagtggctgct
gccagtggcgataagtcgtgtcttaccgggttggactcaagacgatagttaccggataaggcgcagcggtcgggc
tgaacggggggttcgtgcacacagcccagcttggagcgaacgacctacaccgaactgagatacctacagcgtgag
ctatgagaaagcgccacgcttcccgaagggagaaaggcggacaggtatccggtaagcggcagggtcggaacagga
gagcgcacgagggagcttccagggggaaacgcctggtatctttatagtcctgtcgggtttcgccacctctgactt
gagcgtcgatttttgtgatgctcgtcaggggggcggagcctatggaaaaacgccagcaacgcggcctttttacgg
ttcctggccttttgctggccttttgctcacatgt
Construct D. (SEQ ID NO: 48)
gagggcctatttcccatgattccttcatatttgcatatacgatacaaggctgttagagagataattggaattaat
ttgactgtaaacacaaagatattagtacaaaatacgtgacgtagaaagtaataatttcttgggtagtttgcagtt
ttaaaattatgttttaaaatggactatcatatgcttaccgtaacttgaaagtatttcgatttcttggctttatat
atcttgtggaaaggacgaaacaccgggtcttcgagaagacctgttttagagctagaaatagcaagttaaaataag
gctagtccgttatcaacttgaaaaagtggcaccgagtcggtgcttttttgttttagagctagaaatagcaagtta
aaataaggctagtccgtttttagcgcgtgcgccaattctgcagacaaatggctctagaggtacccgttacataac
ttacggtaaatggcccgcctggctgaccgcccaacgacccccgcccattgacgtcaatagtaacgccaataggga
ctttccattgacgtcaatgggtggagtatttacggtaaactgcccacttggcagtacatcaagtgtatcatatgc
caagtacgccccctattgacgtcaatgacggtaaatggcccgcctggcattgtgcccagtacatgaccttatggg
actttcctacttggcagtacatctacgtattagtcatcgctattaccatggtcgaggtgagccccacgttctgct
tcactctccccatctcccccccctccccacccccaattttgtatttatttattttttaattattttgtgcagcga
tgggggcggggggggggggggggcgcgcgccaggcggggcggggcggggcgaggggcggggcggggcgaggcgga
gaggtgcggcggcagccaatcagagcggcgcgctccgaaagtttccttttatggcgaggcggcggcggcggcggc
cctataaaaagcgaagcgcgcggcgggcgggagtcgctgcgcgctgccttcgccccgtgccccgctccgccgccg
cctcgcgccgcccgccccggctctgactgaccgcgttactcccacaggtgagcgggcgggacggcccttctcctc
cgggctgtaattagctgagcaagaggtaagggtttaagggatggttggttggtggggtattaatgtttaattacc
tggagcacctgcctgaaatcactttttttcaggttggaccggtgccaccatgaataacggaactaataacttcca
aaacttcatcgggatcagttccttgcagaaaactctccggaatgctctcatcccaactgagactactcagcagtt
cattgttaagaatggaatcataaaagaggacgagcttaggggggaaaataggcaaatcctcaaggatatcatgga
tgactattataggggctttatatccgagacactgagcagcattgatgatatagactggacctctcttttcgaaaa
gatggaaatacaacttaaaaatggagataacaaggacaccctgataaaggaacagaccgaatataggaaggcaat
tcataaaaagtttgctaacgatgataggtttaaaaacatgttctcagcaaaactcatttcagatatactgcccga
attcgttatccacaacaacaactactccgctagcgaaaaagaggaaaagacccaagtcataaagctgttctctcg
attcgcgacgagttttaaagattatttccgaaatcgcgcaaactgtttctcagctgatgatatcagcagctcatc
ctgtcatcggatcgttaacgataatgctgaaatcttcttctccaatgcacttgtttataggcgcattgttaaatc
tctctcaaacgatgatatcaataagatttccggcgatatgaaggacagtcttaaggagatgagcctcgaagagat
atactcatacgagaaatatggcgaatttatcacccaggaagggatttccttctataatgacatttgcggcaaagt
caattccttcatgaacctgtattgccaaaaaaataaagaaaacaagaacctctataagctgcaaaagttgcataa
gcaaatactttgtatcgcggatacaagctatgaagttccctacaagttcgagagtgatgaggaggtgtatcaatc
tgtcaatggtttccttgataatatttcttctaagcatattgttgaacgactccgaaagataggagacaactataa
tggatacaatttggataaaatctacatcgtgtctaaattttacgagagtgtgtcacaaaaaacatatagagactg
ggagacaattaataccgccctggagatacattacaacaatatacttcccgggaacgggaagtctaaggcagacaa
ggtgaagaaagccgtgaagaacgacttgcaaaagtcaattaccgaaatcaatgagcttgtttcaaactataaact
ttgttcagatgacaatattaaagccgaaacctatattcatgaaatctctcatattctgaataactttgaggcgca
agaactgaaatataacccagaaatacacctcgttgagtccgaactgaaagcaagcgaactgaaaaatgttttgga
cgtgataatgaacgcttttcattggtgctcagtctttatgacagaggagcttgttgacaaggataacaatttcta
tgcggaactggaagagatttacgacgaaatctatccggtcatatccctgtataacctggttcgcaactatgtcac
gcaaaaaccatacagcacgaagaagattaaactgaactttggtattccgacgctggcccgcggatggtcaaaatc
tgttgaatactcacgaaatgccataatcctgatgcgagataacctctactaccttggaatctttaatgctaaaaa
taaacccgataaaaaaattatcgaagggaacacgagtgaaaacaaaggtgattataaaaaaatgatatataatct
gcttccaggaccaaataagatgatacccaaagttttcctttcttcaaagaccggcgtcgagacatataaaccatc
cgcgtacatacttgaaggctacaaacaaaataaacatatcaaatcatctaaggattttgacattacgttctgtca
tgatttgattgactatttcaaaaattgcatagccattcatccagagtggaaaaactttgggtttgacttctctga
taccagtacatatgaagacataagtggattttaccgagaagtagagctccaaggttataaaatagactggaccta
tatatctgaaaaggatatagaccttttgcaagagaagggacagctttatcttttccaaatctacaacaaagactt
cagtaagaaaagtaccgggaatgacaatcttcataccatgtatctgaagaacctgttctccgaagaaaatctgaa
ggacatagtcctgaagcttaatggcgaagcggaaatttttttccgaaagagctctattaagaaccccataataca
taagaagggaagcattctcgttaatcgaacgtatgaggccgaagagaaagatcaatttgggaatatccaaatcgt
tcgaaagaacataccagaaaatatttaccaagaattgtacaaatattttaacgataaaagcgacaaagaactgtc
tgatgaagctgctaagctgaaaaacgtcgtcggccatcatgaggccgcgacgaatatagtcaaggattaccgata
tacatacgataagtatttcctgcatatgcccatcactatcaactttaaggcaaataagactggattcattaatga
cagaatactgcaatacatagctaaagaaaaagatttgcatgttattggcattgacaggggtgagcgcaatcttat
ctatgtaagcgtcattgatacttgcgggaatatcgtagagcagaagtcatttaatattgtaaatgggtacgatta
ccaaatcaagttgaagcagcaagagggagcacgacagattgcccgcaaggagtggaaagagatcggaaagataaa
ggagatcaaggaggggtatttgtcccttgttatacacgaaatttccaagatggtaatcaagtacaacgctataat
tgctatggaggatctctcctatggatttaaaaagggaagatttaaagtcgagcggcaggtatatcagaaatttga
aacaatgcttattaataaacttaattatctcgttttcaaagacattagtatcaccgaaaacggtgggctgttgaa
gggctatcaacttacgtacataccagataagcttaagaatgtgggtcaccaatgcggatgcatattctacgtgcc
cgcagcttatacaagcaaaatcgacccaacaacgggtttcgtaaacatatttaagttcaaggatctcaccgtgga
tgccaagcgagagttcataaaaaaatttgactcaatcagatatgactcagaaaagaatcttttttgttttacctt
cgactacaataatttcattacacaaaatacggttatgagcaagtcatcctggtccgtatatacgtatggagtgcg
cataaagcggagattcgttaacgggcgattttctaatgagtccgatacaatcgatataacaaaggatatggaaaa
aactctggaaatgactgatataaattggagggacggtcatgacctcaggcaagacattatcgattatgagatcgt
gcaacatatttttgagatctttcggttgactgtccaaatgaggaactctctgtctgaattggaagatagggacta
cgatcgcctgataagccccgtgttgaacgagaataacatattctacgattccgcgaaagccggggatgcgctccc
taaggacgccgatgcaaatggggcctattgtattgctttgaaagggctgtacgaaatcaaacagatcaccgaaaa
ctggaaagaagacgggaagtttagtcgggataaactgaagatatccaacaaggactggtttgactttatccaaaa
taagcgatatttgaaaaggccggcggccacgaaaaaggccggccaggcaaaaaagaaaaagggatcctacccata
cgatgttccagattacgcttatccctacgacgtgcctgattatgcatacccatatgatgtccccgactatgccga
gggcagaggaagtctgctaacatgcggtgacgtcgaggagaatcctggcccaatgaccgagtacaagcccacggt
gcgcctcgccacccgcgacgacgtccccagggccgtacgcaccctcgccgccgcgttcgccgactaccccgccac
gcgccacaccgtcgatccggaccgccacatcgagcgggtcaccgagctgcaagaactcttcctcacgcgcgtcgg
gctcgacatcggcaaggtgtgggtcgcggacgacggcgccgcggtggcggtctggaccacgccggagagcgtcga
agcgggggcggtgttcgccgagatcggcccgcgcatggccgagttgagcggttcccggctggccgcgcagcaaca
gatggaaggcctcctggcgccgcaccggcccaaggagcccgcgtggttcctggccaccgtcggagtctcgcccga
ccaccagggcaagggtctgggcagcgccgtcgtgctccccggagtggaggcggccgagcgcgccggggtgcccgc
cttcctggagacctccgcgccccgcaacctccccttctacgagcggctcggcttcaccgtcaccgccgacgtcga
ggtgcccgaaggaccgcgcacctggtgcatgacccgcaagcccggtgcctgaactagtcctgcaggcatgcaagc
ttgatatcaagcttatcgataatcaacctctggattacaaaatttgtgaaagattgactggtattcttaactatg
ttgctccttttacgctatgtggatacgctgctttaatgcctttgtatcatgctattgcttcccgtatggctttca
ttttctcctccttgtataaatcctggttgctgtctctttatgaggagttgtggcccgttgtcaggcaacgtggcg
tggtgtgcactgtgtttgctgacgcaacccccactggttggggcattgccaccacctgtcagctcctttccggga
ctttcgctttccccctccctattgccacggcggaactcatcgccgcctgccttgcccgctgctggacaggggctc
ggctgttgggcactgacaattccgtggtgttgtcggggaaatcatcgtcctttccttggctgctcgcctgtgttg
ccacctggattctgcgcgggacgtccttctgctacgtcccttcggccctcaatccagcggaccttccttcccgcg
gcctgctgccggctctgcggcctcttccgcgtcttcgccttcgccctcagacgagtcggatctccctttgggccg
cctccccgcatcgataccgtcgacctcgagggaattaattcgagctcggtacctttaagaccgatgacttacaag
gcagctgtagatcttagccactttttaaaagaaattaactgtgccttctagttgccagccatctgttgtttgccc
ctcccccgtgccttccttgaccctggaaggtgccactcccactgtcctttcctaataaaatgaggaaattgcatc
gcattgtctgagtaggtgtcattctattctggggggtggggtggggcaggacagcaagggggaggattgggaaga
gaatagcaggcatgctggggagcggccgcaggaacccctagtgatggagttggccactccctctctgcgcgctcg
ctcgctcactgaggccgggcgaccaaaggtcgcccgacgcccgggctttgcccgggcggcctcagtgagcgagcg
agcgcgcagctgcctgcaggggcgcctgatgcggtattttctccttacgcatctgtgcggtatttcacaccgcat
acgtcaaagcaaccatagtacgcgccctgtagcggcgcattaagcgcggcgggtgtggtggttacgcgcagcgtg
accgctacacttgccagcgccttagcgcccgctcctttcgctttcttcccttcctttctcgccacgttcgccggc
tttccccgtcaagctctaaatcgggggctccctttagggttccgatttagtgctttacggcacctcgaccccaaa
aaacttgatttgggtgatggttcacgtagtgggccatcgccctgatagacggtttttcgccctttgacgttggag
tccacgttctttaatagtggactcttgttccaaactggaacaacactcaactctatctcgggctattcttttgat
ttataagggattttgccgatttcggtctattggttaaaaaatgagctgatttaacaaaaatttaacgcgaatttt
aacaaaatattaacgtttacaattttatggtgcactctcagtacaatctgctctgatgccgcatagttaagccag
ccccgacacccgccaacacccgctgacgcgccctgacgggcttgtctgctcccggcatccgcttacagacaagct
gtgaccgtctccgggagctgcatgtgtcagaggttttcaccgtcatcaccgaaacgcgcgagacgaaagggcctc
gtgatacgcctatttttataggttaatgtcatgataataatggtttcttagacgtcaggtggcacttttcgggga
aatgtgcgcggaacccctatttgtttatttttctaaatacattcaaatatgtatccgctcatgagacaataaccc
tgataaatgcttcaataatattgaaaaaggaagagtatgagtattcaacatttccgtgtcgcccttattcccttt
tttgcggcattttgccttcctgtttttgctcacccagaaacgctggtgaaagtaaaagatgctgaagatcagttg
ggtgcacgagtgggttacatcgaactggatctcaacagcggtaagatccttgagagttttcgccccgaagaacgt
tttccaatgatgagcacttttaaagttctgctatgtggcgcggtattatcccgtattgacgccgggcaagagcaa
ctcggtcgccgcatacactattctcagaatgacttggttgagtactcaccagtcacagaaaagcatcttacggat
ggcatgacagtaagagaattatgcagtgctgccataaccatgagtgataacactgcggccaacttacttctgaca
acgatcggaggaccgaaggagctaaccgcttttttgcacaacatgggggatcatgtaactcgccttgatcgttgg
gaaccggagctgaatgaagccataccaaacgacgagcgtgacaccacgatgcctgtagcaatggcaacaacgttg
cgcaaactattaactggcgaactacttactctagcttcccggcaacaattaatagactggatggaggcggataaa
gttgcaggaccacttctgcgctcggcccttccggctggctggtttattgctgataaatctggagccggtgagcgt
ggaagccgcggtatcattgcagcactggggccagatggtaagccctcccgtatcgtagttatctacacgacgggg
agtcaggcaactatggatgaacgaaatagacagatcgctgagataggtgcctcactgattaagcattggtaactg
tcagaccaagtttactcatatatactttagattgatttaaaacttcatttttaatttaaaaggatctaggtgaag
atcctttttgataatctcatgaccaaaatcccttaacgtgagttttcgttccactgagcgtcagaccccgtagaa
aagatcaaaggatcttcttgagatcctttttttctgcgcgtaatctgctgcttgcaaacaaaaaaaccaccgcta
ccagcggtggtttgtttgccggatcaagagctaccaactctttttccgaaggtaactggcttcagcagagcgcag
ataccaaatactgttcttctagtgtagccgtagttaggccaccacttcaagaactctgtagcaccgcctacatac
ctcgctctgctaatcctgttaccagtggctgctgccagtggcgataagtcgtgtcttaccgggttggactcaaga
cgatagttaccggataaggcgcagcggtcgggctgaacggggggttcgtgcacacagcccagcttggagcgaacg
acctacaccgaactgagatacctacagcgtgagctatgagaaagcgccacgcttcccgaagggagaaaggcggac
aggtatccggtaagcggcagggtcggaacaggagagcgcacgagggagcttccagggggaaacgcctggtatctt
tatagtcctgtcgggtttcgccacctctgacttgagcgtcgatttttgtgatgctcgtcaggggggcggagccta
tggaaaaacgccagcaacgcggcctttttacggttcctggccttttgctggccttttgctcacatgt
Construct E (SEQ ID NO: 49)
gagggcctatttcccatgattccttcatatttgcatatacgatacaaggctgttagagagataattggaattaat
ttgactgtaaacacaaagatattagtacaaaatacgtgacgtagaaagtaataatttcttgggtagtttgcagtt
ttaaaattatgttttaaaatggactatcatatgcttaccgtaacttgaaagtatttcgatttcttggctttatat
atcttgtggaaaggacgaaacaccgggtcttcgagaagacctgttttagagctagaaatagcaagttaaaataag
gctagtccgttatcaacttgaaaaagtggcaccgagtcggtgcttttttgttttagagctagaaatagcaagtta
aaataaggctagtccgtttttagcgcgtgcgccaattctgcagacaaatggctctagaggtacccgttacataac
ttacggtaaatggcccgcctggctgaccgcccaacgacccccgcccattgacgtcaatagtaacgccaataggga
ctttccattgacgtcaatgggtggagtatttacggtaaactgcccacttggcagtacatcaagtgtatcatatgc
caagtacgccccctattgacgtcaatgacggtaaatggcccgcctggcattgtgcccagtacatgaccttatggg
actttcctacttggcagtacatctacgtattagtcatcgctattaccatggtcgaggtgagccccacgttctgct
tcactctccccatctcccccccctccccacccccaattttgtatttatttattttttaattattttgtgcagcga
tgggggcggggggggggggggggcgcgcgccaggcggggcggggcggggcgaggggcggggcggggcgaggcgga
gaggtgcggcggcagccaatcagagcggcgcgctccgaaagtttccttttatggcgaggcggcggcggcggcggc
cctataaaaagcgaagcgcgcggcgggcgggagtcgctgcgcgctgccttcgccccgtgccccgctccgccgccg
cctcgcgccgcccgccccggctctgactgaccgcgttactcccacaggtgagcgggcgggacggcccttctcctc
cgggctgtaattagctgagcaagaggtaagggtttaagggatggttggttggtggggtattaatgtttaattacc
tggagcacctgcctgaaatcactttttttcaggttggaccggtgccaccatgacacagttcgagggctttaccaa
cctgtatcaggtgagcaagacactgcggtttgagctgatcccacagggcaagaccctgaagcacatccaggagca
gggcttcatcgaggaggacaaggcccgcaatgatcactacaaggagctgaagcccatcatcgatcggatctacaa
gacctatgccgaccagtgcctgcagctggtgcagctggattgggagaacctgagcgccgccatcgactcctatag
aaaggagaaaaccgaggagacaaggaacgccctgatcgaggagcaggccacatatcgcaatgccatccacgacta
cttcatcggccggacagacaacctgaccgatgccatcaataagagacacgccgagatctacaagggcctgttcaa
ggccgagctgtttaatggcaaggtgctgaagcagctgggcaccgtgaccacaaccgagcacgagaacgccctgct
gcggagcttcgacaagtttacaacctacttctccggcttttatagaaacaggaagaacgtgttcagcgccgagga
tatcagcacagccatcccacaccgcatcgtgcaggacaacttccccaagtttaaggagaattgtcacatcttcac
acgcctgatcaccgccgtgcccagcctgcgggagcactttgagaacgtgaagaaggccatcggcatcttcgtgag
cacctccatcgaggaggtgttttccttccctttttataaccagctgctgacacagacccagatcgacctgtataa
ccagctgctgggaggaatctctcgggaggcaggcaccgagaagatcaagggcctgaacgaggtgctgaatctggc
catccagaagaatgatgagacagcccacatcatcgcctccctgccacacagattcatccccctgtttaagcagat
cctgtccgataggaacaccctgtctttcatcctggaggagtttaagagcgacgaggaagtgatccagtccttctg
caagtacaagacactgctgagaaacgagaacgtgctggagacagccgaggccctgtttaacgagctgaacagcat
cgacctgacacacatcttcatcagccacaagaagctggagacaatcagcagcgccctgtgcgaccactgggatac
actgaggaatgccctgtatgagcggagaatctccgagctgacaggcaagatcaccaagtctgccaaggagaaggt
gcagcgcagcctgaagcacgaggatatcaacctgcaggagatcatctctgccgcaggcaaggagctgagcgaggc
cttcaagcagaaaaccagcgagatcctgtcccacgcacacgccgccctggatcagccactgcctacaaccctgaa
gaagcaggaggagaaggagatcctgaagtctcagctggacagcctgctgggcctgtaccacctgctggactggtt
tgccgtggatgagtccaacgaggtggaccccgagttctctgcccggctgaccggcatcaagctggagatggagcc
ttctctgagcttctacaacaaggccagaaattatgccaccaagaagccctactccgtggagaagttcaagctgaa
ctttcagatgcctacactggccagaggctgggacgtgaatgtggagaagaacagaggcgccatcctgtttgtgaa
gaacggcctgtactatctgggcatcatgccaaagcagaagggcaggtataaggccctgagcttcgagcccacaga
gaaaaccagcgagggctttgataagatgtactatgactacttccctgatgccgccaagatgatcccaaagtgcag
cacccagctgaaggccgtgacagcccactttcagacccacacaacccccatcctgctgtccaacaatttcatcga
gcctctggagatcacaaaggagatctacgacctgaacaatcctgagaaggagccaaagaagtttcagacagccta
cgccaagaaaaccggcgaccagaagggctacagagaggccctgtgcaagtggatcgacttcacaagggattttct
gtccaagtataccaagacaacctctatcgatctgtctagcctgcggccatcctctcagtataaggacctgggcga
gtactatgccgagctgaatcccctgctgtaccacatcagcttccagagaatcgccgagaaggagatcatggatgc
cgtggagacaggcaagctgtacctgttccagatctataacaaggactttgccaagggccaccacggcaagcctaa
tctgcacacactgtattggaccggcctgttttctccagagaacctggccaagacaagcatcaagctgaatggcca
ggccgagctgttctaccgccctaagtccaggatgaagaggatggcacaccggctgggagagaagatgctgaacaa
gaagctgaaggatcagaaaaccccaatccccgacaccctgtaccaggagctgtacgactatgtgaatcacagact
gtcccacgacctgtctgatgaggccagggccctgctgcccaacgtgatcaccaaggaggtgtctcacgagatcat
caaggataggcgctttaccagcgacaagttctttttccacgtgcctatcacactgaactatcaggccgccaattc
cccatctaagttcaaccagagggtgaatgcctacctgaaggagcaccccgagacacctatcatcggcatcgcccg
gggcgagagaaacctgatctatatcacagtgatcgactccaccggcaagatcctggagcagcggagcctgaacac
catccagcagtttgattaccagaagaagctggacaacagggagaaggagagggtggcagcaaggcaggcctggtc
tgtggtgggcacaatcaaggatctgaagcagggctatctgagccaggtcatccacgagatcgtggacctgatgat
ccactaccaggccgtggtggtgctggagaacctgaatttcggctttaagagcaagaggaccggcatcgccgagaa
ggccgtgtaccagcagttcgagaagatgctgatcgataagctgaattgcctggtgctgaaggactatccagcaga
gaaagtgggaggcgtgctgaacccataccagctgacagaccagttcacctcctttgccaagatgggcacccagtc
tggcttcctgttttacgtgcctgccccatatacatctaagatcgatcccctgaccggcttcgtggaccccttcgt
gtggaaaaccatcaagaatcacgagagccgcaagcacttcctggagggcttcgactttctgcactacgacgtgaa
aaccggcgacttcatcctgcactttaagatgaacagaaatctgtccttccagaggggcctgcccggctttatgcc
tgcatgggatatcgtgttcgagaagaacgagacacagtttgacgccaagggcacccctttcatcgccggcaagag
aatcgtgccagtgatcgagaatcacagattcaccggcagataccgggacctgtatcctgccaacgagctgatcgc
cctgctggaggagaagggcatcgtgttcagggatggctccaacatcctgccaaagctgctggagaatgacgattc
tcacgccatcgacaccatggtggccctgatccgcagcgtgctgcagatgcggaactccaatgccgccacaggcga
ggactatatcaacagccccgtgcgcgatctgaatggcgtgtgcttcgactcccggtttcagaacccagagtggcc
catggacgccgatgccaatggcgcctaccacatcgccctgaagggccagctgctgctgaatcacctgaaggagag
caaggatctgaagctgcagaacggcatctccaatcaggactggctggcctacatccaggagctgcgcaacagcgg
cagcgagactcccgggacctcagagtccgccacacccgaaagtcaactggtgaagagcgagctggaagagaagaa
aagcgagctcagacataagctgaagtacgttccccacgaatacattgaactgatagaaatcgctagaaacagtac
gcaagacagaatactggaaatgaaggtgatggagttcttcatgaaggtttacggctatcgtggcaaacacctcgg
gggctcccggaagcccgacggggctatctacaccgtgggcagtcccatcgactatggcgtgatcgtggacaccaa
agcttatagcggcggatataatctccccatcggccaagccgatgagatgcagaggtatgtggaggagaaccaaac
aagaaacaagcatatcaaccccaacgagtggtggaaggtttatcctagctcggtgaccgagtttaagttcctatt
cgtgtctggccacttcaagggcaactataaggcacagctcactagactgaatcatatcacgaattgcaacggcgc
cgtgttatccgtggaggagctactgatcggcggagagatgatcaaagccggcaccctgaccctggaagaggtgag
aagaaagtttaacaatggcgaaataaatttcggcagcggaagtggaagcggctccatcactagaaccaccaaccc
tagaaacgtggtgcccaagatctacatgagcgccggcagcatccccctgaccacccacatcaccaactcaattca
gcccaccctgtggaccatcggcagcatcaacggcgtggcccccctggccaagagcatcaagctgggcatccccgt
gaccggcagcgcctacaccgatcagaccaccgccatggtgagaaagaaggtgagcgtgttcatgggcagcggcag
cgggagcggctcatcgcagctggttaagagcgagttagaagaaaaaaagagcgaactgcggcataaactgaagta
tgtcccacacgagtacatcgaactgatcgagatcgcgagaaactctacccaagacagaattctggagatgaaagt
aatggaatttttcatgaaggtgtatggatatagagggaagcacctgggtggcagcagaaaacccgacggcgccat
ctacactgtggggagccccatagactatggtgtgatcgtggataccaaggcgtatagcggcggttacaatctgcc
cattgggcaagcggacgagatgcaaagatatgtggaagagaatcagacgaggaacaagcacattaaccctaatga
gtggtggaaggtctaccctagctccgttaccgagttcaagttcctgtttgtgagcgggcattttaagggcaacta
caaggcacagctgacccgcctgaaccacataacaaactgcaacggtgccgtgctgagcgtagaagagttgctaat
cggcggcgagatgatcaaggccggcacgctaaccctcgaagaggtgcgcagaaagttcaataacggcgaaatcaa
tttcaaaaggccggcggccacgaaaaaggccggccaggcaaaaaagaaaaagggatcctacccatacgatgttcc
agattacgcttatccctacgacgtgcctgattatgcatacccatatgatgtccccgactatgccgagggcagagg
aagtctgctaacatgcggtgacgtcgaggagaatcctggcccaatgaccgagtacaagcccacggtgcgcctcgc
cacccgcgacgacgtccccagggccgtacgcaccctcgccgccgcgttcgccgactaccccgccacgcgccacac
cgtcgatccggaccgccacatcgagcgggtcaccgagctgcaagaactcttcctcacgcgcgtcgggctcgacat
cggcaaggtgtgggtcgcggacgacggcgccgcggtggcggtctggaccacgccggagagcgtcgaagcgggggc
ggtgttcgccgagatcggcccgcgcatggccgagttgagcggttcccggctggccgcgcagcaacagatggaagg
cctcctggcgccgcaccggcccaaggagcccgcgtggttcctggccaccgtcggagtctcgcccgaccaccaggg
caagggtctgggcagcgccgtcgtgctccccggagtggaggcggccgagcgcgccggggtgcccgccttcctgga
gacctccgcgccccgcaacctccccttctacgagcggctcggcttcaccgtcaccgccgacgtcgaggtgcccga
aggaccgcgcacctggtgcatgacccgcaagcccggtgcctgaactagtcctgcaggcatgcaagcttgatatca
agcttatcgataatcaacctctggattacaaaatttgtgaaagattgactggtattcttaactatgttgctcctt
ttacgctatgtggatacgctgctttaatgcctttgtatcatgctattgcttcccgtatggctttcattttctcct
ccttgtataaatcctggttgctgtctctttatgaggagttgtggcccgttgtcaggcaacgtggcgtggtgtgca
ctgtgtttgctgacgcaacccccactggttggggcattgccaccacctgtcagctcctttccgggactttcgctt
tccccctccctattgccacggcggaactcatcgccgcctgccttgcccgctgctggacaggggctcggctgttgg
gcactgacaattccgtggtgttgtcggggaaatcatcgtcctttccttggctgctcgcctgtgttgccacctgga
ttctgcgcgggacgtccttctgctacgtcccttcggccctcaatccagcggaccttccttcccgcggcctgctgc
cggctctgcggcctcttccgcgtcttcgccttcgccctcagacgagtcggatctccctttgggccgcctccccgc
atcgataccgtcgacctcgagggaattaattcgagctcggtacctttaagaccgatgacttacaaggcagctgta
gatcttagccactttttaaaagaaattaactgtgccttctagttgccagccatctgttgtttgcccctcccccgt
gccttccttgaccctggaaggtgccactcccactgtcctttcctaataaaatgaggaaattgcatcgcattgtct
gagtaggtgtcattctattctggggggtggggtggggcaggacagcaagggggaggattgggaagagaatagcag
gcatgctggggagcggccgcaggaacccctagtgatggagttggccactccctctctgcgcgctcgctcgctcac
tgaggccgggcgaccaaaggtcgcccgacgcccgggctttgcccgggcggcctcagtgagcgagcgagcgcgcag
ctgcctgcaggggcgcctgatgcggtattttctccttacgcatctgtgcggtatttcacaccgcatacgtcaaag
caaccatagtacgcgccctgtagcggcgcattaagcgcggcgggtgtggtggttacgcgcagcgtgaccgctaca
cttgccagcgccttagcgcccgctcctttcgctttcttcccttcctttctcgccacgttcgccggctttccccgt
caagctctaaatcgggggctccctttagggttccgatttagtgctttacggcacctcgaccccaaaaaacttgat
ttgggtgatggttcacgtagtgggccatcgccctgatagacggtttttcgccctttgacgttggagtccacgttc
tttaatagtggactcttgttccaaactggaacaacactcaactctatctcgggctattcttttgatttataaggg
attttgccgatttcggtctattggttaaaaaatgagctgatttaacaaaaatttaacgcgaattttaacaaaata
ttaacgtttacaattttatggtgcactctcagtacaatctgctctgatgccgcatagttaagccagccccgacac
ccgccaacacccgctgacgcgccctgacgggcttgtctgctcccggcatccgcttacagacaagctgtgaccgtc
tccgggagctgcatgtgtcagaggttttcaccgtcatcaccgaaacgcgcgagacgaaagggcctcgtgatacgc
ctatttttataggttaatgtcatgataataatggtttcttagacgtcaggtggcacttttcggggaaatgtgcgc
ggaacccctatttgtttatttttctaaatacattcaaatatgtatccgctcatgagacaataaccctgataaatg
cttcaataatattgaaaaaggaagagtatgagtattcaacatttccgtgtcgcccttattcccttttttgcggca
ttttgccttcctgtttttgctcacccagaaacgctggtgaaagtaaaagatgctgaagatcagttgggtgcacga
gtgggttacatcgaactggatctcaacagcggtaagatccttgagagttttcgccccgaagaacgttttccaatg
atgagcacttttaaagttctgctatgtggcgcggtattatcccgtattgacgccgggcaagagcaactcggtcgc
cgcatacactattctcagaatgacttggttgagtactcaccagtcacagaaaagcatcttacggatggcatgaca
gtaagagaattatgcagtgctgccataaccatgagtgataacactgcggccaacttacttctgacaacgatcgga
ggaccgaaggagctaaccgcttttttgcacaacatgggggatcatgtaactcgccttgatcgttgggaaccggag
ctgaatgaagccataccaaacgacgagcgtgacaccacgatgcctgtagcaatggcaacaacgttgcgcaaacta
ttaactggcgaactacttactctagcttcccggcaacaattaatagactggatggaggcggataaagttgcagga
ccacttctgcgctcggcccttccggctggctggtttattgctgataaatctggagccggtgagcgtggaagccgc
ggtatcattgcagcactggggccagatggtaagccctcccgtatcgtagttatctacacgacggggagtcaggca
actatggatgaacgaaatagacagatcgctgagataggtgcctcactgattaagcattggtaactgtcagaccaa
gtttactcatatatactttagattgatttaaaacttcatttttaatttaaaaggatctaggtgaagatccttttt
gataatctcatgaccaaaatcccttaacgtgagttttcgttccactgagcgtcagaccccgtagaaaagatcaaa
ggatcttcttgagatcctttttttctgcgcgtaatctgctgcttgcaaacaaaaaaaccaccgctaccagcggtg
gtttgtttgccggatcaagagctaccaactctttttccgaaggtaactggcttcagcagagcgcagataccaaat
actgttcttctagtgtagccgtagttaggccaccacttcaagaactctgtagcaccgcctacatacctcgctctg
ctaatcctgttaccagtggctgctgccagtggcgataagtcgtgtcttaccgggttggactcaagacgatagtta
ccggataaggcgcagcggtcgggctgaacggggggttcgtgcacacagcccagcttggagcgaacgacctacacc
gaactgagatacctacagcgtgagctatgagaaagcgccacgcttcccgaagggagaaaggcggacaggtatccg
gtaagcggcagggtcggaacaggagagcgcacgagggagcttccagggggaaacgcctggtatctttatagtcct
gtcgggtttcgccacctctgacttgagcgtcgatttttgtgatgctcgtcaggggggcggagcctatggaaaaac
gccagcaacgcggcctttttacggttcctggccttttgctggccttttgctcacatgt
Construct F (SEQ ID NO: 50)
gagggcctatttcccatgattccttcatatttgcatatacgatacaaggctgttagagagataattggaattaat
ttgactgtaaacacaaagatattagtacaaaatacgtgacgtagaaagtaataatttcttgggtagtttgcagtt
ttaaaattatgttttaaaatggactatcatatgcttaccgtaacttgaaagtatttcgatttcttggctttatat
atcttgtggaaaggacgaaacaccgggtcttcgagaagacctgttttagagctagaaatagcaagttaaaataag
gctagtccgttatcaacttgaaaaagtggcaccgagtcggtgcttttttgttttagagctagaaatagcaagtta
aaataaggctagtccgtttttagcgcgtgcgccaattctgcagacaaatggctctagaggtacccgttacataac
ttacggtaaatggcccgcctggctgaccgcccaacgacccccgcccattgacgtcaatagtaacgccaataggga
ctttccattgacgtcaatgggtggagtatttacggtaaactgcccacttggcagtacatcaagtgtatcatatgc
caagtacgccccctattgacgtcaatgacggtaaatggcccgcctggcattgtgcccagtacatgaccttatggg
actttcctacttggcagtacatctacgtattagtcatcgctattaccatggtcgaggtgagccccacgttctgct
tcactctccccatctcccccccctccccacccccaattttgtatttatttattttttaattattttgtgcagcga
tgggggcggggggggggggggggcgcgcgccaggcggggcggggcggggcgaggggcggggcggggcgaggcgga
gaggtgcggcggcagccaatcagagcggcgcgctccgaaagtttccttttatggcgaggcggcggcggcggcggc
cctataaaaagcgaagcgcgcggcgggcgggagtcgctgcgcgctgccttcgccccgtgccccgctccgccgccg
cctcgcgccgcccgccccggctctgactgaccgcgttactcccacaggtgagcgggcgggacggcccttctcctc
cgggctgtaattagctgagcaagaggtaagggtttaagggatggttggttggtggggtattaatgtttaattacc
tggagcacctgcctgaaatcactttttttcaggttggaccggtgccaccatgcaactggtgaagagcgagctgga
agagaagaaaagcgagctcagacataagctgaagtacgttccccacgaatacattgaactgatagaaatcgctag
aaacagtacgcaagacagaatactggaaatgaaggtgatggagttcttcatgaaggtttacggctatcgtggcaa
acacctcgggggctcccggaagcccgacggggctatctacaccgtgggcagtcccatcgactatggcgtgatcgt
ggacaccaaagcttatagcggcggatataatctccccatcggccaagccgatgagatgcagaggtatgtggagga
gaaccaaacaagaaacaagcatatcaaccccaacgagtggtggaaggtttatcctagctcggtgaccgagtttaa
gttcctattcgtgtctggccacttcaagggcaactataaggcacagctcactagactgaatcatatcacgaattg
caacggcgccgtgttatccgtggaggagctactgatcggcggagagatgatcaaagccggcaccctgaccctgga
agaggtgagaagaaagtttaacaatggcgaaataaatttcggcagcggaagtggaagcggctccatcactagaac
caccaaccctagaaacgtggtgcccaagatctacatgagcgccggcagcatccccctgaccacccacatcaccaa
ctcaattcagcccaccctgtggaccatcggcagcatcaacggcgtggcccccctggccaagagcatcaagctggg
catccccgtgaccggcagcgcctacaccgatcagaccaccgccatggtgagaaagaaggtgagcgtgttcatggg
cagcggcagcgggagcggctcatcgcagctggttaagagcgagttagaagaaaaaaagagcgaactgcggcataa
actgaagtatgtcccacacgagtacatcgaactgatcgagatcgcgagaaactctacccaagacagaattctgga
gatgaaagtaatggaatttttcatgaaggtgtatggatatagagggaagcacctgggtggcagcagaaaacccga
cggcgccatctacactgtggggagccccatagactatggtgtgatcgtggataccaaggcgtatagcggcggtta
caatctgcccattgggcaagcggacgagatgcaaagatatgtggaagagaatcagacgaggaacaagcacattaa
ccctaatgagtggtggaaggtctaccctagctccgttaccgagttcaagttcctgtttgtgagcgggcattttaa
gggcaactacaaggcacagctgacccgcctgaaccacataacaaactgcaacggtgccgtgctgagcgtagaaga
gttgctaatcggcggcgagatgatcaaggccggcacgctaaccctcgaagaggtgcgcagaaagttcaataacgg
cgaaatcaatttcagcggcagcgagactcccgggacctcagagtccgccacacccgaaagtacacagttcgaggg
ctttaccaacctgtatcaggtgagcaagacactgcggtttgagctgatcccacagggcaagaccctgaagcacat
ccaggagcagggcttcatcgaggaggacaaggcccgcaatgatcactacaaggagctgaagcccatcatcgatcg
gatctacaagacctatgccgaccagtgcctgcagctggtgcagctggattgggagaacctgagcgccgccatcga
ctcctatagaaaggagaaaaccgaggagacaaggaacgccctgatcgaggagcaggccacatatcgcaatgccat
ccacgactacttcatcggccggacagacaacctgaccgatgccatcaataagagacacgccgagatctacaaggg
cctgttcaaggccgagctgtttaatggcaaggtgctgaagcagctgggcaccgtgaccacaaccgagcacgagaa
cgccctgctgcggagcttcgacaagtttacaacctacttctccggcttttatagaaacaggaagaacgtgttcag
cgccgaggatatcagcacagccatcccacaccgcatcgtgcaggacaacttccccaagtttaaggagaattgtca
catcttcacacgcctgatcaccgccgtgcccagcctgcgggagcactttgagaacgtgaagaaggccatcggcat
cttcgtgagcacctccatcgaggaggtgttttccttccctttttataaccagctgctgacacagacccagatcga
cctgtataaccagctgctgggaggaatctctcgggaggcaggcaccgagaagatcaagggcctgaacgaggtgct
gaatctggccatccagaagaatgatgagacagcccacatcatcgcctccctgccacacagattcatccccctgtt
taagcagatcctgtccgataggaacaccctgtctttcatcctggaggagtttaagagcgacgaggaagtgatcca
gtccttctgcaagtacaagacactgctgagaaacgagaacgtgctggagacagccgaggccctgtttaacgagct
gaacagcatcgacctgacacacatcttcatcagccacaagaagctggagacaatcagcagcgccctgtgcgacca
ctgggatacactgaggaatgccctgtatgagcggagaatctccgagctgacaggcaagatcaccaagtctgccaa
ggagaaggtgcagcgcagcctgaagcacgaggatatcaacctgcaggagatcatctctgccgcaggcaaggagct
gagcgaggccttcaagcagaaaaccagcgagatcctgtcccacgcacacgccgccctggatcagccactgcctac
aaccctgaagaagcaggaggagaaggagatcctgaagtctcagctggacagcctgctgggcctgtaccacctgct
ggactggtttgccgtggatgagtccaacgaggtggaccccgagttctctgcccggctgaccggcatcaagctgga
gatggagccttctctgagcttctacaacaaggccagaaattatgccaccaagaagccctactccgtggagaagtt
caagctgaactttcagatgcctacactggccagaggctgggacgtgaatgtggagaagaacagaggcgccatcct
gtttgtgaagaacggcctgtactatctgggcatcatgccaaagcagaagggcaggtataaggccctgagcttcga
gcccacagagaaaaccagcgagggctttgataagatgtactatgactacttccctgatgccgccaagatgatccc
aaagtgcagcacccagctgaaggccgtgacagcccactttcagacccacacaacccccatcctgctgtccaacaa
tttcatcgagcctctggagatcacaaaggagatctacgacctgaacaatcctgagaaggagccaaagaagtttca
gacagcctacgccaagaaaaccggcgaccagaagggctacagagaggccctgtgcaagtggatcgacttcacaag
ggattttctgtccaagtataccaagacaacctctatcgatctgtctagcctgcggccatcctctcagtataagga
cctgggcgagtactatgccgagctgaatcccctgctgtaccacatcagcttccagagaatcgccgagaaggagat
catggatgccgtggagacaggcaagctgtacctgttccagatctataacaaggactttgccaagggccaccacgg
caagcctaatctgcacacactgtattggaccggcctgttttctccagagaacctggccaagacaagcatcaagct
gaatggccaggccgagctgttctaccgccctaagtccaggatgaagaggatggcacaccggctgggagagaagat
gctgaacaagaagctgaaggatcagaaaaccccaatccccgacaccctgtaccaggagctgtacgactatgtgaa
tcacagactgtcccacgacctgtctgatgaggccagggccctgctgcccaacgtgatcaccaaggaggtgtctca
cgagatcatcaaggataggcgctttaccagcgacaagttctttttccacgtgcctatcacactgaactatcaggc
cgccaattccccatctaagttcaaccagagggtgaatgcctacctgaaggagcaccccgagacacctatcatcgg
catcgcccggggcgagagaaacctgatctatatcacagtgatcgactccaccggcaagatcctggagcagcggag
cctgaacaccatccagcagtttgattaccagaagaagctggacaacagggagaaggagagggtggcagcaaggca
ggcctggtctgtggtgggcacaatcaaggatctgaagcagggctatctgagccaggtcatccacgagatcgtgga
cctgatgatccactaccaggccgtggtggtgctggagaacctgaatttcggctttaagagcaagaggaccggcat
cgccgagaaggccgtgtaccagcagttcgagaagatgctgatcgataagctgaattgcctggtgctgaaggacta
tccagcagagaaagtgggaggcgtgctgaacccataccagctgacagaccagttcacctcctttgccaagatggg
cacccagtctggcttcctgttttacgtgcctgccccatatacatctaagatcgatcccctgaccggcttcgtgga
ccccttcgtgtggaaaaccatcaagaatcacgagagccgcaagcacttcctggagggcttcgactttctgcacta
cgacgtgaaaaccggcgacttcatcctgcactttaagatgaacagaaatctgtccttccagaggggcctgcccgg
ctttatgcctgcatgggatatcgtgttcgagaagaacgagacacagtttgacgccaagggcacccctttcatcgc
cggcaagagaatcgtgccagtgatcgagaatcacagattcaccggcagataccgggacctgtatcctgccaacga
gctgatcgccctgctggaggagaagggcatcgtgttcagggatggctccaacatcctgccaaagctgctggagaa
tgacgattctcacgccatcgacaccatggtggccctgatccgcagcgtgctgcagatgcggaactccaatgccgc
cacaggcgaggactatatcaacagccccgtgcgcgatctgaatggcgtgtgcttcgactcccggtttcagaaccc
agagtggcccatggacgccgatgccaatggcgcctaccacatcgccctgaagggccagctgctgctgaatcacct
gaaggagagcaaggatctgaagctgcagaacggcatctccaatcaggactggctggcctacatccaggagctgcg
caacaaaaggccggcggccacgaaaaaggccggccaggcaaaaaagaaaaagggatcctacccatacgatgttcc
agattacgcttatccctacgacgtgcctgattatgcatacccatatgatgtccccgactatgccgagggcagagg
aagtctgctaacatgcggtgacgtcgaggagaatcctggcccaatgaccgagtacaagcccacggtgcgcctcgc
cacccgcgacgacgtccccagggccgtacgcaccctcgccgccgcgttcgccgactaccccgccacgcgccacac
cgtcgatccggaccgccacatcgagcgggtcaccgagctgcaagaactcttcctcacgcgcgtcgggctcgacat
cggcaaggtgtgggtcgcggacgacggcgccgcggtggcggtctggaccacgccggagagcgtcgaagcgggggc
ggtgttcgccgagatcggcccgcgcatggccgagttgagcggttcccggctggccgcgcagcaacagatggaagg
cctcctggcgccgcaccggcccaaggagcccgcgtggttcctggccaccgtcggagtctcgcccgaccaccaggg
caagggtctgggcagcgccgtcgtgctccccggagtggaggcggccgagcgcgccggggtgcccgccttcctgga
gacctccgcgccccgcaacctccccttctacgagcggctcggcttcaccgtcaccgccgacgtcgaggtgcccga
aggaccgcgcacctggtgcatgacccgcaagcccggtgcctgaactagtcctgcaggcatgcaagcttgatatca
agcttatcgataatcaacctctggattacaaaatttgtgaaagattgactggtattcttaactatgttgctcctt
ttacgctatgtggatacgctgctttaatgcctttgtatcatgctattgcttcccgtatggctttcattttctcct
ccttgtataaatcctggttgctgtctctttatgaggagttgtggcccgttgtcaggcaacgtggcgtggtgtgca
ctgtgtttgctgacgcaacccccactggttggggcattgccaccacctgtcagctcctttccgggactttcgctt
tccccctccctattgccacggcggaactcatcgccgcctgccttgcccgctgctggacaggggctcggctgttgg
gcactgacaattccgtggtgttgtcggggaaatcatcgtcctttccttggctgctcgcctgtgttgccacctgga
ttctgcgcgggacgtccttctgctacgtcccttcggccctcaatccagcggaccttccttcccgcggcctgctgc
cggctctgcggcctcttccgcgtcttcgccttcgccctcagacgagtcggatctccctttgggccgcctccccgc
atcgataccgtcgacctcgagggaattaattcgagctcggtacctttaagaccgatgacttacaaggcagctgta
gatcttagccactttttaaaagaaattaactgtgccttctagttgccagccatctgttgtttgcccctcccccgt
gccttccttgaccctggaaggtgccactcccactgtcctttcctaataaaatgaggaaattgcatcgcattgtct
gagtaggtgtcattctattctggggggtggggtggggcaggacagcaagggggaggattgggaagagaatagcag
gcatgctggggagcggccgcaggaacccctagtgatggagttggccactccctctctgcgcgctcgctcgctcac
tgaggccgggcgaccaaaggtcgcccgacgcccgggctttgcccgggcggcctcagtgagcgagcgagcgcgcag
ctgcctgcaggggcgcctgatgcggtattttctccttacgcatctgtgcggtatttcacaccgcatacgtcaaag
caaccatagtacgcgccctgtagcggcgcattaagcgcggcgggtgtggtggttacgcgcagcgtgaccgctaca
cttgccagcgccttagcgcccgctcctttcgctttcttcccttcctttctcgccacgttcgccggctttccccgt
caagctctaaatcgggggctccctttagggttccgatttagtgctttacggcacctcgaccccaaaaaacttgat
ttgggtgatggttcacgtagtgggccatcgccctgatagacggtttttcgccctttgacgttggagtccacgttc
tttaatagtggactcttgttccaaactggaacaacactcaactctatctcgggctattcttttgatttataaggg
attttgccgatttcggtctattggttaaaaaatgagctgatttaacaaaaatttaacgcgaattttaacaaaata
ttaacgtttacaattttatggtgcactctcagtacaatctgctctgatgccgcatagttaagccagccccgacac
ccgccaacacccgctgacgcgccctgacgggcttgtctgctcccggcatccgcttacagacaagctgtgaccgtc
tccgggagctgcatgtgtcagaggttttcaccgtcatcaccgaaacgcgcgagacgaaagggcctcgtgatacgc
ctatttttataggttaatgtcatgataataatggtttcttagacgtcaggtggcacttttcggggaaatgtgcgc
ggaacccctatttgtttatttttctaaatacattcaaatatgtatccgctcatgagacaataaccctgataaatg
cttcaataatattgaaaaaggaagagtatgagtattcaacatttccgtgtcgcccttattcccttttttgcggca
ttttgccttcctgtttttgctcacccagaaacgctggtgaaagtaaaagatgctgaagatcagttgggtgcacga
gtgggttacatcgaactggatctcaacagcggtaagatccttgagagttttcgccccgaagaacgttttccaatg
atgagcacttttaaagttctgctatgtggcgcggtattatcccgtattgacgccgggcaagagcaactcggtcgc
cgcatacactattctcagaatgacttggttgagtactcaccagtcacagaaaagcatcttacggatggcatgaca
gtaagagaattatgcagtgctgccataaccatgagtgataacactgcggccaacttacttctgacaacgatcgga
ggaccgaaggagctaaccgcttttttgcacaacatgggggatcatgtaactcgccttgatcgttgggaaccggag
ctgaatgaagccataccaaacgacgagcgtgacaccacgatgcctgtagcaatggcaacaacgttgcgcaaacta
ttaactggcgaactacttactctagcttcccggcaacaattaatagactggatggaggcggataaagttgcagga
ccacttctgcgctcggcccttccggctggctggtttattgctgataaatctggagccggtgagcgtggaagccgc
ggtatcattgcagcactggggccagatggtaagccctcccgtatcgtagttatctacacgacggggagtcaggca
actatggatgaacgaaatagacagatcgctgagataggtgcctcactgattaagcattggtaactgtcagaccaa
gtttactcatatatactttagattgatttaaaacttcatttttaatttaaaaggatctaggtgaagatccttttt
gataatctcatgaccaaaatcccttaacgtgagttttcgttccactgagcgtcagaccccgtagaaaagatcaaa
ggatcttcttgagatcctttttttctgcgcgtaatctgctgcttgcaaacaaaaaaaccaccgctaccagcggtg
gtttgtttgccggatcaagagctaccaactctttttccgaaggtaactggcttcagcagagcgcagataccaaat
actgttcttctagtgtagccgtagttaggccaccacttcaagaactctgtagcaccgcctacatacctcgctctg
ctaatcctgttaccagtggctgctgccagtggcgataagtcgtgtcttaccgggttggactcaagacgatagtta
ccggataaggcgcagcggtcgggctgaacggggggttcgtgcacacagcccagcttggagcgaacgacctacacc
gaactgagatacctacagcgtgagctatgagaaagcgccacgcttcccgaagggagaaaggcggacaggtatccg
gtaagcggcagggtcggaacaggagagcgcacgagggagcttccagggggaaacgcctggtatctttatagtcct
gtcgggtttcgccacctctgacttgagcgtcgatttttgtgatgctcgtcaggggggcggagcctatggaaaaac
gccagcaacgcggcctttttacggttcctggccttttgctggccttttgctcacatgt
Construct G (SEQ ID NO: 51)
gagggcctatttcccatgattccttcatatttgcatatacgatacaaggctgttagagagataattggaattaat
ttgactgtaaacacaaagatattagtacaaaatacgtgacgtagaaagtaataatttcttgggtagtttgcagtt
ttaaaattatgttttaaaatggactatcatatgcttaccgtaacttgaaagtatttcgatttcttggctttatat
atcttgtggaaaggacgaaacaccgggtcttcgagaagacctgttttagagctagaaatagcaagttaaaataag
gctagtccgttatcaacttgaaaaagtggcaccgagtcggtgcttttttgttttagagctagaaatagcaagtta
aaataaggctagtccgtttttagcgcgtgcgccaattctgcagacaaatggctctagaggtacccgttacataac
ttacggtaaatggcccgcctggctgaccgcccaacgacccccgcccattgacgtcaatagtaacgccaataggga
ctttccattgacgtcaatgggtggagtatttacggtaaactgcccacttggcagtacatcaagtgtatcatatgc
caagtacgccccctattgacgtcaatgacggtaaatggcccgcctggcattgtgcccagtacatgaccttatggg
actttcctacttggcagtacatctacgtattagtcatcgctattaccatggtcgaggtgagccccacgttctgct
tcactctccccatctcccccccctccccacccccaattttgtatttatttattttttaattattttgtgcagcga
tgggggcggggggggggggggggcgcgcgccaggcggggcggggcggggcgaggggcggggcggggcgaggcgga
gaggtgcggcggcagccaatcagagcggcgcgctccgaaagtttccttttatggcgaggcggcggcggcggcggc
cctataaaaagcgaagcgcgcggcgggcgggagtcgctgcgcgctgccttcgccccgtgccccgctccgccgccg
cctcgcgccgcccgccccggctctgactgaccgcgttactcccacaggtgagcgggcgggacggcccttctcctc
cgggctgtaattagctgagcaagaggtaagggtttaagggatggttggttggtggggtattaatgtttaattacc
tggagcacctgcctgaaatcactttttttcaggttggaccggtgccaccatgacacagttcgagggctttaccaa
cctgtatcaggtgagcaagacactgcggtttgagctgatcccacagggcaagaccctgaagcacatccaggagca
gggcttcatcgaggaggacaaggcccgcaatgatcactacaaggagctgaagcccatcatcgatcggatctacaa
gacctatgccgaccagtgcctgcagctggtgcagctggattgggagaacctgagcgccgccatcgactcctatag
aaaggagaaaaccgaggagacaaggaacgccctgatcgaggagcaggccacatatcgcaatgccatccacgacta
cttcatcggccggacagacaacctgaccgatgccatcaataagagacacgccgagatctacaagggcctgttcaa
ggccgagctgtttaatggcaaggtgctgaagcagctgggcaccgtgaccacaaccgagcacgagaacgccctgct
gcggagcttcgacaagtttacaacctacttctccggcttttatagaaacaggaagaacgtgttcagcgccgagga
tatcagcacagccatcccacaccgcatcgtgcaggacaacttccccaagtttaaggagaattgtcacatcttcac
acgcctgatcaccgccgtgcccagcctgcgggagcactttgagaacgtgaagaaggccatcggcatcttcgtgag
cacctccatcgaggaggtgttttccttccctttttataaccagctgctgacacagacccagatcgacctgtataa
ccagctgctgggaggaatctctcgggaggcaggcaccgagaagatcaagggcctgaacgaggtgctgaatctggc
catccagaagaatgatgagacagcccacatcatcgcctccctgccacacagattcatccccctgtttaagcagat
cctgtccgataggaacaccctgtctttcatcctggaggagtttaagagcgacgaggaagtgatccagtccttctg
caagtacaagacactgctgagaaacgagaacgtgctggagacagccgaggccctgtttaacgagctgaacagcat
cgacctgacacacatcttcatcagccacaagaagctggagacaatcagcagcgccctgtgcgaccactgggatac
actgaggaatgccctgtatgagcggagaatctccgagctgacaggcaagatcaccaagtctgccaaggagaaggt
gcagcgcagcctgaagcacgaggatatcaacctgcaggagatcatctctgccgcaggcaaggagctgagcgaggc
cttcaagcagaaaaccagcgagatcctgtcccacgcacacgccgccctggatcagccactgcctacaaccctgaa
gaagcaggaggagaaggagatcctgaagtctcagctggacagcctgctgggcctgtaccacctgctggactggtt
tgccgtggatgagtccaacgaggtggaccccgagttctctgcccggctgaccggcatcaagctggagatggagcc
ttctctgagcttctacaacaaggccagaaattatgccaccaagaagccctactccgtggagaagttcaagctgaa
ctttcagatgcctacactggccagaggctgggacgtgaatgtggagaagaacagaggcgccatcctgtttgtgaa
gaacggcctgtactatctgggcatcatgccaaagcagaagggcaggtataaggccctgagcttcgagcccacaga
gaaaaccagcgagggctttgataagatgtactatgactacttccctgatgccgccaagatgatcccaaagtgcag
cacccagctgaaggccgtgacagcccactttcagacccacacaacccccatcctgctgtccaacaatttcatcga
gcctctggagatcacaaaggagatctacgacctgaacaatcctgagaaggagccaaagaagtttcagacagccta
cgccaagaaaaccggcgaccagaagggctacagagaggccctgtgcaagtggatcgacttcacaagggattttct
gtccaagtataccaagacaacctctatcgatctgtctagcctgcggccatcctctcagtataaggacctgggcga
gtactatgccgagctgaatcccctgctgtaccacatcagcttccagagaatcgccgagaaggagatcatggatgc
cgtggagacaggcaagctgtacctgttccagatctataacaaggactttgccaagggccaccacggcaagcctaa
tctgcacacactgtattggaccggcctgttttctccagagaacctggccaagacaagcatcaagctgaatggcca
ggccgagctgttctaccgccctaagtccaggatgaagaggatggcacaccggctgggagagaagatgctgaacaa
gaagctgaaggatcagaaaaccccaatccccgacaccctgtaccaggagctgtacgactatgtgaatcacagact
gtcccacgacctgtctgatgaggccagggccctgctgcccaacgtgatcaccaaggaggtgtctcacgagatcat
caaggataggcgctttaccagcgacaagttctttttccacgtgcctatcacactgaactatcaggccgccaattc
cccatctaagttcaaccagagggtgaatgcctacctgaaggagcaccccgagacacctatcatcggcatcgcccg
gggcgagagaaacctgatctatatcacagtgatcgactccaccggcaagatcctggagcagcggagcctgaacac
catccagcagtttgattaccagaagaagctggacaacagggagaaggagagggtggcagcaaggcaggcctggtc
tgtggtgggcacaatcaaggatctgaagcagggctatctgagccaggtcatccacgagatcgtggacctgatgat
ccactaccaggccgtggtggtgctggagaacctgaatttcggctttaagagcaagaggaccggcatcgccgagaa
ggccgtgtaccagcagttcgagaagatgctgatcgataagctgaattgcctggtgctgaaggactatccagcaga
gaaagtgggaggcgtgctgaacccataccagctgacagaccagttcacctcctttgccaagatgggcacccagtc
tggcttcctgttttacgtgcctgccccatatacatctaagatcgatcccctgaccggcttcgtggaccccttcgt
gtggaaaaccatcaagaatcacgagagccgcaagcacttcctggagggcttcgactttctgcactacgacgtgaa
aaccggcgacttcatcctgcactttaagatgaacagaaatctgtccttccagaggggcctgcccggctttatgcc
tgcatgggatatcgtgttcgagaagaacgagacacagtttgacgccaagggcacccctttcatcgccggcaagag
aatcgtgccagtgatcgagaatcacagattcaccggcagataccgggacctgtatcctgccaacgagctgatcgc
cctgctggaggagaagggcatcgtgttcagggatggctccaacatcctgccaaagctgctggagaatgacgattc
tcacgccatcgacaccatggtggccctgatccgcagcgtgctgcagatgcggaactccaatgccgccacaggcga
ggactatatcaacagccccgtgcgcgatctgaatggcgtgtgcttcgactcccggtttcagaacccagagtggcc
catggacgccgatgccaatggcgcctaccacatcgccctgaagggccagctgctgctgaatcacctgaaggagag
caaggatctgaagctgcagaacggcatctccaatcaggactggctggcctacatccaggagctgcgcaacagcgg
cagcgagactcccgggacctcagagtccgccacacccgaaagtcaactggtgaagagcgagctggaagagaagaa
aagcgagctcagacataagctgaagtacgttccccacgaatacattgaactgatagaaatcgctagaaacagtac
gcaagacagaatactggaaatgaaggtgatggagttcttcatgaaggtttacggctatcgtggcaaacacctcgg
gggctcccggaagcccgccggggctatctacaccgtgggcagtcccatcgactatggcgtgatcgtggacaccaa
agcttatagcggcggatataatctccccatcggccaagccgatgagatgcagaggtatgtggaggagaaccaaac
aagaaacaagcatatcaaccccaacgagtggtggaaggtttatcctagctcggtgaccgagtttaagttcctatt
cgtgtctggccacttcaagggcaactataaggcacagctcactagactgaatcatatcacgaattgcaacggcgc
cgtgttatccgtggaggagctactgatcggcggagagatgatcaaagccggcaccctgaccctggaagaggtgag
aagaaagtttaacaatggcgaaataaatttcggcagcggaagtggaagcggctccatcactagaaccaccaaccc
tagaaacgtggtgcccaagatctacatgagcgccggcagcatccccctgaccacccacatcaccaactcaattca
gcccaccctgtggaccatcggcagcatcaacggcgtggcccccctggccaagagcatcaagctgggcatccccgt
gaccggcagcgcctacaccgatcagaccaccgccatggtgagaaagaaggtgagcgtgttcatgggcagcggcag
cgggagcggctcatcgcagctggttaagagcgagttagaagaaaaaaagagcgaactgcggcataaactgaagta
tgtcccacacgagtacatcgaactgatcgagatcgcgagaaactctacccaagacagaattctggagatgaaagt
aatggaatttttcatgaaggtgtatggatatagagggaagcacctgggtggcagcagaaaacccgacggcgccat
ctacactgtggggagccccatagactatggtgtgatcgtggataccaaggcgtatagcggcggttacaatctgcc
cattgggcaagcggacgagatgcaaagatatgtggaagagaatcagacgaggaacaagcacattaaccctaatga
gtggtggaaggtctaccctagctccgttaccgagttcaagttcctgtttgtgagcgggcattttaagggcaacta
caaggcacagctgacccgcctgaaccacataacaaactgcaacggtgccgtgctgagcgtagaagagttgctaat
cggcggcgagatgatcaaggccggcacgctaaccctcgaagaggtgcgcagaaagttcaataacggcgaaatcaa
tttcaaaaggccggcggccacgaaaaaggccggccaggcaaaaaagaaaaagggatcctacccatacgatgttcc
agattacgcttatccctacgacgtgcctgattatgcatacccatatgatgtccccgactatgccgagggcagagg
aagtctgctaacatgcggtgacgtcgaggagaatcctggcccaatgaccgagtacaagcccacggtgcgcctcgc
cacccgcgacgacgtccccagggccgtacgcaccctcgccgccgcgttcgccgactaccccgccacgcgccacac
cgtcgatccggaccgccacatcgagcgggtcaccgagctgcaagaactcttcctcacgcgcgtcgggctcgacat
cggcaaggtgtgggtcgcggacgacggcgccgcggtggcggtctggaccacgccggagagcgtcgaagcgggggc
ggtgttcgccgagatcggcccgcgcatggccgagttgagcggttcccggctggccgcgcagcaacagatggaagg
cctcctggcgccgcaccggcccaaggagcccgcgtggttcctggccaccgtcggagtctcgcccgaccaccaggg
caagggtctgggcagcgccgtcgtgctccccggagtggaggcggccgagcgcgccggggtgcccgccttcctgga
gacctccgcgccccgcaacctccccttctacgagcggctcggcttcaccgtcaccgccgacgtcgaggtgcccga
aggaccgcgcacctggtgcatgacccgcaagcccggtgcctgaactagtcctgcaggcatgcaagcttgatatca
agcttatcgataatcaacctctggattacaaaatttgtgaaagattgactggtattcttaactatgttgctcctt
ttacgctatgtggatacgctgctttaatgcctttgtatcatgctattgcttcccgtatggctttcattttctcct
ccttgtataaatcctggttgctgtctctttatgaggagttgtggcccgttgtcaggcaacgtggcgtggtgtgca
ctgtgtttgctgacgcaacccccactggttggggcattgccaccacctgtcagctcctttccgggactttcgctt
tccccctccctattgccacggcggaactcatcgccgcctgccttgcccgctgctggacaggggctcggctgttgg
gcactgacaattccgtggtgttgtcggggaaatcatcgtcctttccttggctgctcgcctgtgttgccacctgga
ttctgcgcgggacgtccttctgctacgtcccttcggccctcaatccagcggaccttccttcccgcggcctgctgc
cggctctgcggcctcttccgcgtcttcgccttcgccctcagacgagtcggatctccctttgggccgcctccccgc
atcgataccgtcgacctcgagggaattaattcgagctcggtacctttaagaccgatgacttacaaggcagctgta
gatcttagccactttttaaaagaaattaactgtgccttctagttgccagccatctgttgtttgcccctcccccgt
gccttccttgaccctggaaggtgccactcccactgtcctttcctaataaaatgaggaaattgcatcgcattgtct
gagtaggtgtcattctattctggggggtggggtggggcaggacagcaagggggaggattgggaagagaatagcag
gcatgctggggagcggccgcaggaacccctagtgatggagttggccactccctctctgcgcgctcgctcgctcac
tgaggccgggcgaccaaaggtcgcccgacgcccgggctttgcccgggcggcctcagtgagcgagcgagcgcgcag
ctgcctgcaggggcgcctgatgcggtattttctccttacgcatctgtgcggtatttcacaccgcatacgtcaaag
caaccatagtacgcgccctgtagcggcgcattaagcgcggcgggtgtggtggttacgcgcagcgtgaccgctaca
cttgccagcgccttagcgcccgctcctttcgctttcttcccttcctttctcgccacgttcgccggctttccccgt
caagctctaaatcgggggctccctttagggttccgatttagtgctttacggcacctcgaccccaaaaaacttgat
ttgggtgatggttcacgtagtgggccatcgccctgatagacggtttttcgccctttgacgttggagtccacgttc
tttaatagtggactcttgttccaaactggaacaacactcaactctatctcgggctattcttttgatttataaggg
attttgccgatttcggtctattggttaaaaaatgagctgatttaacaaaaatttaacgcgaattttaacaaaata
ttaacgtttacaattttatggtgcactctcagtacaatctgctctgatgccgcatagttaagccagccccgacac
ccgccaacacccgctgacgcgccctgacgggcttgtctgctcccggcatccgcttacagacaagctgtgaccgtc
tccgggagctgcatgtgtcagaggttttcaccgtcatcaccgaaacgcgcgagacgaaagggcctcgtgatacgc
ctatttttataggttaatgtcatgataataatggtttcttagacgtcaggtggcacttttcggggaaatgtgcgc
ggaacccctatttgtttatttttctaaatacattcaaatatgtatccgctcatgagacaataaccctgataaatg
cttcaataatattgaaaaaggaagagtatgagtattcaacatttccgtgtcgcccttattcccttttttgcggca
ttttgccttcctgtttttgctcacccagaaacgctggtgaaagtaaaagatgctgaagatcagttgggtgcacga
gtgggttacatcgaactggatctcaacagcggtaagatccttgagagttttcgccccgaagaacgttttccaatg
atgagcacttttaaagttctgctatgtggcgcggtattatcccgtattgacgccgggcaagagcaactcggtcgc
cgcatacactattctcagaatgacttggttgagtactcaccagtcacagaaaagcatcttacggatggcatgaca
gtaagagaattatgcagtgctgccataaccatgagtgataacactgcggccaacttacttctgacaacgatcgga
ggaccgaaggagctaaccgcttttttgcacaacatgggggatcatgtaactcgccttgatcgttgggaaccggag
ctgaatgaagccataccaaacgacgagcgtgacaccacgatgcctgtagcaatggcaacaacgttgcgcaaacta
ttaactggcgaactacttactctagcttcccggcaacaattaatagactggatggaggcggataaagttgcagga
ccacttctgcgctcggcccttccggctggctggtttattgctgataaatctggagccggtgagcgtggaagccgc
ggtatcattgcagcactggggccagatggtaagccctcccgtatcgtagttatctacacgacggggagtcaggca
actatggatgaacgaaatagacagatcgctgagataggtgcctcactgattaagcattggtaactgtcagaccaa
gtttactcatatatactttagattgatttaaaacttcatttttaatttaaaaggatctaggtgaagatccttttt
gataatctcatgaccaaaatcccttaacgtgagttttcgttccactgagcgtcagaccccgtagaaaagatcaaa
ggatcttcttgagatcctttttttctgcgcgtaatctgctgcttgcaaacaaaaaaaccaccgctaccagcggtg
gtttgtttgccggatcaagagctaccaactctttttccgaaggtaactggcttcagcagagcgcagataccaaat
actgttcttctagtgtagccgtagttaggccaccacttcaagaactctgtagcaccgcctacatacctcgctctg
ctaatcctgttaccagtggctgctgccagtggcgataagtcgtgtcttaccgggttggactcaagacgatagtta
ccggataaggcgcagcggtcgggctgaacggggggttcgtgcacacagcccagcttggagcgaacgacctacacc
gaactgagatacctacagcgtgagctatgagaaagcgccacgcttcccgaagggagaaaggcggacaggtatccg
gtaagcggcagggtcggaacaggagagcgcacgagggagcttccagggggaaacgcctggtatctttatagtcct
gtcgggtttcgccacctctgacttgagcgtcgatttttgtgatgctcgtcaggggggcggagcctatggaaaaac
gccagcaacgcggcctttttacggttcctggccttttgctggccttttgctcacatgt
Construct H (SEQ ID NO: 52)
gagggcctatttcccatgattccttcatatttgcatatacgatacaaggctgttagagagataattggaattaat
ttgactgtaaacacaaagatattagtacaaaatacgtgacgtagaaagtaataatttcttgggtagtttgcagtt
ttaaaattatgttttaaaatggactatcatatgcttaccgtaacttgaaagtatttcgatttcttggctttatat
atcttgtggaaaggacgaaacaccgggtcttcgagaagacctgttttagagctagaaatagcaagttaaaataag
gctagtccgttatcaacttgaaaaagtggcaccgagtcggtgcttttttgttttagagctagaaatagcaagtta
aaataaggctagtccgtttttagcgcgtgcgccaattctgcagacaaatggctctagaggtacccgttacataac
ttacggtaaatggcccgcctggctgaccgcccaacgacccccgcccattgacgtcaatagtaacgccaataggga
ctttccattgacgtcaatgggtggagtatttacggtaaactgcccacttggcagtacatcaagtgtatcatatgc
caagtacgccccctattgacgtcaatgacggtaaatggcccgcctggcattgtgcccagtacatgaccttatggg
actttcctacttggcagtacatctacgtattagtcatcgctattaccatggtcgaggtgagccccacgttctgct
tcactctccccatctcccccccctccccacccccaattttgtatttatttattttttaattattttgtgcagcga
tgggggcggggggggggggggggcgcgcgccaggcggggcggggcggggcgaggggcggggcggggcgaggcgga
gaggtgcggcggcagccaatcagagcggcgcgctccgaaagtttccttttatggcgaggcggcggcggcggcggc
cctataaaaagcgaagcgcgcggcgggcgggagtcgctgcgcgctgccttcgccccgtgccccgctccgccgccg
cctcgcgccgcccgccccggctctgactgaccgcgttactcccacaggtgagcgggcgggacggcccttctcctc
cgggctgtaattagctgagcaagaggtaagggtttaagggatggttggttggtggggtattaatgtttaattacc
tggagcacctgcctgaaatcactttttttcaggttggaccggtgccaccatgacacagttcgagggctttaccaa
cctgtatcaggtgagcaagacactgcggtttgagctgatcccacagggcaagaccctgaagcacatccaggagca
gggcttcatcgaggaggacaaggcccgcaatgatcactacaaggagctgaagcccatcatcgatcggatctacaa
gacctatgccgaccagtgcctgcagctggtgcagctggattgggagaacctgagcgccgccatcgactcctatag
aaaggagaaaaccgaggagacaaggaacgccctgatcgaggagcaggccacatatcgcaatgccatccacgacta
cttcatcggccggacagacaacctgaccgatgccatcaataagagacacgccgagatctacaagggcctgttcaa
ggccgagctgtttaatggcaaggtgctgaagcagctgggcaccgtgaccacaaccgagcacgagaacgccctgct
gcggagcttcgacaagtttacaacctacttctccggcttttatagaaacaggaagaacgtgttcagcgccgagga
tatcagcacagccatcccacaccgcatcgtgcaggacaacttccccaagtttaaggagaattgtcacatcttcac
acgcctgatcaccgccgtgcccagcctgcgggagcactttgagaacgtgaagaaggccatcggcatcttcgtgag
cacctccatcgaggaggtgttttccttccctttttataaccagctgctgacacagacccagatcgacctgtataa
ccagctgctgggaggaatctctcgggaggcaggcaccgagaagatcaagggcctgaacgaggtgctgaatctggc
catccagaagaatgatgagacagcccacatcatcgcctccctgccacacagattcatccccctgtttaagcagat
cctgtccgataggaacaccctgtctttcatcctggaggagtttaagagcgacgaggaagtgatccagtccttctg
caagtacaagacactgctgagaaacgagaacgtgctggagacagccgaggccctgtttaacgagctgaacagcat
cgacctgacacacatcttcatcagccacaagaagctggagacaatcagcagcgccctgtgcgaccactgggatac
actgaggaatgccctgtatgagcggagaatctccgagctgacaggcaagatcaccaagtctgccaaggagaaggt
gcagcgcagcctgaagcacgaggatatcaacctgcaggagatcatctctgccgcaggcaaggagctgagcgaggc
cttcaagcagaaaaccagcgagatcctgtcccacgcacacgccgccctggatcagccactgcctacaaccctgaa
gaagcaggaggagaaggagatcctgaagtctcagctggacagcctgctgggcctgtaccacctgctggactggtt
tgccgtggatgagtccaacgaggtggaccccgagttctctgcccggctgaccggcatcaagctggagatggagcc
ttctctgagcttctacaacaaggccagaaattatgccaccaagaagccctactccgtggagaagttcaagctgaa
ctttcagatgcctacactggccagaggctgggacgtgaatgtggagaagaacagaggcgccatcctgtttgtgaa
gaacggcctgtactatctgggcatcatgccaaagcagaagggcaggtataaggccctgagcttcgagcccacaga
gaaaaccagcgagggctttgataagatgtactatgactacttccctgatgccgccaagatgatcccaaagtgcag
cacccagctgaaggccgtgacagcccactttcagacccacacaacccccatcctgctgtccaacaatttcatcga
gcctctggagatcacaaaggagatctacgacctgaacaatcctgagaaggagccaaagaagtttcagacagccta
cgccaagaaaaccggcgaccagaagggctacagagaggccctgtgcaagtggatcgacttcacaagggattttct
gtccaagtataccaagacaacctctatcgatctgtctagcctgcggccatcctctcagtataaggacctgggcga
gtactatgccgagctgaatcccctgctgtaccacatcagcttccagagaatcgccgagaaggagatcatggatgc
cgtggagacaggcaagctgtacctgttccagatctataacaaggactttgccaagggccaccacggcaagcctaa
tctgcacacactgtattggaccggcctgttttctccagagaacctggccaagacaagcatcaagctgaatggcca
ggccgagctgttctaccgccctaagtccaggatgaagaggatggcacaccggctgggagagaagatgctgaacaa
gaagctgaaggatcagaaaaccccaatccccgacaccctgtaccaggagctgtacgactatgtgaatcacagact
gtcccacgacctgtctgatgaggccagggccctgctgcccaacgtgatcaccaaggaggtgtctcacgagatcat
caaggataggcgctttaccagcgacaagttctttttccacgtgcctatcacactgaactatcaggccgccaattc
cccatctaagttcaaccagagggtgaatgcctacctgaaggagcaccccgagacacctatcatcggcatcgcccg
gggcgagagaaacctgatctatatcacagtgatcgactccaccggcaagatcctggagcagcggagcctgaacac
catccagcagtttgattaccagaagaagctggacaacagggagaaggagagggtggcagcaaggcaggcctggtc
tgtggtgggcacaatcaaggatctgaagcagggctatctgagccaggtcatccacgagatcgtggacctgatgat
ccactaccaggccgtggtggtgctggagaacctgaatttcggctttaagagcaagaggaccggcatcgccgagaa
ggccgtgtaccagcagttcgagaagatgctgatcgataagctgaattgcctggtgctgaaggactatccagcaga
gaaagtgggaggcgtgctgaacccataccagctgacagaccagttcacctcctttgccaagatgggcacccagtc
tggcttcctgttttacgtgcctgccccatatacatctaagatcgatcccctgaccggcttcgtggaccccttcgt
gtggaaaaccatcaagaatcacgagagccgcaagcacttcctggagggcttcgactttctgcactacgacgtgaa
aaccggcgacttcatcctgcactttaagatgaacagaaatctgtccttccagaggggcctgcccggctttatgcc
tgcatgggatatcgtgttcgagaagaacgagacacagtttgacgccaagggcacccctttcatcgccggcaagag
aatcgtgccagtgatcgagaatcacagattcaccggcagataccgggacctgtatcctgccaacgagctgatcgc
cctgctggaggagaagggcatcgtgttcagggatggctccaacatcctgccaaagctgctggagaatgacgattc
tcacgccatcgacaccatggtggccctgatccgcagcgtgctgcagatgcggaactccaatgccgccacaggcga
ggactatatcaacagccccgtgcgcgatctgaatggcgtgtgcttcgactcccggtttcagaacccagagtggcc
catggacgccgatgccaatggcgcctaccacatcgccctgaagggccagctgctgctgaatcacctgaaggagag
caaggatctgaagctgcagaacggcatctccaatcaggactggctggcctacatccaggagctgcgcaacagcgg
cagcgagactcccgggacctcagagtccgccacacccgaaagtcaactggtgaagagcgagctggaagagaagaa
aagcgagctcagacataagctgaagtacgttccccacgaatacattgaactgatagaaatcgctagaaacagtac
gcaagacagaatactggaaatgaaggtgatggagttcttcatgaaggtttacggctatcgtggcaaacacctcgg
gggctcccggaagcccgacggggctatctacaccgtgggcagtcccatcgactatggcgtgatcgtggacaccaa
agcttatagcggcggatataatctccccatcggccaagccgatgagatgcagaggtatgtggaggagaaccaaac
aagaaacaagcatatcaaccccaacgagtggtggaaggtttatcctagctcggtgaccgagtttaagttcctatt
cgtgtctggccacttcaagggcaactataaggcacagctcactagactgaatcatatcacgaattgcaacggcgc
cgtgttatccgtggaggagctactgatcggcggagagatgatcaaagccggcaccctgaccctggaagaggtgag
aagaaagtttaacaatggcgaaataaatttcggcagcggaagtggaagcggctccatcactagaaccaccaaccc
tagaaacgtggtgcccaagatctacatgagcgccggcagcatccccctgaccacccacatcaccaactcaattca
gcccaccctgtggaccatcggcagcatcaacggcgtggcccccctggccaagagcatcaagctgggcatccccgt
gaccggcagcgcctacaccgatcagaccaccgccatggtgagaaagaaggtgagcgtgttcatgggcagcggcag
cgggagcggctcatcgcagctggttaagagcgagttagaagaaaaaaagagcgaactgcggcataaactgaagta
tgtcccacacgagtacatcgaactgatcgagatcgcgagaaactctacccaagacagaattctggagatgaaagt
aatggaatttttcatgaaggtgtatggatatagagggaagcacctgggtggcagcagaaaacccgccggcgccat
ctacactgtggggagccccatagactatggtgtgatcgtggataccaaggcgtatagcggcggttacaatctgcc
cattgggcaagcggacgagatgcaaagatatgtggaagagaatcagacgaggaacaagcacattaaccctaatga
gtggtggaaggtctaccctagctccgttaccgagttcaagttcctgtttgtgagcgggcattttaagggcaacta
caaggcacagctgacccgcctgaaccacataacaaactgcaacggtgccgtgctgagcgtagaagagttgctaat
cggcggcgagatgatcaaggccggcacgctaaccctcgaagaggtgcgcagaaagttcaataacggcgaaatcaa
tttcaaaaggccggcggccacgaaaaaggccggccaggcaaaaaagaaaaagggatcctacccatacgatgttcc
agattacgcttatccctacgacgtgcctgattatgcatacccatatgatgtccccgactatgccgagggcagagg
aagtctgctaacatgcggtgacgtcgaggagaatcctggcccaatgaccgagtacaagcccacggtgcgcctcgc
cacccgcgacgacgtccccagggccgtacgcaccctcgccgccgcgttcgccgactaccccgccacgcgccacac
cgtcgatccggaccgccacatcgagcgggtcaccgagctgcaagaactcttcctcacgcgcgtcgggctcgacat
cggcaaggtgtgggtcgcggacgacggcgccgcggtggcggtctggaccacgccggagagcgtcgaagcgggggc
ggtgttcgccgagatcggcccgcgcatggccgagttgagcggttcccggctggccgcgcagcaacagatggaagg
cctcctggcgccgcaccggcccaaggagcccgcgtggttcctggccaccgtcggagtctcgcccgaccaccaggg
caagggtctgggcagcgccgtcgtgctccccggagtggaggcggccgagcgcgccggggtgcccgccttcctgga
gacctccgcgccccgcaacctccccttctacgagcggctcggcttcaccgtcaccgccgacgtcgaggtgcccga
aggaccgcgcacctggtgcatgacccgcaagcccggtgcctgaactagtcctgcaggcatgcaagcttgatatca
agcttatcgataatcaacctctggattacaaaatttgtgaaagattgactggtattcttaactatgttgctcctt
ttacgctatgtggatacgctgctttaatgcctttgtatcatgctattgcttcccgtatggctttcattttctcct
ccttgtataaatcctggttgctgtctctttatgaggagttgtggcccgttgtcaggcaacgtggcgtggtgtgca
ctgtgtttgctgacgcaacccccactggttggggcattgccaccacctgtcagctcctttccgggactttcgctt
tccccctccctattgccacggcggaactcatcgccgcctgccttgcccgctgctggacaggggctcggctgttgg
gcactgacaattccgtggtgttgtcggggaaatcatcgtcctttccttggctgctcgcctgtgttgccacctgga
ttctgcgcgggacgtccttctgctacgtcccttcggccctcaatccagcggaccttccttcccgcggcctgctgc
cggctctgcggcctcttccgcgtcttcgccttcgccctcagacgagtcggatctccctttgggccgcctccccgc
atcgataccgtcgacctcgagggaattaattcgagctcggtacctttaagaccgatgacttacaaggcagctgta
gatcttagccactttttaaaagaaattaactgtgccttctagttgccagccatctgttgtttgcccctcccccgt
gccttccttgaccctggaaggtgccactcccactgtcctttcctaataaaatgaggaaattgcatcgcattgtct
gagtaggtgtcattctattctgggggggggggtggggcaggacagcaagggggaggattgggaagagaatagcag
gcatgctggggagcggccgcaggaacccctagtgatggagttggccactccctctctgcgcgctcgctcgctcac
tgaggccgggcgaccaaaggtcgcccgacgcccgggctttgcccgggcggcctcagtgagcgagcgagcgcgcag
ctgcctgcaggggcgcctgatgcggtattttctccttacgcatctgtgcggtatttcacaccgcatacgtcaaag
caaccatagtacgcgccctgtagcggcgcattaagcgcggcgggtgtggtggttacgcgcagcgtgaccgctaca
cttgccagcgccttagcgcccgctcctttcgctttcttcccttcctttctcgccacgttcgccggctttccccgt
caagctctaaatcgggggctccctttagggttccgatttagtgctttacggcacctcgaccccaaaaaacttgat
ttgggtgatggttcacgtagtgggccatcgccctgatagacggtttttcgccctttgacgttggagtccacgttc
tttaatagtggactcttgttccaaactggaacaacactcaactctatctcgggctattcttttgatttataaggg
attttgccgatttcggtctattggttaaaaaatgagctgatttaacaaaaatttaacgcgaattttaacaaaata
ttaacgtttacaattttatggtgcactctcagtacaatctgctctgatgccgcatagttaagccagccccgacac
ccgccaacacccgctgacgcgccctgacgggcttgtctgctcccggcatccgcttacagacaagctgtgaccgtc
tccgggagctgcatgtgtcagaggttttcaccgtcatcaccgaaacgcgcgagacgaaagggcctcgtgatacgc
ctatttttataggttaatgtcatgataataatggtttcttagacgtcaggtggcacttttcggggaaatgtgcgc
ggaacccctatttgtttatttttctaaatacattcaaatatgtatccgctcatgagacaataaccctgataaatg
cttcaataatattgaaaaaggaagagtatgagtattcaacatttccgtgtcgcccttattcccttttttgcggca
ttttgccttcctgtttttgctcacccagaaacgctggtgaaagtaaaagatgctgaagatcagttgggtgcacga
gtgggttacatcgaactggatctcaacagcggtaagatccttgagagttttcgccccgaagaacgttttccaatg
atgagcacttttaaagttctgctatgtggcgcggtattatcccgtattgacgccgggcaagagcaactcggtcgc
cgcatacactattctcagaatgacttggttgagtactcaccagtcacagaaaagcatcttacggatggcatgaca
gtaagagaattatgcagtgctgccataaccatgagtgataacactgcggccaacttacttctgacaacgatcgga
ggaccgaaggagctaaccgcttttttgcacaacatgggggatcatgtaactcgccttgatcgttgggaaccggag
ctgaatgaagccataccaaacgacgagcgtgacaccacgatgcctgtagcaatggcaacaacgttgcgcaaacta
ttaactggcgaactacttactctagcttcccggcaacaattaatagactggatggaggcggataaagttgcagga
ccacttctgcgctcggcccttccggctggctggtttattgctgataaatctggagccggtgagcgtggaagccgc
ggtatcattgcagcactggggccagatggtaagccctcccgtatcgtagttatctacacgacggggagtcaggca
actatggatgaacgaaatagacagatcgctgagataggtgcctcactgattaagcattggtaactgtcagaccaa
gtttactcatatatactttagattgatttaaaacttcatttttaatttaaaaggatctaggtgaagatccttttt
gataatctcatgaccaaaatcccttaacgtgagttttcgttccactgagcgtcagaccccgtagaaaagatcaaa
ggatcttcttgagatcctttttttctgcgcgtaatctgctgcttgcaaacaaaaaaaccaccgctaccagcggtg
gtttgtttgccggatcaagagctaccaactctttttccgaaggtaactggcttcagcagagcgcagataccaaat
actgttcttctagtgtagccgtagttaggccaccacttcaagaactctgtagcaccgcctacatacctcgctctg
ctaatcctgttaccagtggctgctgccagtggcgataagtcgtgtcttaccgggttggactcaagacgatagtta
ccggataaggcgcagcggtcgggctgaacggggggttcgtgcacacagcccagcttggagcgaacgacctacacc
gaactgagatacctacagcgtgagctatgagaaagcgccacgcttcccgaagggagaaaggcggacaggtatccg
gtaagcggcagggtcggaacaggagagcgcacgagggagcttccagggggaaacgcctggtatctttatagtcct
gtcgggtttcgccacctctgacttgagcgtcgatttttgtgatgctcgtcaggggggcggagcctatggaaaaac
gccagcaacgcggcctttttacggttcctggccttttgctggccttttgctcacatgt
Construct I (SEQ ID NO: 53)
gagggcctatttcccatgattccttcatatttgcatatacgatacaaggctgttagagagataattggaattaat
ttgactgtaaacacaaagatattagtacaaaatacgtgacgtagaaagtaataatttcttgggtagtttgcagtt
ttaaaattatgttttaaaatggactatcatatgcttaccgtaacttgaaagtatttcgatttcttggctttatat
atcttgtggaaaggacgaaacaccgggtcttcgagaagacctgttttagagctagaaatagcaagttaaaataag
gctagtccgttatcaacttgaaaaagtggcaccgagtcggtgcttttttgttttagagctagaaatagcaagtta
aaataaggctagtccgtttttagcgcgtgcgccaattctgcagacaaatggctctagaggtacccgttacataac
ttacggtaaatggcccgcctggctgaccgcccaacgacccccgcccattgacgtcaatagtaacgccaataggga
ctttccattgacgtcaatgggtggagtatttacggtaaactgcccacttggcagtacatcaagtgtatcatatgc
caagtacgccccctattgacgtcaatgacggtaaatggcccgcctggcattgtgcccagtacatgaccttatggg
actttcctacttggcagtacatctacgtattagtcatcgctattaccatggtcgaggtgagccccacgttctgct
tcactctccccatctcccccccctccccacccccaattttgtatttatttattttttaattattttgtgcagcga
tgggggcggggggggggggggggcgcgcgccaggcggggcggggcggggcgaggggcggggcggggcgaggcgga
gaggtgcggcggcagccaatcagagcggcgcgctccgaaagtttccttttatggcgaggcggcggcggcggcggc
cctataaaaagcgaagcgcgcggcgggcgggagtcgctgcgcgctgccttcgccccgtgccccgctccgccgccg
cctcgcgccgcccgccccggctctgactgaccgcgttactcccacaggtgagcgggcgggacggcccttctcctc
cgggctgtaattagctgagcaagaggtaagggtttaagggatggttggttggtggggtattaatgtttaattacc
tggagcacctgcctgaaatcactttttttcaggttggaccggtgccaccatgcaactggtgaagagcgagctgga
agagaagaaaagcgagctcagacataagctgaagtacgttccccacgaatacattgaactgatagaaatcgctag
aaacagtacgcaagacagaatactggaaatgaaggtgatggagttcttcatgaaggtttacggctatcgtggcaa
acacctcgggggctcccggaagcccgccggggctatctacaccgtgggcagtcccatcgactatggcgtgatcgt
ggacaccaaagcttatagcggcggatataatctccccatcggccaagccgatgagatgcagaggtatgtggagga
gaaccaaacaagaaacaagcatatcaaccccaacgagtggtggaaggtttatcctagctcggtgaccgagtttaa
gttcctattcgtgtctggccacttcaagggcaactataaggcacagctcactagactgaatcatatcacgaattg
caacggcgccgtgttatccgtggaggagctactgatcggcggagagatgatcaaagccggcaccctgaccctgga
agaggtgagaagaaagtttaacaatggcgaaataaatttcggcagcggaagtggaagcggctccatcactagaac
caccaaccctagaaacgtggtgcccaagatctacatgagcgccggcagcatccccctgaccacccacatcaccaa
ctcaattcagcccaccctgtggaccatcggcagcatcaacggcgtggcccccctggccaagagcatcaagctggg
catccccgtgaccggcagcgcctacaccgatcagaccaccgccatggtgagaaagaaggtgagcgtgttcatggg
cagcggcagcgggagcggctcatcgcagctggttaagagcgagttagaagaaaaaaagagcgaactgcggcataa
actgaagtatgtcccacacgagtacatcgaactgatcgagatcgcgagaaactctacccaagacagaattctgga
gatgaaagtaatggaatttttcatgaaggtgtatggatatagagggaagcacctgggtggcagcagaaaacccga
cggcgccatctacactgtggggagccccatagactatggtgtgatcgtggataccaaggcgtatagcggcggtta
caatctgcccattgggcaagcggacgagatgcaaagatatgtggaagagaatcagacgaggaacaagcacattaa
ccctaatgagtggtggaaggtctaccctagctccgttaccgagttcaagttcctgtttgtgagcgggcattttaa
gggcaactacaaggcacagctgacccgcctgaaccacataacaaactgcaacggtgccgtgctgagcgtagaaga
gttgctaatcggcggcgagatgatcaaggccggcacgctaaccctcgaagaggtgcgcagaaagttcaataacgg
cgaaatcaatttcagcggcagcgagactcccgggacctcagagtccgccacacccgaaagtacacagttcgaggg
ctttaccaacctgtatcaggtgagcaagacactgcggtttgagctgatcccacagggcaagaccctgaagcacat
ccaggagcagggcttcatcgaggaggacaaggcccgcaatgatcactacaaggagctgaagcccatcatcgatcg
gatctacaagacctatgccgaccagtgcctgcagctggtgcagctggattgggagaacctgagcgccgccatcga
ctcctatagaaaggagaaaaccgaggagacaaggaacgccctgatcgaggagcaggccacatatcgcaatgccat
ccacgactacttcatcggccggacagacaacctgaccgatgccatcaataagagacacgccgagatctacaaggg
cctgttcaaggccgagctgtttaatggcaaggtgctgaagcagctgggcaccgtgaccacaaccgagcacgagaa
cgccctgctgcggagcttcgacaagtttacaacctacttctccggcttttatagaaacaggaagaacgtgttcag
cgccgaggatatcagcacagccatcccacaccgcatcgtgcaggacaacttccccaagtttaaggagaattgtca
catcttcacacgcctgatcaccgccgtgcccagcctgcgggagcactttgagaacgtgaagaaggccatcggcat
cttcgtgagcacctccatcgaggaggtgttttccttccctttttataaccagctgctgacacagacccagatcga
cctgtataaccagctgctgggaggaatctctcgggaggcaggcaccgagaagatcaagggcctgaacgaggtgct
gaatctggccatccagaagaatgatgagacagcccacatcatcgcctccctgccacacagattcatccccctgtt
taagcagatcctgtccgataggaacaccctgtctttcatcctggaggagtttaagagcgacgaggaagtgatcca
gtccttctgcaagtacaagacactgctgagaaacgagaacgtgctggagacagccgaggccctgtttaacgagct
gaacagcatcgacctgacacacatcttcatcagccacaagaagctggagacaatcagcagcgccctgtgcgacca
ctgggatacactgaggaatgccctgtatgagcggagaatctccgagctgacaggcaagatcaccaagtctgccaa
ggagaaggtgcagcgcagcctgaagcacgaggatatcaacctgcaggagatcatctctgccgcaggcaaggagct
gagcgaggccttcaagcagaaaaccagcgagatcctgtcccacgcacacgccgccctggatcagccactgcctac
aaccctgaagaagcaggaggagaaggagatcctgaagtctcagctggacagcctgctgggcctgtaccacctgct
ggactggtttgccgtggatgagtccaacgaggtggaccccgagttctctgcccggctgaccggcatcaagctgga
gatggagccttctctgagcttctacaacaaggccagaaattatgccaccaagaagccctactccgtggagaagtt
caagctgaactttcagatgcctacactggccagaggctgggacgtgaatgtggagaagaacagaggcgccatcct
gtttgtgaagaacggcctgtactatctgggcatcatgccaaagcagaagggcaggtataaggccctgagcttcga
gcccacagagaaaaccagcgagggctttgataagatgtactatgactacttccctgatgccgccaagatgatccc
aaagtgcagcacccagctgaaggccgtgacagcccactttcagacccacacaacccccatcctgctgtccaacaa
tttcatcgagcctctggagatcacaaaggagatctacgacctgaacaatcctgagaaggagccaaagaagtttca
gacagcctacgccaagaaaaccggcgaccagaagggctacagagaggccctgtgcaagtggatcgacttcacaag
ggattttctgtccaagtataccaagacaacctctatcgatctgtctagcctgcggccatcctctcagtataagga
cctgggcgagtactatgccgagctgaatcccctgctgtaccacatcagcttccagagaatcgccgagaaggagat
catggatgccgtggagacaggcaagctgtacctgttccagatctataacaaggactttgccaagggccaccacgg
caagcctaatctgcacacactgtattggaccggcctgttttctccagagaacctggccaagacaagcatcaagct
gaatggccaggccgagctgttctaccgccctaagtccaggatgaagaggatggcacaccggctgggagagaagat
gctgaacaagaagctgaaggatcagaaaaccccaatccccgacaccctgtaccaggagctgtacgactatgtgaa
tcacagactgtcccacgacctgtctgatgaggccagggccctgctgcccaacgtgatcaccaaggaggtgtctca
cgagatcatcaaggataggcgctttaccagcgacaagttctttttccacgtgcctatcacactgaactatcaggc
cgccaattccccatctaagttcaaccagagggtgaatgcctacctgaaggagcaccccgagacacctatcatcgg
catcgcccggggcgagagaaacctgatctatatcacagtgatcgactccaccggcaagatcctggagcagcggag
cctgaacaccatccagcagtttgattaccagaagaagctggacaacagggagaaggagagggtggcagcaaggca
ggcctggtctgtggtgggcacaatcaaggatctgaagcagggctatctgagccaggtcatccacgagatcgtgga
cctgatgatccactaccaggccgtggtggtgctggagaacctgaatttcggctttaagagcaagaggaccggcat
cgccgagaaggccgtgtaccagcagttcgagaagatgctgatcgataagctgaattgcctggtgctgaaggacta
tccagcagagaaagtgggaggcgtgctgaacccataccagctgacagaccagttcacctcctttgccaagatggg
cacccagtctggcttcctgttttacgtgcctgccccatatacatctaagatcgatcccctgaccggcttcgtgga
ccccttcgtgtggaaaaccatcaagaatcacgagagccgcaagcacttcctggagggcttcgactttctgcacta
cgacgtgaaaaccggcgacttcatcctgcactttaagatgaacagaaatctgtccttccagaggggcctgcccgg
ctttatgcctgcatgggatatcgtgttcgagaagaacgagacacagtttgacgccaagggcacccctttcatcgc
cggcaagagaatcgtgccagtgatcgagaatcacagattcaccggcagataccgggacctgtatcctgccaacga
gctgatcgccctgctggaggagaagggcatcgtgttcagggatggctccaacatcctgccaaagctgctggagaa
tgacgattctcacgccatcgacaccatggtggccctgatccgcagcgtgctgcagatgcggaactccaatgccgc
cacaggcgaggactatatcaacagccccgtgcgcgatctgaatggcgtgtgcttcgactcccggtttcagaaccc
agagtggcccatggacgccgatgccaatggcgcctaccacatcgccctgaagggccagctgctgctgaatcacct
gaaggagagcaaggatctgaagctgcagaacggcatctccaatcaggactggctggcctacatccaggagctgcg
caacaaaaggccggcggccacgaaaaaggccggccaggcaaaaaagaaaaagggatcctacccatacgatgttcc
agattacgcttatccctacgacgtgcctgattatgcatacccatatgatgtccccgactatgccgagggcagagg
aagtctgctaacatgcggtgacgtcgaggagaatcctggcccaatgaccgagtacaagcccacggtgcgcctcgc
cacccgcgacgacgtccccagggccgtacgcaccctcgccgccgcgttcgccgactaccccgccacgcgccacac
cgtcgatccggaccgccacatcgagcgggtcaccgagctgcaagaactcttcctcacgcgcgtcgggctcgacat
cggcaaggtgtgggtcgcggacgacggcgccgcggtggcggtctggaccacgccggagagcgtcgaagcgggggc
ggtgttcgccgagatcggcccgcgcatggccgagttgagcggttcccggctggccgcgcagcaacagatggaagg
cctcctggcgccgcaccggcccaaggagcccgcgtggttcctggccaccgtcggagtctcgcccgaccaccaggg
caagggtctgggcagcgccgtcgtgctccccggagtggaggcggccgagcgcgccggggtgcccgccttcctgga
gacctccgcgccccgcaacctccccttctacgagcggctcggcttcaccgtcaccgccgacgtcgaggtgcccga
aggaccgcgcacctggtgcatgacccgcaagcccggtgcctgaactagtcctgcaggcatgcaagcttgatatca
agcttatcgataatcaacctctggattacaaaatttgtgaaagattgactggtattcttaactatgttgctcctt
ttacgctatgtggatacgctgctttaatgcctttgtatcatgctattgcttcccgtatggctttcattttctcct
ccttgtataaatcctggttgctgtctctttatgaggagttgtggcccgttgtcaggcaacgtggcgtggtgtgca
ctgtgtttgctgacgcaacccccactggttggggcattgccaccacctgtcagctcctttccgggactttcgctt
tccccctccctattgccacggcggaactcatcgccgcctgccttgcccgctgctggacaggggctcggctgttgg
gcactgacaattccgtggtgttgtcggggaaatcatcgtcctttccttggctgctcgcctgtgttgccacctgga
ttctgcgcgggacgtccttctgctacgtcccttcggccctcaatccagcggaccttccttcccgcggcctgctgc
cggctctgcggcctcttccgcgtcttcgccttcgccctcagacgagtcggatctccctttgggccgcctccccgc
atcgataccgtcgacctcgagggaattaattcgagctcggtacctttaagaccgatgacttacaaggcagctgta
gatcttagccactttttaaaagaaattaactgtgccttctagttgccagccatctgttgtttgcccctcccccgt
gccttccttgaccctggaaggtgccactcccactgtcctttcctaataaaatgaggaaattgcatcgcattgtct
gagtaggtgtcattctattctggggggtggggtggggcaggacagcaagggggaggattgggaagagaatagcag
gcatgctggggagcggccgcaggaacccctagtgatggagttggccactccctctctgcgcgctcgctcgctcac
tgaggccgggcgaccaaaggtcgcccgacgcccgggctttgcccgggcggcctcagtgagcgagcgagcgcgcag
ctgcctgcaggggcgcctgatgcggtattttctccttacgcatctgtgcggtatttcacaccgcatacgtcaaag
caaccatagtacgcgccctgtagcggcgcattaagcgcggcgggtgtggtggttacgcgcagcgtgaccgctaca
cttgccagcgccttagcgcccgctcctttcgctttcttcccttcctttctcgccacgttcgccggctttccccgt
caagctctaaatcgggggctccctttagggttccgatttagtgctttacggcacctcgaccccaaaaaacttgat
ttgggtgatggttcacgtagtgggccatcgccctgatagacggtttttcgccctttgacgttggagtccacgttc
tttaatagtggactcttgttccaaactggaacaacactcaactctatctcgggctattcttttgatttataaggg
attttgccgatttcggtctattggttaaaaaatgagctgatttaacaaaaatttaacgcgaattttaacaaaata
ttaacgtttacaattttatggtgcactctcagtacaatctgctctgatgccgcatagttaagccagccccgacac
ccgccaacacccgctgacgcgccctgacgggcttgtctgctcccggcatccgcttacagacaagctgtgaccgtc
tccgggagctgcatgtgtcagaggttttcaccgtcatcaccgaaacgcgcgagacgaaagggcctcgtgatacgc
ctatttttataggttaatgtcatgataataatggtttcttagacgtcaggtggcacttttcggggaaatgtgcgc
ggaacccctatttgtttatttttctaaatacattcaaatatgtatccgctcatgagacaataaccctgataaatg
cttcaataatattgaaaaaggaagagtatgagtattcaacatttccgtgtcgcccttattcccttttttgcggca
ttttgccttcctgtttttgctcacccagaaacgctggtgaaagtaaaagatgctgaagatcagttgggtgcacga
gtgggttacatcgaactggatctcaacagcggtaagatccttgagagttttcgccccgaagaacgttttccaatg
atgagcacttttaaagttctgctatgtggcgcggtattatcccgtattgacgccgggcaagagcaactcggtcgc
cgcatacactattctcagaatgacttggttgagtactcaccagtcacagaaaagcatcttacggatggcatgaca
gtaagagaattatgcagtgctgccataaccatgagtgataacactgcggccaacttacttctgacaacgatcgga
ggaccgaaggagctaaccgcttttttgcacaacatgggggatcatgtaactcgccttgatcgttgggaaccggag
ctgaatgaagccataccaaacgacgagcgtgacaccacgatgcctgtagcaatggcaacaacgttgcgcaaacta
ttaactggcgaactacttactctagcttcccggcaacaattaatagactggatggaggcggataaagttgcagga
ccacttctgcgctcggcccttccggctggctggtttattgctgataaatctggagccggtgagcgtggaagccgc
ggtatcattgcagcactggggccagatggtaagccctcccgtatcgtagttatctacacgacggggagtcaggca
actatggatgaacgaaatagacagatcgctgagataggtgcctcactgattaagcattggtaactgtcagaccaa
gtttactcatatatactttagattgatttaaaacttcatttttaatttaaaaggatctaggtgaagatccttttt
gataatctcatgaccaaaatcccttaacgtgagttttcgttccactgagcgtcagaccccgtagaaaagatcaaa
ggatcttcttgagatcctttttttctgcgcgtaatctgctgcttgcaaacaaaaaaaccaccgctaccagcggtg
gtttgtttgccggatcaagagctaccaactctttttccgaaggtaactggcttcagcagagcgcagataccaaat
actgttcttctagtgtagccgtagttaggccaccacttcaagaactctgtagcaccgcctacatacctcgctctg
ctaatcctgttaccagtggctgctgccagtggcgataagtcgtgtcttaccgggttggactcaagacgatagtta
ccggataaggcgcagcggtcgggctgaacggggggttcgtgcacacagcccagcttggagcgaacgacctacacc
gaactgagatacctacagcgtgagctatgagaaagcgccacgcttcccgaagggagaaaggcggacaggtatccg
gtaagcggcagggtcggaacaggagagcgcacgagggagcttccagggggaaacgcctggtatctttatagtcct
gtcgggtttcgccacctctgacttgagcgtcgatttttgtgatgctcgtcaggggggcggagcctatggaaaaac
gccagcaacgcggcctttttacggttcctggccttttgctggccttttgctcacatgt

Some of the embodiments, advantages, features, and uses of the technology disclosed herein will be more fully understood from the Examples below. The Examples are intended to illustrate some of the benefits of the present disclosure and to describe particular embodiments but are not intended to exemplify the full scope of the disclosure and, accordingly, do not limit the scope of the disclosure.

### EXAMPLES

### Example 1. Generation of Genetically Engineered Hematopoietic Cells Using Fusion Polypeptides, Including Base Editing Fusion Polypeptides

This example demonstrates generation of fusion polypeptides and their use in generating genetically engineered cells, such as genetically engineered hematopoietic cells. Cas12a/Cpf1 gRNAs are synthesized using gRNA target domains directed to target sequences of interest.

Peripheral blood mononuclear cells are collected from healthy donor subject by apheresis following hematopoietic stem cell mobilization. Alternatively, frozen CD34+ HSCs derived from mobilized peripheral blood (mPB) are purchased, for example, from Hemacare or Fred Hutchinson Cancer Center and thawed according to manufacturer's instructions. ˜1×10⁶ HSCs are thawed and cultured in StemSpan SFEM medium supplemented with StemSpan CC110 cocktail (StemCell Technologies) for 24-48 h before electroporation with RNP.

The donor or purchased CD34+ cells are electroporated with the fusion polypeptide and gRNAs targeting a targeting sequence of interest. To electroporate HSCs, 1.5×10⁵ cells are pelleted and resuspended in 20 μL Lonza P3 solution and mixed with 10 μL RNP comprising the fusion polypeptides and gRNA. CD34+ HSCs are electroporated using the Lonza Nucleofector 2 and the Human P3 Cell Nucleofection Kit (VPA-1002, Lonza).

The edited cells are cultured for less than 48 hours. Upon harvest, the cells are washed, resuspended in the final formulation, and cryopreserved.

A representative sample of the edited HSCs (e.g., a portion of or all cells of the time point aliquots) is evaluated for viability, editing efficiency at the target sequence, and/or expression of exemplary target region genes, or absence thereof, by staining using target-specific antibody and analyzed by flow cytometry. Edited HSC populations may also be assessed for development and differentiation into particular cell types, such as macrophages, T cells, B cells, and myeloid cells.

### Genomic DNA Analysis

For all genomic analysis, DNA is harvested from cells, amplified with primers flanking the target region, purified and the allele modification frequencies are analyzed using appropriate methods known in the art. Analyses are performed using a reference sequence from a mock-transfected sample.

### Flow Cytometry Analysis

The gRNA-edited cells may also be evaluated for surface expression of target gene encoded protein, for example by flow cytometry analysis (FACS). Live CD34+ HSCs are stained for target gene protein using a target-specific antibody and analyzed by flow cytometry on the Attune NxT flow cytometer (Life Technologies).

### Viability of Edited Cells

At 0, 0.5, 1, 2, 3, 4, 5, 6, 7, 8, 9, 10, 11, 12, 15, 18, 21, 24, 27, 30, 33, 36, 39, 42, 45, and/or 48 hours post-ex vivo editing (e.g., 4, 24, and 48 hours post-ex vivo editing), the percentages of viable, edited cells, and control cells are quantified using flow cytometry and the 7AAD viability dye. Cells edited using the exemplary gRNAs or sgRNAs described herein may be viable and remain viable over time following electroporation and gene editing. This is similar to what is observed in the control mock edited cells.

### Example 2: Assessing Genetic Editing In Vitro

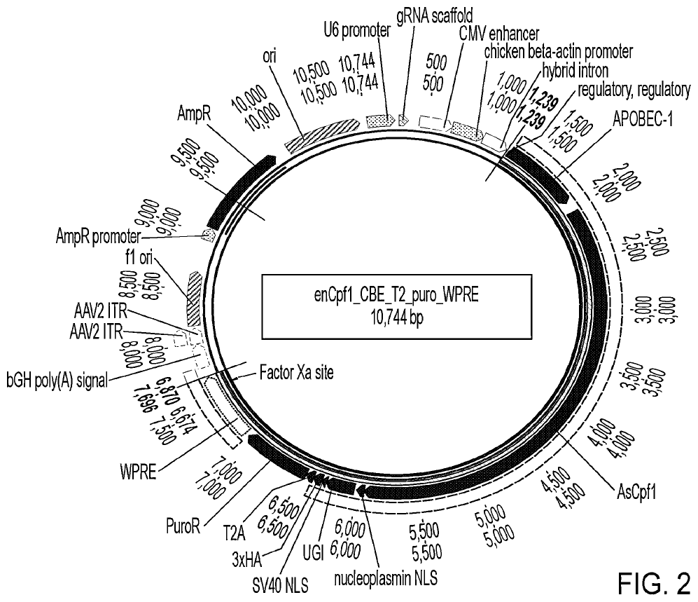

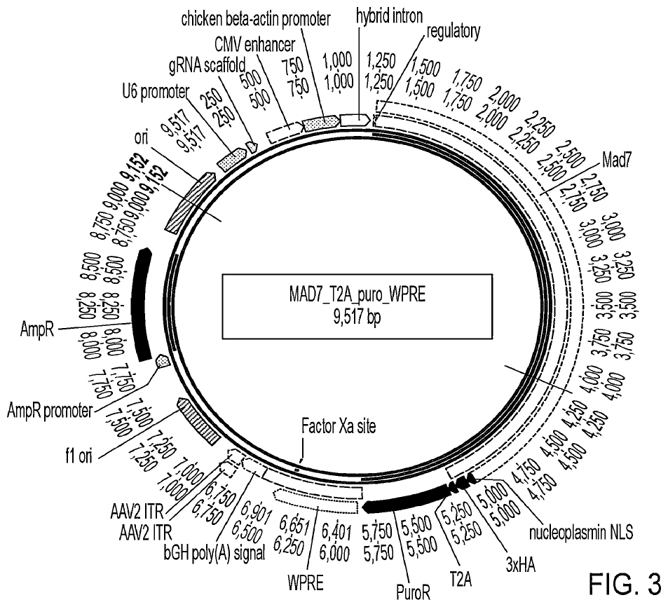

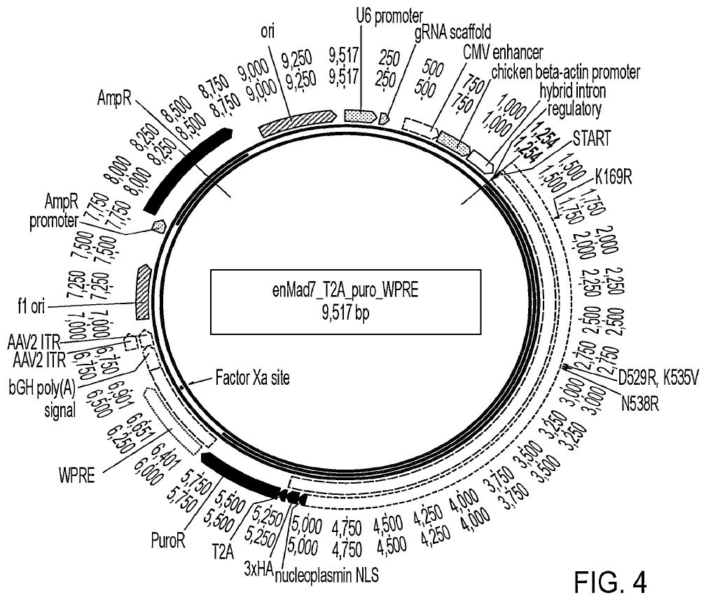

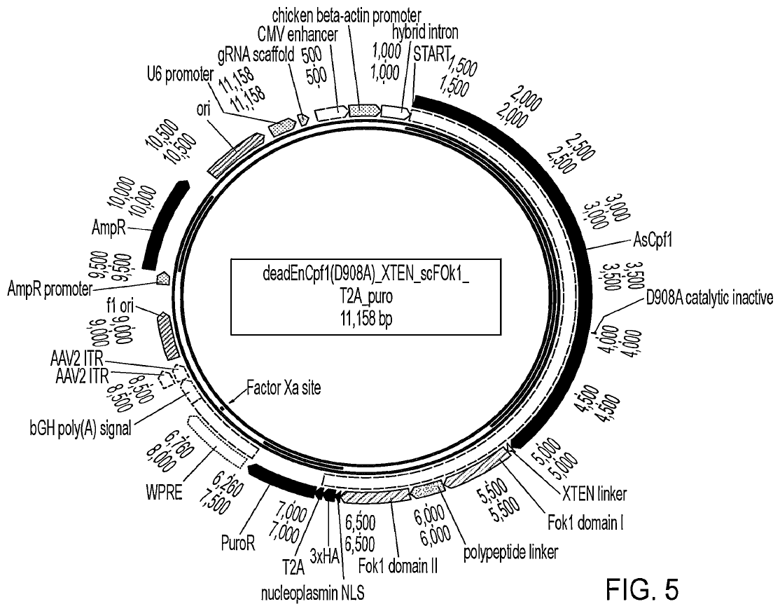

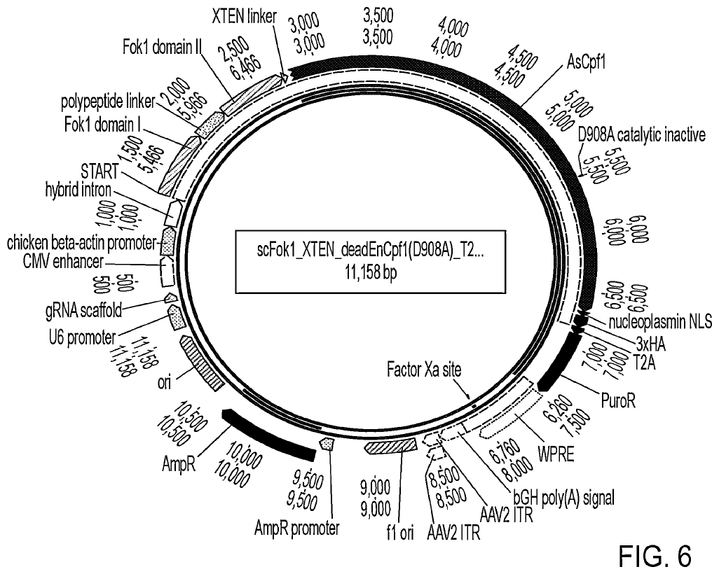

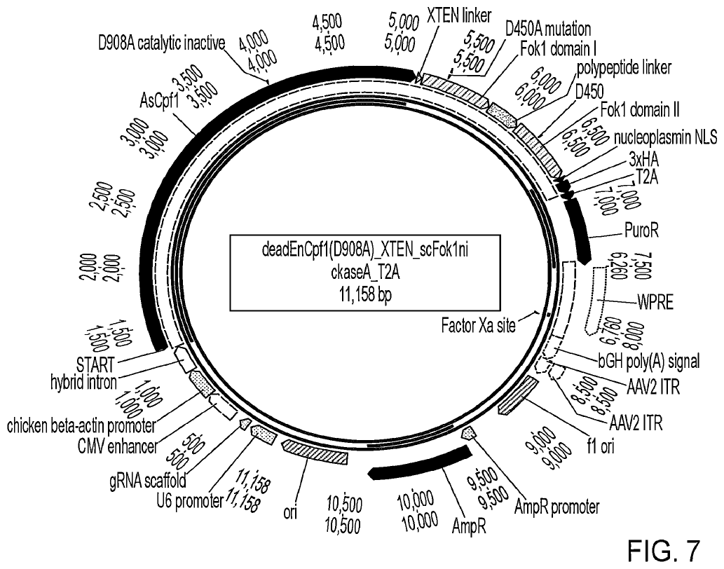

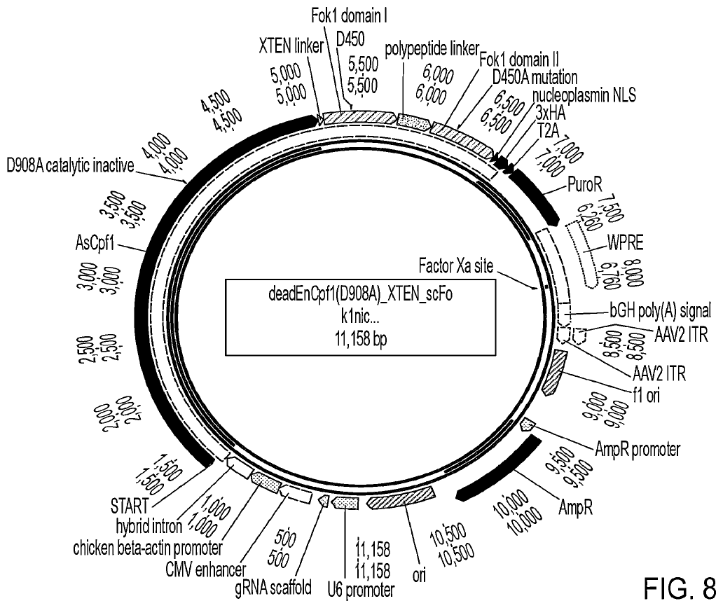

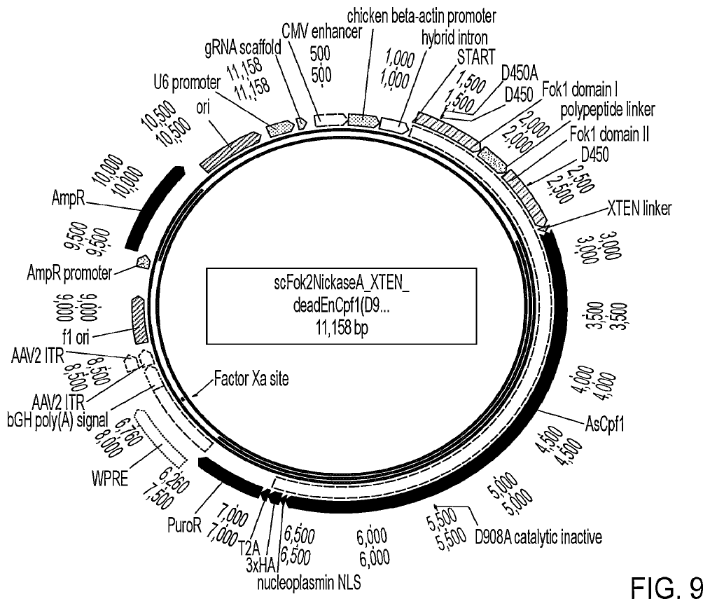

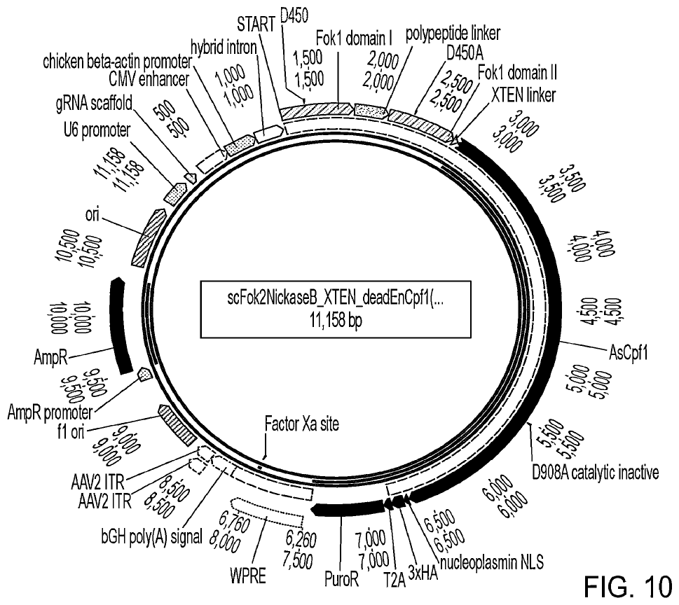

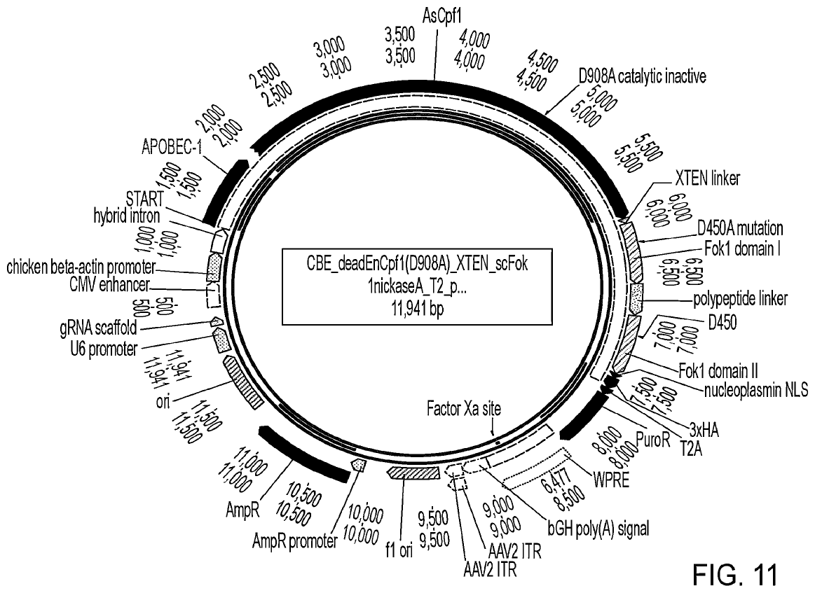

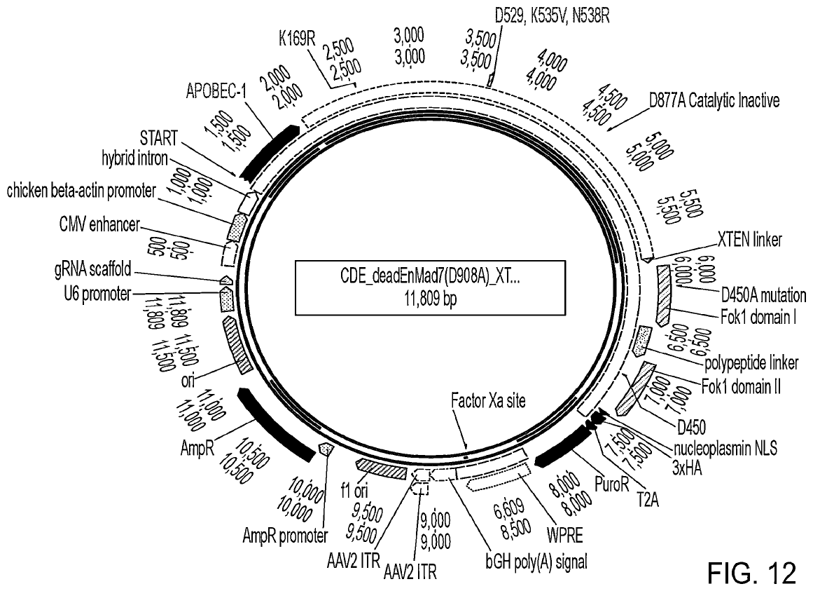

To assess the ability of fusion polypeptides described herein to effect targeted DNA modifications in cultured cells, fusion polypeptides, such as any of the fusion polypeptides described herein (e.g., such as those shown in the plasmids shown in FIGS. 1-12 or provided by any of SEQ ID NOs: 24-31, and gRNAs targeting a desired genetic locus are introduced into appropriate cultured cell populations, such as a cell line or cells sourced from subject, including healthy subjects or patient populations of interest. In some experiments, expression plasmids encoding the fusion protein and gRNAs, such as the plasmids shown in FIGS. 1-12, are generated and used to produce the fusion polypeptides.

The fusion polypeptides may be incubated with gRNAs to form a ribonucleoprotein (RNP) complex, and then used to transfect host cells.

After sufficient time, i.e., 48, 72, or 120 hours after electroporation, genomic DNA is extracted from edited cells and from control (non-edited) cells. Sequencing, such as Sanger sequencing of a target sequence, whole genome sequencing, may be performed to assess the efficiency of genomic modification and determine any off-target editing.

### Example 3: Treating a Subject with Edited Cells

An example treatment regimen using the methods, cells, and agents described herein for acute myeloid leukemia is provided below.
1) Identify a patient with AML who is a candidate for receiving a hematopoietic cell transplant (HCT);
2) Identify a HCT donor with matched HLA haplotypes, using standard methods and techniques;
3) Extract the bone marrow from the donor;
4) Genetically manipulate the donor bone marrow cells ex vivo. Briefly, introduce targeted modifications (deletion, substitution) of a lineage-specific cell-surface antigen using a gRNA and any of the fusion polypeptides described herein. Cells may be evaluated for characteristics to determine their ability to differentiate and the ability to engraft the patient and mediate graft-vs-tumor (GVT) effects.

### Optional Steps 5-7:

In some embodiments, Steps 5-7 provided below may be performed (once or multiple times) in an exemplary treatment method as described herein:
5) Pre-condition the AML patient using standard techniques, such as infusion of chemotherapy agents (e.g., etoposide, cyclophosphamide) and/or irradiation;
6) Administer the engineered donor bone marrow to the AML patient, allowing for successful engraftment;
7) Follow up with a cytotoxic agent, such as immune cells expressing a chimeric receptor (e.g., CAR T cell) or antibody-drug conjugate, wherein the epitope to which the cytotoxic agent binds is the same epitope that was modified and is no longer present on the engineered cells. The targeted therapy should thus specifically target the antigen, without simultaneously eliminating the bone marrow graft, in which the epitope is not present.

### Optional Steps 8-10:

In some embodiments, Steps 8-10 may be performed (once or multiple times) in an exemplary treatment method as described herein:
8) Administer a cytotoxic agent, such as immune cells expressing a chimeric receptor (e.g., CAR T cell) or antibody-drug conjugate that targets an epitope of an antigen. This targeted therapy would be expected to eliminate both cancerous cells as well as the patient's non-cancerous cells;
9) Pre-condition the AML patient using standard techniques, such as infusion of chemotherapy agents;
10) Administer the engineered donor bone marrow to the AML patient, allowing for successful engraftment.

The steps 8-10 result in the elimination of the patient's cancerous and normal cells expressing the targeted protein, while replenishing the normal cell population with donor cells that are resistant to the targeted therapy.

### REFERENCES

All publications, patents, patent applications, publication, and database entries (e.g., sequence database entries) mentioned herein, e.g., in the Background, Summary, Detailed Description, Examples, and/or References sections, are hereby incorporated by reference in their entirety as if each individual publication, patent, patent application, publication, and database entry was specifically and individually incorporated herein by reference. In case of conflict, the present application, including any definitions herein, will control.

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the invention encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods described herein, can be excluded from any one or more claims. For purposes of brevity, all of the embodiments in which one or more elements, features, purposes, or aspects is excluded are not set forth explicitly herein.

What is claimed is:

1. A fusion polypeptide comprising:
a) a Cpf1 domain that lacks nuclease activity, and
b) an endonuclease domain.

2. The fusion polypeptide of claim 1, wherein the endonuclease domain comprises a first DNA-cleavage domain of a restriction endonuclease, wherein the first DNA-cleavage domain is capable of forming a dimer with a second DNA-cleavage domain of a restriction endonuclease.

3. The fusion polypeptide of claim 1, wherein the endonuclease domain comprises a first DNA-cleavage domain of a restriction endonuclease and a second DNA-cleavage domain of a restriction endonuclease, wherein the first DNA-cleavage domain and second DNA-cleavage domain are capable of forming a dimer with one another.

4. The fusion polypeptide of claim 2 or 3, wherein the dimer of the first and second DNA-cleavage domain is capable of producing a single strand break in DNA.

5. The fusion polypeptide of any one of claims 2-4, wherein the restriction endonuclease is a type IIS restriction endonuclease or portion thereof.

6. The fusion polypeptide of any one of claims 1-5, wherein the endonuclease domain comprises FokI or a portion thereof.

7. The fusion polypeptide of any one of claims 2-6, wherein the first and/or second DNA-cleavage domain is a DNA cleavage domain of FokI or derived therefrom.

8. The fusion polypeptide of any one of claims 1-7, wherein the endonuclease domain does not comprise the DNA binding domain of FokI and/or is not capable of forming and/or maintaining a complex with DNA in the absence of an accompanying Cpf1 domain.

9. The fusion polypeptide of any one of claims 2-8, wherein the first DNA-cleavage domain or the second DNA-cleavage domain comprises one or more modifications relative to a corresponding wildtype sequence.

10. The fusion polypeptide of claim 9, wherein the one or more modifications alter activity of the endonuclease domain such that the endonuclease domain does not produce double strand breaks in DNA.

11. The fusion polypeptide of claim 9 or 10, wherein the one or more modifications decrease or eliminate endonuclease activity of the endonuclease domain.

12. The fusion polypeptide of any one of claims 1-11, wherein the endonuclease domain comprises an amino acid sequence of any one of SEQ ID NOs: 13 or 14, or a sequence with at least 80, 85, 90, 95, or 99% identity to any thereof.

13. The fusion polypeptide of any one of claims 1-12, wherein the Cpf1 domain comprises an amino acid sequence of a Cpf1 protein from Prevotella spp., Francisella spp., Acidaminococcus sp. (AsCpf1), Lachnospiraceae bacterium (LpCpf1), Eubacterium rectale, or an engineered Cpf1.

14. The fusion polypeptide of any one of claims 1-13, wherein the Cpf1 domain comprises one or more amino acid modifications relative to a corresponding wildtype Cpf1 amino acid sequence.

15. The fusion polypeptide of claim 14, wherein the one or more modifications comprise one or more amino acid substitutions in the Cpf1 protein relative to the wildtype sequence.

16. The fusion polypeptide of claim 15, wherein the Cpf1 domain comprises a substitution at:
one, two, three, or each of amino acids corresponding to positions 174, 542, 548, or 552 of the Acidaminococcus sp. Cpf1 amino acid sequence; and/or
one, two, three, or each of amino acids corresponding to positions 169, 529, 535, or 538 of the MAD7™ Cpf1 amino acid sequence provided by SEQ ID NO: 1.

17. The fusion polypeptide of claim 16, wherein the one or more substitutions comprise an arginine at the position corresponding to position 174, an arginine at the position corresponding to position 542, a valine at the position corresponding to position 548, and/or an arginine at the position corresponding to position 552 of the Acidaminococcus sp. Cpf1 amino acid sequence provided by SEQ ID NO: 4.

18. The fusion polypeptide of claim 16 or 17, wherein the one or more substitutions comprise an arginine at the position corresponding to position 169, an arginine at the position corresponding to position 529, a valine at the position corresponding to position 535, and/or an arginine at the position corresponding to position 538 of the MAD7™ Cpf1 amino acid sequence provided by SEQ ID NO: 1.

19. The fusion polypeptide of any one of claims 1-18, further comprising c) a genomic modification domain.

20. The fusion polypeptide of claim 19, wherein the genomic modification domain comprises a base editor.

21. The fusion polypeptide of claim 20, wherein the base editor is a cytosine base editor (CBE) or an adenine base editor (ABE).

22. The fusion polypeptide of claim 20 or 21, wherein the base editor comprises a cytidine deaminase or an adenine deaminase.

23. The fusion polypeptide of claim 20, wherein the base editor comprises both a cytidine deaminase and an adenine deaminase.

24. The fusion polypeptide of any one of claims 19-23, wherein the genomic modification domain comprises an epigenetic modifier.

25. The fusion polypeptide of claim 24, wherein the epigenetic modifier comprises a DNA methyltransferase, a DNA methylase, a histone acetyltransferase, a histone deacetylase, a histone methyltransferase, a histone methylase, or a functional portion or combination of any thereof.

26. The fusion polypeptide of any one of claims 19-25, wherein the genomic modification domain comprises an amino acid sequence of SEQ ID NO: 15, or a sequence with at least 80, 85, 90, 95, or 99% identity to any thereof.

27. The fusion polypeptide of any one of claims 1-26, wherein the Cpf1 domain is N-terminal of the endonuclease domain.

28. The fusion polypeptide of any one of claims 1-26, wherein the endonuclease domain is N-terminal of the Cpf1 domain.

29. The fusion polypeptide of any one of claims 19-27, wherein the genomic modification domain is N-terminal of the Cpf1 domain.

30. The fusion polypeptide of any one of claims 19-29, wherein the genomic modification domain is N-terminal of the endonuclease domain.

31. The fusion polypeptide of any one of claims 19-27 or 30, wherein the fusion comprises from N-terminus to C-terminus: the Cpf1 domain, the endonuclease domain, and the genomic modification domain.

32. The fusion polypeptide of any one of claims 19-27 or 30, wherein the fusion comprises from N-terminus to C-terminus: the Cpf1 domain, the genomic modification domain, and the endonuclease domain.

33. The fusion polypeptide of any one of claims 19-26 or 28, wherein the fusion comprises from N-terminus to C-terminus: the endonuclease domain, the Cpf1 domain, and the genomic modification domain.

34. The fusion polypeptide of any one of claims 19-26, 28, or 29, wherein the fusion comprises from N-terminus to C-terminus: the endonuclease domain, the genomic modification domain, and the Cpf1 domain.

35. The fusion polypeptide of any one of claims 19-27, 29 or 30, wherein the fusion comprises from N-terminus to C-terminus: the genomic modification domain, the Cpf1 domain, and the endonuclease domain.

36. The fusion polypeptide of any one of claims 19-26, or 28-30, wherein the fusion comprises from N-terminus to C-terminus: the genomic modification domain, the endonuclease domain, and the Cpf1 domain.

37. The fusion polypeptide of any one of claims 1-36, further comprising one or more linker domains.

38. The fusion polypeptide of claim 37, wherein the linker is an XTEN linker.

39. A nucleic acid comprising a nucleotide sequence encoding the fusion polypeptide of any one of claims 1-38.

40. A vector comprising the nucleic acid of claim 39.

41. A cell comprising the fusion polypeptide of any one of claims 1-38, the nucleic acid of claim 39, or vector of claim 40.

42. A system comprising:
the fusion polypeptide of any one of claims 1-38; and
a first gRNA comprising a targeting domain complementary to a first target sequence in the genome of a cell,
wherein the fusion polypeptide is capable of forming and/or maintaining a ribonucleoprotein (RNP) complex with the first gRNA and the RNP complex is capable of binding the target sequence in the genome of a cell.

43. The system of claim 42, further comprising a second gRNA comprising a targeting domain complementary to a second target sequence in the genome of the cell, wherein the first and second target sequences are not the same.

44. The system of claim 42, further comprising a second fusion polypeptide comprising
a) a Cpf1 domain that lacks nuclease activity, and
b) a second endonuclease domain capable of forming a dimer with the first endonuclease domain.

45. A ribonucleoprotein (RNP) complex comprising:
the fusion polypeptide of any one of claims 1-38; and
a gRNA comprising a targeting domain complementary to a target sequence in the genome of a cell,
wherein RNP complex is capable of binding the target sequence in the genome of a cell.

46. A method, comprising:
i) contacting a cell with the fusion polypeptide of any one of claims 1-38 or the nucleic acid of claim 39; and
ii) contacting the cell with a first gRNA comprising a targeting domain complementary to a first target sequence in the genome of a cell.

47. The method of claim 46, wherein i) and ii) occur simultaneously or in close temporal proximity.

48. The method of claim 46 or 47, further comprising:
iii) contacting the cell with a second gRNA (or nucleic acid encoding the same) comprising a targeting domain complementary to a second target sequence in the genome of a cell.

49. The method of claim 48, further comprising contacting the cell with a second fusion protein of any one of claims 1-38 or the nucleic acid of claim 39.

50. A method, comprising:
i) contacting a cell with a first fusion polypeptide of any one of claims 1-38 and a first gRNA comprising a targeting domain complementary to a first target sequence in the genome of a cell; and
ii) contacting the cell with a second fusion polypeptide of any of claims 1-38 and a second gRNA comprising a targeting domain complementary to a second target sequence in the genome of a cell,
wherein the first target sequence and the second target sequence are not the same and the first fusion polypeptide and second fusion polypeptide are not the same.

51. The method of any one of claims 48-50, wherein the first target sequence and the second target sequence are on different chromosomes of the genome of the cell.

52. The method of any one of claims 48-50, wherein the first target sequence and the second target sequence are on the same chromosome in the genome of the cell.

53. The method of claim 52, wherein the first target sequence and the second target sequence are on the same DNA strand of the chromosome.

54. The method of claim 52, wherein the first target sequence and the second target sequence are on different DNA strands of the chromosome.

55. The method of any one of claims 48-54, wherein the first target sequence and the second target sequence are separated by 10-10,000 nucleotides.

56. The method of any one of claims 46-55, wherein the cell is a hematopoietic cell.

57. The method of any one of claims 46-55, wherein the cell is a hematopoietic stem cell.

58. The method of any one of claims 46-57, wherein the cell is a hematopoietic progenitor cell.

59. The method of any one of claims 46-55, wherein the cell is an immune effector cell.

60. The method of any one of claims 46-55 or 59, wherein the cell is a lymphocyte.

61. The method of any one of claims 46-55, 59, or 60, wherein the cell is a T-lymphocyte.

62. An engineered cell, or descendant thereof, produced by a method of any one of claims 46-61.

63. A cell population, comprising the genetically engineered cell of claim 62.

64. A chimeric polypeptide that lacks nuclease activity, comprising:
a first portion comprising an amino acid sequence of a first Cpf1 protein, and
a second portion comprising an amino acid sequence of a second Cpf1 protein,
wherein the first Cpf1 protein and second Cpf1 protein are not the same.

65. The chimeric polypeptide of claim 64, wherein the first Cpf1 protein is derived from a Cpf1 from Prevotella spp., Francisella spp., Acidaminococcus sp. (AsCpf1), Lachnospiraceae bacterium (LpCpf1), or Eubacterium rectale, or MAD7™ as provided by Inscripta.

66. The chimeric polypeptide of claim 64 or 65, wherein the second Cpf1 protein is derived from a Cpf1 from Prevotella spp., Francisella spp., Acidaminococcus sp. (AsCpf1), Lachnospiraceae bacterium (LpCpf1), or Eubacterium rectale, or MAD7™ as provided by Inscripta.

67. The chimeric polypeptide of any one of claims 64-66, wherein the first Cpf1 protein comprises an Acidaminococcus sp. Cpf1 (AsCpf1) or portion thereof.

68. The chimeric polypeptide of any one of claims 64-67, wherein the second Cpf1 protein comprises MAD7™ or a portion thereof.

69. The chimeric polypeptide of any one of claims 64-68, wherein the first Cpf1 protein and/or second Cpf1 protein comprise one or more modifications relative to the wildtype sequence of the first Cpf1 protein and/or second Cpf1 protein.

70. The chimeric polypeptide of claim 69, wherein the one or more modifications comprise one or more amino acid substitutions in the first Cpf1 protein relative to the wildtype sequence of the first Cpf1 protein.

71. The chimeric polypeptide of any one of claims 64-70, wherein the amino acid sequence comprising the first Cpf1 protein is at least 100 amino acids in length, or 100-1300 amino acids in length.

72. The chimeric polypeptide of any one of claims 64-71, wherein the amino acid sequence comprising the second Cpf1 protein is at least 100 amino acids in length, or 100-1300 amino acids in length.

73. The chimeric polypeptide of any one of claims 64-72, wherein the chimeric polypeptide further comprises a linker between the first portion and second portion.

74. The chimeric polypeptide of any one of claims 64-73, wherein the chimeric polypeptide is at least 800 amino acids in length, or 800-1500 amino acids in length.

75. The chimeric polypeptide of any one of claims 64-74, wherein the amino acid sequence of the first Cpf1 protein comprises any one of SEQ ID NOs: 1-9 or a sequence with at least 80, 85, 90, 95, or 99% identity to any thereof.

76. The chimeric polypeptide of any one of claims 64-75, wherein the amino acid sequence of the second Cpf1 protein comprises any one of SEQ ID NOs: 1-9 or a sequence with at least 80, 85, 90, 95, or 99% identity to any thereof.

77. The chimeric polypeptide of any one of claims 64-76, wherein the chimeric polypeptide comprises an amino acid sequence of any one of SEQ ID NOs: 24-31 or a sequence with at least 80, 85, 90, 95, or 99% identity to any thereof.


SEQUENCE LISTING (57 sequences)

SEQ ID NO: 1 - protein, 1263 aa, synthetic construct
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 2 - protein, 1263 aa, synthetic construct
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFRNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLARGWSKSVEYSRNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 3 - protein, 1263 aa, synthetic construct
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFRNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLARGWSKSVEYSRNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIARGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 4 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 5 - protein, 1306 aa, Acidaminococcus sp.
TQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYRNRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLARGWDVNVEKNRGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIARGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 6 - protein, 1306 aa, unidentified
  source 1..1306: note = Prevotella spp.
MAKNFEDFKRLYPLSKTLRFEAKPIGATLDNIVKSGLLEEDEHRAASYVKVKKLIDEYHKVFIDRVLDNGCLPLDDKGDNNSLAEYYESYVSKAQDEDAIKKFKEIQQNLLSIIAKKLTDDKAYANLFGNKLIESYKDKADKTKLIDSDLIQFINTAESTQLVSMSQDEAKELVKEFWGFTTYFEGFFKNRKNMYTPEEKSTGIAYRLINENLPKFIDNMEAFKKAIARPEIQANMEELYSNFSEYLNVESIQEMFLLDYYNMLLTQKQIDVYNAIIGGKTDDEHDVKIKGINEYINLYNQQHKDDKLPKLKALFKQILSDRNAISWLPEEFNSDQEVLNAIKDCYERLAENVLGDKVLKSLLGSLADYSLDGIFIRNDLQLTDISQKMFGNWGVIQNAIMQNIKHVAPARKHKESEEDYEKRIAGIFKKADSFSISYINDCLNEADPNNAYFVENYFATFGAVNTPTMQRENLFALVQNAYTEVAALLHSDYPTVKHLAQDKANVSKIKALLDAIKSLQHFVKPLLGKGDESDKDERFYGELASLWAELDTVTPLYNMIRNYMTRKPYSQKKIKLNFENPQLLGGWDANKEKDYATIILRRNGLYYLAIMDKDSRKLLGKAMPSDGECYEKMVYKFFKDVTTMIPKCSTQLKDVQAYFKVNTDDYVLNSKAFNRPLTITKEVFDLNNVLYGKYKKFQKGYLTATGDNVGYTHAVNVWIKFCMDFLDSYDSTCIYDFSSLKPESYLSLDSFYQDVNLLLYKLSFTDVSASFIDQLVEEGKMYLFQIYNKDFSEYSKGTPNMHTLYWKALFDERNLADVVYKLNGQAEMFYRKKSIENTHPTHPANHPILNKNKDNKKKESLFEYDLIKDRRYTVDKFMFHVPITMNFKSSGSENINQDVKAYLRHADDMHIIGIDRGERHLLYLVVIDLQGNIKEQFSLNEIVNDYNGNTYHTNYHDLLDVREDERLKARQSWQTIENIKELKEGYLSQVIHKITQLMVRYHAIVVLEDLSKGFMRSRQKVEKQVYQKFEKMLIDKLNYLVDKKTDVSTPGGLLNAYQLTCKSDSSQKLGKQSGFLFYIPAWNTSKIDPVTGFVNLLDTHSLNSKEKIKAFFSKFDAIRYNKDKKWFEFNLDYDKFGKKAEDTRTKWTLCTRGMRIDTFRNKEKNSQWDNQEVDLTTEMKSLLEHYYIDIHGNLKDAISTQTDKAFFTGLLHILKLTLQMRNSITGTETDYLVSPVADENGIFYDSRSCGDQLPENADANGAYNIARKGLMLVEQIKDAEDLDNVKFDISNKAWLNFAQQKPYKNG

SEQ ID NO: 7 - protein, 1300 aa, unidentified
  source 1..1300: note = Francisella spp.
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 8 - protein, 1282 aa, unidentified
  source 1..1282: note = Lachnospiraceae spp.
MNGNRIIVYREFVGVTPVAKTLRNELRPIGHTQEHIIHNGLIQEDELRQEKSTELKNIMDDYYREYIDKSLSGVTDLDFTLLFELMNLVQSSPSKDNKKALEKEQSKMREQICTHMQSDSNYKNIFNAKFLKEILPDFIKNYNQYDAKDKAGKLETLALFNGFSTYFTDFFEKRKNVFTKEAVSTSIAYRIVHENSLTFLANMTSYKKISEKALDEIEVIEKNNQDKMGDWELNQIFNPDFYNMVLIQSGIDFYNEICGVVNAHMNLYCQQTKNNYNLFKMRKLHKQILAYTSTSFEVPKMFEDDMSVYNAVNAFIDETEKGNIIGKLKDIVNKYDELDEKRIYISKDFYETLSCFMSGNWNLITGCVENFYDENIHAKGKSKEEKVKKAVKEDKYKSINDVNDLVEKYIDEKERNEFKNSNAKQYIREISNIITDTETAHLEYDEHISLIESEEKADEMKKRLDMYMNMYHWAKAFIVDEVLDRDEMFYSDIDDIYNILENIVPLYNRVRNYVTQKPYNSKKIKLNFQSPTLANGWSQSKEFDNNAIILIRDNKYYLAIFNAKNKPDKKIIQGNSDKKNDNDYKKMVYNLLPGANKMLPKVFLSKKGIETFKPSDYIISGYNAHKHIKTSENFDISFCRDLIDYFKNSIEKHAEWRKYEFKFSATDSYNDISEFYREVEMQGYRIDWTYISEADINKLDEEGKIYLFQIYNKDFAENSTGKENLHTMYFKNIFSEENLKDIIIKLNGQAELFYRRASVKNPVKHKKDSVLVNKTYKNQLDNGDVVRIPIPDDIYNEIYKMYNGYIKESDLSGAAKEYLDKVEVRTAQKEIVKDYRYTVDKYFIHTPITINYKVAARNNVNDMAVKYIAQNDDIHVIGIDRGERNLIYISVIDSHGNIVKQKSYNILNNYDYKKKLVEKEKTREYARKNWKSIGNIKELKEGYISGVVHEIAMLMVEYNAIIAMEDLNYGFKRGRFKVERQVYQKFESMLINKLNYFASKGKSVDEPGGLLKGYQLTYVPDNIKNLGKQCGVIFYVPAAFTSKIDPSTGFISAFNFKSISTNDSRKQFFMQFDEIRYCAEKDMFSFGFDYNNFDTYNITMGKTQWTVYTNGERLQSEFNNARRTGKTKSINLTETIKLLLEDNEINYADGHDVRIDMEKMDEDKNSEFFAQLLSLYKLTVQMRNSYTEAEEQEKGISYDKIISPVINDEGEFFDSDNYKESDDKECKMPKDADANGAYCIALKGLYEVLKIKSEWTEDGFDRNCLKLPHAEWLDFIQNKRYE

SEQ ID NO: 9 - protein, 1262 aa, Eubacterium rectale
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQAEKRKAIYKKFADDDRFKNMFSAKLISDILEFVIHNNNYSASEKEEKTQVIKLFSRFATSFKDYFKNRANCFSADDISSSSCHRIVNDNAEIFFSNALVYRRIVKNLSNDDINKISGDMKDSLKEMSLDEIYSYEKYGEFITQEGISFYNDICGKVNSFMNLYCQKNKENKNLYKLRKLHKQILCIADTSYEVPYKFESDEEVYQSVNGFLDNISSKHIVERLRKIGDNYNGYNLDKIYIVSRFYESVSQKTYRDWETINTALEIHYNNILPGNGKSKADKVKKAVKNDLQKSITEINELVSNYKLCPDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHLKSSKDFDITFCRDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKTIPENIYQELYKYFNDKSDKELSDEAAKLKNVVGHHEAATNIVKDYRYTYDKYFLHMPITINFKANKTSFINDRILQYIAKEKDLHVIGIDRGERNLIYVSVIDICGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSDKNLFCFTFDYNNFITQNTVMSKSSWSVYTYGVRIKRRFVNGRFSNESDTIDITKDMEKTLEMTDINWRDGHDLRQDIIDYEIVQHIFEIFKLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 10 - protein, 579 aa, synthetic construct
MVSKIRTFGWVQNPGKFENLKRVVQVFDRNSKVHNEVKNIKIPTLVKESKIQKELVAIMNQHDLIYTYKELVGTGTSIRSEAPCDAIIQATIADQGNKKGYIDNWSSDGFLRWAHALGFIEYINKSDSFVITDVGLAYSKSADGSAIEKEILIEAISSYPPAIRILTLLEDGQHLTKFDLGKNLGFSGESGFTSLPEGILLDTLANAMPKDKGEIRNNWEGSSDKYARMIGGWLDKLGLVKQGKKEFIIPTLGKPDNKEFISHAFKITGEGLKVLRRAKGSTKFTRVPKRVYWEMLATNLTDKEYVRTRRALILEILIKAGSLKIEQIQDNLKKLGFDEVIETIENDIKGLINTGIFIEIKGRFYQLKDHILQFVIPNRGVTKQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 11 - protein, 196 aa, synthetic construct
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 12 - protein, 196 aa, synthetic construct
QLVKSELEEKKSELRHKLKYVPHEYIELIEIARNSTQDRILEMKVMEFFMKVYGYRGKHLGGSRKPAGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 13 - protein, 487 aa, synthetic construct
QLVKSELEEKKSELRHKLKYVPHEYIELIEIARNSTQDRILEMKVMEFFMKVYGYRGKHLGGSRKPAGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFGSGSGSGSITRTTNPRNVVPKIYMSAGSIPLTTHITNSIQPTLWTIGSINGVAPLAKSIKLGIPVTGSAYTDQTTAMVRKKVSVFMGSGSGSGSSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 14 - protein, 487 aa, synthetic construct
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFGSGSGSGSITRTTNPRNVVPKIYMSAGSIPLTTHITNSIQPTLWTIGSINGVAPLAKSIKLGIPVTGSAYTDQTTAMVRKKVSVFMGSGSGSGSSQLVKSELEEKKSELRHKLKYVPHEYIELIEIARNSTQDRILEMKVMEFFMKVYGYRGKHLGGSRKPAGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 15 - protein, 229 aa, synthetic construct
GSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLK

SEQ ID NO: 16 - protein, 18 aa, synthetic construct
KESGSVSSEQLAQFRSLD

SEQ ID NO: 17 - protein, 14 aa, synthetic construct
EGKSSGSGSESKST

SEQ ID NO: 18 - protein, 12 aa, synthetic construct
GSAGSAAGSGEF

SEQ ID NO: 19 - skipped

SEQ ID NO: 20 - DNA, 10 bp, synthetic construct
  source 1..10: mol_type = other DNA
ggggsggggs

SEQ ID NO: 21 - DNA, 15 bp, synthetic construct
  source 1..15: mol_type = other DNA
ggggsggggsggggs

SEQ ID NO: 22 - protein, 16 aa, synthetic construct
SGSETPGTSESATPES

SEQ ID NO: 23 - protein, 95 aa, synthetic construct
GSGSGSGSITRTTNPRNVVPKIYMSAGSIPLTTHITNSIQPTLWTIGSINGVAPLAKSIKLGIPVTGSAYTDQTTAMVRKKVSVFMGSGSGSGSS

SEQ ID NO: 24 - protein, 1810 aa, synthetic construct
MQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFGSGSGSGSITRTTNPRNVVPKIYMSAGSIPLTTHITNSIQPTLWTIGSINGVAPLAKSIKLGIPVTGSAYTDQTTAMVRKKVSVFMGSGSGSGSSQLVKSELEEKKSELRHKLKYVPHEYIELIEIARNSTQDRILEMKVMEFFMKVYGYRGKHLGGSRKPAGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPESTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYRNRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLARGWDVNVEKNRGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIARGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 25 - protein, 2070 aa, synthetic construct
GSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGGSSGGSSGSETPGTSESATPESSGGSSGGSTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYRNRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLARGWDVNVEKNRGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIARGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNSGSETPGTSESATPESQLVKSELEEKKSELRHKLKYVPHEYIELIEIARNSTQDRILEMKVMEFFMKVYGYRGKHLGGSRKPAGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFGSGSGSGSITRTTNPRNVVPKIYMSAGSIPLTTHITNSIQPTLWTIGSINGVAPLAKSIKLGIPVTGSAYTDQTTAMVRKKVSVFMGSGSGSGSSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 26 - protein, 2026 aa, synthetic construct
GSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGGSSGGSSGSETPGTSESATPESSGGSSGGSNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFRNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLARGWSKSVEYSRNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIARGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYLSGSETPGTSESATPESQLVKSELEEKKSELRHKLKYVPHEYIELIEIARNSTQDRILEMKVMEFFMKVYGYRGKHLGGSRKPAGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFGSGSGSGSITRTTNPRNVVPKIYMSAGSIPLTTHITNSIQPTLWTIGSINGVAPLAKSIKLGIPVTGSAYTDQTTAMVRKKVSVFMGSGSGSGSSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 27 - protein, 1809 aa, synthetic construct
TQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYRNRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLARGWDVNVEKNRGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIARGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNSGSETPGTSESATPESQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFGSGSGSGSITRTTNPRNVVPKIYMSAGSIPLTTHITNSIQPTLWTIGSINGVAPLAKSIKLGIPVTGSAYTDQTTAMVRKKVSVFMGSGSGSGSSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 28 - protein, 1809 aa, synthetic construct
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFGSGSGSGSITRTTNPRNVVPKIYMSAGSIPLTTHITNSIQPTLWTIGSINGVAPLAKSIKLGIPVTGSAYTDQTTAMVRKKVSVFMGSGSGSGSSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPESTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYRNRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLARGWDVNVEKNRGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIARGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 29 - protein, 1809 aa, synthetic construct
TQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYRNRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLARGWDVNVEKNRGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIARGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNSGSETPGTSESATPESQLVKSELEEKKSELRHKLKYVPHEYIELIEIARNSTQDRILEMKVMEFFMKVYGYRGKHLGGSRKPAGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFGSGSGSGSITRTTNPRNVVPKIYMSAGSIPLTTHITNSIQPTLWTIGSINGVAPLAKSIKLGIPVTGSAYTDQTTAMVRKKVSVFMGSGSGSGSSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 30 - protein, 1809 aa, synthetic construct
TQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYRNRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLARGWDVNVEKNRGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIARGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNSGSETPGTSESATPESQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFGSGSGSGSITRTTNPRNVVPKIYMSAGSIPLTTHITNSIQPTLWTIGSINGVAPLAKSIKLGIPVTGSAYTDQTTAMVRKKVSVFMGSGSGSGSSQLVKSELEEKKSELRHKLKYVPHEYIELIEIARNSTQDRILEMKVMEFFMKVYGYRGKHLGGSRKPAGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 31 - protein, 1810 aa, synthetic construct
MQLVKSELEEKKSELRHKLKYVPHEYIELIEIARNSTQDRILEMKVMEFFMKVYGYRGKHLGGSRKPAGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFGSGSGSGSITRTTNPRNVVPKIYMSAGSIPLTTHITNSIQPTLWTIGSINGVAPLAKSIKLGIPVTGSAYTDQTTAMVRKKVSVFMGSGSGSGSSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPESTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYRNRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLARGWDVNVEKNRGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIARGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 32 - DNA, 5430 bp, synthetic construct
  source 1..5430: mol_type = other DNA
atgcaactggtgaagagcgagctggaagagaagaaaagcgagctcagacataagctgaagtacgttccccacgaatacattgaactgatagaaatcgctagaaacagtacgcaagacagaatactggaaatgaaggtgatggagttcttcatgaaggtttacggctatcgtggcaaacacctcgggggctcccggaagcccgacggggctatctacaccgtgggcagtcccatcgactatggcgtgatcgtggacaccaaagcttatagcggcggatataatctccccatcggccaagccgatgagatgcagaggtatgtggaggagaaccaaacaagaaacaagcatatcaaccccaacgagtggtggaaggtttatcctagctcggtgaccgagtttaagttcctattcgtgtctggccacttcaagggcaactataaggcacagctcactagactgaatcatatcacgaattgcaacggcgccgtgttatccgtggaggagctactgatcggcggagagatgatcaaagccggcaccctgaccctggaagaggtgagaagaaagtttaacaatggcgaaataaatttcggcagcggaagtggaagcggctccatcactagaaccaccaaccctagaaacgtggtgcccaagatctacatgagcgccggcagcatccccctgaccacccacatcaccaactcaattcagcccaccctgtggaccatcggcagcatcaacggcgtggcccccctggccaagagcatcaagctgggcatccccgtgaccggcagcgcctacaccgatcagaccaccgccatggtgagaaagaaggtgagcgtgttcatgggcagcggcagcgggagcggctcatcgcagctggttaagagcgagttagaagaaaaaaagagcgaactgcggcataaactgaagtatgtcccacacgagtacatcgaactgatcgagatcgcgagaaactctacccaagacagaattctggagatgaaagtaatggaatttttcatgaaggtgtatggatatagagggaagcacctgggtggcagcagaaaacccgccggcgccatctacactgtggggagccccatagactatggtgtgatcgtggataccaaggcgtatagcggcggttacaatctgcccattgggcaagcggacgagatgcaaagatatgtggaagagaatcagacgaggaacaagcacattaaccctaatgagtggtggaaggtctaccctagctccgttaccgagttcaagttcctgtttgtgagcgggcattttaagggcaactacaaggcacagctgacccgcctgaaccacataacaaactgcaacggtgccgtgctgagcgtagaagagttgctaatcggcggcgagatgatcaaggccggcacgctaaccctcgaagaggtgcgcagaaagttcaataacggcgaaatcaatttcagcggcagcgagactcccgggacctcagagtccgccacacccgaaagtacacagttcgagggctttaccaacctgtatcaggtgagcaagacactgcggtttgagctgatcccacagggcaagaccctgaagcacatccaggagcagggcttcatcgaggaggacaaggcccgcaatgatcactacaaggagctgaagcccatcatcgatcggatctacaagacctatgccgaccagtgcctgcagctggtgcagctggattgggagaacctgagcgccgccatcgactcctatagaaaggagaaaaccgaggagacaaggaacgccctgatcgaggagcaggccacatatcgcaatgccatccacgactacttcatcggccggacagacaacctgaccgatgccatcaataagagacacgccgagatctacaagggcctgttcaaggccgagctgtttaatggcaaggtgctgaagcagctgggcaccgtgaccacaaccgagcacgagaacgccctgctgcggagcttcgacaagtttacaacctacttctccggcttttatagaaacaggaagaacgtgttcagcgccgaggatatcagcacagccatcccacaccgcatcgtgcaggacaacttccccaagtttaaggagaattgtcacatcttcacacgcctgatcaccgccgtgcccagcctgcgggagcactttgagaacgtgaagaaggccatcggcatcttcgtgagcacctccatcgaggaggtgttttccttccctttttataaccagctgctgacacagacccagatcgacctgtataaccagctgctgggaggaatctctcgggaggcaggcaccgagaagatcaagggcctgaacgaggtgctgaatctggccatccagaagaatgatgagacagcccacatcatcgcctccctgccacacagattcatccccctgtttaagcagatcctgtccgataggaacaccctgtctttcatcctggaggagtttaagagcgacgaggaagtgatccagtccttctgcaagtacaagacactgctgagaaacgagaacgtgctggagacagccgaggccctgtttaacgagctgaacagcatcgacctgacacacatcttcatcagccacaagaagctggagacaatcagcagcgccctgtgcgaccactgggatacactgaggaatgccctgtatgagcggagaatctccgagctgacaggcaagatcaccaagtctgccaaggagaaggtgcagcgcagcctgaagcacgaggatatcaacctgcaggagatcatctctgccgcaggcaaggagctgagcgaggccttcaagcagaaaaccagcgagatcctgtcccacgcacacgccgccctggatcagccactgcctacaaccctgaagaagcaggaggagaaggagatcctgaagtctcagctggacagcctgctgggcctgtaccacctgctggactggtttgccgtggatgagtccaacgaggtggaccccgagttctctgcccggctgaccggcatcaagctggagatggagccttctctgagcttctacaacaaggccagaaattatgccaccaagaagccctactccgtggagaagttcaagctgaactttcagatgcctacactggccagaggctgggacgtgaatgtggagaagaacagaggcgccatcctgtttgtgaagaacggcctgtactatctgggcatcatgccaaagcagaagggcaggtataaggccctgagcttcgagcccacagagaaaaccagcgagggctttgataagatgtactatgactacttccctgatgccgccaagatgatcccaaagtgcagcacccagctgaaggccgtgacagcccactttcagacccacacaacccccatcctgctgtccaacaatttcatcgagcctctggagatcacaaaggagatctacgacctgaacaatcctgagaaggagccaaagaagtttcagacagcctacgccaagaaaaccggcgaccagaagggctacagagaggccctgtgcaagtggatcgacttcacaagggattttctgtccaagtataccaagacaacctctatcgatctgtctagcctgcggccatcctctcagtataaggacctgggcgagtactatgccgagctgaatcccctgctgtaccacatcagcttccagagaatcgccgagaaggagatcatggatgccgtggagacaggcaagctgtacctgttccagatctataacaaggactttgccaagggccaccacggcaagcctaatctgcacacactgtattggaccggcctgttttctccagagaacctggccaagacaagcatcaagctgaatggccaggccgagctgttctaccgccctaagtccaggatgaagaggatggcacaccggctgggagagaagatgctgaacaagaagctgaaggatcagaaaaccccaatccccgacaccctgtaccaggagctgtacgactatgtgaatcacagactgtcccacgacctgtctgatgaggccagggccctgctgcccaacgtgatcaccaaggaggtgtctcacgagatcatcaaggataggcgctttaccagcgacaagttctttttccacgtgcctatcacactgaactatcaggccgccaattccccatctaagttcaaccagagggtgaatgcctacctgaaggagcaccccgagacacctatcatcggcatcgcccggggcgagagaaacctgatctatatcacagtgatcgactccaccggcaagatcctggagcagcggagcctgaacaccatccagcagtttgattaccagaagaagctggacaacagggagaaggagagggtggcagcaaggcaggcctggtctgtggtgggcacaatcaaggatctgaagcagggctatctgagccaggtcatccacgagatcgtggacctgatgatccactaccaggccgtggtggtgctggagaacctgaatttcggctttaagagcaagaggaccggcatcgccgagaaggccgtgtaccagcagttcgagaagatgctgatcgataagctgaattgcctggtgctgaaggactatccagcagagaaagtgggaggcgtgctgaacccataccagctgacagaccagttcacctcctttgccaagatgggcacccagtctggcttcctgttttacgtgcctgccccatatacatctaagatcgatcccctgaccggcttcgtggaccccttcgtgtggaaaaccatcaagaatcacgagagccgcaagcacttcctggagggcttcgactttctgcactacgacgtgaaaaccggcgacttcatcctgcactttaagatgaacagaaatctgtccttccagaggggcctgcccggctttatgcctgcatgggatatcgtgttcgagaagaacgagacacagtttgacgccaagggcacccctttcatcgccggcaagagaatcgtgccagtgatcgagaatcacagattcaccggcagataccgggacctgtatcctgccaacgagctgatcgccctgctggaggagaagggcatcgtgttcagggatggctccaacatcctgccaaagctgctggagaatgacgattctcacgccatcgacaccatggtggccctgatccgcagcgtgctgcagatgcggaactccaatgccgccacaggcgaggactatatcaacagccccgtgcgcgatctgaatggcgtgtgcttcgactcccggtttcagaacccagagtggcccatggacgccgatgccaatggcgcctaccacatcgccctgaagggccagctgctgctgaatcacctgaaggagagcaaggatctgaagctgcagaacggcatctccaatcaggactggctggcctacatccaggagctgcgcaac

SEQ ID NO: 33 - DNA, 6213 bp, synthetic construct
  source 1..6213: mol_type = other DNA
atgggcagctcagagactggcccagtggctgtggaccccacattgagacggcggatcgagccccatgagtttgaggtattcttcgatccgagagagctccgcaaggagacctgcctgctttacgaaattaattgggggggccggcactccatttggcgacatacatcacagaacactaacaagcacgtcgaagtcaacttcatcgagaagttcacgacagaaagatatttctgtccgaacacaaggtgcagcattacctggtttctcagctggagcccatgcggcgaatgtagtagggccatcactgaattcctgtcaaggtatccccacgtcactctgtttatttacatcgcaaggctgtaccaccacgctgacccccgcaatcgacaaggcctgcgggatttgatctcttcaggtgtgactatccaaattatgactgagcaggagtcaggatactgctggagaaactttgtgaattatagcccgagtaatgaagcccactggcctaggtatccccatctgtgggtacgactgtacgttcttgaactgtactgcatcatactgggcctgcctccttgtctcaacattctgagaaggaagcagccacagctgacattctttaccatcgctcttcagtcttgtcattaccagcgactgcccccacacattctctgggccaccgggttgaaatctggtggttcttctggtggttctagcggcagcgagactcccgggacctcagagtccgccacacccgaaagttccggagggagtagcggcgggtctacacagttcgagggctttaccaacctgtatcaggtgagcaagacactgcggtttgagctgatcccacagggcaagaccctgaagcacatccaggagcagggcttcatcgaggaggacaaggcccgcaatgatcactacaaggagctgaagcccatcatcgatcggatctacaagacctatgccgaccagtgcctgcagctggtgcagctggattgggagaacctgagcgccgccatcgactcctatagaaaggagaaaaccgaggagacaaggaacgccctgatcgaggagcaggccacatatcgcaatgccatccacgactacttcatcggccggacagacaacctgaccgatgccatcaataagagacacgccgagatctacaagggcctgttcaaggccgagctgtttaatggcaaggtgctgaagcagctgggcaccgtgaccacaaccgagcacgagaacgccctgctgcggagcttcgacaagtttacaacctacttctccggcttttatagaaacaggaagaacgtgttcagcgccgaggatatcagcacagccatcccacaccgcatcgtgcaggacaacttccccaagtttaaggagaattgtcacatcttcacacgcctgatcaccgccgtgcccagcctgcgggagcactttgagaacgtgaagaaggccatcggcatcttcgtgagcacctccatcgaggaggtgttttccttccctttttataaccagctgctgacacagacccagatcgacctgtataaccagctgctgggaggaatctctcgggaggcaggcaccgagaagatcaagggcctgaacgaggtgctgaatctggccatccagaagaatgatgagacagcccacatcatcgcctccctgccacacagattcatccccctgtttaagcagatcctgtccgataggaacaccctgtctttcatcctggaggagtttaagagcgacgaggaagtgatccagtccttctgcaagtacaagacactgctgagaaacgagaacgtgctggagacagccgaggccctgtttaacgagctgaacagcatcgacctgacacacatcttcatcagccacaagaagctggagacaatcagcagcgccctgtgcgaccactgggatacactgaggaatgccctgtatgagcggagaatctccgagctgacaggcaagatcaccaagtctgccaaggagaaggtgcagcgcagcctgaagcacgaggatatcaacctgcaggagatcatctctgccgcaggcaaggagctgagcgaggccttcaagcagaaaaccagcgagatcctgtcccacgcacacgccgccctggatcagccactgcctacaaccctgaagaagcaggaggagaaggagatcctgaagtctcagctggacagcctgctgggcctgtaccacctgctggactggtttgccgtggatgagtccaacgaggtggaccccgagttctctgcccggctgaccggcatcaagctggagatggagccttctctgagcttctacaacaaggccagaaattatgccaccaagaagccctactccgtggagaagttcaagctgaactttcagatgcctacactggccagaggctgggacgtgaatgtggagaagaacagaggcgccatcctgtttgtgaagaacggcctgtactatctgggcatcatgccaaagcagaagggcaggtataaggccctgagcttcgagcccacagagaaaaccagcgagggctttgataagatgtactatgactacttccctgatgccgccaagatgatcccaaagtgcagcacccagctgaaggccgtgacagcccactttcagacccacacaacccccatcctgctgtccaacaatttcatcgagcctctggagatcacaaaggagatctacgacctgaacaatcctgagaaggagccaaagaagtttcagacagcctacgccaagaaaaccggcgaccagaagggctacagagaggccctgtgcaagtggatcgacttcacaagggattttctgtccaagtataccaagacaacctctatcgatctgtctagcctgcggccatcctctcagtataaggacctgggcgagtactatgccgagctgaatcccctgctgtaccacatcagcttccagagaatcgccgagaaggagatcatggatgccgtggagacaggcaagctgtacctgttccagatctataacaaggactttgccaagggccaccacggcaagcctaatctgcacacactgtattggaccggcctgttttctccagagaacctggccaagacaagcatcaagctgaatggccaggccgagctgttctaccgccctaagtccaggatgaagaggatggcacaccggctgggagagaagatgctgaacaagaagctgaaggatcagaaaaccccaatccccgacaccctgtaccaggagctgtacgactatgtgaatcacagactgtcccacgacctgtctgatgaggccagggccctgctgcccaacgtgatcaccaaggaggtgtctcacgagatcatcaaggataggcgctttaccagcgacaagttctttttccacgtgcctatcacactgaactatcaggccgccaattccccatctaagttcaaccagagggtgaatgcctacctgaaggagcaccccgagacacctatcatcggcatcgcccggggcgagagaaacctgatctatatcacagtgatcgactccaccggcaagatcctggagcagcggagcctgaacaccatccagcagtttgattaccagaagaagctggacaacagggagaaggagagggtggcagcaaggcaggcctggtctgtggtgggcacaatcaaggatctgaagcagggctatctgagccaggtcatccacgagatcgtggacctgatgatccactaccaggccgtggtggtgctggagaacctgaatttcggctttaagagcaagaggaccggcatcgccgagaaggccgtgtaccagcagttcgagaagatgctgatcgataagctgaattgcctggtgctgaaggactatccagcagagaaagtgggaggcgtgctgaacccataccagctgacagaccagttcacctcctttgccaagatgggcacccagtctggcttcctgttttacgtgcctgccccatatacatctaagatcgatcccctgaccggcttcgtggaccccttcgtgtggaaaaccatcaagaatcacgagagccgcaagcacttcctggagggcttcgactttctgcactacgacgtgaaaaccggcgacttcatcctgcactttaagatgaacagaaatctgtccttccagaggggcctgcccggctttatgcctgcatgggatatcgtgttcgagaagaacgagacacagtttgacgccaagggcacccctttcatcgccggcaagagaatcgtgccagtgatcgagaatcacagattcaccggcagataccgggacctgtatcctgccaacgagctgatcgccctgctggaggagaagggcatcgtgttcagggatggctccaacatcctgccaaagctgctggagaatgacgattctcacgccatcgacaccatggtggccctgatccgcagcgtgctgcagatgcggaactccaatgccgccacaggcgaggactatatcaacagccccgtgcgcgatctgaatggcgtgtgcttcgactcccggtttcagaacccagagtggcccatggacgccgatgccaatggcgcctaccacatcgccctgaagggccagctgctgctgaatcacctgaaggagagcaaggatctgaagctgcagaacggcatctccaatcaggactggctggcctacatccaggagctgcgcaacagcggcagcgagactcccgggacctcagagtccgccacacccgaaagtcaactggtgaagagcgagctggaagagaagaaaagcgagctcagacataagctgaagtacgttccccacgaatacattgaactgatagaaatcgctagaaacagtacgcaagacagaatactggaaatgaaggtgatggagttcttcatgaaggtttacggctatcgtggcaaacacctcgggggctcccggaagcccgccggggctatctacaccgtgggcagtcccatcgactatggcgtgatcgtggacaccaaagcttatagcggcggatataatctccccatcggccaagccgatgagatgcagaggtatgtggaggagaaccaaacaagaaacaagcatatcaaccccaacgagtggtggaaggtttatcctagctcggtgaccgagtttaagttcctattcgtgtctggccacttcaagggcaactataaggcacagctcactagactgaatcatatcacgaattgcaacggcgccgtgttatccgtggaggagctactgatcggcggagagatgatcaaagccggcaccctgaccctggaagaggtgagaagaaagtttaacaatggcgaaataaatttcggcagcggaagtggaagcggctccatcactagaaccaccaaccctagaaacgtggtgcccaagatctacatgagcgccggcagcatccccctgaccacccacatcaccaactcaattcagcccaccctgtggaccatcggcagcatcaacggcgtggcccccctggccaagagcatcaagctgggcatccccgtgaccggcagcgcctacaccgatcagaccaccgccatggtgagaaagaaggtgagcgtgttcatgggcagcggcagcgggagcggctcatcgcagctggttaagagcgagttagaagaaaaaaagagcgaactgcggcataaactgaagtatgtcccacacgagtacatcgaactgatcgagatcgcgagaaactctacccaagacagaattctggagatgaaagtaatggaatttttcatgaaggtgtatggatatagagggaagcacctgggtggcagcagaaaacccgacggcgccatctacactgtggggagccccatagactatggtgtgatcgtggataccaaggcgtatagcggcggttacaatctgcccattgggcaagcggacgagatgcaaagatatgtggaagagaatcagacgaggaacaagcacattaaccctaatgagtggtggaaggtctaccctagctccgttaccgagttcaagttcctgtttgtgagcgggcattttaagggcaactacaaggcacagctgacccgcctgaaccacataacaaactgcaacggtgccgtgctgagcgtagaagagttgctaatcggcggcgagatgatcaaggccggcacgctaaccctcgaagaggtgcgcagaaagttcaataacggcgaaatcaatttc

SEQ ID NO: 34 - DNA, 6081 bp, synthetic construct
  source 1..6081: mol_type = other DNA
atgggcagctcagagactggcccagtggctgtggaccccacattgagacggcggatcgagccccatgagtttgaggtattcttcgatccgagagagctccgcaaggagacctgcctgctttacgaaattaattgggggggccggcactccatttggcgacatacatcacagaacactaacaagcacgtcgaagtcaacttcatcgagaagttcacgacagaaagatatttctgtccgaacacaaggtgcagcattacctggtttctcagctggagcccatgcggcgaatgtagtagggccatcactgaattcctgtcaaggtatccccacgtcactctgtttatttacatcgcaaggctgtaccaccacgctgacccccgcaatcgacaaggcctgcgggatttgatctcttcaggtgtgactatccaaattatgactgagcaggagtcaggatactgctggagaaactttgtgaattatagcccgagtaatgaagcccactggcctaggtatccccatctgtgggtacgactgtacgttcttgaactgtactgcatcatactgggcctgcctccttgtctcaacattctgagaaggaagcagccacagctgacattctttaccatcgctcttcagtcttgtcattaccagcgactgcccccacacattctctgggccaccgggttgaaatctggtggttcttctggtggttctagcggcagcgagactcccgggacctcagagtccgccacacccgaaagttccggagggagtagcggcgggtctaataacggaactaataacttccaaaacttcatcgggatcagttccttgcagaaaactctccggaatgctctcatcccaactgagactactcagcagttcattgttaagaatggaatcataaaagaggacgagcttaggggggaaaataggcaaatcctcaaggatatcatggatgactattataggggctttatatccgagacactgagcagcattgatgatatagactggacctctcttttcgaaaagatggaaatacaacttaaaaatggagataacaaggacaccctgataaaggaacagaccgaatataggaaggcaattcataaaaagtttgctaacgatgataggtttaaaaacatgttctcagcaaaactcatttcagatatactgcccgaattcgttatccacaacaacaactactccgctagcgaaaaagaggaaaagacccaagtcataaagctgttctctcgattcgcgacgagttttaaagattatttccgaaatcgcgcaaactgtttctcagctgatgatatcagcagctcatcctgtcatcggatcgttaacgataatgctgaaatcttcttctccaatgcacttgtttataggcgcattgttaaatctctctcaaacgatgatatcaataagatttccggcgatatgaaggacagtcttaaggagatgagcctcgaagagatatactcatacgagaaatatggcgaatttatcacccaggaagggatttccttctataatgacatttgcggcaaagtcaattccttcatgaacctgtattgccaaaaaaataaagaaaacaagaacctctataagctgcaaaagttgcataagcaaatactttgtatcgcggatacaagctatgaagttccctacaagttcgagagtgatgaggaggtgtatcaatctgtcaatggtttccttgataatatttcttctaagcatattgttgaacgactccgaaagataggagacaactataatggatacaatttggataaaatctacatcgtgtctaaattttacgagagtgtgtcacaaaaaacatatagagactgggagacaattaataccgccctggagatacattacaacaatatacttcccgggaacgggaagtctaaggcagacaaggtgaagaaagccgtgaagaacgacttgcaaaagtcaattaccgaaatcaatgagcttgtttcaaactataaactttgttcagatgacaatattaaagccgaaacctatattcatgaaatctctcatattctgaataactttgaggcgcaagaactgaaatataacccagaaatacacctcgttgagtccgaactgaaagcaagcgaactgaaaaatgttttggacgtgataatgaacgcttttcattggtgctcagtctttatgacagaggagcttgttgacaaggataacaatttctatgcggaactggaagagatttacgacgaaatctatccggtcatatccctgtataacctggttcgcaactatgtcacgcaaaaaccatacagcacgaagaagattaaactgaactttggtattccgacgctggcccgcggatggtcaaaatctgttgaatactcacgaaatgccataatcctgatgcgagataacctctactaccttggaatctttaatgctaaaaataaacccgataaaaaaattatcgaagggaacacgagtgaaaacaaaggtgattataaaaaaatgatatataatctgcttccaggaccaaataagatgatacccaaagttttcctttcttcaaagaccggcgtcgagacatataaaccatccgcgtacatacttgaaggctacaaacaaaataaacatatcaaatcatctaaggattttgacattacgttctgtcatgatttgattgactatttcaaaaattgcatagccattcatccagagtggaaaaactttgggtttgacttctctgataccagtacatatgaagacataagtggattttaccgagaagtagagctccaaggttataaaatagactggacctatatatctgaaaaggatatagaccttttgcaagagaagggacagctttatcttttccaaatctacaacaaagacttcagtaagaaaagtaccgggaatgacaatcttcataccatgtatctgaagaacctgttctccgaagaaaatctgaaggacatagtcctgaagcttaatggcgaagcggaaatttttttccgaaagagctctattaagaaccccataatacataagaagggaagcattctcgttaatcgaacgtatgaggccgaagagaaagatcaatttgggaatatccaaatcgttcgaaagaacataccagaaaatatttaccaagaattgtacaaatattttaacgataaaagcgacaaagaactgtctgatgaagctgctaagctgaaaaacgtcgtcggccatcatgaggccgcgacgaatatagtcaaggattaccgatatacatacgataagtatttcctgcatatgcccatcactatcaactttaaggcaaataagactggattcattaatgacagaatactgcaatacatagctaaagaaaaagatttgcatgttattggcattgccaggggtgagcgcaatcttatctatgtaagcgtcattgatacttgcgggaatatcgtagagcagaagtcatttaatattgtaaatgggtacgattaccaaatcaagttgaagcagcaagagggagcacgacagattgcccgcaaggagtggaaagagatcggaaagataaaggagatcaaggaggggtatttgtcccttgttatacacgaaatttccaagatggtaatcaagtacaacgctataattgctatggaggatctctcctatggatttaaaaagggaagatttaaagtcgagcggcaggtatatcagaaatttgaaacaatgcttattaataaacttaattatctcgttttcaaagacattagtatcaccgaaaacggtgggctgttgaagggctatcaacttacgtacataccagataagcttaagaatgtgggtcaccaatgcggatgcatattctacgtgcccgcagcttatacaagcaaaatcgacccaacaacgggtttcgtaaacatatttaagttcaaggatctcaccgtggatgccaagcgagagttcataaaaaaatttgactcaatcagatatgactcagaaaagaatcttttttgttttaccttcgactacaataatttcattacacaaaatacggttatgagcaagtcatcctggtccgtatatacgtatggagtgcgcataaagcggagattcgttaacgggcgattttctaatgagtccgatacaatcgatataacaaaggatatggaaaaaactctggaaatgactgatataaattggagggacggtcatgacctcaggcaagacattatcgattatgagatcgtgcaacatatttttgagatctttcggttgactgtccaaatgaggaactctctgtctgaattggaagatagggactacgatcgcctgataagccccgtgttgaacgagaataacatattctacgattccgcgaaagccggggatgcgctccctaaggacgccgatgcaaatggggcctattgtattgctttgaaagggctgtacgaaatcaaacagatcaccgaaaactggaaagaagacgggaagtttagtcgggataaactgaagatatccaacaaggactggtttgactttatccaaaataagcgatatttgagcggcagcgagactcccgggacctcagagtccgccacacccgaaagtcaactggtgaagagcgagctggaagagaagaaaagcgagctcagacataagctgaagtacgttccccacgaatacattgaactgatagaaatcgctagaaacagtacgcaagacagaatactggaaatgaaggtgatggagttcttcatgaaggtttacggctatcgtggcaaacacctcgggggctcccggaagcccgccggggctatctacaccgtgggcagtcccatcgactatggcgtgatcgtggacaccaaagcttatagcggcggatataatctccccatcggccaagccgatgagatgcagaggtatgtggaggagaaccaaacaagaaacaagcatatcaaccccaacgagtggtggaaggtttatcctagctcggtgaccgagtttaagttcctattcgtgtctggccacttcaagggcaactataaggcacagctcactagactgaatcatatcacgaattgcaacggcgccgtgttatccgtggaggagctactgatcggcggagagatgatcaaagccggcaccctgaccctggaagaggtgagaagaaagtttaacaatggcgaaataaatttcggcagcggaagtggaagcggctccatcactagaaccaccaaccctagaaacgtggtgcccaagatctacatgagcgccggcagcatccccctgaccacccacatcaccaactcaattcagcccaccctgtggaccatcggcagcatcaacggcgtggcccccctggccaagagcatcaagctgggcatccccgtgaccggcagcgcctacaccgatcagaccaccgccatggtgagaaagaaggtgagcgtgttcatgggcagcggcagcgggagcggctcatcgcagctggttaagagcgagttagaagaaaaaaagagcgaactgcggcataaactgaagtatgtcccacacgagtacatcgaactgatcgagatcgcgagaaactctacccaagacagaattctggagatgaaagtaatggaatttttcatgaaggtgtatggatatagagggaagcacctgggtggcagcagaaaacccgacggcgccatctacactgtggggagccccatagactatggtgtgatcgtggataccaaggcgtatagcggcggttacaatctgcccattgggcaagcggacgagatgcaaagatatgtggaagagaatcagacgaggaacaagcacattaaccctaatgagtggtggaaggtctaccctagctccgttaccgagttcaagttcctgtttgtgagcgggcattttaagggcaactacaaggcacagctgacccgcctgaaccacataacaaactgcaacggtgccgtgctgagcgtagaagagttgctaatcggcggcgagatgatcaaggccggcacgctaaccctcgaagaggtgcgcagaaagttcaataacggcgaaatcaatttc

SEQ ID NO: 35 - DNA, 5430 bp, synthetic construct
  source 1..5430: mol_type = other DNA
atgacacagttcgagggctttaccaacctgtatcaggtgagcaagacactgcggtttgagctgatcccacagggcaagaccctgaagcacatccaggagcagggcttcatcgaggaggacaaggcccgcaatgatcactacaaggagctgaagcccatcatcgatcggatctacaagacctatgccgaccagtgcctgcagctggtgcagctggattgggagaacctgagcgccgccatcgactcctatagaaaggagaaaaccgaggagacaaggaacgccctgatcgaggagcaggccacatatcgcaatgccatccacgactacttcatcggccggacagacaacctgaccgatgccatcaataagagacacgccgagatctacaagggcctgttcaaggccgagctgtttaatggcaaggtgctgaagcagctgggcaccgtgaccacaaccgagcacgagaacgccctgctgcggagcttcgacaagtttacaacctacttctccggcttttatagaaacaggaagaacgtgttcagcgccgaggatatcagcacagccatcccacaccgcatcgtgcaggacaacttccccaagtttaaggagaattgtcacatcttcacacgcctgatcaccgccgtgcccagcctgcgggagcactttgagaacgtgaagaaggccatcggcatcttcgtgagcacctccatcgaggaggtgttttccttccctttttataaccagctgctgacacagacccagatcgacctgtataaccagctgctgggaggaatctctcgggaggcaggcaccgagaagatcaagggcctgaacgaggtgctgaatctggccatccagaagaatgatgagacagcccacatcatcgcctccctgccacacagattcatccccctgtttaagcagatcctgtccgataggaacaccctgtctttcatcctggaggagtttaagagcgacgaggaagtgatccagtccttctgcaagtacaagacactgctgagaaacgagaacgtgctggagacagccgaggccctgtttaacgagctgaacagcatcgacctgacacacatcttcatcagccacaagaagctggagacaatcagcagcgccctgtgcgaccactgggatacactgaggaatgccctgtatgagcggagaatctccgagctgacaggcaagatcaccaagtctgccaaggagaaggtgcagcgcagcctgaagcacgaggatatcaacctgcaggagatcatctctgccgcaggcaaggagctgagcgaggccttcaagcagaaaaccagcgagatcctgtcccacgcacacgccgccctggatcagccactgcctacaaccctgaagaagcaggaggagaaggagatcctgaagtctcagctggacagcctgctgggcctgtaccacctgctggactggtttgccgtggatgagtccaacgaggtggaccccgagttctctgcccggctgaccggcatcaagctggagatggagccttctctgagcttctacaacaaggccagaaattatgccaccaagaagccctactccgtggagaagttcaagctgaactttcagatgcctacactggccagaggctgggacgtgaatgtggagaagaacagaggcgccatcctgtttgtgaagaacggcctgtactatctgggcatcatgccaaagcagaagggcaggtataaggccctgagcttcgagcccacagagaaaaccagcgagggctttgataagatgtactatgactacttccctgatgccgccaagatgatcccaaagtgcagcacccagctgaaggccgtgacagcccactttcagacccacacaacccccatcctgctgtccaacaatttcatcgagcctctggagatcacaaaggagatctacgacctgaacaatcctgagaaggagccaaagaagtttcagacagcctacgccaagaaaaccggcgaccagaagggctacagagaggccctgtgcaagtggatcgacttcacaagggattttctgtccaagtataccaagacaacctctatcgatctgtctagcctgcggccatcctctcagtataaggacctgggcgagtactatgccgagctgaatcccctgctgtaccacatcagcttccagagaatcgccgagaaggagatcatggatgccgtggagacaggcaagctgtacctgttccagatctataacaaggactttgccaagggccaccacggcaagcctaatctgcacacactgtattggaccggcctgttttctccagagaacctggccaagacaagcatcaagctgaatggccaggccgagctgttctaccgccctaagtccaggatgaagaggatggcacaccggctgggagagaagatgctgaacaagaagctgaaggatcagaaaaccccaatccccgacaccctgtaccaggagctgtacgactatgtgaatcacagactgtcccacgacctgtctgatgaggccagggccctgctgcccaacgtgatcaccaaggaggtgtctcacgagatcatcaaggataggcgctttaccagcgacaagttctttttccacgtgcctatcacactgaactatcaggccgccaattccccatctaagttcaaccagagggtgaatgcctacctgaaggagcaccccgagacacctatcatcggcatcgcccggggcgagagaaacctgatctatatcacagtgatcgactccaccggcaagatcctggagcagcggagcctgaacaccatccagcagtttgattaccagaagaagctggacaacagggagaaggagagggtggcagcaaggcaggcctggtctgtggtgggcacaatcaaggatctgaagcagggctatctgagccaggtcatccacgagatcgtggacctgatgatccactaccaggccgtggtggtgctggagaacctgaatttcggctttaagagcaagaggaccggcatcgccgagaaggccgtgtaccagcagttcgagaagatgctgatcgataagctgaattgcctggtgctgaaggactatccagcagagaaagtgggaggcgtgctgaacccataccagctgacagaccagttcacctcctttgccaagatgggcacccagtctggcttcctgttttacgtgcctgccccatatacatctaagatcgatcccctgaccggcttcgtggaccccttcgtgtggaaaaccatcaagaatcacgagagccgcaagcacttcctggagggcttcgactttctgcactacgacgtgaaaaccggcgacttcatcctgcactttaagatgaacagaaatctgtccttccagaggggcctgcccggctttatgcctgcatgggatatcgtgttcgagaagaacgagacacagtttgacgccaagggcacccctttcatcgccggcaagagaatcgtgccagtgatcgagaatcacagattcaccggcagataccgggacctgtatcctgccaacgagctgatcgccctgctggaggagaagggcatcgtgttcagggatggctccaacatcctgccaaagctgctggagaatgacgattctcacgccatcgacaccatggtggccctgatccgcagcgtgctgcagatgcggaactccaatgccgccacaggcgaggactatatcaacagccccgtgcgcgatctgaatggcgtgtgcttcgactcccggtttcagaacccagagtggcccatggacgccgatgccaatggcgcctaccacatcgccctgaagggccagctgctgctgaatcacctgaaggagagcaaggatctgaagctgcagaacggcatctccaatcaggactggctggcctacatccaggagctgcgcaacagcggcagcgagactcccgggacctcagagtccgccacacccgaaagtcaactggtgaagagcgagctggaagagaagaaaagcgagctcagacataagctgaagtacgttccccacgaatacattgaactgatagaaatcgctagaaacagtacgcaagacagaatactggaaatgaaggtgatggagttcttcatgaaggtttacggctatcgtggcaaacacctcgggggctcccggaagcccgacggggctatctacaccgtgggcagtcccatcgactatggcgtgatcgtggacaccaaagcttatagcggcggatataatctccccatcggccaagccgatgagatgcagaggtatgtggaggagaaccaaacaagaaacaagcatatcaaccccaacgagtggtggaaggtttatcctagctcggtgaccgagtttaagttcctattcgtgtctggccacttcaagggcaactataaggcacagctcactagactgaatcatatcacgaattgcaacggcgccgtgttatccgtggaggagctactgatcggcggagagatgatcaaagccggcaccctgaccctggaagaggtgagaagaaagtttaacaatggcgaaataaatttcggcagcggaagtggaagcggctccatcactagaaccaccaaccctagaaacgtggtgcccaagatctacatgagcgccggcagcatccccctgaccacccacatcaccaactcaattcagcccaccctgtggaccatcggcagcatcaacggcgtggcccccctggccaagagcatcaagctgggcatccccgtgaccggcagcgcctacaccgatcagaccaccgccatggtgagaaagaaggtgagcgtgttcatgggcagcggcagcgggagcggctcatcgcagctggttaagagcgagttagaagaaaaaaagagcgaactgcggcataaactgaagtatgtcccacacgagtacatcgaactgatcgagatcgcgagaaactctacccaagacagaattctggagatgaaagtaatggaatttttcatgaaggtgtatggatatagagggaagcacctgggtggcagcagaaaacccgacggcgccatctacactgtggggagccccatagactatggtgtgatcgtggataccaaggcgtatagcggcggttacaatctgcccattgggcaagcggacgagatgcaaagatatgtggaagagaatcagacgaggaacaagcacattaaccctaatgagtggtggaaggtctaccctagctccgttaccgagttcaagttcctgtttgtgagcgggcattttaagggcaactacaaggcacagctgacccgcctgaaccacataacaaactgcaacggtgccgtgctgagcgtagaagagttgctaatcggcggcgagatgatcaaggccggcacgctaaccctcgaagaggtgcgcagaaagttcaataacggcgaaatcaatttc

SEQ ID NO: 36 - DNA, 5430 bp, synthetic construct
  source 1..5430: mol_type = other DNA
atgcaactggtgaagagcgagctggaagagaagaaaagcgagctcagacataagctgaagtacgttccccacgaatacattgaactgatagaaatcgctagaaacagtacgcaagacagaatactggaaatgaaggtgatggagttcttcatgaaggtttacggctatcgtggcaaacacctcgggggctcccggaagcccgacggggctatctacaccgtgggcagtcccatcgactatggcgtgatcgtggacaccaaagcttatagcggcggatataatctccccatcggccaagccgatgagatgcagaggtatgtggaggagaaccaaacaagaaacaagcatatcaaccccaacgagtggtggaaggtttatcctagctcggtgaccgagtttaagttcctattcgtgtctggccacttcaagggcaactataaggcacagctcactagactgaatcatatcacgaattgcaacggcgccgtgttatccgtggaggagctactgatcggcggagagatgatcaaagccggcaccctgaccctggaagaggtgagaagaaagtttaacaatggcgaaataaatttcggcagcggaagtggaagcggctccatcactagaaccaccaaccctagaaacgtggtgcccaagatctacatgagcgccggcagcatccccctgaccacccacatcaccaactcaattcagcccaccctgtggaccatcggcagcatcaacggcgtggcccccctggccaagagcatcaagctgggcatccccgtgaccggcagcgcctacaccgatcagaccaccgccatggtgagaaagaaggtgagcgtgttcatgggcagcggcagcgggagcggctcatcgcagctggttaagagcgagttagaagaaaaaaagagcgaactgcggcataaactgaagtatgtcccacacgagtacatcgaactgatcgagatcgcgagaaactctacccaagacagaattctggagatgaaagtaatggaatttttcatgaaggtgtatggatatagagggaagcacctgggtggcagcagaaaacccgacggcgccatctacactgtggggagccccatagactatggtgtgatcgtggataccaaggcgtatagcggcggttacaatctgcccattgggcaagcggacgagatgcaaagatatgtggaagagaatcagacgaggaacaagcacattaaccctaatgagtggtggaaggtctaccctagctccgttaccgagttcaagttcctgtttgtgagcgggcattttaagggcaactacaaggcacagctgacccgcctgaaccacataacaaactgcaacggtgccgtgctgagcgtagaagagttgctaatcggcggcgagatgatcaaggccggcacgctaaccctcgaagaggtgcgcagaaagttcaataacggcgaaatcaatttcagcggcagcgagactcccgggacctcagagtccgccacacccgaaagtacacagttcgagggctttaccaacctgtatcaggtgagcaagacactgcggtttgagctgatcccacagggcaagaccctgaagcacatccaggagcagggcttcatcgaggaggacaaggcccgcaatgatcactacaaggagctgaagcccatcatcgatcggatctacaagacctatgccgaccagtgcctgcagctggtgcagctggattgggagaacctgagcgccgccatcgactcctatagaaaggagaaaaccgaggagacaaggaacgccctgatcgaggagcaggccacatatcgcaatgccatccacgactacttcatcggccggacagacaacctgaccgatgccatcaataagagacacgccgagatctacaagggcctgttcaaggccgagctgtttaatggcaaggtgctgaagcagctgggcaccgtgaccacaaccgagcacgagaacgccctgctgcggagcttcgacaagtttacaacctacttctccggcttttatagaaacaggaagaacgtgttcagcgccgaggatatcagcacagccatcccacaccgcatcgtgcaggacaacttccccaagtttaaggagaattgtcacatcttcacacgcctgatcaccgccgtgcccagcctgcgggagcactttgagaacgtgaagaaggccatcggcatcttcgtgagcacctccatcgaggaggtgttttccttccctttttataaccagctgctgacacagacccagatcgacctgtataaccagctgctgggaggaatctctcgggaggcaggcaccgagaagatcaagggcctgaacgaggtgctgaatctggccatccagaagaatgatgagacagcccacatcatcgcctccctgccacacagattcatccccctgtttaagcagatcctgtccgataggaacaccctgtctttcatcctggaggagtttaagagcgacgaggaagtgatccagtccttctgcaagtacaagacactgctgagaaacgagaacgtgctggagacagccgaggccctgtttaacgagctgaacagcatcgacctgacacacatcttcatcagccacaagaagctggagacaatcagcagcgccctgtgcgaccactgggatacactgaggaatgccctgtatgagcggagaatctccgagctgacaggcaagatcaccaagtctgccaaggagaaggtgcagcgcagcctgaagcacgaggatatcaacctgcaggagatcatctctgccgcaggcaaggagctgagcgaggccttcaagcagaaaaccagcgagatcctgtcccacgcacacgccgccctggatcagccactgcctacaaccctgaagaagcaggaggagaaggagatcctgaagtctcagctggacagcctgctgggcctgtaccacctgctggactggtttgccgtggatgagtccaacgaggtggaccccgagttctctgcccggctgaccggcatcaagctggagatggagccttctctgagcttctacaacaaggccagaaattatgccaccaagaagccctactccgtggagaagttcaagctgaactttcagatgcctacactggccagaggctgggacgtgaatgtggagaagaacagaggcgccatcctgtttgtgaagaacggcctgtactatctgggcatcatgccaaagcagaagggcaggtataaggccctgagcttcgagcccacagagaaaaccagcgagggctttgataagatgtactatgactacttccctgatgccgccaagatgatcccaaagtgcagcacccagctgaaggccgtgacagcccactttcagacccacacaacccccatcctgctgtccaacaatttcatcgagcctctggagatcacaaaggagatctacgacctgaacaatcctgagaaggagccaaagaagtttcagacagcctacgccaagaaaaccggcgaccagaagggctacagagaggccctgtgcaagtggatcgacttcacaagggattttctgtccaagtataccaagacaacctctatcgatctgtctagcctgcggccatcctctcagtataaggacctgggcgagtactatgccgagctgaatcccctgctgtaccacatcagcttccagagaatcgccgagaaggagatcatggatgccgtggagacaggcaagctgtacctgttccagatctataacaaggactttgccaagggccaccacggcaagcctaatctgcacacactgtattggaccggcctgttttctccagagaacctggccaagacaagcatcaagctgaatggccaggccgagctgttctaccgccctaagtccaggatgaagaggatggcacaccggctgggagagaagatgctgaacaagaagctgaaggatcagaaaaccccaatccccgacaccctgtaccaggagctgtacgactatgtgaatcacagactgtcccacgacctgtctgatgaggccagggccctgctgcccaacgtgatcaccaaggaggtgtctcacgagatcatcaaggataggcgctttaccagcgacaagttctttttccacgtgcctatcacactgaactatcaggccgccaattccccatctaagttcaaccagagggtgaatgcctacctgaaggagcaccccgagacacctatcatcggcatcgcccggggcgagagaaacctgatctatatcacagtgatcgactccaccggcaagatcctggagcagcggagcctgaacaccatccagcagtttgattaccagaagaagctggacaacagggagaaggagagggtggcagcaaggcaggcctggtctgtggtgggcacaatcaaggatctgaagcagggctatctgagccaggtcatccacgagatcgtggacctgatgatccactaccaggccgtggtggtgctggagaacctgaatttcggctttaagagcaagaggaccggcatcgccgagaaggccgtgtaccagcagttcgagaagatgctgatcgataagctgaattgcctggtgctgaaggactatccagcagagaaagtgggaggcgtgctgaacccataccagctgacagaccagttcacctcctttgccaagatgggcacccagtctggcttcctgttttacgtgcctgccccatatacatctaagatcgatcccctgaccggcttcgtggaccccttcgtgtggaaaaccatcaagaatcacgagagccgcaagcacttcctggagggcttcgactttctgcactacgacgtgaaaaccggcgacttcatcctgcactttaagatgaacagaaatctgtccttccagaggggcctgcccggctttatgcctgcatgggatatcgtgttcgagaagaacgagacacagtttgacgccaagggcacccctttcatcgccggcaagagaatcgtgccagtgatcgagaatcacagattcaccggcagataccgggacctgtatcctgccaacgagctgatcgccctgctggaggagaagggcatcgtgttcagggatggctccaacatcctgccaaagctgctggagaatgacgattctcacgccatcgacaccatggtggccctgatccgcagcgtgctgcagatgcggaactccaatgccgccacaggcgaggactatatcaacagccccgtgcgcgatctgaatggcgtgtgcttcgactcccggtttcagaacccagagtggcccatggacgccgatgccaatggcgcctaccacatcgccctgaagggccagctgctgctgaatcacctgaaggagagcaaggatctgaagctgcagaacggcatctccaatcaggactggctggcctacatccaggagctgcgcaac

SEQ ID NO: 37 - DNA, 5430 bp, synthetic construct
  source 1..5430: mol_type = other DNA
atgacacagttcgagggctttaccaacctgtatcaggtgagcaagacactgcggtttgagctgatcccacagggcaagaccctgaagcacatccaggagcagggcttcatcgaggaggacaaggcccgcaatgatcactacaaggagctgaagcccatcatcgatcggatctacaagacctatgccgaccagtgcctgcagctggtgcagctggattgggagaacctgagcgccgccatcgactcctatagaaaggagaaaaccgaggagacaaggaacgccctgatcgaggagcaggccacatatcgcaatgccatccacgactacttcatcggccggacagacaacctgaccgatgccatcaataagagacacgccgagatctacaagggcctgttcaaggccgagctgtttaatggcaaggtgctgaagcagctgggcaccgtgaccacaaccgagcacgagaacgccctgctgcggagcttcgacaagtttacaacctacttctccggcttttatagaaacaggaagaacgtgttcagcgccgaggatatcagcacagccatcccacaccgcatcgtgcaggacaacttccccaagtttaaggagaattgtcacatcttcacacgcctgatcaccgccgtgcccagcctgcgggagcactttgagaacgtgaagaaggccatcggcatcttcgtgagcacctccatcgaggaggtgttttccttccctttttataaccagctgctgacacagacccagatcgacctgtataaccagctgctgggaggaatctctcgggaggcaggcaccgagaagatcaagggcctgaacgaggtgctgaatctggccatccagaagaatgatgagacagcccacatcatcgcctccctgccacacagattcatccccctgtttaagcagatcctgtccgataggaacaccctgtctttcatcctggaggagtttaagagcgacgaggaagtgatccagtccttctgcaagtacaagacactgctgagaaacgagaacgtgctggagacagccgaggccctgtttaacgagctgaacagcatcgacctgacacacatcttcatcagccacaagaagctggagacaatcagcagcgccctgtgcgaccactgggatacactgaggaatgccctgtatgagcggagaatctccgagctgacaggcaagatcaccaagtctgccaaggagaaggtgcagcgcagcctgaagcacgaggatatcaacctgcaggagatcatctctgccgcaggcaaggagctgagcgaggccttcaagcagaaaaccagcgagatcctgtcccacgcacacgccgccctggatcagccactgcctacaaccctgaagaagcaggaggagaaggagatcctgaagtctcagctggacagcctgctgggcctgtaccacctgctggactggtttgccgtggatgagtccaacgaggtggaccccgagttctctgcccggctgaccggcatcaagctggagatggagccttctctgagcttctacaacaaggccagaaattatgccaccaagaagccctactccgtggagaagttcaagctgaactttcagatgcctacactggccagaggctgggacgtgaatgtggagaagaacagaggcgccatcctgtttgtgaagaacggcctgtactatctgggcatcatgccaaagcagaagggcaggtataaggccctgagcttcgagcccacagagaaaaccagcgagggctttgataagatgtactatgactacttccctgatgccgccaagatgatcccaaagtgcagcacccagctgaaggccgtgacagcccactttcagacccacacaacccccatcctgctgtccaacaatttcatcgagcctctggagatcacaaaggagatctacgacctgaacaatcctgagaaggagccaaagaagtttcagacagcctacgccaagaaaaccggcgaccagaagggctacagagaggccctgtgcaagtggatcgacttcacaagggattttctgtccaagtataccaagacaacctctatcgatctgtctagcctgcggccatcctctcagtataaggacctgggcgagtactatgccgagctgaatcccctgctgtaccacatcagcttccagagaatcgccgagaaggagatcatggatgccgtggagacaggcaagctgtacctgttccagatctataacaaggactttgccaagggccaccacggcaagcctaatctgcacacactgtattggaccggcctgttttctccagagaacctggccaagacaagcatcaagctgaatggccaggccgagctgttctaccgccctaagtccaggatgaagaggatggcacaccggctgggagagaagatgctgaacaagaagctgaaggatcagaaaaccccaatccccgacaccctgtaccaggagctgtacgactatgtgaatcacagactgtcccacgacctgtctgatgaggccagggccctgctgcccaacgtgatcaccaaggaggtgtctcacgagatcatcaaggataggcgctttaccagcgacaagttctttttccacgtgcctatcacactgaactatcaggccgccaattccccatctaagttcaaccagagggtgaatgcctacctgaaggagcaccccgagacacctatcatcggcatcgcccggggcgagagaaacctgatctatatcacagtgatcgactccaccggcaagatcctggagcagcggagcctgaacaccatccagcagtttgattaccagaagaagctggacaacagggagaaggagagggtggcagcaaggcaggcctggtctgtggtgggcacaatcaaggatctgaagcagggctatctgagccaggtcatccacgagatcgtggacctgatgatccactaccaggccgtggtggtgctggagaacctgaatttcggctttaagagcaagaggaccggcatcgccgagaaggccgtgtaccagcagttcgagaagatgctgatcgataagctgaattgcctggtgctgaaggactatccagcagagaaagtgggaggcgtgctgaacccataccagctgacagaccagttcacctcctttgccaagatgggcacccagtctggcttcctgttttacgtgcctgccccatatacatctaagatcgatcccctgaccggcttcgtggaccccttcgtgtggaaaaccatcaagaatcacgagagccgcaagcacttcctggagggcttcgactttctgcactacgacgtgaaaaccggcgacttcatcctgcactttaagatgaacagaaatctgtccttccagaggggcctgcccggctttatgcctgcatgggatatcgtgttcgagaagaacgagacacagtttgacgccaagggcacccctttcatcgccggcaagagaatcgtgccagtgatcgagaatcacagattcaccggcagataccgggacctgtatcctgccaacgagctgatcgccctgctggaggagaagggcatcgtgttcagggatggctccaacatcctgccaaagctgctggagaatgacgattctcacgccatcgacaccatggtggccctgatccgcagcgtgctgcagatgcggaactccaatgccgccacaggcgaggactatatcaacagccccgtgcgcgatctgaatggcgtgtgcttcgactcccggtttcagaacccagagtggcccatggacgccgatgccaatggcgcctaccacatcgccctgaagggccagctgctgctgaatcacctgaaggagagcaaggatctgaagctgcagaacggcatctccaatcaggactggctggcctacatccaggagctgcgcaacagcggcagcgagactcccgggacctcagagtccgccacacccgaaagtcaactggtgaagagcgagctggaagagaagaaaagcgagctcagacataagctgaagtacgttccccacgaatacattgaactgatagaaatcgctagaaacagtacgcaagacagaatactggaaatgaaggtgatggagttcttcatgaaggtttacggctatcgtggcaaacacctcgggggctcccggaagcccgccggggctatctacaccgtgggcagtcccatcgactatggcgtgatcgtggacaccaaagcttatagcggcggatataatctccccatcggccaagccgatgagatgcagaggtatgtggaggagaaccaaacaagaaacaagcatatcaaccccaacgagtggtggaaggtttatcctagctcggtgaccgagtttaagttcctattcgtgtctggccacttcaagggcaactataaggcacagctcactagactgaatcatatcacgaattgcaacggcgccgtgttatccgtggaggagctactgatcggcggagagatgatcaaagccggcaccctgaccctggaagaggtgagaagaaagtttaacaatggcgaaataaatttcggcagcggaagtggaagcggctccatcactagaaccaccaaccctagaaacgtggtgcccaagatctacatgagcgccggcagcatccccctgaccacccacatcaccaactcaattcagcccaccctgtggaccatcggcagcatcaacggcgtggcccccctggccaagagcatcaagctgggcatccccgtgaccggcagcgcctacaccgatcagaccaccgccatggtgagaaagaaggtgagcgtgttcatgggcagcggcagcgggagcggctcatcgcagctggttaagagcgagttagaagaaaaaaagagcgaactgcggcataaactgaagtatgtcccacacgagtacatcgaactgatcgagatcgcgagaaactctacccaagacagaattctggagatgaaagtaatggaatttttcatgaaggtgtatggatatagagggaagcacctgggtggcagcagaaaacccgacggcgccatctacactgtggggagccccatagactatggtgtgatcgtggataccaaggcgtatagcggcggttacaatctgcccattgggcaagcggacgagatgcaaagatatgtggaagagaatcagacgaggaacaagcacattaaccctaatgagtggtggaaggtctaccctagctccgttaccgagttcaagttcctgtttgtgagcgggcattttaagggcaactacaaggcacagctgacccgcctgaaccacataacaaactgcaacggtgccgtgctgagcgtagaagagttgctaatcggcggcgagatgatcaaggccggcacgctaaccctcgaagaggtgcgcagaaagttcaataacggcgaaatcaatttc

SEQ ID NO: 38 - DNA, 5430 bp, synthetic construct
  source 1..5430: mol_type = other DNA
atgacacagttcgagggctttaccaacctgtatcaggtgagcaagacactgcggtttgagctgatcccacagggcaagaccctgaagcacatccaggagcagggcttcatcgaggaggacaaggcccgcaatgatcactacaaggagctgaagcccatcatcgatcggatctacaagacctatgccgaccagtgcctgcagctggtgcagctggattgggagaacctgagcgccgccatcgactcctatagaaaggagaaaaccgaggagacaaggaacgccctgatcgaggagcaggccacatatcgcaatgccatccacgactacttcatcggccggacagacaacctgaccgatgccatcaataagagacacgccgagatctacaagggcctgttcaaggccgagctgtttaatggcaaggtgctgaagcagctgggcaccgtgaccacaaccgagcacgagaacgccctgctgcggagcttcgacaagtttacaacctacttctccggcttttatagaaacaggaagaacgtgttcagcgccgaggatatcagcacagccatcccacaccgcatcgtgcaggacaacttccccaagtttaaggagaattgtcacatcttcacacgcctgatcaccgccgtgcccagcctgcgggagcactttgagaacgtgaagaaggccatcggcatcttcgtgagcacctccatcgaggaggtgttttccttccctttttataaccagctgctgacacagacccagatcgacctgtataaccagctgctgggaggaatctctcgggaggcaggcaccgagaagatcaagggcctgaacgaggtgctgaatctggccatccagaagaatgatgagacagcccacatcatcgcctccctgccacacagattcatccccctgtttaagcagatcctgtccgataggaacaccctgtctttcatcctggaggagtttaagagcgacgaggaagtgatccagtccttctgcaagtacaagacactgctgagaaacgagaacgtgctggagacagccgaggccctgtttaacgagctgaacagcatcgacctgacacacatcttcatcagccacaagaagctggagacaatcagcagcgccctgtgcgaccactgggatacactgaggaatgccctgtatgagcggagaatctccgagctgacaggcaagatcaccaagtctgccaaggagaaggtgcagcgcagcctgaagcacgaggatatcaacctgcaggagatcatctctgccgcaggcaaggagctgagcgaggccttcaagcagaaaaccagcgagatcctgtcccacgcacacgccgccctggatcagccactgcctacaaccctgaagaagcaggaggagaaggagatcctgaagtctcagctggacagcctgctgggcctgtaccacctgctggactggtttgccgtggatgagtccaacgaggtggaccccgagttctctgcccggctgaccggcatcaagctggagatggagccttctctgagcttctacaacaaggccagaaattatgccaccaagaagccctactccgtggagaagttcaagctgaactttcagatgcctacactggccagaggctgggacgtgaatgtggagaagaacagaggcgccatcctgtttgtgaagaacggcctgtactatctgggcatcatgccaaagcagaagggcaggtataaggccctgagcttcgagcccacagagaaaaccagcgagggctttgataagatgtactatgactacttccctgatgccgccaagatgatcccaaagtgcagcacccagctgaaggccgtgacagcccactttcagacccacacaacccccatcctgctgtccaacaatttcatcgagcctctggagatcacaaaggagatctacgacctgaacaatcctgagaaggagccaaagaagtttcagacagcctacgccaagaaaaccggcgaccagaagggctacagagaggccctgtgcaagtggatcgacttcacaagggattttctgtccaagtataccaagacaacctctatcgatctgtctagcctgcggccatcctctcagtataaggacctgggcgagtactatgccgagctgaatcccctgctgtaccacatcagcttccagagaatcgccgagaaggagatcatggatgccgtggagacaggcaagctgtacctgttccagatctataacaaggactttgccaagggccaccacggcaagcctaatctgcacacactgtattggaccggcctgttttctccagagaacctggccaagacaagcatcaagctgaatggccaggccgagctgttctaccgccctaagtccaggatgaagaggatggcacaccggctgggagagaagatgctgaacaagaagctgaaggatcagaaaaccccaatccccgacaccctgtaccaggagctgtacgactatgtgaatcacagactgtcccacgacctgtctgatgaggccagggccctgctgcccaacgtgatcaccaaggaggtgtctcacgagatcatcaaggataggcgctttaccagcgacaagttctttttccacgtgcctatcacactgaactatcaggccgccaattccccatctaagttcaaccagagggtgaatgcctacctgaaggagcaccccgagacacctatcatcggcatcgcccggggcgagagaaacctgatctatatcacagtgatcgactccaccggcaagatcctggagcagcggagcctgaacaccatccagcagtttgattaccagaagaagctggacaacagggagaaggagagggtggcagcaaggcaggcctggtctgtggtgggcacaatcaaggatctgaagcagggctatctgagccaggtcatccacgagatcgtggacctgatgatccactaccaggccgtggtggtgctggagaacctgaatttcggctttaagagcaagaggaccggcatcgccgagaaggccgtgtaccagcagttcgagaagatgctgatcgataagctgaattgcctggtgctgaaggactatccagcagagaaagtgggaggcgtgctgaacccataccagctgacagaccagttcacctcctttgccaagatgggcacccagtctggcttcctgttttacgtgcctgccccatatacatctaagatcgatcccctgaccggcttcgtggaccccttcgtgtggaaaaccatcaagaatcacgagagccgcaagcacttcctggagggcttcgactttctgcactacgacgtgaaaaccggcgacttcatcctgcactttaagatgaacagaaatctgtccttccagaggggcctgcccggctttatgcctgcatgggatatcgtgttcgagaagaacgagacacagtttgacgccaagggcacccctttcatcgccggcaagagaatcgtgccagtgatcgagaatcacagattcaccggcagataccgggacctgtatcctgccaacgagctgatcgccctgctggaggagaagggcatcgtgttcagggatggctccaacatcctgccaaagctgctggagaatgacgattctcacgccatcgacaccatggtggccctgatccgcagcgtgctgcagatgcggaactccaatgccgccacaggcgaggactatatcaacagccccgtgcgcgatctgaatggcgtgtgcttcgactcccggtttcagaacccagagtggcccatggacgccgatgccaatggcgcctaccacatcgccctgaagggccagctgctgctgaatcacctgaaggagagcaaggatctgaagctgcagaacggcatctccaatcaggactggctggcctacatccaggagctgcgcaacagcggcagcgagactcccgggacctcagagtccgccacacccgaaagtcaactggtgaagagcgagctggaagagaagaaaagcgagctcagacataagctgaagtacgttccccacgaatacattgaactgatagaaatcgctagaaacagtacgcaagacagaatactggaaatgaaggtgatggagttcttcatgaaggtttacggctatcgtggcaaacacctcgggggctcccggaagcccgacggggctatctacaccgtgggcagtcccatcgactatggcgtgatcgtggacaccaaagcttatagcggcggatataatctccccatcggccaagccgatgagatgcagaggtatgtggaggagaaccaaacaagaaacaagcatatcaaccccaacgagtggtggaaggtttatcctagctcggtgaccgagtttaagttcctattcgtgtctggccacttcaagggcaactataaggcacagctcactagactgaatcatatcacgaattgcaacggcgccgtgttatccgtggaggagctactgatcggcggagagatgatcaaagccggcaccctgaccctggaagaggtgagaagaaagtttaacaatggcgaaataaatttcggcagcggaagtggaagcggctccatcactagaaccaccaaccctagaaacgtggtgcccaagatctacatgagcgccggcagcatccccctgaccacccacatcaccaactcaattcagcccaccctgtggaccatcggcagcatcaacggcgtggcccccctggccaagagcatcaagctgggcatccccgtgaccggcagcgcctacaccgatcagaccaccgccatggtgagaaagaaggtgagcgtgttcatgggcagcggcagcgggagcggctcatcgcagctggttaagagcgagttagaagaaaaaaagagcgaactgcggcataaactgaagtatgtcccacacgagtacatcgaactgatcgagatcgcgagaaactctacccaagacagaattctggagatgaaagtaatggaatttttcatgaaggtgtatggatatagagggaagcacctgggtggcagcagaaaacccgccggcgccatctacactgtggggagccccatagactatggtgtgatcgtggataccaaggcgtatagcggcggttacaatctgcccattgggcaagcggacgagatgcaaagatatgtggaagagaatcagacgaggaacaagcacattaaccctaatgagtggtggaaggtctaccctagctccgttaccgagttcaagttcctgtttgtgagcgggcattttaagggcaactacaaggcacagctgacccgcctgaaccacataacaaactgcaacggtgccgtgctgagcgtagaagagttgctaatcggcggcgagatgatcaaggccggcacgctaaccctcgaagaggtgcgcagaaagttcaataacggcgaaatcaatttc

SEQ ID NO: 39 - DNA, 5430 bp, synthetic construct
  source 1..5430: mol_type = other DNA
atgcaactggtgaagagcgagctggaagagaagaaaagcgagctcagacataagctgaagtacgttccccacgaatacattgaactgatagaaatcgctagaaacagtacgcaagacagaatactggaaatgaaggtgatggagttcttcatgaaggtttacggctatcgtggcaaacacctcgggggctcccggaagcccgccggggctatctacaccgtgggcagtcccatcgactatggcgtgatcgtggacaccaaagcttatagcggcggatataatctccccatcggccaagccgatgagatgcagaggtatgtggaggagaaccaaacaagaaacaagcatatcaaccccaacgagtggtggaaggtttatcctagctcggtgaccgagtttaagttcctattcgtgtctggccacttcaagggcaactataaggcacagctcactagactgaatcatatcacgaattgcaacggcgccgtgttatccgtggaggagctactgatcggcggagagatgatcaaagccggcaccctgaccctggaagaggtgagaagaaagtttaacaatggcgaaataaatttcggcagcggaagtggaagcggctccatcactagaaccaccaaccctagaaacgtggtgcccaagatctacatgagcgccggcagcatccccctgaccacccacatcaccaactcaattcagcccaccctgtggaccatcggcagcatcaacggcgtggcccccctggccaagagcatcaagctgggcatccccgtgaccggcagcgcctacaccgatcagaccaccgccatggtgagaaagaaggtgagcgtgttcatgggcagcggcagcgggagcggctcatcgcagctggttaagagcgagttagaagaaaaaaagagcgaactgcggcataaactgaagtatgtcccacacgagtacatcgaactgatcgagatcgcgagaaactctacccaagacagaattctggagatgaaagtaatggaatttttcatgaaggtgtatggatatagagggaagcacctgggtggcagcagaaaacccgacggcgccatctacactgtggggagccccatagactatggtgtgatcgtggataccaaggcgtatagcggcggttacaatctgcccattgggcaagcggacgagatgcaaagatatgtggaagagaatcagacgaggaacaagcacattaaccctaatgagtggtggaaggtctaccctagctccgttaccgagttcaagttcctgtttgtgagcgggcattttaagggcaactacaaggcacagctgacccgcctgaaccacataacaaactgcaacggtgccgtgctgagcgtagaagagttgctaatcggcggcgagatgatcaaggccggcacgctaaccctcgaagaggtgcgcagaaagttcaataacggcgaaatcaatttcagcggcagcgagactcccgggacctcagagtccgccacacccgaaagtacacagttcgagggctttaccaacctgtatcaggtgagcaagacactgcggtttgagctgatcccacagggcaagaccctgaagcacatccaggagcagggcttcatcgaggaggacaaggcccgcaatgatcactacaaggagctgaagcccatcatcgatcggatctacaagacctatgccgaccagtgcctgcagctggtgcagctggattgggagaacctgagcgccgccatcgactcctatagaaaggagaaaaccgaggagacaaggaacgccctgatcgaggagcaggccacatatcgcaatgccatccacgactacttcatcggccggacagacaacctgaccgatgccatcaataagagacacgccgagatctacaagggcctgttcaaggccgagctgtttaatggcaaggtgctgaagcagctgggcaccgtgaccacaaccgagcacgagaacgccctgctgcggagcttcgacaagtttacaacctacttctccggcttttatagaaacaggaagaacgtgttcagcgccgaggatatcagcacagccatcccacaccgcatcgtgcaggacaacttccccaagtttaaggagaattgtcacatcttcacacgcctgatcaccgccgtgcccagcctgcgggagcactttgagaacgtgaagaaggccatcggcatcttcgtgagcacctccatcgaggaggtgttttccttccctttttataaccagctgctgacacagacccagatcgacctgtataaccagctgctgggaggaatctctcgggaggcaggcaccgagaagatcaagggcctgaacgaggtgctgaatctggccatccagaagaatgatgagacagcccacatcatcgcctccctgccacacagattcatccccctgtttaagcagatcctgtccgataggaacaccctgtctttcatcctggaggagtttaagagcgacgaggaagtgatccagtccttctgcaagtacaagacactgctgagaaacgagaacgtgctggagacagccgaggccctgtttaacgagctgaacagcatcgacctgacacacatcttcatcagccacaagaagctggagacaatcagcagcgccctgtgcgaccactgggatacactgaggaatgccctgtatgagcggagaatctccgagctgacaggcaagatcaccaagtctgccaaggagaaggtgcagcgcagcctgaagcacgaggatatcaacctgcaggagatcatctctgccgcaggcaaggagctgagcgaggccttcaagcagaaaaccagcgagatcctgtcccacgcacacgccgccctggatcagccactgcctacaaccctgaagaagcaggaggagaaggagatcctgaagtctcagctggacagcctgctgggcctgtaccacctgctggactggtttgccgtggatgagtccaacgaggtggaccccgagttctctgcccggctgaccggcatcaagctggagatggagccttctctgagcttctacaacaaggccagaaattatgccaccaagaagccctactccgtggagaagttcaagctgaactttcagatgcctacactggccagaggctgggacgtgaatgtggagaagaacagaggcgccatcctgtttgtgaagaacggcctgtactatctgggcatcatgccaaagcagaagggcaggtataaggccctgagcttcgagcccacagagaaaaccagcgagggctttgataagatgtactatgactacttccctgatgccgccaagatgatcccaaagtgcagcacccagctgaaggccgtgacagcccactttcagacccacacaacccccatcctgctgtccaacaatttcatcgagcctctggagatcacaaaggagatctacgacctgaacaatcctgagaaggagccaaagaagtttcagacagcctacgccaagaaaaccggcgaccagaagggctacagagaggccctgtgcaagtggatcgacttcacaagggattttctgtccaagtataccaagacaacctctatcgatctgtctagcctgcggccatcctctcagtataaggacctgggcgagtactatgccgagctgaatcccctgctgtaccacatcagcttccagagaatcgccgagaaggagatcatggatgccgtggagacaggcaagctgtacctgttccagatctataacaaggactttgccaagggccaccacggcaagcctaatctgcacacactgtattggaccggcctgttttctccagagaacctggccaagacaagcatcaagctgaatggccaggccgagctgttctaccgccctaagtccaggatgaagaggatggcacaccggctgggagagaagatgctgaacaagaagctgaaggatcagaaaaccccaatccccgacaccctgtaccaggagctgtacgactatgtgaatcacagactgtcccacgacctgtctgatgaggccagggccctgctgcccaacgtgatcaccaaggaggtgtctcacgagatcatcaaggataggcgctttaccagcgacaagttctttttccacgtgcctatcacactgaactatcaggccgccaattccccatctaagttcaaccagagggtgaatgcctacctgaaggagcaccccgagacacctatcatcggcatcgcccggggcgagagaaacctgatctatatcacagtgatcgactccaccggcaagatcctggagcagcggagcctgaacaccatccagcagtttgattaccagaagaagctggacaacagggagaaggagagggtggcagcaaggcaggcctggtctgtggtgggcacaatcaaggatctgaagcagggctatctgagccaggtcatccacgagatcgtggacctgatgatccactaccaggccgtggtggtgctggagaacctgaatttcggctttaagagcaagaggaccggcatcgccgagaaggccgtgtaccagcagttcgagaagatgctgatcgataagctgaattgcctggtgctgaaggactatccagcagagaaagtgggaggcgtgctgaacccataccagctgacagaccagttcacctcctttgccaagatgggcacccagtctggcttcctgttttacgtgcctgccccatatacatctaagatcgatcccctgaccggcttcgtggaccccttcgtgtggaaaaccatcaagaatcacgagagccgcaagcacttcctggagggcttcgactttctgcactacgacgtgaaaaccggcgacttcatcctgcactttaagatgaacagaaatctgtccttccagaggggcctgcccggctttatgcctgcatgggatatcgtgttcgagaagaacgagacacagtttgacgccaagggcacccctttcatcgccggcaagagaatcgtgccagtgatcgagaatcacagattcaccggcagataccgggacctgtatcctgccaacgagctgatcgccctgctggaggagaagggcatcgtgttcagggatggctccaacatcctgccaaagctgctggagaatgacgattctcacgccatcgacaccatggtggccctgatccgcagcgtgctgcagatgcggaactccaatgccgccacaggcgaggactatatcaacagccccgtgcgcgatctgaatggcgtgtgcttcgactcccggtttcagaacccagagtggcccatggacgccgatgccaatggcgcctaccacatcgccctgaagggccagctgctgctgaatcacctgaaggagagcaaggatctgaagctgcagaacggcatctccaatcaggactggctggcctacatccaggagctgcgcaac

SEQ ID NO: 40 - protein, 107 aa, synthetic construct
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 41 - protein, 24 aa, synthetic construct
IYIWAPLAGTCGVLLLSLVITLYC

SEQ ID NO: 42 - protein, 68 aa, synthetic construct
FWVLVVVGGVLACYSLLVTVAFIIFWVRSKRSRLLHSDYMNMTPRRPGPTRKHYQPYAPPRDFAAYRS

SEQ ID NO: 43 - protein, 41 aa, synthetic construct
KRGRKKLLYIFKQPFMRVQTTQEEDGCSCRFPEEEEGGCEL

SEQ ID NO: 44 - protein, 113 aa, synthetic construct
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 45 - DNA, 11158 bp, synthetic construct
  source 1..11158: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgagaagacctgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatgcaactggtgaagagcgagctggaagagaagaaaagcgagctcagacataagctgaagtacgttccccacgaatacattgaactgatagaaatcgctagaaacagtacgcaagacagaatactggaaatgaaggtgatggagttcttcatgaaggtttacggctatcgtggcaaacacctcgggggctcccggaagcccgacggggctatctacaccgtgggcagtcccatcgactatggcgtgatcgtggacaccaaagcttatagcggcggatataatctccccatcggccaagccgatgagatgcagaggtatgtggaggagaaccaaacaagaaacaagcatatcaaccccaacgagtggtggaaggtttatcctagctcggtgaccgagtttaagttcctattcgtgtctggccacttcaagggcaactataaggcacagctcactagactgaatcatatcacgaattgcaacggcgccgtgttatccgtggaggagctactgatcggcggagagatgatcaaagccggcaccctgaccctggaagaggtgagaagaaagtttaacaatggcgaaataaatttcggcagcggaagtggaagcggctccatcactagaaccaccaaccctagaaacgtggtgcccaagatctacatgagcgccggcagcatccccctgaccacccacatcaccaactcaattcagcccaccctgtggaccatcggcagcatcaacggcgtggcccccctggccaagagcatcaagctgggcatccccgtgaccggcagcgcctacaccgatcagaccaccgccatggtgagaaagaaggtgagcgtgttcatgggcagcggcagcgggagcggctcatcgcagctggttaagagcgagttagaagaaaaaaagagcgaactgcggcataaactgaagtatgtcccacacgagtacatcgaactgatcgagatcgcgagaaactctacccaagacagaattctggagatgaaagtaatggaatttttcatgaaggtgtatggatatagagggaagcacctgggtggcagcagaaaacccgccggcgccatctacactgtggggagccccatagactatggtgtgatcgtggataccaaggcgtatagcggcggttacaatctgcccattgggcaagcggacgagatgcaaagatatgtggaagagaatcagacgaggaacaagcacattaaccctaatgagtggtggaaggtctaccctagctccgttaccgagttcaagttcctgtttgtgagcgggcattttaagggcaactacaaggcacagctgacccgcctgaaccacataacaaactgcaacggtgccgtgctgagcgtagaagagttgctaatcggcggcgagatgatcaaggccggcacgctaaccctcgaagaggtgcgcagaaagttcaataacggcgaaatcaatttcagcggcagcgagactcccgggacctcagagtccgccacacccgaaagtacacagttcgagggctttaccaacctgtatcaggtgagcaagacactgcggtttgagctgatcccacagggcaagaccctgaagcacatccaggagcagggcttcatcgaggaggacaaggcccgcaatgatcactacaaggagctgaagcccatcatcgatcggatctacaagacctatgccgaccagtgcctgcagctggtgcagctggattgggagaacctgagcgccgccatcgactcctatagaaaggagaaaaccgaggagacaaggaacgccctgatcgaggagcaggccacatatcgcaatgccatccacgactacttcatcggccggacagacaacctgaccgatgccatcaataagagacacgccgagatctacaagggcctgttcaaggccgagctgtttaatggcaaggtgctgaagcagctgggcaccgtgaccacaaccgagcacgagaacgccctgctgcggagcttcgacaagtttacaacctacttctccggcttttatagaaacaggaagaacgtgttcagcgccgaggatatcagcacagccatcccacaccgcatcgtgcaggacaacttccccaagtttaaggagaattgtcacatcttcacacgcctgatcaccgccgtgcccagcctgcgggagcactttgagaacgtgaagaaggccatcggcatcttcgtgagcacctccatcgaggaggtgttttccttccctttttataaccagctgctgacacagacccagatcgacctgtataaccagctgctgggaggaatctctcgggaggcaggcaccgagaagatcaagggcctgaacgaggtgctgaatctggccatccagaagaatgatgagacagcccacatcatcgcctccctgccacacagattcatccccctgtttaagcagatcctgtccgataggaacaccctgtctttcatcctggaggagtttaagagcgacgaggaagtgatccagtccttctgcaagtacaagacactgctgagaaacgagaacgtgctggagacagccgaggccctgtttaacgagctgaacagcatcgacctgacacacatcttcatcagccacaagaagctggagacaatcagcagcgccctgtgcgaccactgggatacactgaggaatgccctgtatgagcggagaatctccgagctgacaggcaagatcaccaagtctgccaaggagaaggtgcagcgcagcctgaagcacgaggatatcaacctgcaggagatcatctctgccgcaggcaaggagctgagcgaggccttcaagcagaaaaccagcgagatcctgtcccacgcacacgccgccctggatcagccactgcctacaaccctgaagaagcaggaggagaaggagatcctgaagtctcagctggacagcctgctgggcctgtaccacctgctggactggtttgccgtggatgagtccaacgaggtggaccccgagttctctgcccggctgaccggcatcaagctggagatggagccttctctgagcttctacaacaaggccagaaattatgccaccaagaagccctactccgtggagaagttcaagctgaactttcagatgcctacactggccagaggctgggacgtgaatgtggagaagaacagaggcgccatcctgtttgtgaagaacggcctgtactatctgggcatcatgccaaagcagaagggcaggtataaggccctgagcttcgagcccacagagaaaaccagcgagggctttgataagatgtactatgactacttccctgatgccgccaagatgatcccaaagtgcagcacccagctgaaggccgtgacagcccactttcagacccacacaacccccatcctgctgtccaacaatttcatcgagcctctggagatcacaaaggagatctacgacctgaacaatcctgagaaggagccaaagaagtttcagacagcctacgccaagaaaaccggcgaccagaagggctacagagaggccctgtgcaagtggatcgacttcacaagggattttctgtccaagtataccaagacaacctctatcgatctgtctagcctgcggccatcctctcagtataaggacctgggcgagtactatgccgagctgaatcccctgctgtaccacatcagcttccagagaatcgccgagaaggagatcatggatgccgtggagacaggcaagctgtacctgttccagatctataacaaggactttgccaagggccaccacggcaagcctaatctgcacacactgtattggaccggcctgttttctccagagaacctggccaagacaagcatcaagctgaatggccaggccgagctgttctaccgccctaagtccaggatgaagaggatggcacaccggctgggagagaagatgctgaacaagaagctgaaggatcagaaaaccccaatccccgacaccctgtaccaggagctgtacgactatgtgaatcacagactgtcccacgacctgtctgatgaggccagggccctgctgcccaacgtgatcaccaaggaggtgtctcacgagatcatcaaggataggcgctttaccagcgacaagttctttttccacgtgcctatcacactgaactatcaggccgccaattccccatctaagttcaaccagagggtgaatgcctacctgaaggagcaccccgagacacctatcatcggcatcgcccggggcgagagaaacctgatctatatcacagtgatcgactccaccggcaagatcctggagcagcggagcctgaacaccatccagcagtttgattaccagaagaagctggacaacagggagaaggagagggtggcagcaaggcaggcctggtctgtggtgggcacaatcaaggatctgaagcagggctatctgagccaggtcatccacgagatcgtggacctgatgatccactaccaggccgtggtggtgctggagaacctgaatttcggctttaagagcaagaggaccggcatcgccgagaaggccgtgtaccagcagttcgagaagatgctgatcgataagctgaattgcctggtgctgaaggactatccagcagagaaagtgggaggcgtgctgaacccataccagctgacagaccagttcacctcctttgccaagatgggcacccagtctggcttcctgttttacgtgcctgccccatatacatctaagatcgatcccctgaccggcttcgtggaccccttcgtgtggaaaaccatcaagaatcacgagagccgcaagcacttcctggagggcttcgactttctgcactacgacgtgaaaaccggcgacttcatcctgcactttaagatgaacagaaatctgtccttccagaggggcctgcccggctttatgcctgcatgggatatcgtgttcgagaagaacgagacacagtttgacgccaagggcacccctttcatcgccggcaagagaatcgtgccagtgatcgagaatcacagattcaccggcagataccgggacctgtatcctgccaacgagctgatcgccctgctggaggagaagggcatcgtgttcagggatggctccaacatcctgccaaagctgctggagaatgacgattctcacgccatcgacaccatggtggccctgatccgcagcgtgctgcagatgcggaactccaatgccgccacaggcgaggactatatcaacagccccgtgcgcgatctgaatggcgtgtgcttcgactcccggtttcagaacccagagtggcccatggacgccgatgccaatggcgcctaccacatcgccctgaagggccagctgctgctgaatcacctgaaggagagcaaggatctgaagctgcagaacggcatctccaatcaggactggctggcctacatccaggagctgcgcaacaaaaggccggcggccacgaaaaaggccggccaggcaaaaaagaaaaagggatcctacccatacgatgttccagattacgcttatccctacgacgtgcctgattatgcatacccatatgatgtccccgactatgccgagggcagaggaagtctgctaacatgcggtgacgtcgaggagaatcctggcccaatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaactagtcctgcaggcatgcaagcttgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagggaattaattcgagctcggtacctttaagaccgatgacttacaaggcagctgtagatcttagccactttttaaaagaaattaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 46 - DNA, 11941 bp, synthetic construct
  source 1..11941: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgagaagacctgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatgggcagctcagagactggcccagtggctgtggaccccacattgagacggcggatcgagccccatgagtttgaggtattcttcgatccgagagagctccgcaaggagacctgcctgctttacgaaattaattgggggggccggcactccatttggcgacatacatcacagaacactaacaagcacgtcgaagtcaacttcatcgagaagttcacgacagaaagatatttctgtccgaacacaaggtgcagcattacctggtttctcagctggagcccatgcggcgaatgtagtagggccatcactgaattcctgtcaaggtatccccacgtcactctgtttatttacatcgcaaggctgtaccaccacgctgacccccgcaatcgacaaggcctgcgggatttgatctcttcaggtgtgactatccaaattatgactgagcaggagtcaggatactgctggagaaactttgtgaattatagcccgagtaatgaagcccactggcctaggtatccccatctgtgggtacgactgtacgttcttgaactgtactgcatcatactgggcctgcctccttgtctcaacattctgagaaggaagcagccacagctgacattctttaccatcgctcttcagtcttgtcattaccagcgactgcccccacacattctctgggccaccgggttgaaatctggtggttcttctggtggttctagcggcagcgagactcccgggacctcagagtccgccacacccgaaagttccggagggagtagcggcgggtctacacagttcgagggctttaccaacctgtatcaggtgagcaagacactgcggtttgagctgatcccacagggcaagaccctgaagcacatccaggagcagggcttcatcgaggaggacaaggcccgcaatgatcactacaaggagctgaagcccatcatcgatcggatctacaagacctatgccgaccagtgcctgcagctggtgcagctggattgggagaacctgagcgccgccatcgactcctatagaaaggagaaaaccgaggagacaaggaacgccctgatcgaggagcaggccacatatcgcaatgccatccacgactacttcatcggccggacagacaacctgaccgatgccatcaataagagacacgccgagatctacaagggcctgttcaaggccgagctgtttaatggcaaggtgctgaagcagctgggcaccgtgaccacaaccgagcacgagaacgccctgctgcggagcttcgacaagtttacaacctacttctccggcttttatagaaacaggaagaacgtgttcagcgccgaggatatcagcacagccatcccacaccgcatcgtgcaggacaacttccccaagtttaaggagaattgtcacatcttcacacgcctgatcaccgccgtgcccagcctgcgggagcactttgagaacgtgaagaaggccatcggcatcttcgtgagcacctccatcgaggaggtgttttccttccctttttataaccagctgctgacacagacccagatcgacctgtataaccagctgctgggaggaatctctcgggaggcaggcaccgagaagatcaagggcctgaacgaggtgctgaatctggccatccagaagaatgatgagacagcccacatcatcgcctccctgccacacagattcatccccctgtttaagcagatcctgtccgataggaacaccctgtctttcatcctggaggagtttaagagcgacgaggaagtgatccagtccttctgcaagtacaagacactgctgagaaacgagaacgtgctggagacagccgaggccctgtttaacgagctgaacagcatcgacctgacacacatcttcatcagccacaagaagctggagacaatcagcagcgccctgtgcgaccactgggatacactgaggaatgccctgtatgagcggagaatctccgagctgacaggcaagatcaccaagtctgccaaggagaaggtgcagcgcagcctgaagcacgaggatatcaacctgcaggagatcatctctgccgcaggcaaggagctgagcgaggccttcaagcagaaaaccagcgagatcctgtcccacgcacacgccgccctggatcagccactgcctacaaccctgaagaagcaggaggagaaggagatcctgaagtctcagctggacagcctgctgggcctgtaccacctgctggactggtttgccgtggatgagtccaacgaggtggaccccgagttctctgcccggctgaccggcatcaagctggagatggagccttctctgagcttctacaacaaggccagaaattatgccaccaagaagccctactccgtggagaagttcaagctgaactttcagatgcctacactggccagaggctgggacgtgaatgtggagaagaacagaggcgccatcctgtttgtgaagaacggcctgtactatctgggcatcatgccaaagcagaagggcaggtataaggccctgagcttcgagcccacagagaaaaccagcgagggctttgataagatgtactatgactacttccctgatgccgccaagatgatcccaaagtgcagcacccagctgaaggccgtgacagcccactttcagacccacacaacccccatcctgctgtccaacaatttcatcgagcctctggagatcacaaaggagatctacgacctgaacaatcctgagaaggagccaaagaagtttcagacagcctacgccaagaaaaccggcgaccagaagggctacagagaggccctgtgcaagtggatcgacttcacaagggattttctgtccaagtataccaagacaacctctatcgatctgtctagcctgcggccatcctctcagtataaggacctgggcgagtactatgccgagctgaatcccctgctgtaccacatcagcttccagagaatcgccgagaaggagatcatggatgccgtggagacaggcaagctgtacctgttccagatctataacaaggactttgccaagggccaccacggcaagcctaatctgcacacactgtattggaccggcctgttttctccagagaacctggccaagacaagcatcaagctgaatggccaggccgagctgttctaccgccctaagtccaggatgaagaggatggcacaccggctgggagagaagatgctgaacaagaagctgaaggatcagaaaaccccaatccccgacaccctgtaccaggagctgtacgactatgtgaatcacagactgtcccacgacctgtctgatgaggccagggccctgctgcccaacgtgatcaccaaggaggtgtctcacgagatcatcaaggataggcgctttaccagcgacaagttctttttccacgtgcctatcacactgaactatcaggccgccaattccccatctaagttcaaccagagggtgaatgcctacctgaaggagcaccccgagacacctatcatcggcatcgcccggggcgagagaaacctgatctatatcacagtgatcgactccaccggcaagatcctggagcagcggagcctgaacaccatccagcagtttgattaccagaagaagctggacaacagggagaaggagagggtggcagcaaggcaggcctggtctgtggtgggcacaatcaaggatctgaagcagggctatctgagccaggtcatccacgagatcgtggacctgatgatccactaccaggccgtggtggtgctggagaacctgaatttcggctttaagagcaagaggaccggcatcgccgagaaggccgtgtaccagcagttcgagaagatgctgatcgataagctgaattgcctggtgctgaaggactatccagcagagaaagtgggaggcgtgctgaacccataccagctgacagaccagttcacctcctttgccaagatgggcacccagtctggcttcctgttttacgtgcctgccccatatacatctaagatcgatcccctgaccggcttcgtggaccccttcgtgtggaaaaccatcaagaatcacgagagccgcaagcacttcctggagggcttcgactttctgcactacgacgtgaaaaccggcgacttcatcctgcactttaagatgaacagaaatctgtccttccagaggggcctgcccggctttatgcctgcatgggatatcgtgttcgagaagaacgagacacagtttgacgccaagggcacccctttcatcgccggcaagagaatcgtgccagtgatcgagaatcacagattcaccggcagataccgggacctgtatcctgccaacgagctgatcgccctgctggaggagaagggcatcgtgttcagggatggctccaacatcctgccaaagctgctggagaatgacgattctcacgccatcgacaccatggtggccctgatccgcagcgtgctgcagatgcggaactccaatgccgccacaggcgaggactatatcaacagccccgtgcgcgatctgaatggcgtgtgcttcgactcccggtttcagaacccagagtggcccatggacgccgatgccaatggcgcctaccacatcgccctgaagggccagctgctgctgaatcacctgaaggagagcaaggatctgaagctgcagaacggcatctccaatcaggactggctggcctacatccaggagctgcgcaacagcggcagcgagactcccgggacctcagagtccgccacacccgaaagtcaactggtgaagagcgagctggaagagaagaaaagcgagctcagacataagctgaagtacgttccccacgaatacattgaactgatagaaatcgctagaaacagtacgcaagacagaatactggaaatgaaggtgatggagttcttcatgaaggtttacggctatcgtggcaaacacctcgggggctcccggaagcccgccggggctatctacaccgtgggcagtcccatcgactatggcgtgatcgtggacaccaaagcttatagcggcggatataatctccccatcggccaagccgatgagatgcagaggtatgtggaggagaaccaaacaagaaacaagcatatcaaccccaacgagtggtggaaggtttatcctagctcggtgaccgagtttaagttcctattcgtgtctggccacttcaagggcaactataaggcacagctcactagactgaatcatatcacgaattgcaacggcgccgtgttatccgtggaggagctactgatcggcggagagatgatcaaagccggcaccctgaccctggaagaggtgagaagaaagtttaacaatggcgaaataaatttcggcagcggaagtggaagcggctccatcactagaaccaccaaccctagaaacgtggtgcccaagatctacatgagcgccggcagcatccccctgaccacccacatcaccaactcaattcagcccaccctgtggaccatcggcagcatcaacggcgtggcccccctggccaagagcatcaagctgggcatccccgtgaccggcagcgcctacaccgatcagaccaccgccatggtgagaaagaaggtgagcgtgttcatgggcagcggcagcgggagcggctcatcgcagctggttaagagcgagttagaagaaaaaaagagcgaactgcggcataaactgaagtatgtcccacacgagtacatcgaactgatcgagatcgcgagaaactctacccaagacagaattctggagatgaaagtaatggaatttttcatgaaggtgtatggatatagagggaagcacctgggtggcagcagaaaacccgacggcgccatctacactgtggggagccccatagactatggtgtgatcgtggataccaaggcgtatagcggcggttacaatctgcccattgggcaagcggacgagatgcaaagatatgtggaagagaatcagacgaggaacaagcacattaaccctaatgagtggtggaaggtctaccctagctccgttaccgagttcaagttcctgtttgtgagcgggcattttaagggcaactacaaggcacagctgacccgcctgaaccacataacaaactgcaacggtgccgtgctgagcgtagaagagttgctaatcggcggcgagatgatcaaggccggcacgctaaccctcgaagaggtgcgcagaaagttcaataacggcgaaatcaatttcaaaaggccggcggccacgaaaaaggccggccaggcaaaaaagaaaaagggatcctacccatacgatgttccagattacgcttatccctacgacgtgcctgattatgcatacccatatgatgtccccgactatgccgagggcagaggaagtctgctaacatgcggtgacgtcgaggagaatcctggcccaatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaactagtcctgcaggcatgcaagcttgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagggaattaattcgagctcggtacctttaagaccgatgacttacaaggcagctgtagatcttagccactttttaaaagaaattaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 47 - DNA, 11809 bp, synthetic construct
  source 1..11809: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgagaagacctgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatgggcagctcagagactggcccagtggctgtggaccccacattgagacggcggatcgagccccatgagtttgaggtattcttcgatccgagagagctccgcaaggagacctgcctgctttacgaaattaattgggggggccggcactccatttggcgacatacatcacagaacactaacaagcacgtcgaagtcaacttcatcgagaagttcacgacagaaagatatttctgtccgaacacaaggtgcagcattacctggtttctcagctggagcccatgcggcgaatgtagtagggccatcactgaattcctgtcaaggtatccccacgtcactctgtttatttacatcgcaaggctgtaccaccacgctgacccccgcaatcgacaaggcctgcgggatttgatctcttcaggtgtgactatccaaattatgactgagcaggagtcaggatactgctggagaaactttgtgaattatagcccgagtaatgaagcccactggcctaggtatccccatctgtgggtacgactgtacgttcttgaactgtactgcatcatactgggcctgcctccttgtctcaacattctgagaaggaagcagccacagctgacattctttaccatcgctcttcagtcttgtcattaccagcgactgcccccacacattctctgggccaccgggttgaaatctggtggttcttctggtggttctagcggcagcgagactcccgggacctcagagtccgccacacccgaaagttccggagggagtagcggcgggtctaataacggaactaataacttccaaaacttcatcgggatcagttccttgcagaaaactctccggaatgctctcatcccaactgagactactcagcagttcattgttaagaatggaatcataaaagaggacgagcttaggggggaaaataggcaaatcctcaaggatatcatggatgactattataggggctttatatccgagacactgagcagcattgatgatatagactggacctctcttttcgaaaagatggaaatacaacttaaaaatggagataacaaggacaccctgataaaggaacagaccgaatataggaaggcaattcataaaaagtttgctaacgatgataggtttaaaaacatgttctcagcaaaactcatttcagatatactgcccgaattcgttatccacaacaacaactactccgctagcgaaaaagaggaaaagacccaagtcataaagctgttctctcgattcgcgacgagttttaaagattatttccgaaatcgcgcaaactgtttctcagctgatgatatcagcagctcatcctgtcatcggatcgttaacgataatgctgaaatcttcttctccaatgcacttgtttataggcgcattgttaaatctctctcaaacgatgatatcaataagatttccggcgatatgaaggacagtcttaaggagatgagcctcgaagagatatactcatacgagaaatatggcgaatttatcacccaggaagggatttccttctataatgacatttgcggcaaagtcaattccttcatgaacctgtattgccaaaaaaataaagaaaacaagaacctctataagctgcaaaagttgcataagcaaatactttgtatcgcggatacaagctatgaagttccctacaagttcgagagtgatgaggaggtgtatcaatctgtcaatggtttccttgataatatttcttctaagcatattgttgaacgactccgaaagataggagacaactataatggatacaatttggataaaatctacatcgtgtctaaattttacgagagtgtgtcacaaaaaacatatagagactgggagacaattaataccgccctggagatacattacaacaatatacttcccgggaacgggaagtctaaggcagacaaggtgaagaaagccgtgaagaacgacttgcaaaagtcaattaccgaaatcaatgagcttgtttcaaactataaactttgttcagatgacaatattaaagccgaaacctatattcatgaaatctctcatattctgaataactttgaggcgcaagaactgaaatataacccagaaatacacctcgttgagtccgaactgaaagcaagcgaactgaaaaatgttttggacgtgataatgaacgcttttcattggtgctcagtctttatgacagaggagcttgttgacaaggataacaatttctatgcggaactggaagagatttacgacgaaatctatccggtcatatccctgtataacctggttcgcaactatgtcacgcaaaaaccatacagcacgaagaagattaaactgaactttggtattccgacgctggcccgcggatggtcaaaatctgttgaatactcacgaaatgccataatcctgatgcgagataacctctactaccttggaatctttaatgctaaaaataaacccgataaaaaaattatcgaagggaacacgagtgaaaacaaaggtgattataaaaaaatgatatataatctgcttccaggaccaaataagatgatacccaaagttttcctttcttcaaagaccggcgtcgagacatataaaccatccgcgtacatacttgaaggctacaaacaaaataaacatatcaaatcatctaaggattttgacattacgttctgtcatgatttgattgactatttcaaaaattgcatagccattcatccagagtggaaaaactttgggtttgacttctctgataccagtacatatgaagacataagtggattttaccgagaagtagagctccaaggttataaaatagactggacctatatatctgaaaaggatatagaccttttgcaagagaagggacagctttatcttttccaaatctacaacaaagacttcagtaagaaaagtaccgggaatgacaatcttcataccatgtatctgaagaacctgttctccgaagaaaatctgaaggacatagtcctgaagcttaatggcgaagcggaaatttttttccgaaagagctctattaagaaccccataatacataagaagggaagcattctcgttaatcgaacgtatgaggccgaagagaaagatcaatttgggaatatccaaatcgttcgaaagaacataccagaaaatatttaccaagaattgtacaaatattttaacgataaaagcgacaaagaactgtctgatgaagctgctaagctgaaaaacgtcgtcggccatcatgaggccgcgacgaatatagtcaaggattaccgatatacatacgataagtatttcctgcatatgcccatcactatcaactttaaggcaaataagactggattcattaatgacagaatactgcaatacatagctaaagaaaaagatttgcatgttattggcattgccaggggtgagcgcaatcttatctatgtaagcgtcattgatacttgcgggaatatcgtagagcagaagtcatttaatattgtaaatgggtacgattaccaaatcaagttgaagcagcaagagggagcacgacagattgcccgcaaggagtggaaagagatcggaaagataaaggagatcaaggaggggtatttgtcccttgttatacacgaaatttccaagatggtaatcaagtacaacgctataattgctatggaggatctctcctatggatttaaaaagggaagatttaaagtcgagcggcaggtatatcagaaatttgaaacaatgcttattaataaacttaattatctcgttttcaaagacattagtatcaccgaaaacggtgggctgttgaagggctatcaacttacgtacataccagataagcttaagaatgtgggtcaccaatgcggatgcatattctacgtgcccgcagcttatacaagcaaaatcgacccaacaacgggtttcgtaaacatatttaagttcaaggatctcaccgtggatgccaagcgagagttcataaaaaaatttgactcaatcagatatgactcagaaaagaatcttttttgttttaccttcgactacaataatttcattacacaaaatacggttatgagcaagtcatcctggtccgtatatacgtatggagtgcgcataaagcggagattcgttaacgggcgattttctaatgagtccgatacaatcgatataacaaaggatatggaaaaaactctggaaatgactgatataaattggagggacggtcatgacctcaggcaagacattatcgattatgagatcgtgcaacatatttttgagatctttcggttgactgtccaaatgaggaactctctgtctgaattggaagatagggactacgatcgcctgataagccccgtgttgaacgagaataacatattctacgattccgcgaaagccggggatgcgctccctaaggacgccgatgcaaatggggcctattgtattgctttgaaagggctgtacgaaatcaaacagatcaccgaaaactggaaagaagacgggaagtttagtcgggataaactgaagatatccaacaaggactggtttgactttatccaaaataagcgatatttgagcggcagcgagactcccgggacctcagagtccgccacacccgaaagtcaactggtgaagagcgagctggaagagaagaaaagcgagctcagacataagctgaagtacgttccccacgaatacattgaactgatagaaatcgctagaaacagtacgcaagacagaatactggaaatgaaggtgatggagttcttcatgaaggtttacggctatcgtggcaaacacctcgggggctcccggaagcccgccggggctatctacaccgtgggcagtcccatcgactatggcgtgatcgtggacaccaaagcttatagcggcggatataatctccccatcggccaagccgatgagatgcagaggtatgtggaggagaaccaaacaagaaacaagcatatcaaccccaacgagtggtggaaggtttatcctagctcggtgaccgagtttaagttcctattcgtgtctggccacttcaagggcaactataaggcacagctcactagactgaatcatatcacgaattgcaacggcgccgtgttatccgtggaggagctactgatcggcggagagatgatcaaagccggcaccctgaccctggaagaggtgagaagaaagtttaacaatggcgaaataaatttcggcagcggaagtggaagcggctccatcactagaaccaccaaccctagaaacgtggtgcccaagatctacatgagcgccggcagcatccccctgaccacccacatcaccaactcaattcagcccaccctgtggaccatcggcagcatcaacggcgtggcccccctggccaagagcatcaagctgggcatccccgtgaccggcagcgcctacaccgatcagaccaccgccatggtgagaaagaaggtgagcgtgttcatgggcagcggcagcgggagcggctcatcgcagctggttaagagcgagttagaagaaaaaaagagcgaactgcggcataaactgaagtatgtcccacacgagtacatcgaactgatcgagatcgcgagaaactctacccaagacagaattctggagatgaaagtaatggaatttttcatgaaggtgtatggatatagagggaagcacctgggtggcagcagaaaacccgacggcgccatctacactgtggggagccccatagactatggtgtgatcgtggataccaaggcgtatagcggcggttacaatctgcccattgggcaagcggacgagatgcaaagatatgtggaagagaatcagacgaggaacaagcacattaaccctaatgagtggtggaaggtctaccctagctccgttaccgagttcaagttcctgtttgtgagcgggcattttaagggcaactacaaggcacagctgacccgcctgaaccacataacaaactgcaacggtgccgtgctgagcgtagaagagttgctaatcggcggcgagatgatcaaggccggcacgctaaccctcgaagaggtgcgcagaaagttcaataacggcgaaatcaatttcaaaaggccggcggccacgaaaaaggccggccaggcaaaaaagaaaaagggatcctacccatacgatgttccagattacgcttatccctacgacgtgcctgattatgcatacccatatgatgtccccgactatgccgagggcagaggaagtctgctaacatgcggtgacgtcgaggagaatcctggcccaatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaactagtcctgcaggcatgcaagcttgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagggaattaattcgagctcggtacctttaagaccgatgacttacaaggcagctgtagatcttagccactttttaaaagaaattaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 48 - DNA, 9517 bp, synthetic construct
  source 1..9517: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgagaagacctgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatgaataacggaactaataacttccaaaacttcatcgggatcagttccttgcagaaaactctccggaatgctctcatcccaactgagactactcagcagttcattgttaagaatggaatcataaaagaggacgagcttaggggggaaaataggcaaatcctcaaggatatcatggatgactattataggggctttatatccgagacactgagcagcattgatgatatagactggacctctcttttcgaaaagatggaaatacaacttaaaaatggagataacaaggacaccctgataaaggaacagaccgaatataggaaggcaattcataaaaagtttgctaacgatgataggtttaaaaacatgttctcagcaaaactcatttcagatatactgcccgaattcgttatccacaacaacaactactccgctagcgaaaaagaggaaaagacccaagtcataaagctgttctctcgattcgcgacgagttttaaagattatttccgaaatcgcgcaaactgtttctcagctgatgatatcagcagctcatcctgtcatcggatcgttaacgataatgctgaaatcttcttctccaatgcacttgtttataggcgcattgttaaatctctctcaaacgatgatatcaataagatttccggcgatatgaaggacagtcttaaggagatgagcctcgaagagatatactcatacgagaaatatggcgaatttatcacccaggaagggatttccttctataatgacatttgcggcaaagtcaattccttcatgaacctgtattgccaaaaaaataaagaaaacaagaacctctataagctgcaaaagttgcataagcaaatactttgtatcgcggatacaagctatgaagttccctacaagttcgagagtgatgaggaggtgtatcaatctgtcaatggtttccttgataatatttcttctaagcatattgttgaacgactccgaaagataggagacaactataatggatacaatttggataaaatctacatcgtgtctaaattttacgagagtgtgtcacaaaaaacatatagagactgggagacaattaataccgccctggagatacattacaacaatatacttcccgggaacgggaagtctaaggcagacaaggtgaagaaagccgtgaagaacgacttgcaaaagtcaattaccgaaatcaatgagcttgtttcaaactataaactttgttcagatgacaatattaaagccgaaacctatattcatgaaatctctcatattctgaataactttgaggcgcaagaactgaaatataacccagaaatacacctcgttgagtccgaactgaaagcaagcgaactgaaaaatgttttggacgtgataatgaacgcttttcattggtgctcagtctttatgacagaggagcttgttgacaaggataacaatttctatgcggaactggaagagatttacgacgaaatctatccggtcatatccctgtataacctggttcgcaactatgtcacgcaaaaaccatacagcacgaagaagattaaactgaactttggtattccgacgctggcccgcggatggtcaaaatctgttgaatactcacgaaatgccataatcctgatgcgagataacctctactaccttggaatctttaatgctaaaaataaacccgataaaaaaattatcgaagggaacacgagtgaaaacaaaggtgattataaaaaaatgatatataatctgcttccaggaccaaataagatgatacccaaagttttcctttcttcaaagaccggcgtcgagacatataaaccatccgcgtacatacttgaaggctacaaacaaaataaacatatcaaatcatctaaggattttgacattacgttctgtcatgatttgattgactatttcaaaaattgcatagccattcatccagagtggaaaaactttgggtttgacttctctgataccagtacatatgaagacataagtggattttaccgagaagtagagctccaaggttataaaatagactggacctatatatctgaaaaggatatagaccttttgcaagagaagggacagctttatcttttccaaatctacaacaaagacttcagtaagaaaagtaccgggaatgacaatcttcataccatgtatctgaagaacctgttctccgaagaaaatctgaaggacatagtcctgaagcttaatggcgaagcggaaatttttttccgaaagagctctattaagaaccccataatacataagaagggaagcattctcgttaatcgaacgtatgaggccgaagagaaagatcaatttgggaatatccaaatcgttcgaaagaacataccagaaaatatttaccaagaattgtacaaatattttaacgataaaagcgacaaagaactgtctgatgaagctgctaagctgaaaaacgtcgtcggccatcatgaggccgcgacgaatatagtcaaggattaccgatatacatacgataagtatttcctgcatatgcccatcactatcaactttaaggcaaataagactggattcattaatgacagaatactgcaatacatagctaaagaaaaagatttgcatgttattggcattgacaggggtgagcgcaatcttatctatgtaagcgtcattgatacttgcgggaatatcgtagagcagaagtcatttaatattgtaaatgggtacgattaccaaatcaagttgaagcagcaagagggagcacgacagattgcccgcaaggagtggaaagagatcggaaagataaaggagatcaaggaggggtatttgtcccttgttatacacgaaatttccaagatggtaatcaagtacaacgctataattgctatggaggatctctcctatggatttaaaaagggaagatttaaagtcgagcggcaggtatatcagaaatttgaaacaatgcttattaataaacttaattatctcgttttcaaagacattagtatcaccgaaaacggtgggctgttgaagggctatcaacttacgtacataccagataagcttaagaatgtgggtcaccaatgcggatgcatattctacgtgcccgcagcttatacaagcaaaatcgacccaacaacgggtttcgtaaacatatttaagttcaaggatctcaccgtggatgccaagcgagagttcataaaaaaatttgactcaatcagatatgactcagaaaagaatcttttttgttttaccttcgactacaataatttcattacacaaaatacggttatgagcaagtcatcctggtccgtatatacgtatggagtgcgcataaagcggagattcgttaacgggcgattttctaatgagtccgatacaatcgatataacaaaggatatggaaaaaactctggaaatgactgatataaattggagggacggtcatgacctcaggcaagacattatcgattatgagatcgtgcaacatatttttgagatctttcggttgactgtccaaatgaggaactctctgtctgaattggaagatagggactacgatcgcctgataagccccgtgttgaacgagaataacatattctacgattccgcgaaagccggggatgcgctccctaaggacgccgatgcaaatggggcctattgtattgctttgaaagggctgtacgaaatcaaacagatcaccgaaaactggaaagaagacgggaagtttagtcgggataaactgaagatatccaacaaggactggtttgactttatccaaaataagcgatatttgaaaaggccggcggccacgaaaaaggccggccaggcaaaaaagaaaaagggatcctacccatacgatgttccagattacgcttatccctacgacgtgcctgattatgcatacccatatgatgtccccgactatgccgagggcagaggaagtctgctaacatgcggtgacgtcgaggagaatcctggcccaatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaactagtcctgcaggcatgcaagcttgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagggaattaattcgagctcggtacctttaagaccgatgacttacaaggcagctgtagatcttagccactttttaaaagaaattaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 49 - DNA, 11158 bp, synthetic construct
  source 1..11158: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgagaagacctgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatgacacagttcgagggctttaccaacctgtatcaggtgagcaagacactgcggtttgagctgatcccacagggcaagaccctgaagcacatccaggagcagggcttcatcgaggaggacaaggcccgcaatgatcactacaaggagctgaagcccatcatcgatcggatctacaagacctatgccgaccagtgcctgcagctggtgcagctggattgggagaacctgagcgccgccatcgactcctatagaaaggagaaaaccgaggagacaaggaacgccctgatcgaggagcaggccacatatcgcaatgccatccacgactacttcatcggccggacagacaacctgaccgatgccatcaataagagacacgccgagatctacaagggcctgttcaaggccgagctgtttaatggcaaggtgctgaagcagctgggcaccgtgaccacaaccgagcacgagaacgccctgctgcggagcttcgacaagtttacaacctacttctccggcttttatagaaacaggaagaacgtgttcagcgccgaggatatcagcacagccatcccacaccgcatcgtgcaggacaacttccccaagtttaaggagaattgtcacatcttcacacgcctgatcaccgccgtgcccagcctgcgggagcactttgagaacgtgaagaaggccatcggcatcttcgtgagcacctccatcgaggaggtgttttccttccctttttataaccagctgctgacacagacccagatcgacctgtataaccagctgctgggaggaatctctcgggaggcaggcaccgagaagatcaagggcctgaacgaggtgctgaatctggccatccagaagaatgatgagacagcccacatcatcgcctccctgccacacagattcatccccctgtttaagcagatcctgtccgataggaacaccctgtctttcatcctggaggagtttaagagcgacgaggaagtgatccagtccttctgcaagtacaagacactgctgagaaacgagaacgtgctggagacagccgaggccctgtttaacgagctgaacagcatcgacctgacacacatcttcatcagccacaagaagctggagacaatcagcagcgccctgtgcgaccactgggatacactgaggaatgccctgtatgagcggagaatctccgagctgacaggcaagatcaccaagtctgccaaggagaaggtgcagcgcagcctgaagcacgaggatatcaacctgcaggagatcatctctgccgcaggcaaggagctgagcgaggccttcaagcagaaaaccagcgagatcctgtcccacgcacacgccgccctggatcagccactgcctacaaccctgaagaagcaggaggagaaggagatcctgaagtctcagctggacagcctgctgggcctgtaccacctgctggactggtttgccgtggatgagtccaacgaggtggaccccgagttctctgcccggctgaccggcatcaagctggagatggagccttctctgagcttctacaacaaggccagaaattatgccaccaagaagccctactccgtggagaagttcaagctgaactttcagatgcctacactggccagaggctgggacgtgaatgtggagaagaacagaggcgccatcctgtttgtgaagaacggcctgtactatctgggcatcatgccaaagcagaagggcaggtataaggccctgagcttcgagcccacagagaaaaccagcgagggctttgataagatgtactatgactacttccctgatgccgccaagatgatcccaaagtgcagcacccagctgaaggccgtgacagcccactttcagacccacacaacccccatcctgctgtccaacaatttcatcgagcctctggagatcacaaaggagatctacgacctgaacaatcctgagaaggagccaaagaagtttcagacagcctacgccaagaaaaccggcgaccagaagggctacagagaggccctgtgcaagtggatcgacttcacaagggattttctgtccaagtataccaagacaacctctatcgatctgtctagcctgcggccatcctctcagtataaggacctgggcgagtactatgccgagctgaatcccctgctgtaccacatcagcttccagagaatcgccgagaaggagatcatggatgccgtggagacaggcaagctgtacctgttccagatctataacaaggactttgccaagggccaccacggcaagcctaatctgcacacactgtattggaccggcctgttttctccagagaacctggccaagacaagcatcaagctgaatggccaggccgagctgttctaccgccctaagtccaggatgaagaggatggcacaccggctgggagagaagatgctgaacaagaagctgaaggatcagaaaaccccaatccccgacaccctgtaccaggagctgtacgactatgtgaatcacagactgtcccacgacctgtctgatgaggccagggccctgctgcccaacgtgatcaccaaggaggtgtctcacgagatcatcaaggataggcgctttaccagcgacaagttctttttccacgtgcctatcacactgaactatcaggccgccaattccccatctaagttcaaccagagggtgaatgcctacctgaaggagcaccccgagacacctatcatcggcatcgcccggggcgagagaaacctgatctatatcacagtgatcgactccaccggcaagatcctggagcagcggagcctgaacaccatccagcagtttgattaccagaagaagctggacaacagggagaaggagagggtggcagcaaggcaggcctggtctgtggtgggcacaatcaaggatctgaagcagggctatctgagccaggtcatccacgagatcgtggacctgatgatccactaccaggccgtggtggtgctggagaacctgaatttcggctttaagagcaagaggaccggcatcgccgagaaggccgtgtaccagcagttcgagaagatgctgatcgataagctgaattgcctggtgctgaaggactatccagcagagaaagtgggaggcgtgctgaacccataccagctgacagaccagttcacctcctttgccaagatgggcacccagtctggcttcctgttttacgtgcctgccccatatacatctaagatcgatcccctgaccggcttcgtggaccccttcgtgtggaaaaccatcaagaatcacgagagccgcaagcacttcctggagggcttcgactttctgcactacgacgtgaaaaccggcgacttcatcctgcactttaagatgaacagaaatctgtccttccagaggggcctgcccggctttatgcctgcatgggatatcgtgttcgagaagaacgagacacagtttgacgccaagggcacccctttcatcgccggcaagagaatcgtgccagtgatcgagaatcacagattcaccggcagataccgggacctgtatcctgccaacgagctgatcgccctgctggaggagaagggcatcgtgttcagggatggctccaacatcctgccaaagctgctggagaatgacgattctcacgccatcgacaccatggtggccctgatccgcagcgtgctgcagatgcggaactccaatgccgccacaggcgaggactatatcaacagccccgtgcgcgatctgaatggcgtgtgcttcgactcccggtttcagaacccagagtggcccatggacgccgatgccaatggcgcctaccacatcgccctgaagggccagctgctgctgaatcacctgaaggagagcaaggatctgaagctgcagaacggcatctccaatcaggactggctggcctacatccaggagctgcgcaacagcggcagcgagactcccgggacctcagagtccgccacacccgaaagtcaactggtgaagagcgagctggaagagaagaaaagcgagctcagacataagctgaagtacgttccccacgaatacattgaactgatagaaatcgctagaaacagtacgcaagacagaatactggaaatgaaggtgatggagttcttcatgaaggtttacggctatcgtggcaaacacctcgggggctcccggaagcccgacggggctatctacaccgtgggcagtcccatcgactatggcgtgatcgtggacaccaaagcttatagcggcggatataatctccccatcggccaagccgatgagatgcagaggtatgtggaggagaaccaaacaagaaacaagcatatcaaccccaacgagtggtggaaggtttatcctagctcggtgaccgagtttaagttcctattcgtgtctggccacttcaagggcaactataaggcacagctcactagactgaatcatatcacgaattgcaacggcgccgtgttatccgtggaggagctactgatcggcggagagatgatcaaagccggcaccctgaccctggaagaggtgagaagaaagtttaacaatggcgaaataaatttcggcagcggaagtggaagcggctccatcactagaaccaccaaccctagaaacgtggtgcccaagatctacatgagcgccggcagcatccccctgaccacccacatcaccaactcaattcagcccaccctgtggaccatcggcagcatcaacggcgtggcccccctggccaagagcatcaagctgggcatccccgtgaccggcagcgcctacaccgatcagaccaccgccatggtgagaaagaaggtgagcgtgttcatgggcagcggcagcgggagcggctcatcgcagctggttaagagcgagttagaagaaaaaaagagcgaactgcggcataaactgaagtatgtcccacacgagtacatcgaactgatcgagatcgcgagaaactctacccaagacagaattctggagatgaaagtaatggaatttttcatgaaggtgtatggatatagagggaagcacctgggtggcagcagaaaacccgacggcgccatctacactgtggggagccccatagactatggtgtgatcgtggataccaaggcgtatagcggcggttacaatctgcccattgggcaagcggacgagatgcaaagatatgtggaagagaatcagacgaggaacaagcacattaaccctaatgagtggtggaaggtctaccctagctccgttaccgagttcaagttcctgtttgtgagcgggcattttaagggcaactacaaggcacagctgacccgcctgaaccacataacaaactgcaacggtgccgtgctgagcgtagaagagttgctaatcggcggcgagatgatcaaggccggcacgctaaccctcgaagaggtgcgcagaaagttcaataacggcgaaatcaatttcaaaaggccggcggccacgaaaaaggccggccaggcaaaaaagaaaaagggatcctacccatacgatgttccagattacgcttatccctacgacgtgcctgattatgcatacccatatgatgtccccgactatgccgagggcagaggaagtctgctaacatgcggtgacgtcgaggagaatcctggcccaatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaactagtcctgcaggcatgcaagcttgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagggaattaattcgagctcggtacctttaagaccgatgacttacaaggcagctgtagatcttagccactttttaaaagaaattaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 50 - DNA, 11158 bp, synthetic construct
  source 1..11158: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgagaagacctgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatgcaactggtgaagagcgagctggaagagaagaaaagcgagctcagacataagctgaagtacgttccccacgaatacattgaactgatagaaatcgctagaaacagtacgcaagacagaatactggaaatgaaggtgatggagttcttcatgaaggtttacggctatcgtggcaaacacctcgggggctcccggaagcccgacggggctatctacaccgtgggcagtcccatcgactatggcgtgatcgtggacaccaaagcttatagcggcggatataatctccccatcggccaagccgatgagatgcagaggtatgtggaggagaaccaaacaagaaacaagcatatcaaccccaacgagtggtggaaggtttatcctagctcggtgaccgagtttaagttcctattcgtgtctggccacttcaagggcaactataaggcacagctcactagactgaatcatatcacgaattgcaacggcgccgtgttatccgtggaggagctactgatcggcggagagatgatcaaagccggcaccctgaccctggaagaggtgagaagaaagtttaacaatggcgaaataaatttcggcagcggaagtggaagcggctccatcactagaaccaccaaccctagaaacgtggtgcccaagatctacatgagcgccggcagcatccccctgaccacccacatcaccaactcaattcagcccaccctgtggaccatcggcagcatcaacggcgtggcccccctggccaagagcatcaagctgggcatccccgtgaccggcagcgcctacaccgatcagaccaccgccatggtgagaaagaaggtgagcgtgttcatgggcagcggcagcgggagcggctcatcgcagctggttaagagcgagttagaagaaaaaaagagcgaactgcggcataaactgaagtatgtcccacacgagtacatcgaactgatcgagatcgcgagaaactctacccaagacagaattctggagatgaaagtaatggaatttttcatgaaggtgtatggatatagagggaagcacctgggtggcagcagaaaacccgacggcgccatctacactgtggggagccccatagactatggtgtgatcgtggataccaaggcgtatagcggcggttacaatctgcccattgggcaagcggacgagatgcaaagatatgtggaagagaatcagacgaggaacaagcacattaaccctaatgagtggtggaaggtctaccctagctccgttaccgagttcaagttcctgtttgtgagcgggcattttaagggcaactacaaggcacagctgacccgcctgaaccacataacaaactgcaacggtgccgtgctgagcgtagaagagttgctaatcggcggcgagatgatcaaggccggcacgctaaccctcgaagaggtgcgcagaaagttcaataacggcgaaatcaatttcagcggcagcgagactcccgggacctcagagtccgccacacccgaaagtacacagttcgagggctttaccaacctgtatcaggtgagcaagacactgcggtttgagctgatcccacagggcaagaccctgaagcacatccaggagcagggcttcatcgaggaggacaaggcccgcaatgatcactacaaggagctgaagcccatcatcgatcggatctacaagacctatgccgaccagtgcctgcagctggtgcagctggattgggagaacctgagcgccgccatcgactcctatagaaaggagaaaaccgaggagacaaggaacgccctgatcgaggagcaggccacatatcgcaatgccatccacgactacttcatcggccggacagacaacctgaccgatgccatcaataagagacacgccgagatctacaagggcctgttcaaggccgagctgtttaatggcaaggtgctgaagcagctgggcaccgtgaccacaaccgagcacgagaacgccctgctgcggagcttcgacaagtttacaacctacttctccggcttttatagaaacaggaagaacgtgttcagcgccgaggatatcagcacagccatcccacaccgcatcgtgcaggacaacttccccaagtttaaggagaattgtcacatcttcacacgcctgatcaccgccgtgcccagcctgcgggagcactttgagaacgtgaagaaggccatcggcatcttcgtgagcacctccatcgaggaggtgttttccttccctttttataaccagctgctgacacagacccagatcgacctgtataaccagctgctgggaggaatctctcgggaggcaggcaccgagaagatcaagggcctgaacgaggtgctgaatctggccatccagaagaatgatgagacagcccacatcatcgcctccctgccacacagattcatccccctgtttaagcagatcctgtccgataggaacaccctgtctttcatcctggaggagtttaagagcgacgaggaagtgatccagtccttctgcaagtacaagacactgctgagaaacgagaacgtgctggagacagccgaggccctgtttaacgagctgaacagcatcgacctgacacacatcttcatcagccacaagaagctggagacaatcagcagcgccctgtgcgaccactgggatacactgaggaatgccctgtatgagcggagaatctccgagctgacaggcaagatcaccaagtctgccaaggagaaggtgcagcgcagcctgaagcacgaggatatcaacctgcaggagatcatctctgccgcaggcaaggagctgagcgaggccttcaagcagaaaaccagcgagatcctgtcccacgcacacgccgccctggatcagccactgcctacaaccctgaagaagcaggaggagaaggagatcctgaagtctcagctggacagcctgctgggcctgtaccacctgctggactggtttgccgtggatgagtccaacgaggtggaccccgagttctctgcccggctgaccggcatcaagctggagatggagccttctctgagcttctacaacaaggccagaaattatgccaccaagaagccctactccgtggagaagttcaagctgaactttcagatgcctacactggccagaggctgggacgtgaatgtggagaagaacagaggcgccatcctgtttgtgaagaacggcctgtactatctgggcatcatgccaaagcagaagggcaggtataaggccctgagcttcgagcccacagagaaaaccagcgagggctttgataagatgtactatgactacttccctgatgccgccaagatgatcccaaagtgcagcacccagctgaaggccgtgacagcccactttcagacccacacaacccccatcctgctgtccaacaatttcatcgagcctctggagatcacaaaggagatctacgacctgaacaatcctgagaaggagccaaagaagtttcagacagcctacgccaagaaaaccggcgaccagaagggctacagagaggccctgtgcaagtggatcgacttcacaagggattttctgtccaagtataccaagacaacctctatcgatctgtctagcctgcggccatcctctcagtataaggacctgggcgagtactatgccgagctgaatcccctgctgtaccacatcagcttccagagaatcgccgagaaggagatcatggatgccgtggagacaggcaagctgtacctgttccagatctataacaaggactttgccaagggccaccacggcaagcctaatctgcacacactgtattggaccggcctgttttctccagagaacctggccaagacaagcatcaagctgaatggccaggccgagctgttctaccgccctaagtccaggatgaagaggatggcacaccggctgggagagaagatgctgaacaagaagctgaaggatcagaaaaccccaatccccgacaccctgtaccaggagctgtacgactatgtgaatcacagactgtcccacgacctgtctgatgaggccagggccctgctgcccaacgtgatcaccaaggaggtgtctcacgagatcatcaaggataggcgctttaccagcgacaagttctttttccacgtgcctatcacactgaactatcaggccgccaattccccatctaagttcaaccagagggtgaatgcctacctgaaggagcaccccgagacacctatcatcggcatcgcccggggcgagagaaacctgatctatatcacagtgatcgactccaccggcaagatcctggagcagcggagcctgaacaccatccagcagtttgattaccagaagaagctggacaacagggagaaggagagggtggcagcaaggcaggcctggtctgtggtgggcacaatcaaggatctgaagcagggctatctgagccaggtcatccacgagatcgtggacctgatgatccactaccaggccgtggtggtgctggagaacctgaatttcggctttaagagcaagaggaccggcatcgccgagaaggccgtgtaccagcagttcgagaagatgctgatcgataagctgaattgcctggtgctgaaggactatccagcagagaaagtgggaggcgtgctgaacccataccagctgacagaccagttcacctcctttgccaagatgggcacccagtctggcttcctgttttacgtgcctgccccatatacatctaagatcgatcccctgaccggcttcgtggaccccttcgtgtggaaaaccatcaagaatcacgagagccgcaagcacttcctggagggcttcgactttctgcactacgacgtgaaaaccggcgacttcatcctgcactttaagatgaacagaaatctgtccttccagaggggcctgcccggctttatgcctgcatgggatatcgtgttcgagaagaacgagacacagtttgacgccaagggcacccctttcatcgccggcaagagaatcgtgccagtgatcgagaatcacagattcaccggcagataccgggacctgtatcctgccaacgagctgatcgccctgctggaggagaagggcatcgtgttcagggatggctccaacatcctgccaaagctgctggagaatgacgattctcacgccatcgacaccatggtggccctgatccgcagcgtgctgcagatgcggaactccaatgccgccacaggcgaggactatatcaacagccccgtgcgcgatctgaatggcgtgtgcttcgactcccggtttcagaacccagagtggcccatggacgccgatgccaatggcgcctaccacatcgccctgaagggccagctgctgctgaatcacctgaaggagagcaaggatctgaagctgcagaacggcatctccaatcaggactggctggcctacatccaggagctgcgcaacaaaaggccggcggccacgaaaaaggccggccaggcaaaaaagaaaaagggatcctacccatacgatgttccagattacgcttatccctacgacgtgcctgattatgcatacccatatgatgtccccgactatgccgagggcagaggaagtctgctaacatgcggtgacgtcgaggagaatcctggcccaatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaactagtcctgcaggcatgcaagcttgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagggaattaattcgagctcggtacctttaagaccgatgacttacaaggcagctgtagatcttagccactttttaaaagaaattaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 51 - DNA, 11158 bp, synthetic construct
  source 1..11158: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgagaagacctgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatgacacagttcgagggctttaccaacctgtatcaggtgagcaagacactgcggtttgagctgatcccacagggcaagaccctgaagcacatccaggagcagggcttcatcgaggaggacaaggcccgcaatgatcactacaaggagctgaagcccatcatcgatcggatctacaagacctatgccgaccagtgcctgcagctggtgcagctggattgggagaacctgagcgccgccatcgactcctatagaaaggagaaaaccgaggagacaaggaacgccctgatcgaggagcaggccacatatcgcaatgccatccacgactacttcatcggccggacagacaacctgaccgatgccatcaataagagacacgccgagatctacaagggcctgttcaaggccgagctgtttaatggcaaggtgctgaagcagctgggcaccgtgaccacaaccgagcacgagaacgccctgctgcggagcttcgacaagtttacaacctacttctccggcttttatagaaacaggaagaacgtgttcagcgccgaggatatcagcacagccatcccacaccgcatcgtgcaggacaacttccccaagtttaaggagaattgtcacatcttcacacgcctgatcaccgccgtgcccagcctgcgggagcactttgagaacgtgaagaaggccatcggcatcttcgtgagcacctccatcgaggaggtgttttccttccctttttataaccagctgctgacacagacccagatcgacctgtataaccagctgctgggaggaatctctcgggaggcaggcaccgagaagatcaagggcctgaacgaggtgctgaatctggccatccagaagaatgatgagacagcccacatcatcgcctccctgccacacagattcatccccctgtttaagcagatcctgtccgataggaacaccctgtctttcatcctggaggagtttaagagcgacgaggaagtgatccagtccttctgcaagtacaagacactgctgagaaacgagaacgtgctggagacagccgaggccctgtttaacgagctgaacagcatcgacctgacacacatcttcatcagccacaagaagctggagacaatcagcagcgccctgtgcgaccactgggatacactgaggaatgccctgtatgagcggagaatctccgagctgacaggcaagatcaccaagtctgccaaggagaaggtgcagcgcagcctgaagcacgaggatatcaacctgcaggagatcatctctgccgcaggcaaggagctgagcgaggccttcaagcagaaaaccagcgagatcctgtcccacgcacacgccgccctggatcagccactgcctacaaccctgaagaagcaggaggagaaggagatcctgaagtctcagctggacagcctgctgggcctgtaccacctgctggactggtttgccgtggatgagtccaacgaggtggaccccgagttctctgcccggctgaccggcatcaagctggagatggagccttctctgagcttctacaacaaggccagaaattatgccaccaagaagccctactccgtggagaagttcaagctgaactttcagatgcctacactggccagaggctgggacgtgaatgtggagaagaacagaggcgccatcctgtttgtgaagaacggcctgtactatctgggcatcatgccaaagcagaagggcaggtataaggccctgagcttcgagcccacagagaaaaccagcgagggctttgataagatgtactatgactacttccctgatgccgccaagatgatcccaaagtgcagcacccagctgaaggccgtgacagcccactttcagacccacacaacccccatcctgctgtccaacaatttcatcgagcctctggagatcacaaaggagatctacgacctgaacaatcctgagaaggagccaaagaagtttcagacagcctacgccaagaaaaccggcgaccagaagggctacagagaggccctgtgcaagtggatcgacttcacaagggattttctgtccaagtataccaagacaacctctatcgatctgtctagcctgcggccatcctctcagtataaggacctgggcgagtactatgccgagctgaatcccctgctgtaccacatcagcttccagagaatcgccgagaaggagatcatggatgccgtggagacaggcaagctgtacctgttccagatctataacaaggactttgccaagggccaccacggcaagcctaatctgcacacactgtattggaccggcctgttttctccagagaacctggccaagacaagcatcaagctgaatggccaggccgagctgttctaccgccctaagtccaggatgaagaggatggcacaccggctgggagagaagatgctgaacaagaagctgaaggatcagaaaaccccaatccccgacaccctgtaccaggagctgtacgactatgtgaatcacagactgtcccacgacctgtctgatgaggccagggccctgctgcccaacgtgatcaccaaggaggtgtctcacgagatcatcaaggataggcgctttaccagcgacaagttctttttccacgtgcctatcacactgaactatcaggccgccaattccccatctaagttcaaccagagggtgaatgcctacctgaaggagcaccccgagacacctatcatcggcatcgcccggggcgagagaaacctgatctatatcacagtgatcgactccaccggcaagatcctggagcagcggagcctgaacaccatccagcagtttgattaccagaagaagctggacaacagggagaaggagagggtggcagcaaggcaggcctggtctgtggtgggcacaatcaaggatctgaagcagggctatctgagccaggtcatccacgagatcgtggacctgatgatccactaccaggccgtggtggtgctggagaacctgaatttcggctttaagagcaagaggaccggcatcgccgagaaggccgtgtaccagcagttcgagaagatgctgatcgataagctgaattgcctggtgctgaaggactatccagcagagaaagtgggaggcgtgctgaacccataccagctgacagaccagttcacctcctttgccaagatgggcacccagtctggcttcctgttttacgtgcctgccccatatacatctaagatcgatcccctgaccggcttcgtggaccccttcgtgtggaaaaccatcaagaatcacgagagccgcaagcacttcctggagggcttcgactttctgcactacgacgtgaaaaccggcgacttcatcctgcactttaagatgaacagaaatctgtccttccagaggggcctgcccggctttatgcctgcatgggatatcgtgttcgagaagaacgagacacagtttgacgccaagggcacccctttcatcgccggcaagagaatcgtgccagtgatcgagaatcacagattcaccggcagataccgggacctgtatcctgccaacgagctgatcgccctgctggaggagaagggcatcgtgttcagggatggctccaacatcctgccaaagctgctggagaatgacgattctcacgccatcgacaccatggtggccctgatccgcagcgtgctgcagatgcggaactccaatgccgccacaggcgaggactatatcaacagccccgtgcgcgatctgaatggcgtgtgcttcgactcccggtttcagaacccagagtggcccatggacgccgatgccaatggcgcctaccacatcgccctgaagggccagctgctgctgaatcacctgaaggagagcaaggatctgaagctgcagaacggcatctccaatcaggactggctggcctacatccaggagctgcgcaacagcggcagcgagactcccgggacctcagagtccgccacacccgaaagtcaactggtgaagagcgagctggaagagaagaaaagcgagctcagacataagctgaagtacgttccccacgaatacattgaactgatagaaatcgctagaaacagtacgcaagacagaatactggaaatgaaggtgatggagttcttcatgaaggtttacggctatcgtggcaaacacctcgggggctcccggaagcccgccggggctatctacaccgtgggcagtcccatcgactatggcgtgatcgtggacaccaaagcttatagcggcggatataatctccccatcggccaagccgatgagatgcagaggtatgtggaggagaaccaaacaagaaacaagcatatcaaccccaacgagtggtggaaggtttatcctagctcggtgaccgagtttaagttcctattcgtgtctggccacttcaagggcaactataaggcacagctcactagactgaatcatatcacgaattgcaacggcgccgtgttatccgtggaggagctactgatcggcggagagatgatcaaagccggcaccctgaccctggaagaggtgagaagaaagtttaacaatggcgaaataaatttcggcagcggaagtggaagcggctccatcactagaaccaccaaccctagaaacgtggtgcccaagatctacatgagcgccggcagcatccccctgaccacccacatcaccaactcaattcagcccaccctgtggaccatcggcagcatcaacggcgtggcccccctggccaagagcatcaagctgggcatccccgtgaccggcagcgcctacaccgatcagaccaccgccatggtgagaaagaaggtgagcgtgttcatgggcagcggcagcgggagcggctcatcgcagctggttaagagcgagttagaagaaaaaaagagcgaactgcggcataaactgaagtatgtcccacacgagtacatcgaactgatcgagatcgcgagaaactctacccaagacagaattctggagatgaaagtaatggaatttttcatgaaggtgtatggatatagagggaagcacctgggtggcagcagaaaacccgacggcgccatctacactgtggggagccccatagactatggtgtgatcgtggataccaaggcgtatagcggcggttacaatctgcccattgggcaagcggacgagatgcaaagatatgtggaagagaatcagacgaggaacaagcacattaaccctaatgagtggtggaaggtctaccctagctccgttaccgagttcaagttcctgtttgtgagcgggcattttaagggcaactacaaggcacagctgacccgcctgaaccacataacaaactgcaacggtgccgtgctgagcgtagaagagttgctaatcggcggcgagatgatcaaggccggcacgctaaccctcgaagaggtgcgcagaaagttcaataacggcgaaatcaatttcaaaaggccggcggccacgaaaaaggccggccaggcaaaaaagaaaaagggatcctacccatacgatgttccagattacgcttatccctacgacgtgcctgattatgcatacccatatgatgtccccgactatgccgagggcagaggaagtctgctaacatgcggtgacgtcgaggagaatcctggcccaatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaactagtcctgcaggcatgcaagcttgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagggaattaattcgagctcggtacctttaagaccgatgacttacaaggcagctgtagatcttagccactttttaaaagaaattaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 52 - DNA, 11158 bp, synthetic construct
  source 1..11158: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgagaagacctgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatgacacagttcgagggctttaccaacctgtatcaggtgagcaagacactgcggtttgagctgatcccacagggcaagaccctgaagcacatccaggagcagggcttcatcgaggaggacaaggcccgcaatgatcactacaaggagctgaagcccatcatcgatcggatctacaagacctatgccgaccagtgcctgcagctggtgcagctggattgggagaacctgagcgccgccatcgactcctatagaaaggagaaaaccgaggagacaaggaacgccctgatcgaggagcaggccacatatcgcaatgccatccacgactacttcatcggccggacagacaacctgaccgatgccatcaataagagacacgccgagatctacaagggcctgttcaaggccgagctgtttaatggcaaggtgctgaagcagctgggcaccgtgaccacaaccgagcacgagaacgccctgctgcggagcttcgacaagtttacaacctacttctccggcttttatagaaacaggaagaacgtgttcagcgccgaggatatcagcacagccatcccacaccgcatcgtgcaggacaacttccccaagtttaaggagaattgtcacatcttcacacgcctgatcaccgccgtgcccagcctgcgggagcactttgagaacgtgaagaaggccatcggcatcttcgtgagcacctccatcgaggaggtgttttccttccctttttataaccagctgctgacacagacccagatcgacctgtataaccagctgctgggaggaatctctcgggaggcaggcaccgagaagatcaagggcctgaacgaggtgctgaatctggccatccagaagaatgatgagacagcccacatcatcgcctccctgccacacagattcatccccctgtttaagcagatcctgtccgataggaacaccctgtctttcatcctggaggagtttaagagcgacgaggaagtgatccagtccttctgcaagtacaagacactgctgagaaacgagaacgtgctggagacagccgaggccctgtttaacgagctgaacagcatcgacctgacacacatcttcatcagccacaagaagctggagacaatcagcagcgccctgtgcgaccactgggatacactgaggaatgccctgtatgagcggagaatctccgagctgacaggcaagatcaccaagtctgccaaggagaaggtgcagcgcagcctgaagcacgaggatatcaacctgcaggagatcatctctgccgcaggcaaggagctgagcgaggccttcaagcagaaaaccagcgagatcctgtcccacgcacacgccgccctggatcagccactgcctacaaccctgaagaagcaggaggagaaggagatcctgaagtctcagctggacagcctgctgggcctgtaccacctgctggactggtttgccgtggatgagtccaacgaggtggaccccgagttctctgcccggctgaccggcatcaagctggagatggagccttctctgagcttctacaacaaggccagaaattatgccaccaagaagccctactccgtggagaagttcaagctgaactttcagatgcctacactggccagaggctgggacgtgaatgtggagaagaacagaggcgccatcctgtttgtgaagaacggcctgtactatctgggcatcatgccaaagcagaagggcaggtataaggccctgagcttcgagcccacagagaaaaccagcgagggctttgataagatgtactatgactacttccctgatgccgccaagatgatcccaaagtgcagcacccagctgaaggccgtgacagcccactttcagacccacacaacccccatcctgctgtccaacaatttcatcgagcctctggagatcacaaaggagatctacgacctgaacaatcctgagaaggagccaaagaagtttcagacagcctacgccaagaaaaccggcgaccagaagggctacagagaggccctgtgcaagtggatcgacttcacaagggattttctgtccaagtataccaagacaacctctatcgatctgtctagcctgcggccatcctctcagtataaggacctgggcgagtactatgccgagctgaatcccctgctgtaccacatcagcttccagagaatcgccgagaaggagatcatggatgccgtggagacaggcaagctgtacctgttccagatctataacaaggactttgccaagggccaccacggcaagcctaatctgcacacactgtattggaccggcctgttttctccagagaacctggccaagacaagcatcaagctgaatggccaggccgagctgttctaccgccctaagtccaggatgaagaggatggcacaccggctgggagagaagatgctgaacaagaagctgaaggatcagaaaaccccaatccccgacaccctgtaccaggagctgtacgactatgtgaatcacagactgtcccacgacctgtctgatgaggccagggccctgctgcccaacgtgatcaccaaggaggtgtctcacgagatcatcaaggataggcgctttaccagcgacaagttctttttccacgtgcctatcacactgaactatcaggccgccaattccccatctaagttcaaccagagggtgaatgcctacctgaaggagcaccccgagacacctatcatcggcatcgcccggggcgagagaaacctgatctatatcacagtgatcgactccaccggcaagatcctggagcagcggagcctgaacaccatccagcagtttgattaccagaagaagctggacaacagggagaaggagagggtggcagcaaggcaggcctggtctgtggtgggcacaatcaaggatctgaagcagggctatctgagccaggtcatccacgagatcgtggacctgatgatccactaccaggccgtggtggtgctggagaacctgaatttcggctttaagagcaagaggaccggcatcgccgagaaggccgtgtaccagcagttcgagaagatgctgatcgataagctgaattgcctggtgctgaaggactatccagcagagaaagtgggaggcgtgctgaacccataccagctgacagaccagttcacctcctttgccaagatgggcacccagtctggcttcctgttttacgtgcctgccccatatacatctaagatcgatcccctgaccggcttcgtggaccccttcgtgtggaaaaccatcaagaatcacgagagccgcaagcacttcctggagggcttcgactttctgcactacgacgtgaaaaccggcgacttcatcctgcactttaagatgaacagaaatctgtccttccagaggggcctgcccggctttatgcctgcatgggatatcgtgttcgagaagaacgagacacagtttgacgccaagggcacccctttcatcgccggcaagagaatcgtgccagtgatcgagaatcacagattcaccggcagataccgggacctgtatcctgccaacgagctgatcgccctgctggaggagaagggcatcgtgttcagggatggctccaacatcctgccaaagctgctggagaatgacgattctcacgccatcgacaccatggtggccctgatccgcagcgtgctgcagatgcggaactccaatgccgccacaggcgaggactatatcaacagccccgtgcgcgatctgaatggcgtgtgcttcgactcccggtttcagaacccagagtggcccatggacgccgatgccaatggcgcctaccacatcgccctgaagggccagctgctgctgaatcacctgaaggagagcaaggatctgaagctgcagaacggcatctccaatcaggactggctggcctacatccaggagctgcgcaacagcggcagcgagactcccgggacctcagagtccgccacacccgaaagtcaactggtgaagagcgagctggaagagaagaaaagcgagctcagacataagctgaagtacgttccccacgaatacattgaactgatagaaatcgctagaaacagtacgcaagacagaatactggaaatgaaggtgatggagttcttcatgaaggtttacggctatcgtggcaaacacctcgggggctcccggaagcccgacggggctatctacaccgtgggcagtcccatcgactatggcgtgatcgtggacaccaaagcttatagcggcggatataatctccccatcggccaagccgatgagatgcagaggtatgtggaggagaaccaaacaagaaacaagcatatcaaccccaacgagtggtggaaggtttatcctagctcggtgaccgagtttaagttcctattcgtgtctggccacttcaagggcaactataaggcacagctcactagactgaatcatatcacgaattgcaacggcgccgtgttatccgtggaggagctactgatcggcggagagatgatcaaagccggcaccctgaccctggaagaggtgagaagaaagtttaacaatggcgaaataaatttcggcagcggaagtggaagcggctccatcactagaaccaccaaccctagaaacgtggtgcccaagatctacatgagcgccggcagcatccccctgaccacccacatcaccaactcaattcagcccaccctgtggaccatcggcagcatcaacggcgtggcccccctggccaagagcatcaagctgggcatccccgtgaccggcagcgcctacaccgatcagaccaccgccatggtgagaaagaaggtgagcgtgttcatgggcagcggcagcgggagcggctcatcgcagctggttaagagcgagttagaagaaaaaaagagcgaactgcggcataaactgaagtatgtcccacacgagtacatcgaactgatcgagatcgcgagaaactctacccaagacagaattctggagatgaaagtaatggaatttttcatgaaggtgtatggatatagagggaagcacctgggtggcagcagaaaacccgccggcgccatctacactgtggggagccccatagactatggtgtgatcgtggataccaaggcgtatagcggcggttacaatctgcccattgggcaagcggacgagatgcaaagatatgtggaagagaatcagacgaggaacaagcacattaaccctaatgagtggtggaaggtctaccctagctccgttaccgagttcaagttcctgtttgtgagcgggcattttaagggcaactacaaggcacagctgacccgcctgaaccacataacaaactgcaacggtgccgtgctgagcgtagaagagttgctaatcggcggcgagatgatcaaggccggcacgctaaccctcgaagaggtgcgcagaaagttcaataacggcgaaatcaatttcaaaaggccggcggccacgaaaaaggccggccaggcaaaaaagaaaaagggatcctacccatacgatgttccagattacgcttatccctacgacgtgcctgattatgcatacccatatgatgtccccgactatgccgagggcagaggaagtctgctaacatgcggtgacgtcgaggagaatcctggcccaatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaactagtcctgcaggcatgcaagcttgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagggaattaattcgagctcggtacctttaagaccgatgacttacaaggcagctgtagatcttagccactttttaaaagaaattaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 53 - DNA, 11158 bp, synthetic construct
  source 1..11158: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgagaagacctgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatgcaactggtgaagagcgagctggaagagaagaaaagcgagctcagacataagctgaagtacgttccccacgaatacattgaactgatagaaatcgctagaaacagtacgcaagacagaatactggaaatgaaggtgatggagttcttcatgaaggtttacggctatcgtggcaaacacctcgggggctcccggaagcccgccggggctatctacaccgtgggcagtcccatcgactatggcgtgatcgtggacaccaaagcttatagcggcggatataatctccccatcggccaagccgatgagatgcagaggtatgtggaggagaaccaaacaagaaacaagcatatcaaccccaacgagtggtggaaggtttatcctagctcggtgaccgagtttaagttcctattcgtgtctggccacttcaagggcaactataaggcacagctcactagactgaatcatatcacgaattgcaacggcgccgtgttatccgtggaggagctactgatcggcggagagatgatcaaagccggcaccctgaccctggaagaggtgagaagaaagtttaacaatggcgaaataaatttcggcagcggaagtggaagcggctccatcactagaaccaccaaccctagaaacgtggtgcccaagatctacatgagcgccggcagcatccccctgaccacccacatcaccaactcaattcagcccaccctgtggaccatcggcagcatcaacggcgtggcccccctggccaagagcatcaagctgggcatccccgtgaccggcagcgcctacaccgatcagaccaccgccatggtgagaaagaaggtgagcgtgttcatgggcagcggcagcgggagcggctcatcgcagctggttaagagcgagttagaagaaaaaaagagcgaactgcggcataaactgaagtatgtcccacacgagtacatcgaactgatcgagatcgcgagaaactctacccaagacagaattctggagatgaaagtaatggaatttttcatgaaggtgtatggatatagagggaagcacctgggtggcagcagaaaacccgacggcgccatctacactgtggggagccccatagactatggtgtgatcgtggataccaaggcgtatagcggcggttacaatctgcccattgggcaagcggacgagatgcaaagatatgtggaagagaatcagacgaggaacaagcacattaaccctaatgagtggtggaaggtctaccctagctccgttaccgagttcaagttcctgtttgtgagcgggcattttaagggcaactacaaggcacagctgacccgcctgaaccacataacaaactgcaacggtgccgtgctgagcgtagaagagttgctaatcggcggcgagatgatcaaggccggcacgctaaccctcgaagaggtgcgcagaaagttcaataacggcgaaatcaatttcagcggcagcgagactcccgggacctcagagtccgccacacccgaaagtacacagttcgagggctttaccaacctgtatcaggtgagcaagacactgcggtttgagctgatcccacagggcaagaccctgaagcacatccaggagcagggcttcatcgaggaggacaaggcccgcaatgatcactacaaggagctgaagcccatcatcgatcggatctacaagacctatgccgaccagtgcctgcagctggtgcagctggattgggagaacctgagcgccgccatcgactcctatagaaaggagaaaaccgaggagacaaggaacgccctgatcgaggagcaggccacatatcgcaatgccatccacgactacttcatcggccggacagacaacctgaccgatgccatcaataagagacacgccgagatctacaagggcctgttcaaggccgagctgtttaatggcaaggtgctgaagcagctgggcaccgtgaccacaaccgagcacgagaacgccctgctgcggagcttcgacaagtttacaacctacttctccggcttttatagaaacaggaagaacgtgttcagcgccgaggatatcagcacagccatcccacaccgcatcgtgcaggacaacttccccaagtttaaggagaattgtcacatcttcacacgcctgatcaccgccgtgcccagcctgcgggagcactttgagaacgtgaagaaggccatcggcatcttcgtgagcacctccatcgaggaggtgttttccttccctttttataaccagctgctgacacagacccagatcgacctgtataaccagctgctgggaggaatctctcgggaggcaggcaccgagaagatcaagggcctgaacgaggtgctgaatctggccatccagaagaatgatgagacagcccacatcatcgcctccctgccacacagattcatccccctgtttaagcagatcctgtccgataggaacaccctgtctttcatcctggaggagtttaagagcgacgaggaagtgatccagtccttctgcaagtacaagacactgctgagaaacgagaacgtgctggagacagccgaggccctgtttaacgagctgaacagcatcgacctgacacacatcttcatcagccacaagaagctggagacaatcagcagcgccctgtgcgaccactgggatacactgaggaatgccctgtatgagcggagaatctccgagctgacaggcaagatcaccaagtctgccaaggagaaggtgcagcgcagcctgaagcacgaggatatcaacctgcaggagatcatctctgccgcaggcaaggagctgagcgaggccttcaagcagaaaaccagcgagatcctgtcccacgcacacgccgccctggatcagccactgcctacaaccctgaagaagcaggaggagaaggagatcctgaagtctcagctggacagcctgctgggcctgtaccacctgctggactggtttgccgtggatgagtccaacgaggtggaccccgagttctctgcccggctgaccggcatcaagctggagatggagccttctctgagcttctacaacaaggccagaaattatgccaccaagaagccctactccgtggagaagttcaagctgaactttcagatgcctacactggccagaggctgggacgtgaatgtggagaagaacagaggcgccatcctgtttgtgaagaacggcctgtactatctgggcatcatgccaaagcagaagggcaggtataaggccctgagcttcgagcccacagagaaaaccagcgagggctttgataagatgtactatgactacttccctgatgccgccaagatgatcccaaagtgcagcacccagctgaaggccgtgacagcccactttcagacccacacaacccccatcctgctgtccaacaatttcatcgagcctctggagatcacaaaggagatctacgacctgaacaatcctgagaaggagccaaagaagtttcagacagcctacgccaagaaaaccggcgaccagaagggctacagagaggccctgtgcaagtggatcgacttcacaagggattttctgtccaagtataccaagacaacctctatcgatctgtctagcctgcggccatcctctcagtataaggacctgggcgagtactatgccgagctgaatcccctgctgtaccacatcagcttccagagaatcgccgagaaggagatcatggatgccgtggagacaggcaagctgtacctgttccagatctataacaaggactttgccaagggccaccacggcaagcctaatctgcacacactgtattggaccggcctgttttctccagagaacctggccaagacaagcatcaagctgaatggccaggccgagctgttctaccgccctaagtccaggatgaagaggatggcacaccggctgggagagaagatgctgaacaagaagctgaaggatcagaaaaccccaatccccgacaccctgtaccaggagctgtacgactatgtgaatcacagactgtcccacgacctgtctgatgaggccagggccctgctgcccaacgtgatcaccaaggaggtgtctcacgagatcatcaaggataggcgctttaccagcgacaagttctttttccacgtgcctatcacactgaactatcaggccgccaattccccatctaagttcaaccagagggtgaatgcctacctgaaggagcaccccgagacacctatcatcggcatcgcccggggcgagagaaacctgatctatatcacagtgatcgactccaccggcaagatcctggagcagcggagcctgaacaccatccagcagtttgattaccagaagaagctggacaacagggagaaggagagggtggcagcaaggcaggcctggtctgtggtgggcacaatcaaggatctgaagcagggctatctgagccaggtcatccacgagatcgtggacctgatgatccactaccaggccgtggtggtgctggagaacctgaatttcggctttaagagcaagaggaccggcatcgccgagaaggccgtgtaccagcagttcgagaagatgctgatcgataagctgaattgcctggtgctgaaggactatccagcagagaaagtgggaggcgtgctgaacccataccagctgacagaccagttcacctcctttgccaagatgggcacccagtctggcttcctgttttacgtgcctgccccatatacatctaagatcgatcccctgaccggcttcgtggaccccttcgtgtggaaaaccatcaagaatcacgagagccgcaagcacttcctggagggcttcgactttctgcactacgacgtgaaaaccggcgacttcatcctgcactttaagatgaacagaaatctgtccttccagaggggcctgcccggctttatgcctgcatgggatatcgtgttcgagaagaacgagacacagtttgacgccaagggcacccctttcatcgccggcaagagaatcgtgccagtgatcgagaatcacagattcaccggcagataccgggacctgtatcctgccaacgagctgatcgccctgctggaggagaagggcatcgtgttcagggatggctccaacatcctgccaaagctgctggagaatgacgattctcacgccatcgacaccatggtggccctgatccgcagcgtgctgcagatgcggaactccaatgccgccacaggcgaggactatatcaacagccccgtgcgcgatctgaatggcgtgtgcttcgactcccggtttcagaacccagagtggcccatggacgccgatgccaatggcgcctaccacatcgccctgaagggccagctgctgctgaatcacctgaaggagagcaaggatctgaagctgcagaacggcatctccaatcaggactggctggcctacatccaggagctgcgcaacaaaaggccggcggccacgaaaaaggccggccaggcaaaaaagaaaaagggatcctacccatacgatgttccagattacgcttatccctacgacgtgcctgattatgcatacccatatgatgtccccgactatgccgagggcagaggaagtctgctaacatgcggtgacgtcgaggagaatcctggcccaatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaactagtcctgcaggcatgcaagcttgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagggaattaattcgagctcggtacctttaagaccgatgacttacaaggcagctgtagatcttagccactttttaaaagaaattaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 54 - protein, 8 aa, synthetic construct
GGGGGGGG

SEQ ID NO: 55 - protein, 4 aa, synthetic construct
  source 1..4: note = May be repeated
GGGS

SEQ ID NO: 56 - protein, 20 aa, synthetic construct
GGGGSGGGGSGGGGSGGGGS

SEQ ID NO: 57 - protein, 65 aa, synthetic construct
  VARIANT 2..10: note = May be absent
  VARIANT 12..65: note = May be absent
GGGGGGGGGGSSSGGGGGGGGGGSSSGGGGGGGGGGSSSGGGGGGGGGGSSSGGGGGGGGGGSSS